(19) United States
(12) Patent Application Publication
(10) Pub. No.: US 2024/0145038 A1
(43) Pub. Date: May 2, 2024

(54) cfDNA FRAGMENTOMIC DETECTION OF CANCER

(71) Applicant: Wisconsin Alumni Research Foundation, Madison, WI (US)

(72) Inventors: Shuang Zhao, Verona, WI (US); Kyle Helzer, Madison, WI (US)

(73) Assignee: Wisconsin Alumni Research Foundation, Madison, WI (US)

(21) Appl. No.: 18/498,382

(22) Filed: Oct. 31, 2023

Related U.S. Application Data
(60) Provisional application No. 63/520,761, filed on Aug. 21, 2023.
(60) Provisional application No. 63/421,803, filed on Nov. 2, 2022.

(51) Int. Cl. G16B 30/00; G16B 40/20

(52) U.S. Cl. CPC G16B 30/00; G16B 40/20

(57) ABSTRACT

Methods of detecting cancer or a particular type or subtype thereof in a subject and treating the cancer or particular type or subtype thereof. The detection can comprise determining fragmentation patterns of classifier cell-free deoxyribonucleic acid (cfDNA) from the subject and classifying the fragmentation patterns to identify the subject as being negative or positive for the cancer or the particular type or subtype thereof. The classifier cfDNA can comprise cfDNA corresponding to at least a portion of at least one exon of one or more classifier genes. The exon can comprise the first exons of the classifier genes. The treating can comprise the specific type of subtype of cancer that is detected.

### FIELD OF THE INVENTION

The invention is directed to the detection of cancer, including specific types and/or subtypes of cancer, in a subject using cell-free deoxyribonucleic acid (cfDNA) fragmentomic methods and, optionally, additional testing and treatments of the detected cancer.

### BACKGROUND

Profiling of genomic driver alterations in cancer has become increasingly important, not only for studying the biological underpinnings of cancer, but also in identifying clinically actionable alterations for targeted therapies in clinical trials and practice. Historically, tumor samples have been required, but obtaining tissue specimens for molecular profiling is not always feasible, and can be especially challenging in the metastatic setting. Cell-free DNA (cfDNA) from cancer patients provides a non-invasive approach for assessing circulating tumor DNA (ctDNA) for alterations (Diaz L A Jr, Bardelli A. Liquid biopsies: genotyping circulating tumor DNA. J Clin Oncol. 2014 Feb. 20;32(6):579-86). This is a mature technology, with multiple commercially available next-generation sequencing (NGS) ctDNA panels. These mainly profile the coding regions of selected oncogenes and tumor suppressors for DNA alterations in order to assist with clinical decision making.

In order to remain stable in circulation, cfDNA usually must be bound to a protein. Most often, this is the nucleosome complex, which is reflected in size distribution of cfDNA fragments showing the largest peak at 167 bp (Lo Y M, Chan K C, Sun H, Chen E Z, Jiang P, Lun F M, Zheng Y W, Leung T Y, Lau T K, Cantor C R, Chiu R W. Maternal plasma DNA sequencing reveals the genome-wide genetic and mutational profile of the fetus. Sci Transl Med. 2010 Dec. 8;2(61):61ra91) (Snyder M W, Kircher M, Hill A J, Daza R M, Shendure J. Cell-free DNA Comprises an In Vivo Nucleosome Footprint that Informs Its Tissues-Of-Origin. Cell. 2016 Jan. 14;164(1-2):57-68) corresponding to one nucleosome, a smaller peak at 334 bp corresponding to two nucleosomes, and so on. Other studies have also described smaller peaks representing transcription factor binding (Ulz P, Perakis S, Zhou Q, Moser T, Belic J, Lazzeri I, Wölfler A, Zebisch A, Gerger A, Pristauz G, Petru E, White B, Roberts C E S, John JS, Schimek M G, Geigl J B, Bauernhofer T, Sill H, Bock C, Heitzer E, Speicher MR. Inference of transcription factor binding from cell-free DNA enables tumor subtype prediction and early detection. Nat Commun. 2019 Oct. 11;10(1):4666). Distinct fragmentation patterns around the transcription start site have been shown to reflect binding of the transcriptional machinery, and correlate with gene expression (Esfahani M S, Hamilton E G, Mehrmohamadi M, Nabet B Y, Alig S K, King D A, Steen C B, Macaulay C W, Schultz A, Nesselbush M C, Soo J, Schroers-Martin J G, Chen B, Binkley M S, Stehr H, Chabon J J, Sworder B J, Hui A B, Frank M J, Moding E J, Liu C L, Newman A M, Isbell J M, Rudin C M, Li B T, Kurtz D M, Diehn M, Alizadeh A A. Inferring gene expression from cell-free DNA fragmentation profiles. Nat Biotechnol. 2022 April;40(4):585-597). The study of cfDNA fragmentation patterns has been referred to as “fragmentomics” and cancer patients show distinct fragmentomic patterns that have been used to non-invasively inform the biology of the tumor.

Because cfDNA fragmentation patterns are a genome-wide phenomenon, almost all clinical fragmentomic studies to date have used whole-genome sequencing (WGS) for fragmentomic analysis. The breadth advantage of cfDNA WGS is traded off against low depth of sequencing compared to cfDNA targeted panels. WGS is generally unsuitable for cfDNA somatic alteration detection as it has poor sensitivity, especially at low ctDNA fractions. However, the field has focused on WGS as traditional coding targeted cfDNA panels would not capture the majority of known fragmentomic regions of interest which predominantly occur in non-coding regions of the genome.

Targeted cfDNA fragmentomic methods that do not require WGS and thereby permit high sequencing depth and/or higher sensitivity and specificity for the detection of diseases such as cancer are needed.

### SUMMARY OF THE INVENTION

Some aspects of the invention are directed to methods of detecting cancer or a particular type or subtype thereof in a subject and, optionally, treating the cancer or particular type or subtype thereof.

In some versions, the methods comprise: determining fragmentation patterns of classifier cell-free deoxyribonucleic acid (cfDNA) from the subject, wherein the classifier cfDNA comprises cfDNA from the subject corresponding to at least a portion of at least one exon of at least one classifier gene in a panel of one or more classifier genes; and classifying the fragmentation patterns to identify the subject as being negative or positive for the cancer or the particular type or subtype thereof.

In some versions, the classifier cfDNA comprises cfDNA from the subject corresponding to at least a portion of at least one exon of at least one classifier gene.

In some versions, the classifier cfDNA comprises cfDNA from the subject corresponding to at least a portion of a coding sequence of at least one exon of at least one classifier gene.

In some versions, the at least the portion of the at least one exon of the at least one classifier gene comprises a coding sequence of a first exon of the at least one classifier gene.

In some versions, at least the portion of the at least one exon of the at least one classifier gene comprises one or more predefined exon regions. In some versions, the predefined exon regions are selected from the group consisting of transcription factor binding sites, regions of open chromatin, and specific motifs.

In some versions, the classifier cfDNA excludes cfDNA from the subject corresponding to one or more exons of the at least one classifier gene other than the at least one exon.

In some versions, the classifier cfDNA corresponds to less than 2,500 Mb of a genome of the subject.

In some versions, the method further comprises isolating from the subject a biological sample comprising the classifier cfDNA. In some versions, the method further comprises isolating the classifier cfDNA from at least some non-classifier cfDNA, wherein the non-classifier cfDNA is cfDNA that is not classifier cfDNA.

In some versions, the method further comprises sequencing the classifier cfDNA. In some versions, the sequencing comprises sequencing the classifier cfDNA at a deduplicated sequencing depth of at least 100×. In some versions, the method excludes sequencing at least some non-classifier cfDNA from the subject. In some versions, the method sequences cfDNA corresponding to no more than 2,500 Mb of a genome of the subject.

In some versions, the determining the fragmentation patterns comprises determining a fragment size distribution of the classifier cfDNA. In some versions, determining the fragmentation patterns comprises determining a separate fragment size distribution of the classifier cfDNA corresponding to each classifier gene. In some versions, each classifier gene comprises a coding region of an exon and the determining the fragmentation patterns comprises determining a separate fragment size distribution of the classifier cfDNA corresponding to the coding region of each exon. In some versions, each classifier gene comprises a coding region of a first exon and the determining the fragmentation patterns comprises determining a separate fragment size distribution of the classifier cfDNA corresponding to the coding region of each first exon. In some versions, each classifier gene comprises a coding region of multiple exons and the determining the fragmentation patterns comprises determining a separate fragment size distribution of the classifier cfDNA corresponding to the coding region of each of the multiple exons.

In some versions, the determining the fragmentation patterns comprises quantitating each fragment size distribution. In some versions, the determining the fragmentation patterns comprises quantitating each fragment size distribution using size bins. In some versions, the quantitating comprises quantitating an entropy value for each fragment size distribution. In some versions, the quantitating comprises quantitating the number of reads (depth) for each fragment size distribution. In some versions, the determining the fragmentation patterns comprises examining the sequence motifs found on each fragment. In some versions, the determining the fragmentation patterns comprises determining a motif diversity score. In some versions, the determining the fragmentation patterns comprises determining the fragmentation patterns of one or more predefined exon regions. In some versions, the predefined exon regions are selected from the group consisting of transcription factor binding sites, regions of open chromatin, and specific motifs. In some versions, the determining the fragmentation patterns comprises determining a separate fragment size distribution of the classifier cfDNA corresponding to each predefined exon region.

In some versions, the classifier genes comprise cancer genes. In some versions, the one or more classifier genes comprise at least 50 genes from Gene Set 1. In some versions, the one or more classifier genes comprise at least 1 gene from Gene Set 2.

In some versions, the classifying identifies the subject as being negative or positive for at least one type of cancer. In some versions, at least one type of cancer comprises one or more tumor sites of origin. In some versions, the one or more tumor sites of origin comprise one or more of breast, bladder, lung, kidney, and prostate.

In some versions, the method is capable of identifying the subject as being positive for cancer at an accuracy of at least 90% in a biological sample from the subject having a ct-fraction from 0.0001 to 0.001. In some versions, the method is capable of identifying the subject as being positive for a cancer selected from the group consisting of breast cancer, bladder cancer, lung cancer, prostate cancer, and metastatic neuroendocrine prostate cancer at an accuracy of at least 70% in a biological sample from the subject having a ct-fraction from 0.001 to 0.01

In some versions, the method further comprises identifying the subject as having a cancer of a particular tissue of origin and subjecting the subject to imaging or biopsy of the particular tissue of origin. In some versions, the particular tissue of origin is a solid tissue and wherein the imaging or biopsy is of the solid tissue.

In some versions, the method further comprises identifying the subject as having cancer and treating the cancer. In some versions, the method further comprises identifying the subject as having a cancer of a particular tissue of origin and subjecting the subject to surgery on the particular tissue of origin. In some versions, the particular tissue of origin is a solid tissue and wherein the surgery is on the solid tissue.

The objects and advantages of the invention will appear more fully from the following detailed description of the preferred embodiment of the invention made in conjunction with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Schematic of fragmentomics experimental setup. Liquid biopsies from patients from two independent cohorts with various cancer types are collected and cfDNA is isolated using targeted exon panels. Unique histone distributions across cancer types lead to variable fragmentation patterns at targeted exons. Exon 1 shows particular variability due to its proximity to promoter regions and is correlated with gene expression. The diversity of fragmentation distributions at each coding exon 1 are measured via Shannon entropy for each sample. Machine learning models are built to predict tumor type for each cohort, with training performed on 70% of the data and 30% withheld for validation. Ten-fold cross validation performed on the training data. In the UW cohort, samples are randomly selected for training and validation, while the GRAIL cohort is trained on high ctDNA samples and validated on low ctDNA samples.

FIGS. 2A-2H. cfDNA fragmentation patterns from targeted panels. Average total fragment distribution across tumor types in the (FIG. 2A) GRAIL and (FIG. 2B) UW datasets respectively. Heatmap of the fragment size distributions at exon 1 across all genes from the GRAIL targeted panel (FIG. 2C) and UW targeted panel (FIG. 2D) in a single representative sample from each cohort. Genes are ordered by exon 1 Shannon entropy (E1SE) with high E1SE genes at the top and low E1SE genes at the bottom. Fragment size proportions are normalized within each fragment size across all genes analyzed. Plot demonstrates that genes with high E1SE are depleted for fragments near the mono-nucleosome peak (167bp) and enriched for fragments at lower (<120 bp) and higher (>200 bp) sizes, while genes with low E1SE display the opposite pattern. (FIG. 2E) Copy number calls from the UW cohort compared to Shannon entropy. Copy number was calculated for each gene for each patient. Each point represents a single gene-patient pair. Copy number data was binned as shown, and Shannon entropy distributions are shown for each bin. E1SE was normalized by centering and scaling on a per-gene basis before plotting. This transforms the E1SE distribution for each gene such that the mean is zero and the standard deviation is one, eliminating inter-gene variability. Data from all genes and patients are plotted. Only the UW cohort was used because the exact panel design was required to accurately determine CN, but this was not available for the GRAIL cohort (FIG. 2F) Shannon entropy as a function of fragments per exon in the UW cohort at copy number neutral regions (Log2 ratio between −0.5 and 0.5). Correlation between GC content and mean Shannon entropy at each exon analyzed in the (FIG. 2G) GRAIL cohort and (FIG. 2H) UW cohort.

FIGS. 3A-3D. Predicting tumor type in the UW panel and cohort. The UW data was split into 70% training and 30% independent validation, the latter of which is shown. Performance was assessed by (FIG. 3A) confusion matrix of classifier accuracy in CV data comparing predicted vs. actual phenotypes and (FIG. 3B) ROC curves of classifier AUCs in CV data. (FIG. 3C) Accuracy as a function of ctDNA fraction in CV data. ctDNA fractions ranged from 0.003-0.771. NEPC samples are not shown due to the lack of germline sequencing for this cohort which are required for ctDNA fraction estimation. Only samples with available germline sequencing, and thus ctDNA fraction estimation, are shown. The number of samples in each ctDNA fraction bin are: <0.01: n=10; 0.01-0.1: n=21; 0.1-1.0: n=26. (FIG. 3D) Radar plots depicting the prediction score, where each plot represents one pathologic diagnosis (noted in bold above the plot), and each line in the plot represents model prediction for a single patient. The vertices of each graph represent the continuous prediction scores from the EISE models for each of the predicted phenotypes, with the outer ring denoting a prediction score of 1 and the inner ring a prediction score of 0. For each patient, the final model prediction is the highest-scoring predicted phenotype which is correct in the majority of cases. The number of predictions for each tumor type are noted next to the label of each vertex (matching panel A). Correctly predicted patients are represented by colored lines, whereas incorrectly predicted patients are represented by light gray lines.

FIGS. 4A-4D. Predicting tumor type in the GRAIL panel and cohort. The GRAIL data was split into 70% training and 30% independent validation, the latter of which is shown. The validation data contained the lowest ctDNA fraction samples, all <0.05. Performance was assessed by (FIG. 4A) confusion matrix of classifier accuracy in validation data and (FIG. 4B) ROC curves of classifier AUCs in validation data. (FIG. 4C) Accuracy as a function of ctDNA fraction in validation data. ctDNA fractions ranged from 0.0003-0.925 for cancer samples. Light grey bars represent normal samples with a ctDNA fraction of 0. The number of samples in each ctDNA fraction bin are: 0 (Normal): n=33; <0.25: n=28; 0.25-1.0: n=32. (FIG. 4D) Radar plots depicting the prediction score, where each plot represents one specific pathologic diagnosis (noted in bold above the plot), and each line in the plot represents the model prediction for a single patient. The vertices of each graph represent the continuous prediction scores from the EISE models for each of the predicted phenotypes, with the outer ring denoting a prediction score of 1 and the inner ring a prediction score of 0. For each patient, the final model prediction is the highest-scoring predicted phenotype which is correct in the majority of cases. The number of predictions for each tumor type are noted next to the label of each vertex (matching FIG. 3A). Correctly predicted patients are represented by colored lines, whereas incorrectly predicted patients are represented by light gray lines.

FIGS. 5A and 5B. Effect of downsampling on model performance in the GRAIL cohort. Downsampling of the GRAIL cohort was performed to levels ranging from 100 M to 1 M reads 10 times for each downsampling level. For each replicate and downsampling level, Shannon entropies were calculated for the fragment distributions at the first exon of each gene in the panel as described previously. Training and validation using the new downsampled feature tables was performed and results for (FIG. 5A) ROC AUC and (FIG. 5B) accuracy are shown for each phenotype in the cohort. Small points represent individual values, large solid points represent mean values, and error bars represent +/−1 standard deviation.

FIGS. 6A and 6B. Fragment distribution of GRAIL cohort samples stratified by ctDNA fraction. (FIG. 6A) Distribution of cfDNA fragments from individual samples colored by low ctDNA (<10% ctDNA fraction) or high ctDNA (ctDNA fraction >=10%). Red line represents the median of all normal healthy samples. (FIG. 6B) Proportion of fragments below 150 bp in healthy, low ctDNA, and high ctDNA samples. A Kruskal-Wallis test was performed to compare all three categories, and a Wilcoxon rank sum test was performed for individual comparisons (*p<0.05; **** p<0.0001)

FIG. 7. Relative fragment coverage in first coding exon by gene expression decile. Average plasma cell-free DNA fragment coverage near the exon 1 coding sequence (CDS) of 11748 genes annotated in MANE version 0.93, calculated across 41 whole genome sequenced ctDNA-positive samples from the NCT02125357 trial (Herberts et al. Nature 2022). Genes were separated into ten quantile groups based on their average expression in prostate cancer tissue samples. Fragment coverage is normalized relative to 1 kb distant flanks. Only multi-exon genes with a CDS containing exon 1 were included in the analysis. Gene orientation and exon 1 CDS length were normalized between the genes for visualization. One kilobase of upstream and downstream flanking region is also shown (without normalization).

FIGS. 8A and 8B. Exon 1 Shannon entropy of the AR by cancer type. Normalized Shannon entropy was calculated for the first coding exon of the androgen receptor gene (AR) for all samples in the GRAIL cohort (FIG. 8A) and UW cohort (FIG. 8B). AR E1SE displays significantly higher normalized Shannon entropy in prostate cancer samples compared to other cancer types and healthy normal samples. Two-sided Student's t-test was used for significance testing (**** p<0.0001).

FIG. 9. Normalized AR Shannon entropy stratified by ctDNA fraction. Within each cancer type, samples were stratified into low and high ctDNA fraction using the median ctDNA fraction as the cutoff. Normalized Shannon entropy at the first coding exon of AR was calculated and plotted by cancer type and ctDNA level. High ctDNA fraction prostate cancer samples were found to have significantly higher AR E1SE compared to low ctDNA fraction prostate cancer samples only. Two-sided Student's t-test was used for significance testing (*p<0.05; n.s. —non significant)

FIG. 10. Model performance using alternative exons. Model performance was assessed using Shannon entropies calculated from reads overlapping either the first, middle (mid), or last exons of the genes in each panel (see bottom schematic). For genes with an even number of exons, the exon closest to the TSS of the two middle exons was used. Accuracy was calculated for the UW cohort (left) and the GRAIL cohort (right). In both cohorts, Shannon entropies calculated from the first exon had the highest accuracy.

FIGS. 11A-11D. ROC curves for E1SE models to identify RCC in the UW cohort using all genes (FIG. 11A), genes overlapping with the Tempus xF panel (FIG. 11B), genes overlapping with the Guardant 360 CDx panel (FIG. 11C), genes overlapping with the Foundation One Liquid CDx panel (FIG. 11D).

FIGS. 12A-12D. ROC curves for E1SE models to identify hormone receptor positive vs. negative breast cancer in the UW cohort using all genes (FIG. 12A), genes overlapping with the Tempus xF panel (FIG. 12B), genes overlapping with the Guardant 360 CDx panel (FIG. 12C), genes overlapping with the Foundation One Liquid CDx panel (FIG. 12D).

FIGS. 13A-13H. ROC curves for E1SE models to identify tumor types and subtypes in the UW cohort using all genes (FIG. 13A), genes overlapping with the Tempus xF panel (FIG. 13B), genes overlapping with the Guardant 360 CDx panel (FIG. 13C), genes overlapping with the Foundation One Liquid CDx panel (FIG. 13D). ROC curves for E1SE models to identify tumor types and subtypes in the GRAIL cohort using all genes (FIG. 13E), genes overlapping with the Tempus xF panel (FIG. 13F), genes overlapping with the Guardant 360 CDx panel (FIG. 13G), genes overlapping with the Foundation One Liquid CDx panel (FIG. 13H).

FIGS. 14A-14H. ROC curves for exon 1 depth models to identify tumor types and subtypes in the UW cohort using all genes (FIG. 14A), genes overlapping with the Tempus XF panel (FIG. 14B), genes overlapping with the Guardant 360 CDx panel (FIG. 14C), genes overlapping with the Foundation One Liquid CDx panel (FIG. 14D). ROC curves for exon 1 depth models to identify tumor types and subtypes in the GRAIL cohort using all genes (FIG. 14E), genes overlapping with the Tempus xF panel (FIG. 14F), genes overlapping with the Guardant 360 CDx panel (FIG. 14G), genes overlapping with the Foundation One Liquid CDx panel (FIG. 14H).

FIGS. 15A-15H. ROC curves for full gene depth models to identify tumor types and subtypes in the UW cohort using all genes (FIG. 15A), genes overlapping with the Tempus xF panel (B), genes overlapping with the Guardant 360 CDx panel (FIG. 15C), genes overlapping with the Foundation One Liquid CDx panel (FIG. 15D). ROC curves for full gene depth models to identify tumor types and subtypes in the GRAIL cohort using all genes (FIG. 15E), genes overlapping with the Tempus xF panel (FIG. 15F), genes overlapping with the Guardant 360 CDx panel (FIG. 15G), genes overlapping with the Foundation One Liquid CDx panel (FIG. 15H).

FIGS. 16A-16H. ROC curves for exon 1 motif diversity score models to identify tumor types and subtypes in the UW cohort using all genes (FIG. 16A), genes overlapping with the Tempus xF panel (FIG. 16B), genes overlapping with the Guardant 360 CDx panel (FIG. 16C), genes overlapping with the Foundation One Liquid CDx panel (FIG. 16D). ROC curves for exon 1 motif diversity score models to identify tumor types and subtypes in the GRAIL cohort using all genes (FIG. 16E), genes overlapping with the Tempus xF panel (FIG. 16F), genes overlapping with the Guardant 360 CDx panel (FIG. 16G), genes overlapping with the Foundation One Liquid CDx panel (FIG. 16H).

FIGS. 17A-17H. ROC curves for exon 1 fragment size bin models to identify tumor types and subtypes in the UW cohort using all genes (FIG. 17A), genes overlapping with the Tempus xF panel (FIG. 17B), genes overlapping with the Guardant 360 CDx panel (FIG. 17C), genes overlapping with the Foundation One Liquid CDx panel (FIG. 17D). ROC curves for exon 1 fragment size bin models to identify tumor types and subtypes in the GRAIL cohort using all genes (FIG. 17E), genes overlapping with the Tempus xF panel (FIG. 17F), genes overlapping with the Guardant 360 CDx panel (FIG. 17G), genes overlapping with the Foundation One Liquid CDx panel (FIG. 17H).

FIGS. 18A-18H. ROC curves for exon 1 small fragment proportion models to identify tumor types and subtypes in the UW cohort using all genes (FIG. 18A), genes overlapping with the Tempus xF panel (FIG. 18B), genes overlapping with the Guardant 360 CDx panel (FIG. 18C), genes overlapping with the Foundation One Liquid CDx panel (FIG. 18D). ROC curves for exon 1 small fragment proportion models to identify tumor types and subtypes in the GRAIL cohort using all genes (FIG. 18E), genes overlapping with the Tempus xF panel (FIG. 18F), genes overlapping with the Guardant 360 CDx panel (FIG. 18G), genes overlapping with the Foundation One Liquid CDx panel (FIG. 18H).

FIGS. 19A-19H. ROC curves for all exon Shannon Entropy models to identify tumor types and subtypes in the UW cohort using all genes (FIG. 19A), genes overlapping with the Tempus xF panel (FIG. 19B), genes overlapping with the Guardant 360 CDx panel (FIG. 19C), genes overlapping with the Foundation One Liquid CDx panel (FIG. 19D). ROC curves for all exon Shannon Entropy models to identify tumor types and subtypes in the GRAIL cohort using all genes (FIG. 19E), genes overlapping with the Tempus xF panel (FIG. 19F), genes overlapping with the Guardant 360 CDx panel (FIG. 19G), genes overlapping with the Foundation One Liquid CDx panel (FIG. 19H).

FIGS. 20A-20H. ROC curves for all exon depth models to identify tumor types and subtypes in the UW cohort using all genes (FIG. 20A), genes overlapping with the Tempus xF panel (FIG. 20B), genes overlapping with the Guardant 360 CDx panel (FIG. 20C), genes overlapping with the Foundation One Liquid CDx panel (FIG. 20D). ROC curves for all exon depth models to identify tumor types and subtypes in the GRAIL cohort using all genes (FIG. 20E), genes overlapping with the Tempus XF panel (FIG. 20F), genes overlapping with the Guardant 360 CDx panel (FIG. 20G), genes overlapping with the Foundation One Liquid CDx panel (FIG. 20H).

FIGS. 21A-21H. ROC curves for all exon motif diversity score models to identify tumor types and subtypes in the UW cohort using all genes (FIG. 21A), genes overlapping with the Tempus xF panel (FIG. 21B), genes overlapping with the Guardant 360 CDx panel (FIG. 21C), genes overlapping with the Foundation One Liquid CDx panel (FIG. 21D). ROC curves for all exon motif diversity score models to identify tumor types and subtypes in the GRAIL cohort using all genes (FIG. 21E), genes overlapping with the Tempus xF panel (FIG. 21F), genes overlapping with the Guardant 360 CDx panel (FIG. 21G), genes overlapping with the Foundation One Liquid CDx panel (FIG. 21H).

FIGS. 22A-22H. ROC curves for all exons small fragment proportion models to identify tumor types and subtypes in the UW cohort using all genes (FIG. 22A), genes overlapping with the Tempus xF panel (FIG. 22B), genes overlapping with the Guardant 360 CDx panel (FIG. 22C), genes overlapping with the Foundation One Liquid CDx panel (FIG. 22D). ROC curves for all exons small fragment proportion models to identify tumor types and subtypes in the GRAIL cohort using all genes (FIG. 22E), genes overlapping with the Tempus xF panel (FIG. 22F), genes overlapping with the Guardant 360 CDx panel (FIG. 22G), genes overlapping with the Foundation One Liquid CDx panel (FIG. 22H).

FIGS. 23A-23H. ROC curves for E1SE +depth models to identify tumor types and subtypes in the UW cohort using all genes (FIG. 23A), genes overlapping with the Tempus xF panel (FIG. 23B), genes overlapping with the Guardant 360 CDx panel (FIG. 23C), genes overlapping with the Foundation One Liquid CDx panel (FIG. 23D). ROC curves for E1SE +depth models to identify tumor types and subtypes in the GRAIL cohort using all genes (FIG. 23E), genes overlapping with the Tempus xF panel (FIG. 23F), genes overlapping with the Guardant 360 CDx panel (FIG. 23G), genes overlapping with the Foundation One Liquid CDx panel (FIG. 23H).

FIGS. 24A-24H. ROC curves for all exons Shannon Entropy +depth models to identify tumor types and subtypes in the UW cohort using all genes (FIG. 24A), genes overlapping with the Tempus xF panel (FIG. 24B), genes overlapping with the Guardant 360 CDx panel (FIG. 24C), genes overlapping with the Foundation One Liquid CDx panel (FIG. 24D). ROC curves for all exons Shannon Entropy +depth models to identify tumor types and subtypes in the GRAIL cohort using all genes (FIG. 24E), genes overlapping with the Tempus xF panel (FIG. 24F), genes overlapping with the Guardant 360 CDx panel (FIG. 24G), genes overlapping with the Foundation

One Liquid CDx panel (FIG. 24H).

FIGS. 25A-25B. ROC curves depicting the prediction of high or low ctDNA fraction (CTF) in cancer samples using exon 1 Shannon entropy (E1SE) in the UW cohort (FIG. 25A) and the GRAIL cohort (FIG. 25B) using a 10-fold cross validation approach. The cutoff for “low” and “high” ctDNA fraction was 0.05.

FIGS. 26A-26H. ROC curves for the Transcription Factor Binding Site Shannon Entropy models to identify tumor types and subtypes in the UW cohort using all genes (FIG. 26A), genes overlapping with the Tempus xF panel (FIG. 26B), genes overlapping with the Guardant 360 CDx panel (FIG. 26C), genes overlapping with the Foundation One Liquid CDx panel (FIG. 26D). ROC curves for the Transcription Factor Binding Site Shannon Entropy models to identify tumor types and subtypes in the GRAIL cohort using all genes (FIG. 26E), genes overlapping with the Tempus xF panel (FIG. 26F), genes overlapping with the Guardant 360 CDx panel (FIG. 26G), genes overlapping with the Foundation One Liquid CDx panel (FIG. 26H).

FIGS. 27A-27H. ROC curves for the open chromatin region (ATAC-seq) Shannon entropy models to identify tumor types and subtypes in the UW cohort using all genes (FIG. 27A), genes overlapping with the Tempus XF panel (FIG. 27B), genes overlapping with the Guardant 360 CDx panel (FIG. 27C), genes overlapping with the Foundation One Liquid CDx panel (FIG. 27D). ROC curves for the open chromatin region (ATAC-seq) Shannon entropy models to identify tumor types and subtypes in the GRAIL cohort using all genes (FIG. 27E), genes overlapping with the Tempus xF panel (FIG. 27F), genes overlapping with the Guardant 360 CDx panel (FIG. 27G), genes overlapping with the Foundation One Liquid CDx panel (FIG. 27H).

FIGS. 28A-28L. AUROC values of model performance in the UW cohort across E1SE, exon 1 depth, E1SE and exon 1 depth, all exons Shannon entropy (SE), all exons depth, combining all exons depth and Shannon entropy, full gene depth, exon 1 MDS, all exon MDS, exon 1 small fragment proportions, all exons small fragment proportions, fragment size bins, TFBS entropy, and ATAC region entropy. The UW cohort comprises bladder cancer, breast cancer, lung cancer, renal cell cancer (RCC), prostate adenocarcinoma (Prostate), and neuroendocrine prostate cancer (NEPC). UW breast cancer samples were further split into ER positive (ERpos) and ER negative (ERneg) samples. UW lung cancer samples were further split into small cell lung cancer (SCLC) and non-small cell lung cancer (NSCLC). Ten replicates of the 10-fold cross-validation model were performed and boxplots using all ten replicates are shown. Model performance to identify tumor types and subtypes was determined using all genes in the UW panel (FIGS. 28A-28C), genes overlapping with the Tempus XF panel (FIGS. 28D-28F), genes overlapping with the Guardant 360 CDx panel (FIGS. 28G-28I), genes overlapping with the Foundation One Liquid CDx panel (FIGS. 28J-28L).

FIGS. 29A-29H. AUROC values of model performance in the GRAIL cohort across E1SE, exon 1 depth, E1SE and exon 1 depth, all exons Shannon entropy (SE), all exons depth, combining all exons depth and Shannon entropy, full gene depth, exon 1 MDS, all exon MDS, exon 1 small fragment proportions, all exons small fragment proportions, fragment size bins, TFBS entropy, and ATAC region entropy. Ten replicates of the 10-fold cross-validation model were performed and boxplots using all ten replicates are shown. Model performance to identify tumor types and subtypes was determined using all genes in the GRAIL panel (FIGS. 29A and 29B), genes overlapping with the Tempus XF panel (FIGS. 29C and 29D), genes overlapping with the Guardant 360 CDx panel (FIGS. 29E and 29F), genes overlapping with the Foundation One Liquid CDx panel (FIGS. 29G and 29H).

FIGS. 30A-30L. AUROC values of model performance in the UW cohort split by ctDNA fraction bin across E1SE, exon 1 depth, E1SE and exon 1 depth, all exons Shannon entropy (SE), all exons depth, combining all exons depth and Shannon entropy, full gene depth, exon 1 MDS, all exon MDS, exon 1 small fragment proportions, all exons small fragment proportions, fragment size bins, TFBS entropy, and ATAC region entropy. Samples were split into low ctDNA fraction samples (0-0.05) and high ctDNA fraction samples (0.05-1). Cancer types with an insufficient number of samples (less than 3) within each ctDNA fraction bin were excluded from analysis. The cancer types which fit these criteria were bladder cancer, ER positive breast cancer, NSCLC, prostate adenocarcinoma, and RCC. Ten replicates of the 10-fold cross-validation model were performed and boxplots using all ten replicates are shown. Model performance to identify tumor types and subtypes was determined using all genes in the UW panel (FIGS. 30-30C), genes overlapping with the Tempus XF panel (FIGS. 30D-30F), genes overlapping with the Guardant 360 CDx panel (FIGS. 30G-30I), genes overlapping with the Foundation One Liquid CDx panel (FIGS. 30J-30L).

FIGS. 31A-31L. AUROC values of model performance in the GRAIL cohort split by ctDNA fraction bin across E1SE, exon 1 depth, E1SE and exon 1 depth, all exons Shannon entropy (SE), all exons depth, combining all exons depth and Shannon entropy, full gene depth, exon 1 MDS, all exon MDS, exon 1 small fragment proportions, all exons small fragment proportions, fragment size bins, TFBS entropy, and ATAC region entropy. Samples were split into low ctDNA fraction samples (0-0.05) and high ctDNA fraction samples (0.05-1). Only cancer types with a sufficient number of samples (greater than or equal to 3) within each ctDNA fraction bin were excluded from analysis. The cancer types which fit these criteria were prostate cancer, breast cancer, and lung cancer. Ten replicates of the 10-fold cross-validation model were performed and boxplots using all ten replicates are shown. Model performance to identify tumor types and subtypes was determined using all genes in the GRAIL panel (FIGS. 31A-31C), genes overlapping with the Tempus XF panel (FIGS. 31D-31F), genes overlapping with the Guardant 360 CDx panel (FIGS. 31G-31I), genes overlapping with the Foundation One Liquid CDx panel (FIGS. 31J-31L).

FIGS. 32A-32H. AUROC values from models trained on the UW cohort to predict ctDNA fraction across E1SE, exon 1 depth, E1SE and exon 1 depth, all exons Shannon entropy (SE), all exons depth, combining all exons depth and Shannon entropy, full gene depth, exon 1 MDS, all exon MDS, exon 1 small fragment proportions, all exons small fragment proportions, fragment size bins, TFBS entropy, and ATAC region entropy. Samples were split into low ctDNA fraction (0-0.01), mid ctDNA fraction (0.01-0.1), high ctDNA fraction (0.1-1), and healthy samples. Ten replicates of the 10-fold cross-validation model were performed and boxplots using all ten replicates are shown. Model performance to identify ctDNA fraction bin was determined using all genes in the UW panel (FIGS. 32A and 32B), genes overlapping with the Tempus xF panel (FIGS. 32C and 32D), genes overlapping with the Guardant 360 CDx panel (FIGS. 32E and 32F), genes overlapping with the Foundation One Liquid CDx panel (FIGS. 32G and 32H).

FIGS. 33A-33H. AUROC values from models trained on the GRAIL cohort to predict ctDNA fraction across E1SE, exon 1 depth, E1SE and exon 1 depth, all exons Shannon entropy (SE), all exons depth, combining all exons depth and Shannon entropy, full gene depth, exon 1 MDS, all exon MDS, exon 1 small fragment proportions, all exons small fragment proportions, fragment size bins, TFBS entropy, and ATAC region entropy. Samples were split into low ctDNA fraction (0-0.01), mid ctDNA fraction (0.01-0.1), high ctDNA fraction (0.1-1), and healthy samples. Ten replicates of the 10-fold cross-validation model were performed and boxplots using all ten replicates are shown. Model performance to identify ctDNA fraction bin was determined using all genes in the GRAIL panel (FIGS. 32A and 32B), genes overlapping with the Tempus xF panel (FIGS. 32C and 32D), genes overlapping with the Guardant 360 CDx panel (FIGS. 32E and 32F), genes overlapping with the Foundation One Liquid CDx panel (FIGS. 32G and 32H).

### DETAILED DESCRIPTION OF THE INVENTION

One aspect of the invention is directed to methods of detecting cancer in a subject. “Detecting cancer” as used herein refers to detecting cancer or any particular type thereof. The term “subject,” as used herein, generally refers to any animal, mammal, or human. In some embodiments, the subject has, potentially has, or is suspected of having cancer or a symptom(s) associated with cancer. In some embodiments, the subject asymptomatic with respect to cancer. In some embodiments, the subject is undiagnosed (e.g., not diagnosed for cancer).

The methods of detecting cancer can comprise various isolation, sequencing, and/or analysis steps with classifier cell-free deoxyribonucleic acid (cell-free DNA or cfDNA) from the subject.

cfDNA comprises nucleic acid fragments not contained within a cell that circulate in an subject's body (e.g., bloodstream). cfDNA can originate from one or more healthy cells and/or from one or more cancerous cells of the subject's body. cfDNA may come from other sources such as viruses, fetuses, etc. cfDNA can include circulating tumor DNA (ctDNA). ctDNA is cfDNA that originates from tumor cells. ctDNA may be released into a subject's bloodstream as result of biological processes such as apoptosis or necrosis of dying tumor cells or by active release by viable tumor cells.

Classifier cfDNA is cfDNA that is analyzed for classification according to the methods described herein. Classifier cfDNA is distinguished from non-classifier cfDNA, the latter of which is cfDNA that is not classifier cfDNA. The classifier cfDNA can comprise cfDNA that corresponds to one or more regions of a genome. The term “corresponds” (or grammatical variants thereof) refers to a relationship between a first nucleic acid (e.g., a cfDNA, a probe) and at least a region of a second nucleic acid (e.g., a defined region in a chromosome of a genome) such that the first nucleic acid comprises at least one base that aligns (overlaps) with at least one base in the region when the sequence of the first nucleic acid is aligned to that of the second nucleic acid. The regions of the genome to which the classifier cfDNA corresponds are referred to herein as “classifier regions.” Classifier regions are distinguished from non-classifier regions, the latter of which are region that are not classifier regions. The classifier regions can comprise genic regions of the genome, intergenic regions of the genome, or a combination thereof. In some embodiments, the classifier regions comprise genes or specific parts thereof (e.g, exons, introns, promoters, coding regions, regulatory regions, enhancers, untranslated regions (5′ untranslated region, 3′ untranslated region, etc.). A gene to which classifier cfDNA corresponds (i.e., a gene that comprises at least one base that aligns to at least one base of classifier cfDNA) is referred to herein as a “classifier gene.” Classifier genes are distinguished from non-classifier genes, the latter of which are genes that are not classifier genes. In some embodiments, the classifier regions comprise exons. As is known in the art, exons are contiguous portions of genes that form the final mature RNA produced by genes after introns have been removed by RNA splicing. Exons of classifier genes that correspond to classifier cfDNA are referred to herein as “classifier exons.” Classifier exons are distinguished from non-classifier exons, the latter of which are exons that are not classifier exons. In some embodiments, the classifier exons comprise particular exons, such as first exons. In some embodiments, the classifier exons comprise coding regions of particular exons, such as coding regions of first exons. “First exon” as used herein refers to a contiguous portion of a given gene that forms the furthest 5′ part of a final mature RNA produced by that gene after introns have been removed by RNA splicing. In some cases, a given gene can have multiple first exons depending on the various isoforms it is capable of generating due to alternative splicing or alternative transcription start sites.

In some embodiments, the classifier regions comprise at least a portion of at least one exon of at least one classifier gene. In some embodiments, the classifier regions comprise at least a portion of the coding sequence of at least one exon of at least one classifier gene. In some embodiments, the classifier regions comprise at least a portion of the first exon of at least one classifier gene. In some embodiments, the classifier regions comprise at least a portion of the coding sequence of the first exon of at least one classifier gene. In some embodiments, the classifier regions comprise the entirety of at least one exon of at least one classifier gene. In some embodiments, the classifier regions comprise the entirety of the coding sequence of at least one exon of at least one classifier gene. In some embodiments, the classifier regions comprise the entirety of the first exon of at least one classifier gene. In some embodiments, the classifier regions comprise the entirety of the coding sequence of the first exon of at least one classifier gene. In some embodiments, the classifier regions comprise the entirety of at least one exon of each classifier gene. In some embodiments, the classifier regions comprise the entirety of the coding sequence of at least one exon of each classifier gene. In some embodiments, the classifier regions comprise, consist, or consist essentially of the entirety of the first exon of each classifier gene. In some embodiments, the classifier regions comprise, consist, or consist essentially of the entirety of the coding sequence of the first exon of each classifier gene. Accordingly, the classifier cfDNA of the invention can correspond to any of the above-described classifier regions

In some embodiments, the non-classifier regions comprise intergenic regions of the genome. In some embodiments, the non-classifier regions comprise at least one intron of at least one classifier gene. In some embodiments, the non-classifier regions comprise at least one intron of each classifier gene. In some embodiments, the non-classifier regions comprise each intron of each classifier gene. In some embodiments, the non-classifier regions comprise at least one exon in at least one classifier gene. In some embodiments, the non-classifier regions comprise at least one exon in each classifier gene. In some embodiments, the non-classifier regions comprise at least one exon other than the first exon in at least one classifier gene. In some embodiments, the non-classifier regions comprise at least one exon other than the first exon in each classifier gene. In some embodiments, the non-classifier regions comprise each exon other than the first exon in at least one classifier gene. In some embodiments, the non-classifier regions comprise each exon other than the first exon in each classifier gene. Accordingly, the classifier cfDNA of the invention can exclude cfDNA corresponding to any of the above-described non-classifier regions.

In various embodiments, the classifier regions constitute less than 2,999 Mb, less than 2,750 Mb, less than 2,500 Mb, less than 2,250 Mb, less than 2,000 Mb, than 1,750 Mb, less than 1,500 Mb, less than 1,250 Mb, less than 1,000 Mb, than 750 Mb, less than 500 Mb, less than 250 Mb, less than 200 Mb, less than 150 Mb, less than 100 Mb, less than 50 Mb, less than 25 Mb, less than 10 Mb, or less than 5 Mb of a reference genome or a genome of the subject. Accordingly, the classifier cfDNA of the invention can correspond to any of the above-referenced portions of the genome.

In various embodiments, the classifier gene(s) in total constitute less than 2,999 Mb, less than 2,750 Mb, less than 2,500 Mb, less than 2,250 Mb, less than 2,000 Mb, than 1,750 Mb, less than 1,500 Mb, less than 1,250 Mb, less than 1,000 Mb, than 750 Mb, less than 500 Mb, less than 250 Mb, less than 200 Mb, less than 150 Mb, less than 100 Mb, less than 50 Mb, less than 25 Mb, less than 10 Mb, or less than 5 Mb of a reference genome or a genome of the subject. Accordingly, the classifier cfDNA of the invention can correspond to classifier gene(s) constituting any of the above-referenced portions of the genome.

In various embodiments, the number of classifier gene(s) is at least 1, at least 5, at least 25, at least 50, at least 75, at least 100, at least 125, at least 150, at least 175, at least 200, at least 250, at least 250, at least 275, at least 300, at least 325, at least 350, at least 375, at least 400, at least 450, at least 475, or at least 500. In various embodiments, the number of classifier gene(s) is no more than 25,000, no more than 20,000, no more than 15,000, no more than 10,000, no more than 5,000, no more than 2,500, no more than 2,000, no more than 1,750, no more than 1,500, no more than 1,250, or no more than 1,000.

In preferred embodiments, the classifier gene(s) comprise, consist, or consist essentially of cancer genes. Cancer genes are genes involved in the etiology, maintenance, or progression of cancer. In some embodiments, the cancer genes comprise or consist of genes in which one or more mutations in those genes are associated with cancer, such as in a statistically significant manner. Exemplary types of cancer genes include oncogenes, tumor suppressor genes, and DNA repair genes. A number of databases are available that catalog cancer genes. The COSMIC (the Catalogue of Somatic Mutations in Cancer) database (cancer.sanger.ac.uk/cosmic), for example, is a database of somatically acquired mutations found in human cancer (Tate J G, et al. COSMIC: the catalogue of somatic mutations in cancer. Nucleic Acids Res. 2019;47:D941-D947). The DisGeNET (disgenet.org) database is a platform containing one of the largest publicly available collections of genes and variants associated with human diseases (Piñero J, Saüch J, Sanz F, Furlong L I. The DisGeNET cytoscape app: Exploring and visualizing disease genomics data. Comput Struct Biotechnol J. 2021 May 11;19:2960-2967) (Piñero J, Ramirez-Anguita J M, Saüch-Pitarch J, Ronzano F, Centeno E, Sanz F, Furlong L I. The DisGeNET knowledge platform for disease genomics: 2019 update. Nucleic Acids Res. 2020 Jan 8;48(D1):D845-D855) (Piñero J, Bravo À, Queralt-Rosinach N, Gutiérrez-Sacristán A, Deu-Pons J, Centeno E, García-García J, Sanz F, Furlong L I. DisGeNET: a comprehensive platform integrating information on human disease-associated genes and variants. Nucleic Acids Res. 2017 Jan 4;45(D1):D833-D839) (Piñero J, Queralt-Rosinach N, Bravo À, Deu-Pons J, Bauer-Mehren A, Baron M, Sanz F, Furlong L I. DisGeNET: a discovery platform for the dynamical exploration of human diseases and their genes. Database (Oxford). 2015 Apr. 15;2015:bav028). A large number of other databases are available (Babbi G, Martelli P L, Profiti G, Bovo S, Savojardo C, Casadio R. eDGAR: a database of Disease-Gene Associations with annotated Relationships among genes. BMC Genomics. 2017 Aug. 11;18(Suppl 5):554) (Grissa D, Junge A, Oprea T I, Jensen L J. Diseases 2.0: a weekly updated database of disease-gene associations from text mining and data integration. Database (Oxford). 2022 Mar. 28;2022:baac019).

In some embodiments, the classifier gene(s) comprise, consist, or consist essentially of one, some, or all of the genes in Gene Set 1. In some embodiments, the classifier gene(s) comprise, consist, or consist essentially of one, some, or all of the genes in Gene Set 2. In some embodiments, the classifier gene(s) comprise, consist, or consist essentially of one, some, or all of the genes in Gene Set 3. In some embodiments, the classifier gene(s) comprise, consist, or consist essentially of one, some, or all of the genes in Gene Set 4. In various embodiments, the classifier gene(s) comprise, consist, or consist essentially of at least 1, at least 5, at least 25, at least 50, at least 75, at least 100, at least 125, at least 150, at least 175, at least 200, at least 250, at least 250, at least 275, at least 300, at least 325, at least 350, at least 375, at least 400, at least 450, at least 475, or at least 500 of the genes in any of Gene Set 1, Gene Set 2, Gene Set 3, or Gene Set 4.

Gene Set 1 is ABL1, ABL2, ABRAXAS1, ACKR3, ACSL3, ACSL6, ACVR1, ACVR1B, ACVR2A, ADAMTS20, ADGRA2, ADGRB3, ADGRL3, AFDN, AFF1, AFF3, AFF4, AKAP9, AKT1, AKT2, AKT3, ALDH2, ALK, ALOX12B, AMER1, ANK1, ANKRD11, ANKRD26, APC, APOBEC3B, AR, ARAF, ARFRP1, ARHGAP26, ARHGAP5, ARHGEF10, ARHGEF10L, ARHGEF12, ARID1A, ARID1B, ARID2, ARID5B, ARNT, ASPSCR1, ASXL1, ASXL2, ATF1, ATIC, ATM, ATP1A1, ATP2B3, ATR, ATRX, AURKA, AURKB, AURKC, AXIN1, AXIN2, AXL, B2M, BAP1, BARD1, BAX, BAZ1A, BBC3, BCL10, BCL11A, BCL11B, BCL2, BCL2L1, BCL2L11, BCL2L12, BCL2L2, BCL3, BCL6, BCL7A, BCL9, BCL9L, BCLAF1, BCOR, BCORL1, BCR, BIRC2, BIRC3, BIRC5, BIRC6, BLM, BLNK, BMP5, BMPR1A, BRAF, BRCA1, BRCA2, BRD3, BRD4, BRIP1, BTG1, BTG2, BTK, BUB1B, C15orf65, CACNA1D, CALR, CAMTA1, CANT1, CARD11, CARS1, CASP3, CASP8, CASP9, CBFA2T3, CBFB, CBL, CBLB, CBLC, CCDC6, CCN6, CCNB1IP1, CCNC, CCND1, CCND2, CCND3, CCNE1, CCR4, CCR7, CD209, CD22, CD274, CD276, CD28, CD70, CD74, CD79A, CD79B, CDC73, CDH1, CDH10, CDH11, CDH17, CDH2, CDH20, CDH5, CDK12, CDK4, CDK6, CDK8, CDKN1A, CDKN1B, CDKN2A, CDKN2B, CDKN2C, CDX2, CEBPA, CENPA, CEP43, CEP89, CHCHD7, CHD2, CHD4, CHEK1, CHEK2, CHIC2, CHST11, CIC, CIITA, CILK1, CKS1B, CLIP1, CLP1, CLTC, CLTCL1, CMPK1, CNBD1, CNBP, CNOT3, CNTNAP2, CNTRL, COLIA1, COL2A1, COL3A1, COP1, COX6C, CPEB3, CRBN, CREB1, CREB3L1, CREB3L2, CREBBP, CRKL, CRLF2, CRNKL1, CRTC1, CRTC3, CSFIR, CSF3R, CSMD3, CSNK1A1, CTCF, CTLA4, CTNNA1, CTNNA2, CTNNB1, CTNND1, CTNND2, CUL3, CUL4A, CUX1, CXCR4, CYLD, CYP17A1, CYP2C19, CYP2C8, CYP2D6, CYSLTR2, DAXX, DCAF12L2, DCC, DCTN1, DCUN1D1, DDB2, DDIT3, DDR1, DDR2, DDX10, DDX3×, DDX41, DDX5, DDX6, DEK, DGCR8, DHX15, DICER1, DIS3, DNAJB1, DNM2, DNMT1, DNMT3A, DNMT3B, DOT1L, DPYD, DROSHA, DST, E2F3, EBF1, ECT2L, EED, EGFL7, EGFR, EIF1AX, EIF3E, EIF4A2, EIF4E, ELF3, ELF4, ELK4, ELL, ELN, ELOC, EML4, EMSY, EP300, EP400, EPAS1, EPCAM, EPHA3, EPHA5, EPHA7, EPHB1, EPHB4, EPHB6, EPS15, ERBB2, ERBB3, ERBB4, ERC1, ERCC1, ERCC2, ERCC3, ERCC4, ERCC5, ERG, ERRFI1, ESR1, ETNK1, ETS1, ETV1, ETV4, ETV5, ETV6, EWSR1, EXT1, EXT2, EZH2, EZR, FAM131B, FAM135B, FAM47C, FANCA, FANCC, FANCD2, FANCE, FANCF, FANCG, FANCI, FANCL, FAS, FAT1, FAT3, FAT4, FBLN2, FBXO11, FBXW7, FCGR2B, FCRL4, FEN1, FES, FEV, FGF1, FGF10, FGF12, FGF14, FGF19, FGF2, FGF23, FGF3, FGF4, FGF5, FGF6, FGF7, FGF8, FGF9, FGFR1, FGFR2, FGFR3, FGFR4, FGR, FH, FHIT, FIP1L1, FKBP9, FLCN, FLI1, FLNA, FLT1, FLT3, FLT4, FN1, FNBP1, FOXA1, FOXL2, FOXO1, FOXO3, FOXO4, FOXP1, FOXP4, FOXR1, FRS2, FSTL3, FUBP1, FUS, FYN, FZR1, G6PD, GABRA6, GAS7, GATA1, GATA2, GATA3, GATA4, GATA6, GDNF, GEN1, GFRA1, GID4, GLI1, GMPS, GNA11, GNA13, GNAQ, GNAS, GOLGA5, GOPC, GPC3, GPC5, GPHN, GPS2, GREM1, GRIN2A, GRM3, GRM8, GSK3B, GUCY1A2, H1-2, H2BC5, H3-3A, H3-3B, H3-4, H3-5, H3C1, H3C10, H3C11, H3C12, H3C13, H3C14, H3C15, H3C2, H3C3, H3C4, H3C6, H3C7, H3C8, H4C9, HCAR1, HDAC1, HERPUD1, HEY1, HGF, HIF1A, HIP1, HLA-A, HLA-B, HLA-C, HLF, HMGA1, HMGA2, HNF1A, HNRNPA2B1, HNRNPK, HOOK3, HOXA11, HOXA13, HOXA9, HOXB13, HOXC11, HOXC13, HOXD11, HOXD13, HRAS, HSD3B1, HSP90AA1, HSP90AB1, ICOSLG, ID3, IDH1, IDH2, IFNGR1, IGF1, IGF1R, IGF2, IKBKE, IKZF1, IL10, IL2, IL21R, IL6ST, IL7R, ING4, INHA, INHBA, INPP4A, INPP4B, INSR, IRF2, IRF4, IRS1, IRS2, IRS4, ISX, ITGA10, ITGA9, ITGAV, ITGB2, ITGB3, ITK, JAK1, JAK2, JAK3, JAZF1, JUN, KAT6A, KAT6B, KAT7, KCNJ5, KDM5A, KDM5C, KDM6A, KDR, KDSR, KEAP1, KEL, KIAA1549, KIFSB, KIT, KLF4, KLF6, KLHL6, KLK2, KMT2A, KMT2B, KMT2C, KMT2D, KNL1, KNSTRN, KRAS, KTN1, LAMP1, LARP4B, LASP1, LATS1, LATS2, LCK, LCP1, LEF1, LEPROTL1, LHFPL6, LIFR, LMNA, LMO1, LMO2, LPP, LRIG3, LRP1B, LSM14A, LTF, LTK, LYL1, LYN, LZTR1, MACC1, MAF, MAFB, MAGEA1, MAGI1, MAGI2, MALT1, MAML2, MAP2K1, MAP2K2, MAP2K4, MAP2K7, MAP3KI1, MAP3K13, MAP3K14, MAP3K4, MAP3K7, MAPK1, MAPK3, MAPK8, MARK1, MARK4, MAX, MB21D2, MBD1, MCL1, MDC1, MDM2, MDM4, MECOM, MED12, MEF2B, MENI, MERTK, MET, MGA, MGMT, MITF, MKNK1, MLF1, MLH1, MLLT1, MLLT10, MLLT11, MLLT3, MLLT6, MMP2, MN1, MNX1, MPL, MRE11, MRTFA, MSH2, MSH3, MSH6, MSI2, MSN, MST1, MST1R, MTAP, MTCP1, MTOR, MTR, MTRR, MUC1, MUC16, MUC4, MUTYH, MYB, MYBL1, MYC, MYCL, MYCN, MYD88, MYH11, MYH9, MYO5A, MYOD1, N4BP2, NAB2, NACA, NBEA, NBN, NCKIPSD, NCOAI, NCOA2, NCOA3, NCOA4, NCOR1, NCOR2, NDRG1, NEGR1, NF1, NF2, NFATC2, NFE2L2, NFIB, NFKB1, NFKB2, NFKBIA, NFKBIE, NIN, NKX2-1, NKX3-1, NLRP1, NONO, NOTCH1, NOTCH2, NOTCH3, NOTCH4, NPM1, NR4A3, NRAS, NRG1, NSD1, NSD2, NSD3, NT5C2, NTHL1, NTRK1, NTRK2, NTRK3, NUMA1, NUP214, NUP93, NUP98, NUTM1, NUTM2A, NUTM2B, NUTM2D, OLIG2, OMD, P2RY8, PABPCI, PAFAHIB2, PAK1, PAK3, PAK5, PALB2, PARP1, PARP2, PARP3, PATZ1, PAX3, PAX5, PAX7, PAX8, PBRM1, PBX1, PCBP1, PCM1, PDCD1, PDCD1LG2, PDE4DIP, PDGFB, PDGFRA, PDGFRB, PDK1, PDPK1, PER1, PGAP3, PGR, PHF6, PHOX2B, PICALM, PIK3C2B, PIK3C2G, PIK3C3, PIK3CA, PIK3CB, PIK3CD, PIK3CG, PIK3R1, PIK3R2, PIK3R3, PIM1, PKHD1, PLAG1, PLCG1, PLCG2, PLEKHG5, PLK2, PMAIPI, PML, PMS1, PMS2, PNRC1, POLD1, POLE, POLG, POLQ, POT1, POU2AF1, POUSF1, PPARG, PPFIBP1, PPM1D, PPP2R1A, PPP2R2A, PPP6C, PRCC, PRDM1, PRDM16, PRDM2, PREX2, PRF1, PRKACA, PRKACB, PRKAR1A, PRKCB, PRKC1, PRKDC, PRKN, PRPF40B, PRRX1, PRSS8, PSIP1, PTCH1, PTEN, PTGS2, PTK6, PTPN11, PTPN13, PTPN6, PTPRB, PTPRC, PTPRD, PTPRK, PTPRO, PTPRS, PTPRT, PWWP2A, QK1, RAB35, RABEP1, RAC1, RAD17, RAD21, RAD50, RAD51, RAD51B, RAD51C, RAD51D, RAD52, RAD54L, RAF1, RALGDS, RANBP2, RAP1GDS1, RARA, RASA1, RB1, RBM10, RBM15, RECQL4, REL, RELA, RET, RFWD3, RGPD3, RGS7, RHEB, RHOA, RHOH, RICTOR, RIT1, RMI2, RNASEL, RNF2, RNF213, RNF43, ROBO2, ROS1, RPL10, RPL22, RPL5, RPN1, RPS6KA2, RPS6KA4, RPS6KB1, RPS6KB2, RPTOR, RRM1, RSPO2, RSPO3, RUNX1, RUNX1T1, RYBP, S100A7, SALL4, SAMD9, SBDS, SDC4, SDHA, SDHAF2, SDHB, SDHC, SDHD, SEPTIN5, SEPTIN6, SEPTIN9, SET, SETBP1, SETDIB, SETD2, SETDB1, SF3B1, SFPQ, SFRP4, SGK1, SH2B3, SH2DIA, SH3GL1, SHQ1, SHTN1, SIRPA, SIX1, SIX2, SK1, SLC34A2, SLC45A3, SLIT2, SLX4, SMAD2, SMAD3, SMAD4, SMARCA4, SMARCB1, SMARCD1, SMARCE1, SMC1A, SMC3, SMO, SMUG1, SNCAIP, SND1, SNX29, SOCS1, SOX10, SOX11, SOX17, SOX2, SOX21, SOX9, SPECC1, SPEN, SPOP, SPTA1, SRC, SRGAP3, SRSF2, SRSF3, SS18, SS18L1, SSX1, SSX2, SSX4, STAG1, STAG2, STAT3, STAT4, STATSA, STAT5B, STAT6, STIL, STK11, STK36, STK40, STRN, SUFU, SUZ12, SYK, SYNEI, TAF1, TAF15, TAFIL, TAL1, TAL2, TBLIXR1, TBX22, TBX3, TCEA1, TCF12, TCF3, TCF7L1, TCF7L2, TCL1A, TEC, TEK, TENTSC, TERC, TERT, TET1, TET2, TFE3, TFEB, TFG, TFPT, TFRC, TGFBR1, TGFBR2, TGM7, THBS1, THRAP3, TIMP3, TIPARP, TLR4, TLX1, TLX3, TMEM127, TMPRSS2, TNC, TNFAIP3, TNFRSF14, TNFRSF17, TNK2, TOP1, TOP2A, TP53, TP63, TPM3, TPM4, TPR, TRAF2, TRAF7, TRIM24, TRIM27TRIM33, TRIP11, TRRAP, TSCI, TSC2, TSHR, TYRO3, U2AF1, UBR5, UGT1A1, USP44, USP6, USP8, USP9×, VAVI, VEGFA, VHL, VTCN1, VTI1A, WAS, WDCP, WIF1, WNK2, WRN, WT1, WWTR1, XIAP, XPA, XPC, XPO1, XRCC2, YAPI, YES1, YWHAE, ZBTB16, ZBTB2, ZBTB7A, ZCCHC8, ZEB1, ZFHX3, ZMYM2, ZMYM3, ZNF217, ZNF331, ZNF384, ZNF429, ZNF479, ZNF521, ZNF703, ZNRF3, and ZRSR2.

Gene Set 2 is: ACKR3, ACSL3, ACSL6, ACVR2A, ADAMTS20, ADGRB3, ADGRL3, AFDN, AFF1, AFF3, AFF4, AKAP9, ALDH2, ANK1, APOBEC3B, ARHGAP26, ARHGAP5, ARHGEF10, ARHGEF10L, ARHGEF12, ARNT, ASPSCR1, ATF1, ATIC, ATP1A1, ATP2B3, AURKC, BAX, BAZ1A, BCL11A, BCL11B, BCL2L12, BCL3, BCL7A, BCL9, BCL9L, BCLAF1, BIRC2, BIRC5, BIRC6, BLNK, BMP5, BRD3, BUB1B, C15orf65, CACNA1D, CAMTA1, CANT1, CARS1, CASP3, CASP9, CBFA2T3, CBLB, CBLC, CCDC6, CCNB1IP1, CCNC, CCR4, CCR7, CD209, CD28, CDH10, CDH11, CDH17, CDH2, CDH20, CDH5, CDX2, CEP43, CEP89, CHCHD7, CHIC2, CHST11, CIITA, CILK1, CKS1B, CLIP1, CLP1, CLTC, CLTCL1, CMPK1, CNBD1, CNBP, CNOT3, CNTNAP2, CNTRL, COL1A1, COL2A1, COL3A1, COX6C, CPEB3, CRBN, CREB1, CREB3L1, CREB3L2, CRNKL1, CRTC1, CRTC3, CSMD3, CTNNA2, CTNND1, CTNND2, CYP2C19, CYP2C8, CYP2D6, CYSLTR2, DCAF12L2, DCC, DCTN1, DDB2, DDIT3, DDX10, DDX3×, DDX5, DDX6, DEK, DGCR8, DNM2, DROSHA, DST, EBF1, ECT2L, EIF3E, ELF3, ELF4, ELK4, ELL, ELN, EP400, EPAS1, EPHB6, EPS15, ERC1, ETNK1, EXT1, EXT2, EZR, FAM131B, FAM135B, FAM47C, FAT3, FAT4, FBLN2, FBXO11, FCGR2B, FCRL4, FEN1, FES, FEV, FGR, FHIT, FIP1L1, FKBP9, FLNA, FN1, FNBP1, FOXO3, FOXO4, FOXP4, FOXR1, FSTL3, FUS, FZR1, G6PD, GAS7, GDNF, GFRA1, GMPS, GOLGA5, GOPC, GPC3, GPC5, GPHN, GRM8, GUCY1A2, H4C9, HCAR1, HERPUD1, HEY1, HIF1A, HIP1, HLF, HMGA1, HMGA2, HNRNPA2B1, HOOK3, HOXA11, HOXA13, HOXA9, HOXC11, HOXC13, HOXD11, HOXD13, HSP90ABI, IL2, IL21R, IL6ST, ING4, IRS4, ISX, ITGA10, ITGA9, ITGAV, ITGB2, ITGB3, ITK, JAZF1, KAT6B, KAT7, KCNJ5, KDSR, KIAA1549, KLF6, KLK2, KNL1, KNSTRN, KTN1, LARP4B, LASP1, LCK, LCP1, LEF1, LEPROTL1, LHFPL6, LIFR, LMNA, LMO2, LPP, LRIG3, LSM14A, LTF, LYL1, MACC1, MAFB, MAGEA1, MAGI1, MAML2, MAP2K7, MAP3K7, MAPK8, MARK1, MARK4, MB21D2, MBD1, MECOM, MGMT, MLF1, MLLT1, MLLT10, MLLT11, MLLT6, MMP2, MN1, MNX1, MRTFA, MSI2, MSN, MTCP1, MTR, MTRR, MUC1, MUC16, MUC4, MYBL1, MYH11, MYH9, MYO5A, N4BP2, NACA, NBEA, NCKIPSD, NCOA1, NCOA2, NCOA4, NCOR2, NDRG1, NFATC2, NFIB, NFKB1, NFKB2, NFKBIE, NIN, NLRP1, NONO, NR4A3, NTHL1, NUMA1, NUP214, NUP98, NUTM2A, NUTM2B, NUTM2D, OLIG2, OMD, PABPC1, PAFAHIB2, PATZ1, PBX1, PCBP1, PCM1, PDE4DIP, PDGFB, PER1, PGAP3, PICALM, PKHD1, PLAG1, PLCG1, PLEKHG5, PML, POLG, POLQ, POT1, POU2AF1, POU5F1, PPFIBP1, PRCC, PRDM16, PRDM2, PRF1, PRKACA, PRKACB, PRKCB, PRPF40B, PRRX1, PSIP1, PTGS2, PTK6, PTPN13, PTPN6, PTPRB, PTPRC, PTPRK, PWWP2A, RABEP1, RAD17, RALGDS, RAP1GDS1, RBM15, RELA, RFWD3, RGPD3, RGS7, RHOH, RMI2, RNASEL, RNF2, RNF213, ROBO2, RPL10, RPL22, RPL5, RPN1, RPS6KA2, RRM1, RSPO2, RSPO3, S100A7, SALL4, SAMD9, SBDS, SDC4, SEPTIN5, SEPTIN6, SEPTIN9, SET, SETD1B, SETDB1, SFPQ, SFRP4, SH3GL1, SHTN1, SIRPA, SIX1, SIX2, SK1, SLC34A2, SLC45A3, SMARCE1, SMUG1, SND1, SNX29, SOX11, SOX21, SPECC1, SRGAP3, SRSF3, SS18, SS18L1, SSX1, SSX2, SSX4, STAT6, STIL, STK36, STRN, SYNE1, TAF15, TAFIL, TAL1, TAL2, TBLIXR1, TBX22, TCEA1, TCF12, TCF7L1, TCL1A, TEC, TFEB, TFG, TFPT, TGM7, THBS1, THRAP3, TIMP3, TLR4, TLX1, TLX3, TNC, TNFRSF17, TNK2, TPM3, TPM4, TPR, TRIM24, TRIM27, TRIM33, TRIP11, TRRAP, UBR5, USP44, USP6, USP8, USP9×, VAVI, VTI1A, WAS, WDCP, WIF1, WNK2, WRN, WWTR1, XPA, XPC, YWHAE, ZBTB16, ZCCHC8, ZEBI, ZMYM2, ZMYM3, ZNF331, ZNF384, ZNF429, ZNF479, ZNF521, and ZNRF3.

Gene Set 3 is: ABL1, ABL2, ACKR3, ACSL3, ACSL6, ACVR1, ACVR2A, ADAMTS20, ADGRA2, ADGRB3, ADGRL3, AFDN, AFF1, AFF3, AFF4, AKAP9, AKT1, AKT2, AKT3, ALDH2, ALK, AMER1, ANK1, APC, APOBEC3B, AR, ARAF, ARHGAP26, ARHGAP5, ARHGEF10, ARHGEF10L, ARHGEF12, ARID1A, ARID1B, ARID2, ARNT, ASPSCR1, ASXL1, ASXL2, ATF1, ATIC, ATM, ATP1A1, ATP2B3, ATR, ATRX, AURKA, AURKB, AURKC, AXIN1, AXIN2, AXL, B2M, BAPI, BARD1, BAX, BAZ1A, BCL10, BCL11A, BCL11B, BCL2, BCL2L1, BCL2L12, BCL2L2, BCL3, BCL6, BCL7A, BCL9, BCL9L, BCLAF1, BCOR, BCORL1, BCR, BIRC2, BIRC3, BIRC5, BIRC6, BLM, BLNK, BMP5, BMPR1A, BRAF, BRCA1, BRCA2, BRD3, BRD4, BRIPI, BTG1, BTK, BUB1B, C15orf65, CACNA1D, CALR, CAMTA1, CANT1, CARDII, CARS1, CASP3, CASP8, CASP9, CBFA2T3, CBFB, CBL, CBLB, CBLC, CCDC6, CCNB1IP1, CCNC, CCND1, CCND2, CCND3, CCNEI, CCR4, CCR7, CD209, CD274, CD28, CD74, CD79A, CD79B, CDC73, CDH1, CDH10, CDH11, CDH17, CDH2, CDH20, CDH5, CDK12, CDK4, CDK6, CDK8, CDKN1A, CDKN1B, CDKN2A, CDKN2B, CDKN2C, CDX2, CEBPA, CEP43, CEP89, CHCHD7, CHD2, CHD4, CHEK1, CHEK2, CHIC2, CHST11, CIC, CIITA, CILK1, CKS1B, CLIP1, CLPI, CLTC, CLTCL1, CMPK1, CNBD1, CNBP, CNOT3, CNTNAP2, CNTRL, COL1A1, COL2A1, COL3A1, COX6C, CPEB3, CRBN, CREB1, CREB3L1, CREB3L2, CREBBP, CRKL, CRLF2, CRNKL1, CRTC1, CRTC3, CSFIR, CSF3R, CSMD3, CTCF, CTNNA1, CTNNA2, CTNNB1, CTNND1, CTNND2, CUL3, CUX1, CXCR4, CYLD, CYP2C19, CYP2C8, CYP2D6, CYSLTR2, DAXX, DCAF12L2, DCC, DCTN1, DDB2, DDIT3, DDR2, DDX10, DDX3×, DDX5, DDX6, DEK, DGCR8, DICER1, DNAJB1, DNM2, DNMT3A, DPYD, DROSHA, DST, EBF1, ECT2L, EED, EGFR, EIF1AX, EIF3E, EIF4A2, ELF3, ELF4, ELK4, ELL, ELN, EML4, EP300, EP400, EPAS1, EPHA3, EPHA7, EPHB1, EPHB4, EPHB6, EPS15, ERBB2, ERBB3, ERBB4, ERC1, ERCC1, ERCC2, ERCC3, ERCC4, ERCC5, ERG, ESR1, ETNK1, ETS1, ETV1, ETV4, ETV5, ETV6, EWSR1, EXT1, EXT2, EZH2, EZR, FAM131B, FAM135B, FAM47C, FANCA, FANCC, FANCD2, FANCE, FANCF, FANCG, FAS, FAT1, FAT3, FAT4, FBLN2, FBXO11, FBXW7, FCGR2B, FCRL4, FEN1, FES, FEV, FGFR1, FGFR2, FGFR3, FGFR4, FGR, FH, FHIT, FIP1L1, FKBP9, FLCN, FLI1, FLNA, FLT1, FLT3, FLT4, FN1, FNBP1, FOXA1, FOXL2, FOXO1, FOXO3, FOXO4, FOXP1, FOXP4, FOXR1, FSTL3, FUBPI, FUS, FZR1, G6PD, GAS7, GATA1, GATA2, GATA3, GDNF, GFRA1, GLI1, GMPS, GNA11, GNAQ, GNAS, GOLGA5, GOPC, GPC3, GPC5, GPHN, GRIN2A, GRM3, GRM8, GUCY1A2, H3-3A, H3-3B, H3C2, H4C9, HCAR1, HERPUD1, HEY1, HIF1A, HIP1, HLA-A, HLF, HMGA1, HMGA2, HNF1A, HNRNPA2B1, HOOK3, HOXA11, HOXA13, HOXA9, HOXCII, HOXC13, HOXD11, HOXD13, HRAS, HSP90AA1, HSP90AB1, ID3, IDH1, IDH2, IGF1R, IKZF1, IL2, IL21R, IL6ST, IL7R, ING4, IRF4, IRS2, IRS4, ISX, ITGA10, ITGA9, ITGAV, ITGB2, ITGB3, ITK, JAK1, JAK2, JAK3, JAZF1, JUN, KAT6A, KAT6B, KAT7, KCNJ5, KDM5A, KDM5C, KDM6A, KDR, KDSR, KEAP1, KIAA1549, KIF5B, KIT, KLF4, KLF6, KLK2, KMT2A, KMT2C, KMT2D, KNL1, KNSTRN, KRAS, KTN1, LAMP1, LARP4B, LASP1, LATS1, LATS2, LCK, LCP1, LEF1, LEPROTL1, LHFPL6, LIFR, LMNA, LMO1, LMO2, LPP, LRIG3, LRP1B, LSM14A, LTF, LTK, LYL1, LZTR1, MACC1, MAF, MAFB, MAGEA1, MAGI1, MALT1, MAML2, MAP2K1, MAP2K2, MAP2K4, MAP2K7, MAP3K1, MAP3K13, MAP3K7, MAPK1, MAPK8, MARK1, MARK4, MAX, MB21D2, MBD1, MCL1, MDM2, MDM4, MECOM, MED12, MEN1, MET, MGMT, MITF, MLF1, MLH1, MLLT1, MLLT10, MLLT11, MLLT3, MLLT6, MMP2, MN1, MNX1, MPL, MRE11, MRTFA, MSH2, MSH6, MSI2, MSN, MTCP1, MTOR, MTR, MTRR, MUC1, MUC16, MUC4, MUTYH, MYB, MYBL1, MYC, MYCL, MYCN, MYD88, MYH11, MYH9, MYOSA, MYOD1, N4BP2, NAB2, NACA, NBEA, NBN, NCKIPSD, NCOA1, NCOA2, NCOA4, NCOR1, NCOR2, NDRG1, NF1, NF2, NFATC2, NFE2L2, NFIB, NFKB1, NFKB2, NFKBIE, NIN, NKX2-1, NLRP1, NONO, NOTCH1, NOTCH2, NOTCH4, NPMI, NR4A3, NRAS, NRG1, NSD1, NSD2, NSD3, NT5C2, NTHL1, NTRK1, NTRK2, NTRK3, NUMA1, NUP214, NUP98, NUTM1, NUTM2A, NUTM2B, NUTM2D, OLIG2, OMD, P2RY8, PABPC1, PAFAHIB2, PAK3, PALB2, PARP1, PATZ1, PAX3, PAX5, PAX7, PAX8, PBRM1, PBX1, PCBP1, PCM1, PDCD1LG2, PDE4DIP, PDGFB, PDGFRA, PDGFRB, PER1, PGAP3, PHF6, PHOX2B, PICALM, PIK3C2B, PIK3CA, PIK3CB, PIK3CD, PIK3CG, PIK3R1, PIK3R2, PIM1, PKHD1, PLAG1, PLCG1, PLEKHG5, PML, PMS1, PMS2, POLD1, POLE, POLG, POLQ, POT1, POU2AF1, POU5F1, PPARG, PPFIBP1, PPM1D, PPP2R1A, PPP6C, PRCC, PRDM1, PRDM16, PRDM2, PREX2, PRF1, PRKACA, PRKACB, PRKAR1A, PRKCB, PRKDC, PRPF40B, PRRX1, PSIP1, PTCH1, PTEN, PTGS2, PTK6, PTPN11, PTPN13, PTPN6, PTPRB, PTPRC, PTPRD, PTPRK, PTPRT, PWWP2A, QK1, RABEP1, RAC1, RAD17, RAD21, RAD50, RAD51B, RAF1, RALGDS, RANBP2, RAP1GDS1, RARA, RB1, RBM10, RBM15, RECQL4, REL, RELA, RET, RFWD3, RGPD3, RGS7, RHOA, RHOH, RIT1, RMI2, RNASEL, RNF2, RNF213, RNF43, ROBO2, ROS1, RPL10, RPL22, RPL5, RPN1, RPS6KA2, RRM1, RSPO2, RSPO3, RUNX1, RUNX1T1, S100A7, SALL4, SAMD9, SBDS, SDC4, SDHA, SDHAF2, SDHB, SDHC, SDHD, SEPTIN5, SEPTIN6, SEPTIN9, SET, SETBP1, SETD1B, SETD2, SETDB1, SF3B1, SFPQ, SFRP4, SGK1, SH2B3, SH2D1A, SH3GL1, SHTN1, SIRPA, SIX1, SIX2, SK1, SLC34A2, SLC45A3, SMAD2, SMAD3, SMAD4, SMARCA4, SMARCBI, SMARCD1, SMARCE1, SMC1A, SMO, SMUG1, SND1, SNX29, SOCS1, SOX11, SOX2, SOX21, SPECC1, SPEN, SPOP, SRC, SRGAP3, SRSF2, SRSF3, SS18, SS18L1, SSX1, SSX2, SSX4, STAG1, STAG2, STAT3, STAT5B, STAT6, STIL, STK11, STK36, STRN, SUFU, SUZ12, SYK, SYNEI, TAF1, TAF15, TAFIL, TAL1, TAL2, TBLIXR1, TBX22, TBX3, TCEA1, TCF12, TCF3, TCF7L1, TCF7L2, TCL1A, TEC, TENTSC, TERT, TET1, TET2, TFE3, TFEB, TFG, TFPT, TFRC, TGFBR2, TGM7, THBS1, THRAP3, TIMP3, TLR4, TLX1, TLX3, TMEM127, TMPRSS2, TNC, TNFAIP3, TNFRSF14, TNFRSF17, TNK2, TOP1, TP53, TP63, TPM3, TPM4, TPR, TRAF7, TRIM24, TRIM27, TRIM33, TRIP11, TRRAP, TSC1, TSC2, TSHR, U2AF1, UBR5, UGT1A1, USP44, USP6, USP8, USP9×, VAVI, VHL, VTI1A, WAS, WDCP, WIF1, WNK2, WRN, WT1, WWTR1, XPA, XPC, XPO1, XRCC2, YWHAE, ZBTB16, ZCCHC8, ZEB1, ZFHX3, ZMYM2, ZMYM3, ZNF331, ZNF384, ZNF429, ZNF479, ZNF521, ZNRF3, and ZRSR2.

Gene Set 4 is: ABL1, ABL2, ABRAXAS1, ACVR1, ACVR1B, ADGRA2, AKT1, AKT2, AKT3, ALK, ALOX12B, AMER1, ANKRD11, APC, AR, ARAF, ARFRP1, ARID1A, ARID1B, ARID2, ARID5B, ASXL1, ASXL2, ATM, ATR, ATRX, AURKA, AURKB, AXIN1, AXIN2, AXL, B2M, BAP1, BARD1, BBC3, BCL10, BCL2, BCL2L1, BCL2L11, BCL2L2, BCL6, BCOR, BCORL1, BCR, BIRC3, BLM, BMPR1A, BRAF, BRCA1, BRCA2, BRD4, BRIP1, BTG1, BTK, CALR, CARD11, CASP8, CBFB, CBL, CCN6, CCND1, CCND2, CCND3, CCNEI, CD274, CD276, CD74, CD79A, CD79B, CDC73, CDH1, CDK12, CDK4, CDK6, CDK8, CDKN1A, CDKN1B, CDKN2A, CDKN2B, CDKN2C, CEBPA, CENPA, CHD2, CHD4, CHEK1, CHEK2, CIC, COP1, CREBBP, CRKL, CRLF2, CSFIR, CSF3R, CTCF, CTLA4, CTNNA1, CTNNB1, CUL3, CXCR4, CYLD, DAXX, DCUN1D1, DDR2, DICER1, DIS3, DNAJB1, DNMT1, DNMT3A, DNMT3B, DOT1L, E2F3, EED, EGFL7, EGFR, EIF1AX, EIF4A2, EIF4E, ELOC, EML4, EMSY, EP300, EPCAM, EPHA3, EPHA5, EPHA7, EPHBI, ERBB2, ERBB3, ERBB4, ERCC1, ERCC2, ERCC3, ERCC4, ERCC5, ERG, ERRFI1, ESR1, ETS1, ETV1, ETV4, ETV5, ETV6, EWSR1, EZH2, FANCA, FANCC, FANCD2, FANCE, FANCF, FANCG, FANC1, FANCL, FAS, FAT1, FBXW7, FGF1, FGF10, FGF14, FGF19, FGF2, FGF23, FGF3, FGF4, FGF5, FGF6, FGF7, FGF8, FGF9, FGFR1, FGFR2, FGFR3, FGFR4, FH, FLCN, FLI1, FLT1, FLT3, FLT4, FOXA1, FOXL2, FOXO1, FOXP1, FRS2, FUBP1, FYN, GABRA6, GATA1, GATA2, GATA3, GATA4, GATA6, GEN1, GID4, GLI1, GNA11, GNA13, GNAQ, GNAS, GPS2, GREM1, GRIN2A, GRM3, GSK3B, H1-2, H2BC5, H3-3A, H3-3B, H3-4, H3-5, H3C1, H3C10, H3C11, H3C12, H3C13, H3C14, H3C2, H3C3, H3C4, H3C6, H3C7, H3C8, HGF, HLA-A, HNF1A, HOXB13, HRAS, HSD3B1, HSP90AA1, ICOSLG, ID3, IDH1, IDH2, IFNGR1, IGF1, IGF1R, IGF2, IKBKE, IKZF1, IL10, IL7R, INHA, INHBA, INPP4A, INPP4B, INSR, IRF2, IRF4, IRS1, IRS2, JAK1, JAK2, JAK3, JUN, KAT6A, KDM5A, KDM5C, KDM6A, KDR, KEAP1, KEL, KIF5B, KIT, KLF4, KLHL6, KMT2A, KMT2B, KMT2C, KMT2D, KRAS, LAMP1, LATS1, LATS2, LMOI, LRP1B, LYN, LZTR1, MAGI2, MALT1, MAP2K1, MAP2K2, MAP2K4, MAP3K1, MAP3K13, MAP3K14, MAP3K4, MAPK1, MAPK3, MAX, MCL1, MDC1, MDM2, MDM4, MED12, MEF2B, MEN1, MET, MGA, MITF, MLH1, MLLT3, MPL, MRE11, MSH2, MSH3, MSH6, MST1, MST1R, MTOR, MUTYH, MYB, MYC, MYCL, MYCN, MYD88, MYOD1, NAB2, NBN, NCOA3, NCOR1, NEGR1, NF1, NF2, NFE2L2, NFKB1A, NKX2-1, NKX3-1, NOTCH1, NOTCH2, NOTCH3, NOTCH4, NPMI, NRAS, NRG1, NSD1, NTRK1, NTRK2, NTRK3, NUP93, NUTM1, PAK1, PAK3, PAK5, PALB2, PARP1, PAX3, PAX5, PAX7, PAX8, PBRM1, PDCD1, PDCD1LG2, PDGFRA, PDGFRB, PDK1, PDPK1, PGR, PHOX2B, PIK3C2B, PIK3C2G, PIK3C3, PIK3CA, PIK3CB, PIK3CD, PIK3CG, PIK3R1, PIK3R2, PIK3R3, PIM1, PLCG2, PLK2, PMAIP1, PMS1, PMS2, PNRC1, POLD1, POLE, PPARG, PPM1D, PPP2R1A, PPP2R2A, PPP6C, PRDM1, PREX2, PRKAR1A, PRKC1, PRKDC, PRKN, PRSS8, PTCH1, PTEN, PTPN11, PTPRD, PTPRS, PTPRT, QK1, RAB35, RAC1, RAD21, RAD50, RAD51, RAD51B, RAD51C, RAD51D, RAD52, RAD54L, RAF1, RANBP2, RARA, RASA1, RB1, RBM10, RECQL4, REL, RET, RHEB, RHOA, RICTOR, RIT1, RNF43, ROS1, RPS6KA4, RPS6KB1, RPS6KB2, RPTOR, RUNX1, RUNX1T1, RYBP, SDHA, SDHAF2, SDHB, SDHC, SDHD, SETD2, SF3B1, SH2B3, SH2D1A, SHQ1, SLIT2, SLX4, SMAD2, SMAD3, SMAD4, SMARCA4, SMARCB1, SMARCD1, SMO, SNCAIP, SOCS1, SOX10, SOX17, SOX2, SOX9, SPEN, SPOP, SPTA1, SRC, SRSF2, STAG2, STAT3, STAT4, STAT5A, STAT5B, STK11, STK40, SUFU, SUZ12, SYK, TAF1, TBX3, TCF3, TCF7L2, TENT5C, TERC, TERT, TET1, TET2, TFE3, TFRC, TGFBR1, TGFBR2, TMEM127, TMPRSS2, TNFAIP3, TNFRSF14, TOP1, TOP2A, TP53, TP63, TRAF2, TRAF7, TSC1, TSC2, TSHR, U2AF1, VEGFA, VHL, VTCN1, WT1, XIAP, XPO1, XRCC2, YAP1, YES1, ZBTB2, ZFHX3, ZNF217, ZNF703, and ZRSR2.

The classifier gene(s) in some embodiments comprise one or more genes tested in commercially available gene panel assays, such as, for example, the GUARDANT360® CDx assay from Guardant Health (Palo Alto, CA), the Spotlight 59 oncology panel from Fluxion Biosciences (Alameda, CA), the UltraSEEK lung cancer panel from Agena Bioscience (San Diego, CA), the FoundationACT liquid biopsy assay from Foundation Medicine (Beverly, MA), the PlasmaSELECT assay from Personal Genome Diagnostics (Baltimore, MD), the TruSight Oncology 500 ctDNA assay from Illumina (San Diego, CA), the FOUNDATION ONE® Liquid CDx assay from Foundation Medicine, the Galleri assay from GRAIL (Menlo Park, CA), and the Tempus xT and xF tests from Tempus (Chicago IL). These panels can be used for other steps described herein, including select steps in isolation and sequencing.

Some embodiments of the invention comprise steps of isolating and/or sequencing the classifier cfDNA. The steps of isolating and/or sequencing the cfDNA can comprise isolating and/or sequencing at least the classifier cfDNA but may also comprise isolating and/or sequencing at least some non-classifier cfDNA.

The methods of isolating the classifier cfDNA can comprise isolating cfDNA corresponding to one or more target regions of the genome. The target regions preferably comprise at least the classifier regions but may also comprise non-classifier regions. Target regions are distinguished from non-target regions, the latter of which are regions that are not target regions. The isolating can comprise using capture nucleic acid probes having hybridization sequences corresponding to the target regions to hybridize to cfDNA from a subject. The hybridized constructs can then be isolated from non-hybridized cfDNA and other elements to thereby “fish out” or “pull down” the desired cfDNA. Methods of isolating targeted cfDNA is known in the art. See, e.g., US 2019/0287645 A1, US 2022/0259647 A1, and US 2022/0090207 A1, which are incorporated herein by reference in their entireties.

The isolated cfDNA can then be sequenced. The sequencing can be performed using a first-generation sequencing method, such as Maxam-Gilbert or Sanger sequencing, or a high-throughput sequencing (e.g., next-generation sequencing or NGS) method. A high-throughput sequencing method may sequence simultaneously (or substantially simultaneously) at least 10,000, 100,000, 1 million, 10 million, 100 million, 1 billion, or more polynucleotide molecules. Sequencing methods may include, but are not limited to: pyrosequencing, sequencing-by-synthesis, single-molecule sequencing, nanopore sequencing, semiconductor sequencing, sequencing-by-ligation, sequencing-by-hybridization, Digital Gene Expression (Helicos), massively parallel sequencing, e.g., Helicos, Clonal Single Molecule Array (Solexa/Illumina), sequencing using PacBio, SOLID, Ion Torrent, or Nanopore platforms.

The target regions can comprise genic regions of the genome, intergenic regions of the genome, or a combination thereof. In some embodiments, the target regions comprise genes or specific parts thereof (e.g, exons, introns, promoters, coding regions, untranslated regions (5′ untranslated region, 3′ untranslated region, etc.)). A gene comprising at least one base of a target region is referred to herein as a “target gene.” The target genes preferably comprise at least the classifier gene(s) but may also comprise one or more non-classifier gene(s). Target genes are distinguished from non-target genes, the latter of which are genes that are not target genes. In some embodiments, the target regions comprise exons. Exons comprising at least one base of a target region are referred to herein as “target exons.” Target exons are distinguished from non-target exons, the latter of which are exons that are not target exons. In some embodiments, the target exons comprise particular exons, such as first exons.

In some embodiments, the target regions comprise at least a portion of at least one exon of at least one target gene. In some embodiments, the target regions comprise at least a portion of the coding sequence of at least one exon of at least one target gene. In some embodiments, the target regions comprise at least a portion of the first exon of at least one target gene. In some embodiments, the target regions comprise at least a portion of the coding sequence of the first exon of at least one target gene. In some embodiments, the target regions comprise the entirety of at least one exon of at least one target gene. In some embodiments, the target regions comprise the entirety of the coding sequence of at least one exon of at least one target gene. In some embodiments, the target regions comprise the entirety of the first exon of at least one target gene. In some embodiments, the target regions comprise the entirety of the coding sequence of the first exon of at least one target gene. In some embodiments, the target regions comprise the entirety of at least one exon of each target gene. In some embodiments, the target regions comprise the entirety of the coding sequence of at least one exon of each target gene. In some embodiments, the target regions comprise, consist, or consist essentially of the entirety of the first exon of each target gene. In some embodiments, the target regions comprise, consist, or consist essentially of the entirety of the coding sequence of the first exon of each target gene. Accordingly, the target cfDNA of the invention can correspond to any of the above-described target regions.

In some embodiments, the non-target regions comprise intergenic regions of the genome. In some embodiments, the non-target regions comprise at least one intron in a genome. In some embodiments, the non-target regions comprise all introns in a genome. In some embodiments, the non-target regions comprise at least one intron of at least one target gene. In some embodiments, the non-target regions comprise at least one intron of each target gene. In some embodiments, the non-target regions comprise each intron of each target gene. In some embodiments, the non-target regions comprise at least one exon in at least one target gene. In some embodiments, the non-target regions comprise at least one exon in each target gene. In some embodiments, the non-target regions comprise at least one exon other than the first exon in at least one target gene. In some embodiments, the non-target regions comprise at least one exon other than the first exon in each target gene. In some embodiments, the non-target regions comprise each exon other than the first exon in at least one target gene. In some embodiments, the non-target regions comprise each exon other than the first exon in each target gene. Accordingly, the isolated and/or sequenced cfDNA of the invention can exclude cfDNA corresponding to any of the above-described non-target regions.

In various embodiments, the target regions constitute less than 2,999 Mb, less than 2,750 Mb, less than 2,500 Mb, less than 2,250 Mb, less than 2,000 Mb, than 1,750 Mb, less than 1,500 Mb, less than 1,250 Mb, less than 1,000 Mb, than 750 Mb, less than 500 Mb, less than 250 Mb, less than 200 Mb, less than 150 Mb, less than 100 Mb, less than 50 Mb, less than 25 Mb, less than 10 Mb, or less than 5 Mb of a reference genome or a genome of the subject. Accordingly, the isolated and/or sequenced cfDNA of the invention can correspond to any of the above-referenced portions of the genome.

In various embodiments, the target genes in total constitute less than 2,999 Mb, less than 2,750 Mb, less than 2,500 Mb, less than 2,250 Mb, less than 2,000 Mb, than 1,750 Mb, less than 1,500 Mb, less than 1,250 Mb, less than 1,000 Mb, than 750 Mb, less than 500 Mb, less than 250 Mb, less than 200 Mb, less than 150 Mb, less than 100 Mb, less than 50 Mb, less than 25 Mb, less than 10 Mb, or less than 5 Mb of a reference genome or a genome of the subject. Accordingly, the isolated and/or sequenced cfDNA of the invention can correspond to target genes constituting any of the above-referenced portions of the genome.

In various embodiments, the number of target genes is at least 1, at least 5, at least 25, at least 50, at least 75, at least 100, at least 125, at least 150, at least 175, at least 200, at least 250, at least 250, at least 275, at least 300, at least 325, at least 350, at least 375, at least 400, at least 450, at least 475, or at least 500. In various embodiments, the number of target genes is no more than 25,000, no more than 20,000, no more than 15,000, no more than 10,000, no more than 5,000, no more than 2,500, no more than 2,000, no more than 1,750, no more than 1,500, no more than 1,250, or no more than 1,000.

In preferred embodiments, the target genes comprise, consist, or consist essentially of cancer genes. In some embodiments, the target genes comprise, consist, or consist essentially of one, some, or all of the genes in Gene Set 1. In some embodiments, the target genes comprise, consist, or consist essentially of one, some, or all of the genes in Gene Set 2. In some embodiments, the target genes comprise, consist, or consist essentially of one, some, or all of the genes in Gene Set 3. In some embodiments, the target genes comprise, consist, or consist essentially of one, some, or all of the genes in Gene Set 4. In various embodiments, the target genes comprise, consist, or consist essentially of at least 1, at least 5, at least 25, at least 50, at least 75, at least 100, at least 125, at least 150, at least 175, at least 200, at least 250, at least 250, at least 275, at least 300, at least 325, at least 350, at least 375, at least 400, at least 450, at least 475, or at least 500 of the genes in any of Gene Set 1, Gene Set 2, Gene Set 3, or Gene Set 4.

In some embodiments, the classifier cfDNA is sequenced at a deduplicated sequencing depth of at least 1×, at least 5×, at least 10×, at least 15×, at least 20×, at least 25×, at least 30×, at least 35×, at least 40×, at least 45×, at least 50×, at least 75×, at least 100×, at least 125×, at least 150×, at least 175×, at least 200×, at least 225×, at least 250×, at least 275×, at least 300×, at least 325×, at least 350×, at least 375×, at least 400×, at least 425×, at least 450×, at least 475×, at least 500×, at least 525×, at least 550×, at least 575×, at least 600×, at least 1,000×, at least 2,000×, at least 3,000×, at least 4,000×, at least 5,000×, at least 10,000×, at least 25,000×, or at least 50,000×. In some embodiments, the classifier cfDNA is sequenced at a deduplicated sequencing depth of no more than 25×, no more than 50×, no more than 100×, no more than 200×, no more than 300×, no more than 400×, no more than 500×, no more than 600×, no more than 700×, no more than 800×, no more than 900×, no more than 1,000×, no more than 2,000×, no more than 3,000×, no more than 4,000×, no more than 5,000×, no more than 10,000×, no more than 25,000×, no more than 50,000×, no more than 75,000×, or no more than 100,000×. In some embodiments, cfDNA corresponding to the target regions is sequenced at a deduplicated sequencing depth of at least 1×, at least 5×, at least 10×, at least 15×, at least 20×, at least 25×, at least 30×, at least 35×, at least 40×, at least 45×, at least 50×, at least 75×, at least 100×, at least 125×, at least 150×, at least 175×, at least 200×, at least 225×, at least 250×, at least 275×, at least 300×, at least 325×, at least 350×, at least 375×, at least 400×, at least 425×, at least 450×, at least 475×, at least 500×, at least 525×, at least 550×, at least 575×, at least 600×, at least 1,000×, at least 2,000×, at least 3,000×, at least 4,000×, at least 5,000×, at least 10,000×, at least 25,000×, or at least 50,000×. In some embodiments, the cfDNA corresponding to the target regions is sequenced at a deduplicated sequencing depth of no more than 25×, no more than 50×, no more than 100×, no more than 200×, no more than 300×, no more than 400×, no more than 500×, no more than 600×, no more than 700×, no more than 800×, no more than 900×, no more than 1,000×, no more than 2,000×, no more than 3,000×, no more than 4,000×, no more than 5,000×, no more than 10,000×, no more than 25,000×, no more than 50,000×, no more than 75,000×, or no more than 100,000×.

The term “deduplicated sequencing depth” as used herein refers to the total number of sequenced bases among all the sequenced classifier cfDNA molecules divided by the total number of bases in the defined classifier regions (e.g., the coding regions of the first exons of the classifier genes). The total number of sequenced bases among all the sequenced classifier cfDNA molecules in some versions can be determined by deduplicating raw sequence reads (the output of a DNA sequencer), e.g., by generating a “consensus” read for each sequenced cfDNA using start-stop position and/or unique molecular identifiers and/or any other methods to generate consensus reads, and multiplying the number of deduplicated sequence reads by the average read length. Other methods can be used. The total number of bases in the defined classifier regions can be determined by counting the number of bases in the defined classifier regions. If the entire genome is defined as the classifier region, the total number of bases in the defined classifier region will be the length of the genome (˜3.2 billion for exemplary reference genomes). If subregions of the genome are defined as the classifier regions (e.g., coding sequences of first exons of select cancer genes, as outlined in the following examples), the total number of bases in the defined classifier region will be much smaller (for example, 2.4 Mbp as covered in the custom panel of the following examples).

Consensus sequences are sequences derived from redundant sequences of a parent molecule intended to represent the sequence of the original parent molecule. Consensus sequences can be produced by voting (wherein each majority nucleotide, e.g., the most commonly observed nucleotide at a given base position, among the sequences is the consensus nucleotide) or other approaches such as comparing to a reference genome. Consensus sequences can be produced by tagging original parent molecules with unique or non-unique molecular tags, which allow tracking of the progeny sequences (e.g., after amplification) by tracking of the tag and/or use of sequence read internal information. Examples of tagging or barcoding, and uses of tags or barcodes, are provided in, for example, U.S. Patent Pub. Nos. 2015/0368708, 2015/0299812, 2016/0040229, and 2016/0046986, which are entirely incorporated herein by reference.

cfDNA from a subject may be obtained by isolating a biological sample comprising the cfDNA from the subject. The term “biological sample,” as used herein, generally refers to a tissue or fluid sample derived from a subject. A biological sample may be directly obtained from the subject. A biological sample may optionally be processed before being used in downstream steps described herein. The biological sample can be derived from any organ, tissue or biological fluid. A biological sample can comprise, for example, a bodily fluid or a solid tissue sample. An example of a solid tissue sample is a tumor sample, e.g., from a solid tumor biopsy. Bodily fluids include, for example, blood, serum, plasma, tumor cells, saliva, urine, lymphatic fluid, prostatic fluid, seminal fluid, milk, sputum, stool, tears, and derivatives of these. Preferred samples are samples derived from bodily fluids.

The methods of the invention can comprise a step of determining fragmentation patterns of the classifier cfDNA. Fragmentations patterns of cfDNA can include any quantifiable fragmentation characteristic of the cfDNA. Nonlimiting examples of such characteristics include the length of cfDNA fragments that align with one or more regions of a genome, a number of cfDNA fragments that align with one or more regions of a genome, a number of cfDNA fragments that start or end at each of one or more regions of a genome, a number of cfDNA fragments outside a nucleosome region, a number of cfDNA fragments within a nucleosome region, a size peak distribution of cfDNA fragments relative to a mappable genomic location, a particular location of a size peak of cfDNA fragments, a particular range of cfDNA fragment sizes associated with a size peak, or any combination thereof. Exemplary methods of determining such characteristics are described in further detail below or are otherwise known in the art.

An exemplary fragmentation pattern that can be used for analysis and classification is a fragment size distribution. “Fragment size distribution” as used herein with respect to cfDNA refers to a quantitation of the number of cfDNAs within each of one or more different size intervals. The quantitation can be an absolute or relative quantitation. The size of the cfDNA is the length of the cfDNA, and each size interval can be a single value (a single length) or range of values (a range of lengths). In some embodiments, a single fragment size distribution is determined for all the classifier cfDNAs. In some embodiments separate fragment size distributions are determined for different subsets of the cfDNAs. Separate size distributions, for example, can be determined for cfDNAs corresponding to any of the various classifier regions described herein. In some embodiments, separate fragment size distributions are determined for the classifier cfDNA corresponding to at least some of the classifier genes. In some embodiments, a separate fragment size distribution is determined for the classifier cfDNA corresponding to each classifier gene. In some embodiments, separate fragment size distributions are determined for the classifier cfDNA corresponding to at least some of the classifier exons. In some embodiments, a separate fragment size distribution is determined for the classifier cfDNA corresponding to each classifier exon. In some embodiments, separate fragment size distributions are determined for the classifier cfDNA corresponding to at least some of the first exons of at least some of the classifier genes. In some embodiments, a separate fragment size distribution is determined for the classifier cfDNA corresponding to the first exon of each classifier gene. In some versions, at least the portion of the at least one exon of the at least one classifier gene comprises one or more predefined exon regions. Exemplary predefined exon regions comprise transcription factor binding sites, regions of open chromatin, and specific motifs. Other predefined exon regions can be used.

For downstream classification, the fragmentation size distributions can be quantitated. “Quantitate” (and grammatical variants thereof) in this context refers to characterizing the fragmentation size distributions with a quantitative value. The quantitative value can be an absolute or relative value and can be, without limitation, a number, a statistical value (e.g., frequency, mean, median, standard deviation, or quantile), or a degree or a relative quantity (e.g., high, medium, and low). A quantitative value can be a ratio of two quantitative values. A quantitative value can be a linear combination of quantitative values. A quantitative value may be a normalized value. Any of a number of distribution quantitations can be used. These include but are not limited to quantitation of entropy, sum, minimum, maximum, interquartile range, mean, median, mode, variance, standard deviation, kurtosis, diversity, depth of sequencing, bins, and/or Kolmogorov-Smirnov statistic. The DNA sequence motifs present in fragments can also inform the fragmentation patterns. In some versions, the determining the fragmentation patterns comprises determining a motif diversity score. In some versions, the determining the fragmentation patterns comprises determining the fragmentation patterns of one or more predefined exon regions. In some versions, the predefined exon regions are selected from the group consisting of transcription factor binding sites, regions of open chromatin, and specific motifs. In some versions, the determining the fragmentation patterns comprises determining a separate fragment size distribution of the classifier cfDNA corresponding to each predefined exon region.

Exemplary versions of the invention employ an entropy quantitation (Roach TNF. Use and Abuse of Entropy in Biology: A Case for Caliber. Entropy (Basel). 2020 Nov 25;22(12):1335). An exemplary entropy quantitation is Shannon entropy quantitation (Shannon, Claude E. (July 1948). “A Mathematical Theory of Communication”. Bell System Technical Journal. 27 (3): 379-423) (Shannon, Claude E. (October 1948). “A Mathematical Theory of Communication”. Bell System Technical Journal. 27 (4): 623-656). Other suitable entropy quantitations include Rényi entropy (Rényi, Alfréd (1961). “On measures of information and entropy” (PDF). Proceedings of the fourth Berkeley Symposium on Mathematics, Statistics and Probability 1960. pp. 547-561) and Tsallis entropy (Tsallis, C. (1988). “Possible generalization of Boltzmann-Gibbs statistics”. Journal of Statistical Physics. 52 (1-2): 47-487), among others.

Exemplary versions of the invention employ depth of sequencing, as well as quantitation of the fragment size distribution by measuring the number of fragments that fall into various fragment size bins. Exemplary versions of the invention employ motif diversity scores (Jiang P, Sun K, Peng W et al. Plasma DNA End-Motif Profiling as a Fragmentomic Marker in Cancer, Pregnancy, and Transplantation. Cancer Discov 2020; 10 (5): 664-673). Once the fragmentation patterns of the classifier cfDNAs are determined, the fragmentation patterns can be used to determine a particular disease state of the subject from which the cfDNAs are derived. The fragmentation patterns, for example, can be classified to identify the subject as being negative or positive for cancer or being negative or positive for a particular type of cancer. “Type of cancer” (or “cancer type”) as used herein generally refers to a cancer having a particular characteristic that is distinct from other cancers, such as a particular tissue of origin, etiological characteristic, phenotypic characteristic, genotypic characteristic, anatomical characteristic, physiological characteristic, clinical characteristic, and/or treatment-response characteristic. The term “tissue of origin” as used herein refers to the organ, organ group, body region, or cell type that a cancer arises or originates from. The identification of a tissue of origin typically allows for identification of the most appropriate next steps in the care continuum of cancer to further diagnose, stage, and decide on treatment. The term “subtype of cancer” (or “cancer subtype”) generally refers to a cancer of a particular cancer type having a particular characteristic that distinguishes it from another cancer of the particular cancer type. An example of a cancer subtype is a cancer from a particular tissue of origin that has an etiological, phenotypic, genotypic, anatomical, physiological characteristic, clinical characteristic, and/or treatment-response characteristic that differs from another cancer of the particular tissue of origin. The identification of a cancer type or subtype typically allows for identification of the most appropriate next steps in the care continuum of cancer to further diagnose, stage, and decide on treatment. The identification of a subject as being negative or positive for cancer or a particular type or subtype thereof will typically occur by determining a probability or numerical score from the fragmentation patterns and classifying the subject based on certain thresholds thereof.

Various exemplary types of cancer include acute lymphoblastic leukemia (ALL), acute myeloid leukemia (AML), adrenocortical carcinoma, AIDS-related cancers (Kaposi sarcoma (soft tissue sarcoma), AIDS-related lymphoma, primary CNS lymphoma), anal cancer, appendix cancer, astrocytomas (a type of brain cancer), atypical teratoid/rhabdoid tumor (a type of brain cancer), basal cell carcinoma of the skin (see skin cancer), bile duct cancer, bladder cancer, bone cancer (includes Ewing sarcoma, osteosarcoma, and malignant fibrous histiocytoma), brain cancer, breast cancer, bronchial tumors (lung cancer), Burkitt lymphoma (non-Hodgkin lymphoma), carcinoid tumor (type of gastrointestinal caner), central nervous system cancer (atypical teratoid/rhabdoid tumor (brain cancer), medulloblastoma and other CNS embryonal tumors (brain cancer), germ cell tumor (brain cancer)), primary CNS lymphoma, cervical cancer, cholangiocarcinoma (bile duct cancer), chordoma (bone cancer), chronic lymphocytic leukemia (CLL), chronic myelogenous leukemia (CML), chronic myeloproliferative neoplasms, colorectal cancer, craniopharyngioma (brain cancer), cutaneous T-cell lymphoma (mycosis fungoides and Sézary syndrome), ductal carcinoma In Situ (DCIS) (type of breast cancer), endometrial cancer (uterine cancer), ependymoma (brain cancer), esophageal cancer, esthesioneuroblastoma (head and neck cancer), Ewing sarcoma (bone cancer), extracranial germ cell tumor, extragonadal germ cell tumor, eye cancer (intraocular melanoma, retinoblastoma, fallopian tube cancer, gallbladder cancer, gastric cancer (stomach cancer), gastrointestinal carcinoid tumor, gastrointestinal stromal tumors (GIST) (soft tissue sarcoma), germ cell tumors (childhood central nervous system germ cell tumors (brain cancer), childhood extracranial germ cell tumors, extragonadal germ cell tumors, ovarian germ cell tumors, testicular cancer), gestational trophoblastic disease, hairy cell leukemia, head and neck cancer, heart tumors, hepatocellular cancer (liver cancer), histiocytosis, (langerhans cell), Hodgkin lymphoma, hypopharyngeal cancer (head and neck cancer), intraocular melanoma, islet cell tumors (pancreatic neuroendocrine tumors, Kaposi sarcoma (soft tissue sarcoma), kidney (renal cell) cancer, Langerhans cell histiocytosis, laryngeal cancer (head and neck cancer), leukemia, lip and oral cavity cancer (head and neck cancer), liver cancer, lung cancer (non-small cell, small cell, pleuropulmonary blastoma, pulmonary inflammatory myofibroblastic tumor, and tracheobronchial tumor), lymphoma, male breast cancer, melanoma, intraocular melanoma (eye cancer), Merkel cell carcinoma (skin cancer), mesothelioma, metastatic cancer, metastatic squamous neck cancer with occult primary (head and neck cancer), midline tract carcinoma with NUT gene changes, mouth cancer (head and neck cancer), multiple endocrine neoplasia syndromes, multiple myeloma/plasma cell neoplasms, mycosis fungoides (lymphoma), myelodysplastic syndromes, myelodysplastic/myeloproliferative neoplasms, myelogenous leukemia, chronic (CML), acute myeloid leukemia (AML), myeloproliferative neoplasms, nasal cavity and paranasal sinus cancer (head and neck cancer), nasopharyngeal cancer (head and neck cancer), neuroblastoma, non-Hodgkin lymphoma, non-small cell lung cancer, oral cancer, lip and oral cavity cancer (head and neck cancer), oropharyngeal cancer (head and neck cancer), osteosarcoma, undifferentiated pleomorphic sarcoma of bone treatment, ovarian cancer, pancreatic cancer, pancreatic neuroendocrine tumors (islet cell tumors), papillomatosis (childhood laryngeal), paraganglioma, paranasal sinus cancer (head and neck cancer), nasal cavity cancer (head and neck cancer), parathyroid cancer, penile cancer, pharyngeal cancer, (head and neck cancer), pheochromocytoma, pituitary tumor, plasma cell neoplasm/multiple myeloma, pleuropulmonary blastoma (lung cancer), pregnancy and breast cancer, primary central nervous system (CNS) lymphoma, primary peritoneal cancer, prostate cancer (such as metastatic neuroendocrine prostate cancer), pulmonary inflammatory myofibroblastic tumor (lung cancer), rectal cancer, recurrent cancer, renal cell (kidney) cancer, retinoblastoma, rhabdomyosarcoma, salivary gland cancer (head and neck cancer), sarcoma (childhood rhabdomyosarcoma (soft tissue sarcoma), childhood vascular tumors (soft tissue sarcoma), Ewing sarcoma (bone cancer), Kaposi sarcoma (soft tissue sarcoma), osteosarcoma (bone cancer), soft tissue sarcoma, uterine sarcoma), Sézary syndrome (lymphoma), skin cancer, small cell lung cancer, small intestine cancer, soft tissue sarcoma, squamous cell carcinoma of the skin (skin cancer), squamous neck cancer with occult primary (head and neck cancer), stomach (gastric) cancer, T-cell lymphoma (mycosis fungoides and Sèzary syndrome), testicular cancer, throat cancer (head and neck cancer), nasopharyngeal cancer, oropharyngeal cancer, hypopharyngeal cancer, thymoma and thymic carcinoma, thyroid cancer, tracheobronchial tumors (lung cancer), transitional cell cancer of the renal pelvis and ureter (kidney (renal cell) cancer), ureter cancer, renal pelvis cancer, transitional cell cancer (kidney (renal cell) cancer), urethral cancer, uterine cancer, uterine sarcoma, vaginal cancer, vascular tumors (soft tissue sarcoma), and vulvar cancer. In some embodiments, classifying the fragmentation patterns identifies the subject as being positive or negative for one or more of any one or more of the above-referenced cancer types.

In some embodiments, classifying the fragmentation patterns identifies the subject as being positive or negative for one or more of breast cancer (including hormone receptor-positive or negative breast cancer), bladder cancer, lung cancer, kidney cancer, prostate cancer, and metastatic neuroendocrine prostate cancer. In some embodiments, classifying the fragmentation patterns identifies the subject as being positive or negative for one or more of breast cancer, bladder cancer, lung cancer, prostate cancer, and metastatic neuroendocrine prostate cancer.

In some embodiments, classifying the fragmentation patterns identifies the subject as being positive or negative for a cancer treatable with a certain drug. Exemplary drugs in this regard include any one or more of the following drugs, in any combination: Abiraterone, Enzalutamide, Apalutamide, Darolutamide, Anastrozole, Erlotinib, Rapamycin, Sunitinib, PHA-665752, MG-132, Paclitaxel, Cyclopamine, AZ628, Sorafenib, Tozasertib, Imatinib, NVP-TAE684, Crizotinib, Saracatinib, S-Trityl-L-cysteine, Z-LLNle-CHO, Dasatinib, GNF-2, CGP-60474, CGP-082996, A-770041, WH-4-023, WZ-1-84, BI-2536, BMS-536924, BMS-509744, CMK, Pyrimethamine, JW-7-52-1, A-443654, GW843682×, Entinostat, Parthenolide, GSK319347A, TGX221, Bortezomib, XMD8-85, Seliciclib, Salubrinal, Lapatinib, GSK269962A, Doxorubicin, Etoposide, Gemcitabine, Mitomycin-C, Vinorelbine, NSC-87877, Bicalutamide, QS11, CP466722, Midostaurin, CHIR-99021, Ponatinib, AZD6482, JNK-9L, PF-562271, HG6-64-1, JQ1, JQ12, DMOG, FTI-277, OSU-03012, Shikonin, AKT inhibitor, VIII, Embelin, FH535, PAC-1 IPA-3, GSK650394, BAY-61-3606, 5-Fluorouracil, Thapsigargin, Obatoclax, Mesylate, BMS-754807, Linsitinib, Bexarotene, Bleomycin, LFM-A13, GW-2580, Luminespib, Phenformin, Bryostatin 1, Pazopanib, Dacinostat, Epothilone B, GSK1904529A, BMS-345541, Tipifarnib, Avagacestat, Ruxolitinib, AS601245, Ispinesib, Mesylate, TL-2-105, AT-7519, TAK-715, BX-912, ZSTK474, AS605240, Genentech, Cpd 10, GSK1070916, Enzastaurin, GSK429286A, FMK, QL-XII-47, IC-87114, Idelalisib, UNC0638, Cabozantinib, WZ3105, XMD14-99, Quizartinib, CP724714, JW-7-24-1, NPK76-II-72-1, STF-62247, NG-25, TL-1-85, VX-1le, FR-180204, ACY-1215, Tubastatin,A Zibotentan, Sepantronium bromide, NSC-207895, VNLG/124, AR-42, CUDC-101, Belinostat, I-BET-762, CAY10603, Linifanib, BIX02189, Alectinib, Pelitinib, Omipalisib, JNJ38877605, SU11274, KIN001-236, KIN001-244, WHI-P97, KIN001-042, KIN001-260, KIN001-266, Masitinib, Amuvatinib, MPS-1-IN-1, NVP-BHG712 OSI-930, OSI-027, CX-5461, PHA-793887, PI-103, PIK-93, SB52334, TPCA-1, Fedratinib, Foretinib, Y-39983, YM201636, Tivozanib, WYE-125132, GSK690693, SNX-2112, QL-XI-92, XMD13-2, QL-X-138, XMD15-27, T0901317, Selisistat, Tenovin-6, THZ-2-49, KIN001-270, THZ-2-102-1, AT7867, CI-1033, PF-00299804, TWS119, Torin 2, Pilaralisib, GSK1059615, Voxtalisib, Brivanib, BMS-540215, BIBF-1120, AST-1306, Apitolisib, LIMK1,inhibitor, BMS4, kb NB 142-70, Sphingosine Kinase 1 Inhibitor II, eEF2K Inhibitor A-484954, MetAP2 Inhibitor A832234, Venotoclax, CPI-613, CAY10566, Ara-G, Pemetrexed, Alisertib, Flavopiridol, C-75, CAP-232 (CAP-232, TT-232, TLN-232), Trichostatin A, Panobinostat, LCL161, IMD-0354, MIMI, ETP-45835, CD532

NSC319726, ARRY-520, SB505124, A-83-01, LDN-193189, FTY-720, BAM7 AGI-6780, Kobe2602, LGK974, Wnt-C59, RU-SKI 43, AICA Ribonucleotide, Vinblastine, Cisplatin, Cytarabine, Docetaxel, Methotrexate, Tretinoin, Gefitinib, Navitoclax, Vorinostat, Nilotinib, Refametinib, CI-1040, Temsirolimus, Olaparib, Veliparib, Bosutinib, Lenalidomide, Axitinib, AZD7762, GW441756, Lestaurtinib, SB216763, Tanespimycin, VX-702, Motesanib, KU-55933, Elesclomol, Afatinib, Vismodegib, PLX-4720, BX795, NU7441, SL0101, Doramapimod, JNK Inhibitor VIII, Weel Inhibitor, Nutlin-3a (-), Mirin, PD173074, ZM447439, RO-3306, MK-2206, Palbociclib, Dactolisib, Pictilisib, AZD8055, PD0325901, SB590885, Selumetinib, CCT007093, EHT-1864, Cetuximab, PF-4708671, Serdemetan, AZD4547, Capivasertib, HG-5-113-01, HG-5-88-01, TW 37, XMD11-85h, ZG-10, XMD8-92, QL-VIII-58, CCT-018159, Rucaparib, AZ20, KU-60019, Tamoxifen, QL-XII-61, PFI-1, IOX2, YK-4-279, (5Z)-7-Oxozeaenol, Piperlongumine, Daporinad, Talazoparib, rTRAIL, UNC1215, UNC0642, SGC0946, ICL1100013, XAV939, Trametinib, Dabrafenib, Temozolomide, Bleomycin (50 uM), AZD3514, Bleomycin (10 uM), AZD6738, AZD5438, AZD6094, Dyrklb_0191, AZD4877, EphB4_9721, Fulvestrant, AZD8931, FEN1_3940, FGFR_0939, FGFR_3831, BPTES, AZD7969, AZD5582, IAP_5620, IAP_7638, IGFR_3801, AZD1480, JAK1_3715, JAK3_7406, MCTI_6447, MCT4 1422, AZD2014, AZD8186, AZD8835, PI3Ka 4409, AZD1208, PLK_6522, RAF 9304, PARP 9495, PARP 0108, PARP 9482, TANK 1366, AZD1332, TTK 3146, SN-38, Pevonedistat, PFI-3, Camptothecin, Staurosporine, Irinotecan, Oxaliplatin, PRIMA-1MET, Niraparib, MK-1775, Dinaciclib, EPZ004777, AZ960, Epirubicin, Cyclophosphamide, Sapitinib, Uprosertib, Alpelisib, Taselisib, EPZ5676, SCH772984, IWP-2,Leflunomide, VE-822, WZ4003, CZC24832, GSK2606414, PFI3, PCI-34051, RVX-208, OTX015, GSK343, ML323, Entospletinib, PRT062607, Ribociclib, Picolinici-acid, AZD5153, CDK9_5576, CDK9_5038, Eg5_9814, ERK_2440, ERK_6604, IRAK4_4710, JAK1_8709, AZD5991, PAK_5339, TAF1 5496, ULK1 4989, VSP34_8731, IGF1R_3801, JAK_8517, Ibrutinib, Zoledronate, Acetalax, Carmustine, Topotecan, Teniposide, Mitoxantrone, Dactinomycin, Fludarabine, Nelarabine, Vincristine, Podophyllotoxin bromide, Dihydrorotenone, Gallibiscoquinazole, Elephantin, Sinularin, Sabutoclax, LY2109761, OF-1, MN-64, KRAS (G12C) Inhibitor-12, BDP-00009066, Buparlisib, Ulixertinib, Venetoclax, ABT737, Afuresertib, AGI-5198, AZD3759, AZD5363, Osimertinib, Cediranib, Ipatasertib, GDC0810, GNE-317, GSK2578215A, I-BRD9, Telomerase Inhibitor IX, MIRA-1, NVP-ADW742, P22077, Savolitinib, UMI-77, WIKI4, WEHI-539, BPD-00008900, BIBR-1532, Pyridostatin, AMG-319, MK-8776, LJI308, AZ6102, GSK591, VE821, and AT13148.

Classifying the fragmentation patterns can be performed with a classifier. A classifier is an algorithm computer code that receives, as input, test data and produces, as output, a classification of the input data as belonging to one or another class. A classifier can be trained for the purposes herein by determining the fragmentation patterns of cfDNA from test subjects having particular known disease states (e.g., cancer or particular types of cancer) and control subjects not having those disease states, or model samples representative of same. The classifier cfDNA can be cfDNA corresponding to any classifier region or set of classifier regions described herein, such as first exons of a set of cancer genes. Machine learning can then be used to distinguish the fragmentation patterns of the cfDNA from subjects having particular disease states from cfDNA from subjects not having those particular disease states. Machine learning employs algorithms, executed by computer, that automate analytical model building, e.g., for clustering, classification or pattern recognition. Machine learning algorithms may be supervised or unsupervised. Machine learning algorithms include, for example, artificial neural networks (e.g., back propagation networks), discriminant analyses (e.g., Bayesian classifier or Fischer analysis), support vector machines, decision trees (e.g., recursive partitioning processes such as CART - classification and regression trees, or random forests), linear classifiers (e.g., multiple linear regression (MLR), partial least squares (PLS) regression, and principal components regression), hierarchical clustering, and cluster analysis.

In various embodiments of the invention, the methods described herein are capable of identifying a subject as being positive for cancer at an accuracy of at least about 50%, at least about 55%, at least about 60%, at least about 65%, at least about 70%, at least about 75%, at least about 80%, at least about 85%, at least about 90%, at least about 95%, at least about 99%, or about 100% using a sample from the subject having a ct-fraction from about 0.000001 to about 0.01, such as about 0.000005 to about 0.01, about 0.00001 to about 0.01, about 0.00005 to about 0.01, about 0.0001 to about 0.01, about 0.0005 to about 0.01, 0.000001 to about 0.005, about 0.000005 to about 0.005, about 0.00005 to about 0.005, about 0.00005 to about 0.005, about 0.0001 to about 0.005, about 0.0005 to about 0.005, 0.000001 to about 0.001, about 0.000005 to about 0.001, about 0.00001 to about 0.001, about 0.00005 to about 0.001, about 0.0001 to about 0.001, about 0.0005 to about 0.001, about 0.000001 to about 0.0005, about 0.000005 to about 0.0005, about 0.00001 to about 0.0005, about 0.00005 to about 0.0005, about 0.0001 to about 0.0005, about 0.0005 to about 0.0005, about 0.000001 to about 0.0001, about 0.000005 to about 0.0001, about 0.00001 to about 0.0001, about 0.00005 to about 0.0001, about 0.000001 to about 0.00005, about 0.000005 to about 0.00005, about 0.00001 to about 0.00005, about 0.000001 to about 0.00001, about 0.000005 to about 0.00001, or about 0.000001 to about 0.000005.

In various embodiments of the invention, the methods described herein are capable of identifying a subject as being positive for a particular type of cancer (e.g., breast cancer, bladder cancer, lung cancer, prostate cancer, and/or metastatic neuroendocrine prostate cancer), at an accuracy of at least about 50%, at least about 55%, at least about 60%, at least about 65%, at least about 70%, at least about 75%, at least about 80%, at least about 85%, at least about 90%, at least about 95%, at least about 99%, or about 100% using a sample from the subject having a ct-fraction from about 0.001 to about 0.25, such as from about 0.005 to about 0.25, from about 0.01 to about 0.25, from about 0.05 to about 0.25, from about 0.1 to about 0.25, from about 0.001 to about 0.1, from about 0.005 to about 0.1, from about 0.01 to about 0.1, from about 0.05 to about 0.1, from about 0.001 to about 0.05, from about 0.005 to about 0.05, from about 0.01 to about 0.05, from about 0.001 to about 0.01, from about 0.005 to about 0.01, or from about 0.001 to about 0.005.

“Accuracy” as used herein is defined as the number of correct identifications (e.g., correct identification of subjects as being positive for cancer or a particular type of cancer according to the methods described herein) divided by the total number of identifications made. A correct identification is an identification that matches the true condition of the subject. Methods of calculating ct-fractions can be performed according to the method of Vandekerkhove et al. 2021 (Vandekerkhove G, Lavoie J M, Annala M, Murtha A J, Sundahl N, Walz S, Sano T, Taavitsainen S, Ritch E, Fazli L, Hurtado-Coll A, Wang G, Nykter M, Black P C, Todenhöfer T, Ost P, Gibb E A, Chi K N, Eigl B J, Wyatt A W. Plasma ctDNA is a tumor tissue surrogate and enables clinical-genomic stratification of metastatic bladder cancer. Nat Commun. 2021 Jan. 8;12(1):184).

The methods described herein can be used for screening subjects to identify those for diagnostic testing and/or treatment. Exemplary types of diagnostic testing include imaging and biopsy. Exemplary types of imaging include computerized tomography scans (CT or CAT scans), magnetic resonance imaging (MRI), nuclear scans, bone scans, positron emission tomography (PET) scans, ultrasounds, X-rays, endoscopy (e.g., colonoscopy, bronchoscopy). Biopsies include removal of tissue from the subject, typically with a needle or surgery. Biopsies include solid tissue biopsy and bodily fluid biopsy (liquid biopsy). In some embodiments, the methods described herein identify a subject as having a cancer of a particular tissue of origin, and the subject then undergoes imaging or biopsy of the particular tissue of origin. In some embodiments, the particular tissue of origin is a solid tissue, and the subject undergoes imaging and/or biopsy of the solid tissue.

In some embodiments, the subject is treated for the cancer after being identified as being positive for cancer. In some embodiments, the subject is treated for a particular type of cancer after being identified as being positive for that particular type of cancer. The treatment in some versions is a treatment specific for that particular type of cancer, such as a treatment that targets a particular tissue or specific cancer type. Exemplary treatments include surgeries (e.g., resection surgeries), radiation therapies, and drug therapies.

Exemplary drug therapies include treatments with chemotherapy agents, targeted cancer therapy agents, differentiating therapy agents, hormone therapy agents, and immunotherapy agents. Exemplary chemotherapy agents include alkylating agents, antimetabolites, anthracyclines, anti-tumor antibiotics, cytoskeletal disruptors (taxans), topoisomerase inhibitors, mitotic inhibitors, corticosteroids, kinase inhibitors, nucleotide analogs, and platinum-based agents. Exemplary targeted cancer therapy agents include signal transduction inhibitors (e.g. tyrosine kinase and growth factor receptor inhibitors), histone deacetylase (HDAC) inhibitors, retinoic receptor agonists, proteosome inhibitors, angiogenesis inhibitors, and monoclonal antibody conjugates. Exemplary differentiating therapy agents including retinoids, such as tretinoin, alitretinoin and bexarotene. Exemplary hormone therapy agents include anti-estrogens, aromatase inhibitors, progestins, estrogens, anti-androgens, and GnRH agonists or analogs. Exemplary immunotherapy agents include monoclonal antibody therapies such as rituximab (RITUXAN) and alemtuzumab (CAIVIPATH), non-specific immunotherapies and adjuvants, such as BCG, interleukin-2 (IL-2), and interferon-alfa, and immunomodulating drugs, for instance, thalidomide and lenalidomide (REVLIMID). Exemplary drugs that can be used in treatment include any one or more of the following drugs, in any combination: Abiraterone, Enzalutamide, Apalutamide, Darolutamide, Anastrozole, Erlotinib, Rapamycin, Sunitinib, PHA-665752, MG-132, Paclitaxel, Cyclopamine, AZ628, Sorafenib, Tozasertib, Imatinib, NVP-TAE684, Crizotinib, Saracatinib, S-Trityl-L-cysteine, Z-LLNle-CHO, Dasatinib, GNF-2, CGP-60474, CGP-082996, A-770041, WH-4-023, WZ-1-84, BI-2536, BMS-536924, BMS-509744, CMK, Pyrimethamine, JW-7-52-1, A-443654, GW843682×, Entinostat, Parthenolide, GSK319347A, TGX221, Bortezomib, XMD8-85, Seliciclib, Salubrinal, Lapatinib, GSK269962A, Doxorubicin, Etoposide, Gemcitabine, Mitomycin-C, Vinorelbine, NSC-87877, Bicalutamide, QS11, CP466722, Midostaurin, CHIR-99021, Ponatinib, AZD6482, JNK-9L, PF-562271, HG6-64-1, JQ1, JQ12, DMOG, FTI-277, OSU-03012, Shikonin, AKT inhibitor, VIII, Embelin, FH535, PAC-1 IPA-3, GSK650394, BAY-61-3606, 5-Fluorouracil, Thapsigargin, Obatoclax, Mesylate, BMS-754807, Linsitinib, Bexarotene, Bleomycin, LFM-A13, GW-2580, Luminespib, Phenformin, Bryostatin 1, Pazopanib, Dacinostat, Epothilone B, GSK1904529A, BMS-345541, Tipifarnib, Avagacestat, Ruxolitinib, AS601245, Ispinesib, Mesylate, TL-2-105, AT-7519, TAK-715, BX-912, ZSTK474, AS605240, Genentech, Cpd 10, GSK1070916, Enzastaurin, GSK429286A, FMK, QL-XII-47, IC-87114, Idelalisib, UNC0638, Cabozantinib, WZ3105, XMD14-99, Quizartinib, CP724714, JW-7-24-1, NPK76-II-72-1, STF-62247, NG-25, TL-1-85, VX-1le, FR-180204, ACY-1215, Tubastatin,A Zibotentan, Sepantronium bromide, NSC-207895, VNLG/124, AR-42, CUDC-101, Belinostat, I-BET-762, CAY10603, Linifanib, BIX02189, Alectinib, Pelitinib, Omipalisib, JNJ38877605, SU11274, KIN001-236, KIN001-244, WHI-P97, KIN001-042, KIN001-260, KIN001-266, Masitinib, Amuvatinib, MPS-1-IN-1, NVP-BHG712 OSI-930, OSI-027, CX-5461, PHA-793887, PI-103, PIK-93, SB52334, TPCA-1, Fedratinib, Foretinib, Y-39983, YM201636, Tivozanib, WYE-125132, GSK690693, SNX-2112, QL-XI-92, XMD13-2, QL-X-138, XMD15-27, T0901317, Selisistat, Tenovin-6, THZ-2-49, KIN001-270, THZ-2-102-1, AT7867, CI-1033, PF-00299804, TWS119, Torin 2, Pilaralisib, GSK1059615, Voxtalisib, Brivanib, BMS-540215, BIBF-1120, AST-1306, Apitolisib, LIMK1,inhibitor, BMS4, kb NB 142-70, Sphingosine Kinase 1 Inhibitor II, eEF2K Inhibitor A-484954, MetAP2 Inhibitor A832234, Venotoclax, CPI-613, CAY10566, Ara-G, Pemetrexed, Alisertib, Flavopiridol, C-75, CAP-232 (CAP-232, TT-232, TLN-232), Trichostatin A, Panobinostat, LCL161, IMD-0354, MIMI, ETP-45835, CD532

NSC319726, ARRY-520, SB505124, A-83-01, LDN-193189, FTY-720, BAM7 AGI-6780, Kobe2602, LGK974, Wnt-C59, RU-SKI 43, AICA Ribonucleotide, Vinblastine, Cisplatin, Cytarabine, Docetaxel, Methotrexate, Tretinoin, Gefitinib, Navitoclax, Vorinostat, Nilotinib, Refametinib, CI-1040, Temsirolimus, Olaparib, Veliparib, Bosutinib, Lenalidomide, Axitinib, AZD7762, GW441756, Lestaurtinib, SB216763, Tanespimycin, VX-702, Motesanib, KU-55933, Elesclomol, Afatinib, Vismodegib, PLX-4720, BX795, NU7441, SL0101, Doramapimod, JNK Inhibitor VIII, Weel Inhibitor, Nutlin-3a (-), Mirin, PD173074, ZM447439, RO-3306, MK-2206, Palbociclib, Dactolisib, Pictilisib, AZD8055, PD0325901, SB590885, Selumetinib, CCT007093, EHT-1864, Cetuximab, PF-4708671, Serdemetan, AZD4547, Capivasertib, HG-5-113-01, HG-5-88-01, TW 37, XMD11-85h, ZG-10, XMD8-92, QL-VIII-58, CCT-018159, Rucaparib, AZ20, KU-60019, Tamoxifen, QL-XII-61, PFI-1, IOX2, YK-4-279, (5Z)-7-Oxozeaenol, Piperlongumine, Daporinad, Talazoparib, rTRAIL, UNC1215, UNC0642, SGC0946, ICL1100013, XAV939, Trametinib, Dabrafenib, Temozolomide, Bleomycin (50 uM), AZD3514, Bleomycin (10 uM), AZD6738, AZD5438, AZD6094, Dyrklb_0191, AZD4877, EphB4_9721, Fulvestrant, AZD8931, FEN1_3940, FGFR_0939, FGFR_3831, BPTES, AZD7969, AZD5582, IAP_5620, IAP_7638, IGFR_3801, AZD1480, JAK1_3715, JAK3_7406, MCTI_6447, MCT4 1422, AZD2014, AZD8186, AZD8835, PI3Ka 4409, AZD1208, PLK_6522, RAF_9304, PARP 9495, PARP 0108, PARP 9482, TANK 1366, AZD1332, TTK 3146, SN-38, Pevonedistat, PFI-3, Camptothecin, Staurosporine, Irinotecan, Oxaliplatin, PRIMA-1MET,

Niraparib, MK-1775, Dinaciclib, EPZ004777, AZ960, Epirubicin, Cyclophosphamide, Sapitinib, Uprosertib, Alpelisib, Taselisib, EPZ5676, SCH772984, IWP-2,Leflunomide, VE-822, WZ4003, CZC24832, GSK2606414, PFI3, PCI-34051, RVX-208, OTX015, GSK343, ML323, Entospletinib, PRT062607, Ribociclib, Picolinici-acid, AZD5153, CDK9_5576, CDK9_5038, Eg5_9814, ERK_2440, ERK_6604, IRAK4 4710, JAK1_8709, AZD5991, PAK_5339, TAF1 5496, ULK1 4989, VSP34_8731, IGF1R_3801, JAK_8517, Ibrutinib, Zoledronate, Acetalax, Carmustine, Topotecan, Teniposide, Mitoxantrone, Dactinomycin, Fludarabine, Nelarabine, Vincristine, Podophyllotoxin bromide, Dihydrorotenone, Gallibiscoquinazole, Elephantin, Sinularin, Sabutoclax, LY2109761, OF-1, MN-64, KRAS (G12C) Inhibitor-12, BDP-00009066, Buparlisib, Ulixertinib, Venetoclax, ABT737, Afuresertib, AGI-5198, AZD3759, AZD5363, Osimertinib, Cediranib, Ipatasertib, GDC0810, GNE-317, GSK2578215A, I-BRD9, Telomerase Inhibitor IX, MIRA-1, NVP-ADW742, P22077, Savolitinib, UMI-77, WIKI4, WEHI-539, BPD-00008900, BIBR-1532, Pyridostatin, AMG-319, MK-8776, LJI308, AZ6102, GSK591, VE821, and AT13148.

It is within the capabilities of a skilled physician or oncologist to select an appropriate cancer therapeutic agent based on characteristics such as the type of tumor, cancer stage, previous exposure to cancer treatment or therapeutic agent, and other characteristics of the cancer.

In some embodiments, the methods described herein identify a subject as having a cancer of a particular tissue of origin, and the subject then undergoes surgery on the particular tissue of origin. In some embodiments, the particular tissue of origin is a solid tissue, and the subject undergoes surgery the solid tissue.

The correspondence of various elements described herein can be determined by alignment of sequences using an alignment algorithm, for example, Needleman-Wunsch algorithm (see e.g., the EMBOSS Needle aligner available at the URL ebi.ac.uk/Tools/psa/emboss_needle/nucleotide.html, optionally with default settings), the BLAST algorithm (see e.g., the BLAST alignment tool available at the URL blast.ncbi.nlm.nih.gov/Blast.cgi, optionally with default settings), or the Smith-Waterman algorithm (see e.g., the EMBOSS Water aligner available at the URL ebi.ac.uk/Tools/psa/emboss_water/nucleotide.html, optionally with default settings). Optimal alignment may be assessed using any suitable parameters of a chosen algorithm, including default parameters.

In some cases, a sequence may be aligned to a reference genome or a reference sequence. A reference genome (sometimes referred to as an “assembly”) is assembled from genetic data and intended to represent the genome of a species. Typically, reference genomes are haploid. Typically, reference genomes do not represent the genome of a single individual of the species but rather are mosaics of the genomes of several individuals. A reference genome can be publicly available or be a private reference genome. Human reference genomes include, for example, hgl9 or NCBI Build 37 or Build 38. A reference sequence is generally a nucleotide sequence against which a subject's nucleotide sequences are compared. Typically, a reference sequence is derived from a reference genome.

Any element disclosed or claimed herein can comprise, consist of, or consist essentially of the characteristics herein described with respect thereto.

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### EXAMPLES

### Fragmentomics of Targeted Circulating Tumor DNA Sequencing Panels

### Summary

The isolation of cell-free DNA (cfDNA) from the bloodstream can be used to detect and analyze somatic alterations in circulating tumor DNA (ctDNA), and multiple cfDNA targeted sequencing panels are now commercially available for FDA-approved biomarker indications to guide treatment. More recently, cfDNA fragmentation patterns have emerged as a tool to infer epigenomic and transcriptomic information. However, most of these analyses used whole-genome sequencing, which is insufficient to identify FDA-approved biomarker indications in a cost-effective manner. We used machine-learning models of fragmentation patterns at the first coding exon in targeted cancer gene cfDNA sequencing panels to distinguish between cancer vs. non-cancer patients, as well as the specific tumor type and subtype. We assessed this approach in two independent cohorts: a published cohort from GRAIL (breast, lung, and prostate cancers, non-cancer, N=198) and an institutional cohort from the University of Wisconsin (UW; breast, lung, prostate, bladder cancers, N=320). Each cohort was split 70/30% into training and validation sets. In the UW cohort, training cross validated accuracy was 82.1%, and accuracy in the independent validation cohort was 86.6% despite a median ctDNA fraction of only 0.06. In the GRAIL cohort, to assess how this approach performs in very low ctDNA fractions, training and independent validation were split based on ctDNA fraction. Training cross validated accuracy was 80.6%, and accuracy in the independent validation cohort was 76.3%. In the validation cohort where the ctDNA fractions were all <0.05 and as low as 0.0003, the cancer vs. non-cancer AUC was 0.99. To our knowledge, this is the first study to demonstrate that sequencing from targeted cfDNA panels can be utilized to analyze fragmentation patterns to classify cancer types, dramatically expanding the potential capabilities of clinical panels at minimal additional cost.

### Introduction

Profiling of genomic driver alterations in cancer has become increasingly important, not only for studying the biological underpinnings of cancer, but also in identifying clinically actionable alterations for targeted therapies in clinical trials and practice. Historically, tumor samples have been required, but obtaining tissue specimens for molecular profiling is not always feasible and can be especially challenging in the metastatic setting. Cell-free DNA (cfDNA) from cancer patients provides a minimally invasive approach for assessing molecular events in the tumor by detecting alterations in the tumor-derived cfDNA, also called circulating tumor DNA (ctDNA)¹. This is a mature technology, with multiple commercially available next-generation sequencing (NGS) ctDNA panels².

The stability of cfDNA in circulation is dependent on its association with proteins and protein complexes which offer protection against DNAses found in the blood³⁻⁴. The nucleosome complex is the most common protector of cfDNA which is reflected in the size distribution of cfDNA fragments showing a mode fragment size of 167 bp corresponding to the wrapping of DNA around a single nucleosome, with a smaller proportion of fragments at 334 bp corresponding to a di-nucleosome complex⁵⁻⁸. Other studies have also described smaller peaks at a periodicity of approximately 10 bp at lower fragment sizes representing the accessibility of DNA minor grooves to endonuclease cleavage as it wraps around the histone complex, as well as the binding of transcription factors or other small DNA binding proteins⁷⁻¹¹.The study of cfDNA fragmentation patterns has been referred to as “fragmentomics.”

Almost all clinical fragmentomic studies to date have utilized whole-genome sequencing (WGS) to assess fragmentation patterns across the genome in an unbiased manner²⁷. While WGS has the advantage of breadth of coverage, there is generally low sequencing depth making it unsuitable for cfDNA somatic alteration detection as it has poor sensitivity, especially at low ctDNA fractions²⁸. Conversely, cfDNA targeted panels allow for deeper sequencing at areas of interest, which are typically coding regions of important cancer genes. Previous cfDNA fragmentomics analyses have generally focused on WGS which affords probing of fragmentation patterns at all genomic regions in an unbiased manner, as the investigated biological phenomena are typically not unique to regions profiled by target panels (e.g. exonic regions). For example, many analyses of fragmentation patterns have focused on the assessment of histone binding, which requires relatively uniform read support across large areas of the genome^{7,9,17,20,23}. This type of read support is not provided by targeted panel sequencing.

While previous studies have focused on fragmentation patterns across the whole genome, we hypothesized that cfDNA fragmentation patterns in the coding regions of important oncogenes and tumor suppressors could provide important insights for distinguishing between tumor and normal samples, as well as between different tumor types and subtypes. We specifically focused on fragmentation patterns overlapping the first coding exon of targeted genes. To evaluate this, we examined the fragmentomic patterns in both a publicly available multi-cancer cfDNA dataset profiled using the GRAIL cfDNA assay²⁹, as well as an institutional multi-cancer cohort profiled using a custom cfDNA panel. We found that analysis of the fragmentation patterns of first coding exons could distinguish between cancer types as well as between cancer vs. normal. The use of fragmentation patterns from targeted cfDNA panels would allow for the advantages of both variant calling and fragmentomics in a single assay which could be leveraged on any existing panels that are already commercially available.

### Methods

### UW Patient Cohort

Peripheral blood samples were collected from patients with metastatic cancer enrolled in an IRB-approved liquid biopsy collection protocol at the University of Wisconsin-Madison (2014-1214), as well as from two ongoing clinical trials (NCT03090165, NCT03725761).

UW cfDNA Sample Collection, Preparation, and Sequencing

Blood was collected in 10 mL K2 EDTA (BD Vacutainer) or CellSave™ preservative blood collection tubes (Menarini Silicon Biosystems). Whole blood was processed within 4 hours (EDTA) or 36 hours (CellSave) from time of collection and was centrifuged at 300× g for 10 minutes. Plasma (3-6 mL) was harvested and centrifuged at 1500× g for 10 minutes, then stored at −80° C. cfDNA was isolated from 2-6mL plasma using the QIAamp Circulating Nucleic Acid kit (Qiagen). Germline DNA (gDNA) was isolated from matched peripheral blood mononuclear cells using the DNeasy blood and tissue kit (Qiagen) and fragmented using the NEBNext Ultra II FS DNA module (New England Biolabs). The Agilent Bioanalyzer high sensitivity DNA chip was used to quantify and assess cfDNA and fragmented gDNA quality. 50 ng cfDNA or 50 ng fragmented gDNA were subjected to library preparation with unique molecular indexes using the xGen Prism DNA library preparation kit (Integrated DNA technologies). For samples with less than 50 ng available cfDNA, 1, 10, or 25 ng DNA input was used. 8-12 libraries were pooled at 500 ng per library followed by hybridization and capture with a custom 822-gene panel using the xGen hybridization capture of DNA libraries kit (Integrated DNA technologies). Paired end sequencing (2×150 bp) was performed on a NovaSeq 6000 at the University of Wisconsin sequencing core, with a target depth of 20 million reads per germline sample and 50 million reads per cfDNA sample.

### Sequencing Data Processing

UW sequencing was aligned to the hg38 genome using BWA-mem³⁰ (v0.7.17) followed by deduplication of the aligned BAM files with Connor v0.6.1 (https://github.com/umich-bref-bioinf/Connor) which uses both start-stop position and UMIs along with filtering of low quality reads. A minimum family size threshold of 1 (-s 1) was used to keep all unique reads. BAM files were filtered for properly paired reads (samtools flags -f3 -F2308), sorted by read name, then converted to BEDPE files using bedtools³¹ (v2.30.0) bamtobed using the -bedpe flag. The start and stop positions of each read were extracted from the BEDPE file to yield a BED file of the sequencing reads to use for subsequent overlaps. GRAIL cfDNA sequencing data and metadata²⁹ were accessed and downloaded through the European Genome Archive (Dataset ID EGAD00001005302). As raw FASTQ files were not available, the hg19-prealigned BAM files were deduplicated using start-stop position and UMI followed by BAM to BED conversion as described above for the UW samples.

### Fragmentomics

For each sample, a global fragmentation distribution was calculated from the BED file by extracting the read insert size from the mapped end of the template and the mapped start of the template (stop-start) and then counting the number of reads at each size. The number of reads at each size was divided by the total number of reads in the sample to return the proportion of reads at each fragment size. Individual fragment distributions were plotted using the proportion of reads at each fragment size.

### Shannon Entropy for First Coding Exon

Canonical exon coordinates were downloaded as BED files from the UCSC Genome Browser using the Table Browser tool for both hg38 and hg19 (https://genome.ucsc.edu/cgi-bin/hgTables).

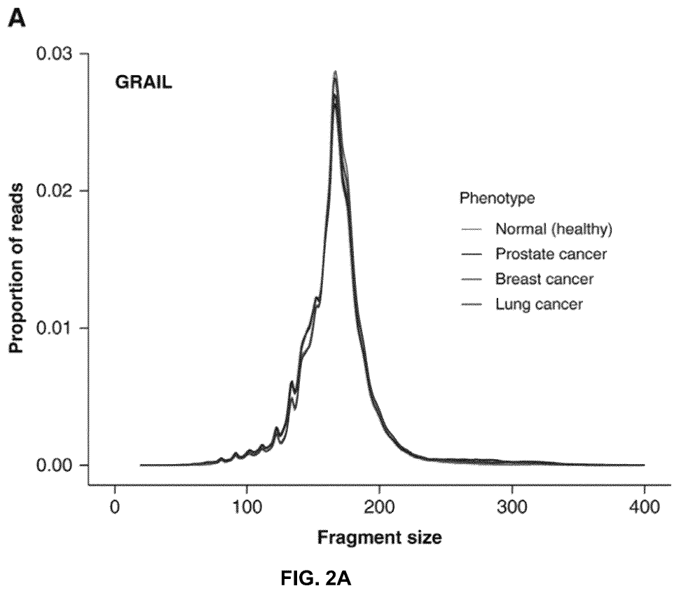

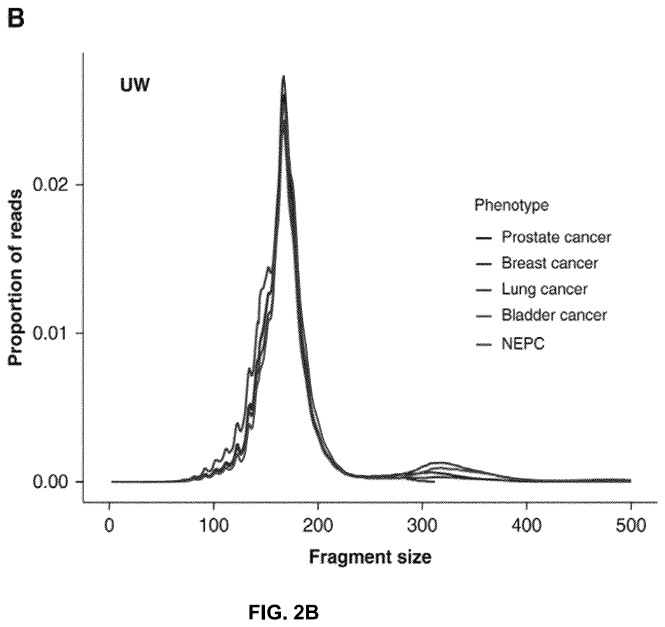

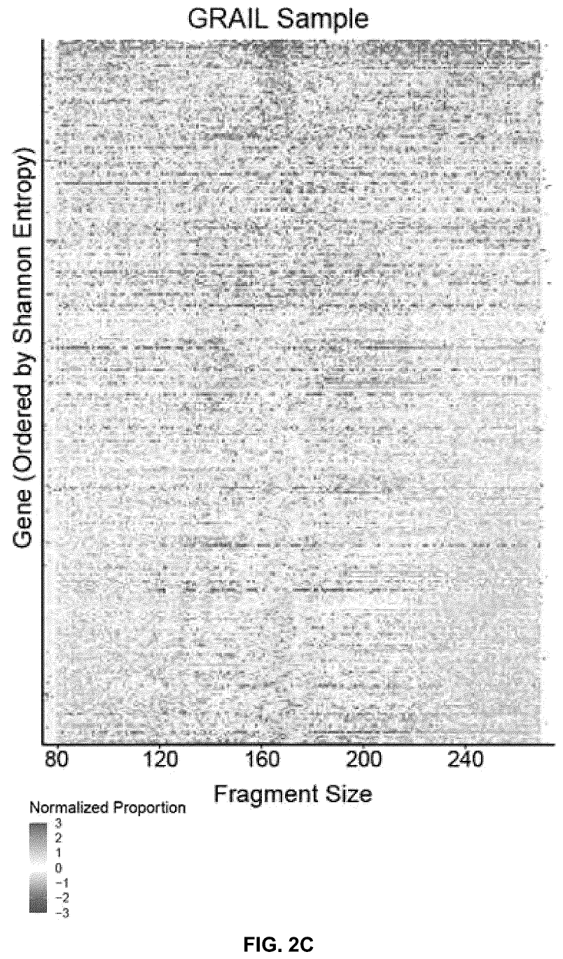

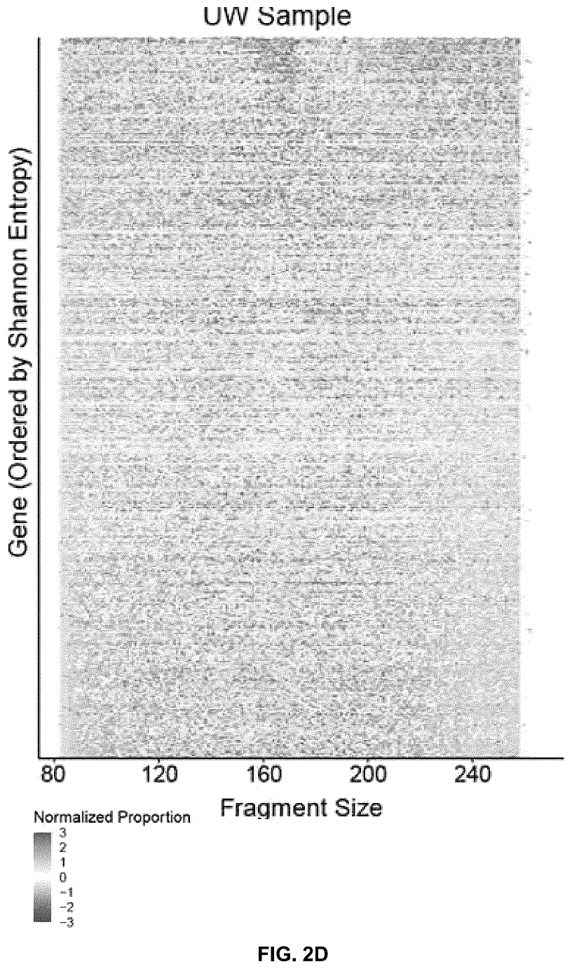

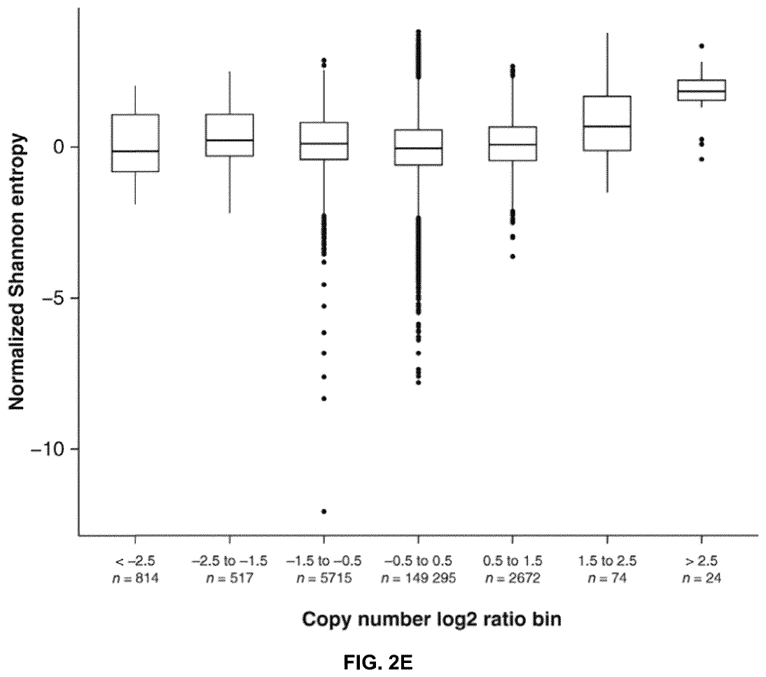

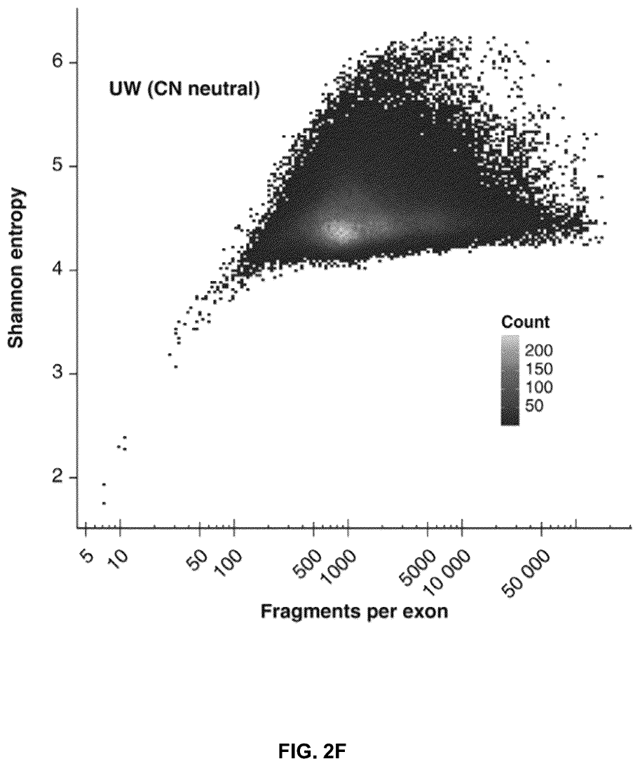

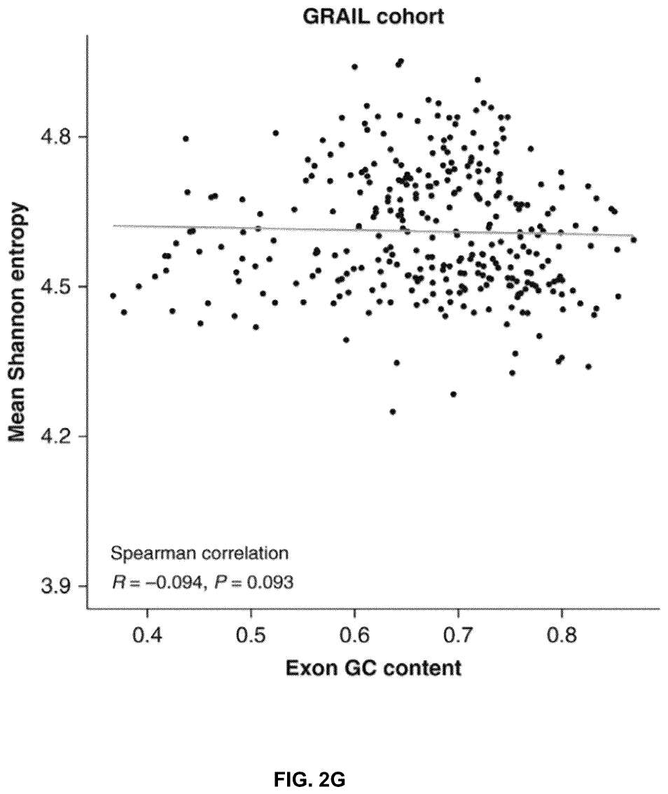

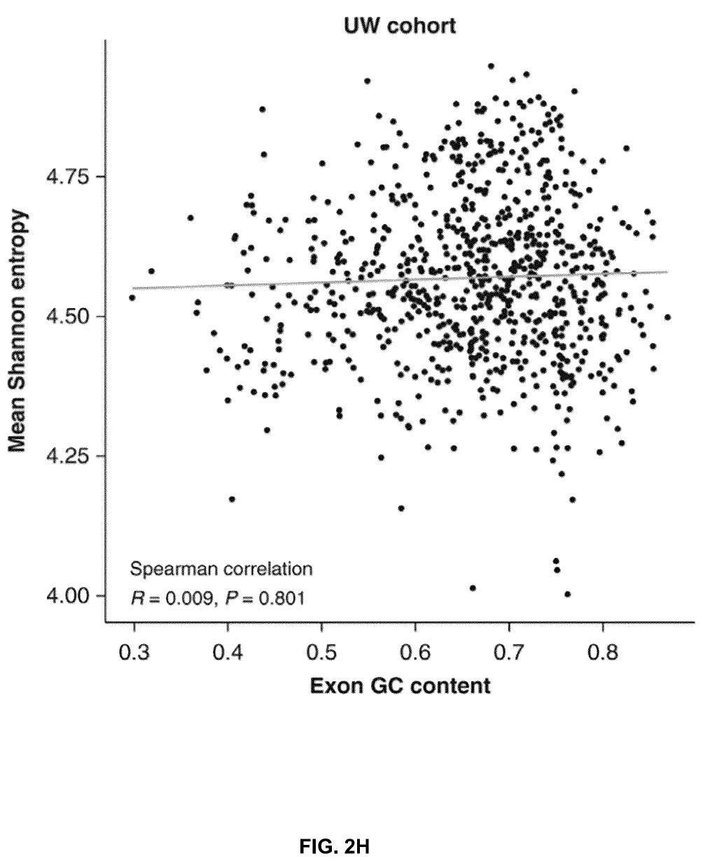

The BED file of each cfDNA sample was then overlapped with the respective exon file (hg38 for UW data, hg19 for GRAIL data) using bedtools intersect (v2.30.0) to yield reads overlapping with canonical exons. A minimum of 1 bp overlap was required for a read to be considered overlapped with an exon of interest. Reads overlapping the first coding exon of each gene were extracted, and a fragment size distribution was calculated for each gene using only the reads overlapping exon 1. Throughout the manuscript, references to “exon 1” or “E1SE” refer to the first coding exon of the respective gene or genes. Shannon entropy was calculated with the entropy function from the “entropy” package (v1.3.1) in R (v4.0.4) using the count of read fragments at each fragment size. This returned a single Shannon entropy value for reads overlapping the first exon of each gene in each sample. Given the association between the number of fragments analyzed and Shannon entropy (FIG. 2F), with low fragment count leading to a less accurate estimation of Shannon entropy, we required a minimum of 500 reads to overlap an exon across all samples to be included in the final dataset.

### GRAIL, Training, Cross Validation, and Independent Validation

Using the E1SE values for each gene in the GRAIL panel as features, multinomial regression using a generalized linear model with elastic net penalty (GLMNET) was used to predict cancer types. Samples were split into 70% training and 30% validation with low ctDNA fraction samples placed in the validation cohort. For all model training, a range of α and λ values were selected using latin hypercube sampling, and the best AUC on 10-fold cross validation was used select the final parameters. To estimate performance in the training cohort, 10-fold cross validation was performed, and training and parameter fitting (using 10-fold cross validation nested within the training set of each fold) was performed within each fold separately to avoid any information leakage. Predictions from the hold-out test sets for each fold were combined to calculate accuracy and ROC curves. A final model was then trained using the full training cohort. The independent validation cohort was then entered into the model to yield prediction scores, again with no information leakage between training and validation. These prediction scores were used to calculate accuracy and ROC curves.

UW training, Cross Validation, and Independent Validation

A similar approach was used for the UW cohort, which was also split into 70% training and 30% training. However, due to more missing ctDNA fraction data and imbalanced tumor types, the split was random while stratifying by tumor type, such that the relative proportions were similar across training and validation. Otherwise, training, cross validation, and independent validation were all performed the same as in GRAIL.

### Identification of Somatic Mutations in the UW Cohort

Somatic variant identification was performed using VarDictJava v1.8.3³² in paired sample mode using standard filter settings. Somatic mutations were required to have a minimum of 10 supporting reads, a minimum of 20 total reads covering the position, and up to 2 mismatches in the cfDNA samples, and a minimum of 20 total reads in the matched gDNA samples. For SNVs, the average mapping quality of mutation supporting reads was required to be at least 50 and the average distance of the mutant allele from the nearest read end was required to be at least 15 bases. We then conservatively removed germline mutations and somatic mutations related to clonal hematopoiesis of indeterminate potential (CHIP) by removing mutations to have more than 1 supporting read in any gDNA sample and removing any of 4,938 CHIP related mutations compiled by Bick et al.³³. Lastly, mutations in the low-complexity genomic regions and shared common mutations in dbSNP (dbSNP_G5) were discarded.

### Copy Number Analysis in the UW Cohort

Deduplicated BAM files were further filtered for uniquely mapped reads with high mapping quality using sambamba v0.8.2 (-F “mapping quality >=30 and not ([XA] !=null or [SA] !=null)”. Using the deduplicated, filtered, sorted, and indexed bam files as input, we ran CNVkit v0.9.9³⁴ to call somatic copy number alterations. CNVkit is a read-depth approach and utilizes both targeted and non-targeted regions to infer copy number more evenly across the genome. An accessibility bed file was created (cnvkit.py access -s 10000) to remove unmappable regions (i.e. large stretches of “N” characters) from the reference genome. CNVkit was run in batch mode for all cfDNA samples with a flat reference, which assumes equal coverage in all bins. Bin-level read depth was corrected for GC content, sequence repeats, and target density, and individually compared with the flat reference to calculate read depth ratio (log2). Genes with copy number gain or loss were identified using the genemetrics command with minimum absolute log2 copy ratio threshold (log2) of 0.5. Genes with less than three bins (probes) and read depth (depth) less than 1000 in each sample were discarded. CN was only used to compare against E1SE in our analysis. As ctDNA fraction impacts both fragmentomic patterns and copy number, copy number was therefore not corrected for tumor content.

Estimation of ctDNA Fraction in the UW Cohort

The proportion of tumor-derived cfDNA (ctDNA fraction) was estimated based on VAF of autosomal somatic mutations. VAF in autosomes is elevated if a mutant allele is accompanied by deletion of the other allele (i.e., loss of heterozygosity, LOH). Assuming a diploid tumor model and that the mutation with the highest VAF displays LOH, ctDNA fraction and the highest VAF can be related as

$$ct DNA fraction = \frac{2}{\frac{1}{VAF} + 1} .$$

To account for stochastic variation, we modeled the can be related as mutant allele read count with a binomial distribution as suggested by Vandekerkhove et al.³⁵ and calculated what the true VAF would be if the observed mutant allele read count was a 95% quantile outlier. After calculating ctDNA fraction for each somatic mutation in a given sample, the highest estimate of ctDNA fraction was used for the given sample as the mutation with the highest VAF is the most likely to be clonal. While the classification of LOH for the highest VAF is an assumption, many other reports utilize this method when analyzing targeted cfDNA sequencing^{15, 35-41}. Data for ctDNA fraction for samples from the GRAIL cohort were obtained from their previously published report²⁹ in the supplemental data (Source Data FIG. 2; tab “FIG. 2f”)

### Summary of Differences Between GRAIL and UW Cohorts

Patients:

GRAIL: Patients with metastatic cancer who were progressing on stable doses of treatment. The normal (non-cancer) blood samples were obtained from the San Diego Blood Bank.
UW: Patients with metastatic cancer. While in general, patients who were treatment naïve or progressing were preferred, this also included patients who were responding to treatment. Neuroendocrine prostate cancer and bladder cancer were also included, which were not in the GRAIL dataset. No normal blood samples were included, as this was not allowed on the institutional blood collection protocol.

### Sample Tubes:

GRAIL: Streck tubes were used
UW: EDTA or CellSave tubes were used
cfDNA Extraction:

### GRAIL: QIAamp Circulating Nucleic Acid Kit (Qiagen)

### UW: QIAamp Circulating Nucleic Acid kit (Qiagen)

### Library Preparation:

GRAIL: Illumina TruSeq DNA nano protocol with 6 mer UMIs (Illumina)
UW: xGen Prism DNA library preparation kit with 8 mer UMIs (Integrated DNA Technologies)

### Target Capture:

GRAIL: Custom 2.1 Mb panel with 508 cancer genes using Illumina Nextera Rapid Capture protocol (Illumina)
UW: Custom 2.4 Mb panel with 822 cancer genes using the xGen hybridization capture kit (Integrated DNA Technologies)

### Sequencing Depth:

GRAIL: average raw cfDNA sequencing depth 71,749×
UW: average raw cfDNA sequencing depth 3,042×

### Results

Overview of Two Independent Targeted ctDNA Panels and Cohorts

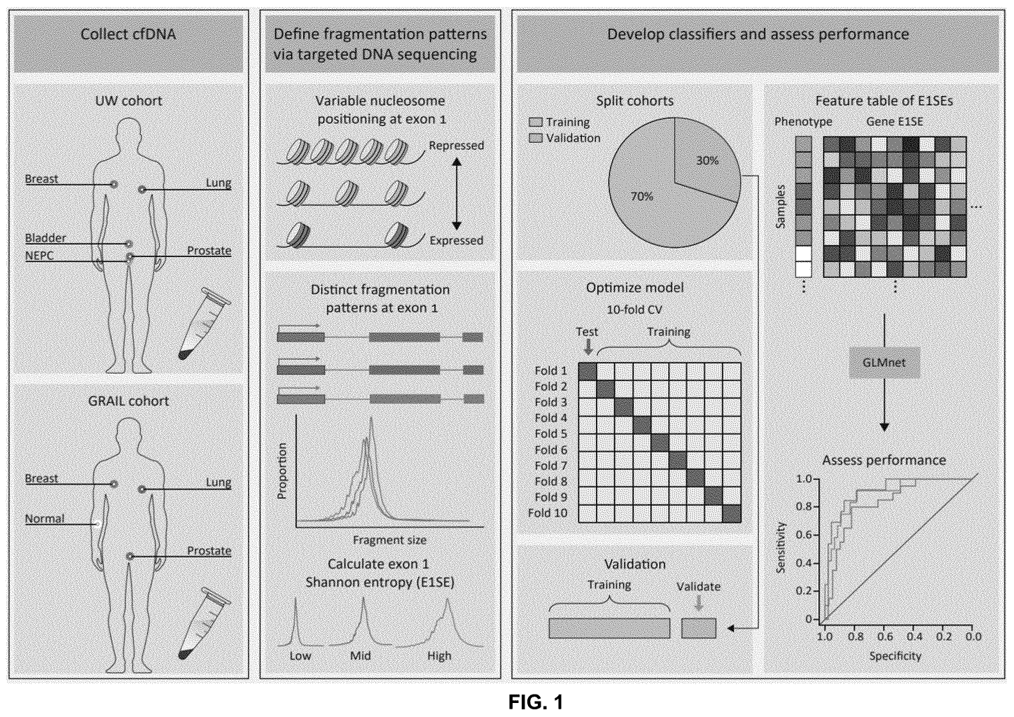

We examined two cohorts of cfDNA profiled using targeted cancer gene exon panels. The first was a previously published multi-cancer cohort of 198 cfDNA samples assessed using the commercial assay from GRAIL, covering 508 genes (˜2 MB) at a sequencing depth of >60,000× across breast, lung, and prostate cancer patients along with healthy donors²⁹. The second cohort was an institutional multi-cancer cohort from the University of Wisconsin (UW) with 320 samples across breast, lung, bladder, prostate, and neuroendocrine prostate cancers. Profiling was performed using a custom panel broadly covering the exons of 822 cancer genes, covering ˜2.4 MB of the genome at an average sequencing depth of 3,042×. We hypothesized that cfDNA fragmentation patterns at transcription start sites (TSSs), such as exon 1 of genes, could be used to inform tumor of origin using cfDNA sequencing from targeted panels which cover these regions in greater depth. To quantify the cfDNA fragmentation patterns at each exon 1 analyzed, the exon 1 Shannon entropy (E1SE) of the distribution was calculated which summarizes the diversity of fragments in the region. We then used these E1SEs to train models to predict tumor type. Both the UW and GRAIL cohorts were split into 70% training in which cross-validation was used to assess performance, and 30% independent validation In the GRAIL cohort, training was specifically performed on the 70% samples with the highest ctDNA fraction, and validation was performed on the lowest 30% by ctDNA fraction (FIG. 1).

### Fragment Distributions in Targeted Panels

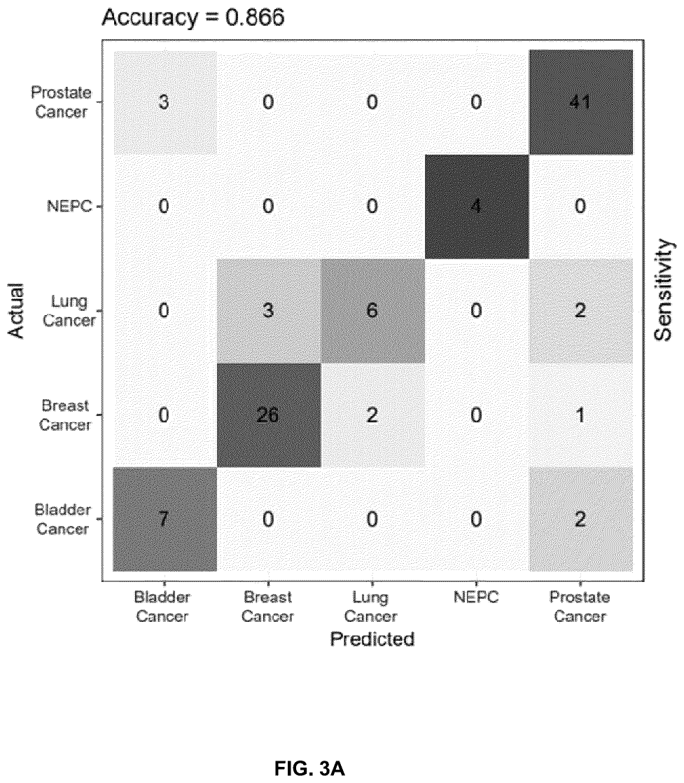

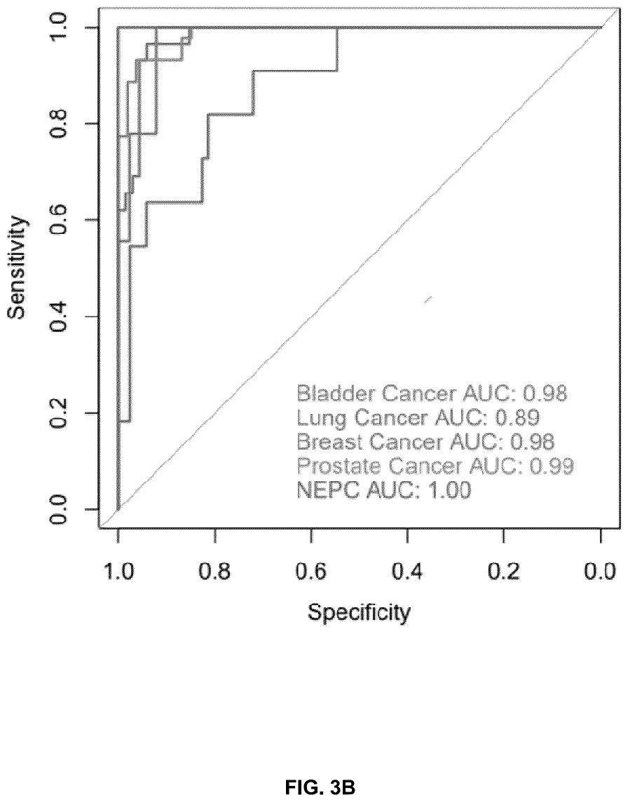

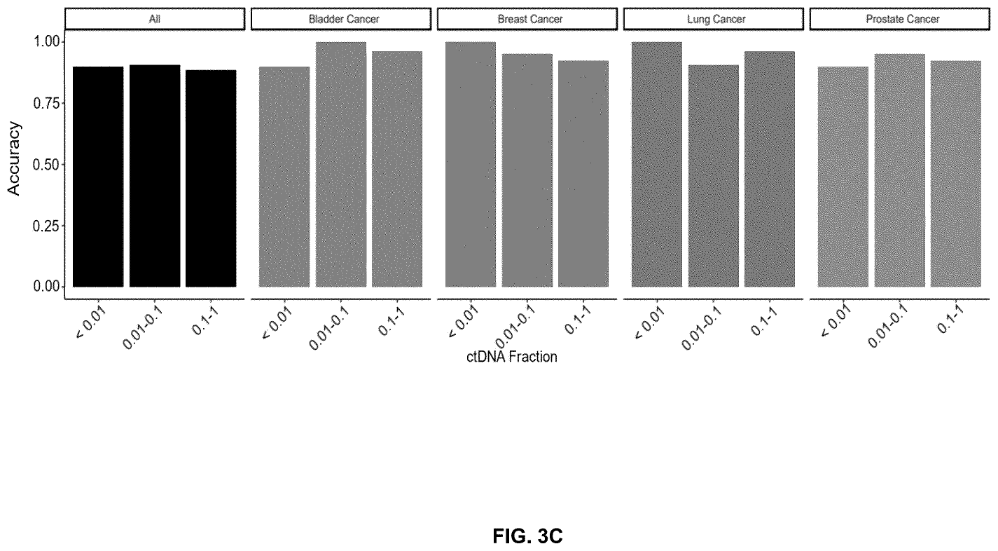

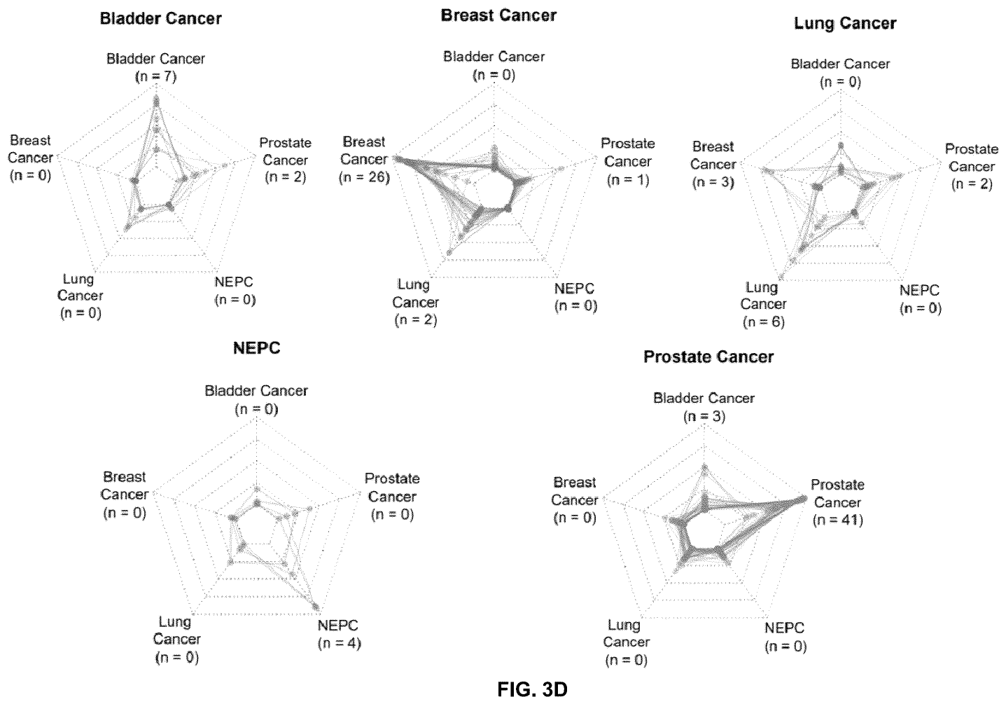

The narrow breadth of genomic coverage in targeted panels compared to WGS may bias fragmentomic patterns. When we assessed the total distribution of fragment sizes from each targeted panel, the average global fragment distributions within each phenotype across both cohorts and assays were similar. In both, we observed a main peak at 167 bp corresponding to a single nucleosome, as well as a smaller peak at 334 bp corresponding to two nucleosomes. In addition, we observed subnucleosomal peaks at smaller fragment sizes with roughly 10 bp periodicity which likely corresponding to the accessibility of DNA minor grooves to endonuclease digestion as the DNA wraps around the histone core, as well as the binding of transcription factors and other DNA-binding proteins^{7, 8} (FIGS. 2A, 2B). The fragment distribution from these targeted panels was similar to previously published cfDNA fragment patterns which used WGS^{8, 12, 14, 17, 21, 26, 42}, suggesting that fragmentomics might be successfully applied to targeted exon panels (FIGS. 3A, 3B).

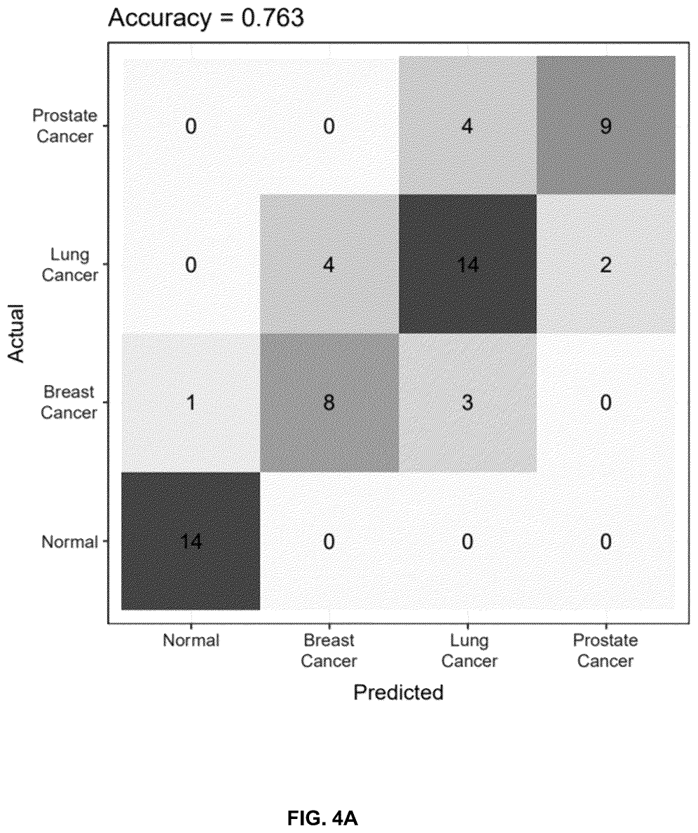

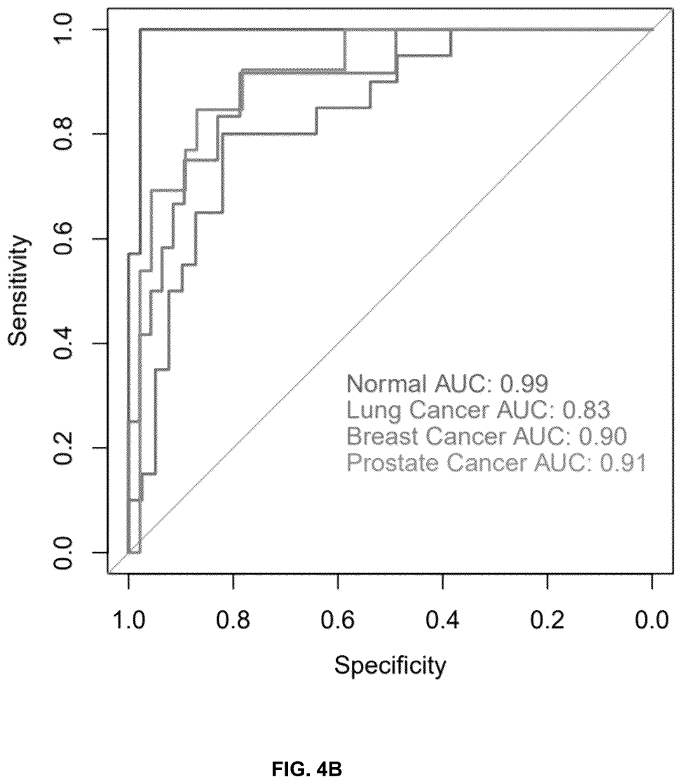

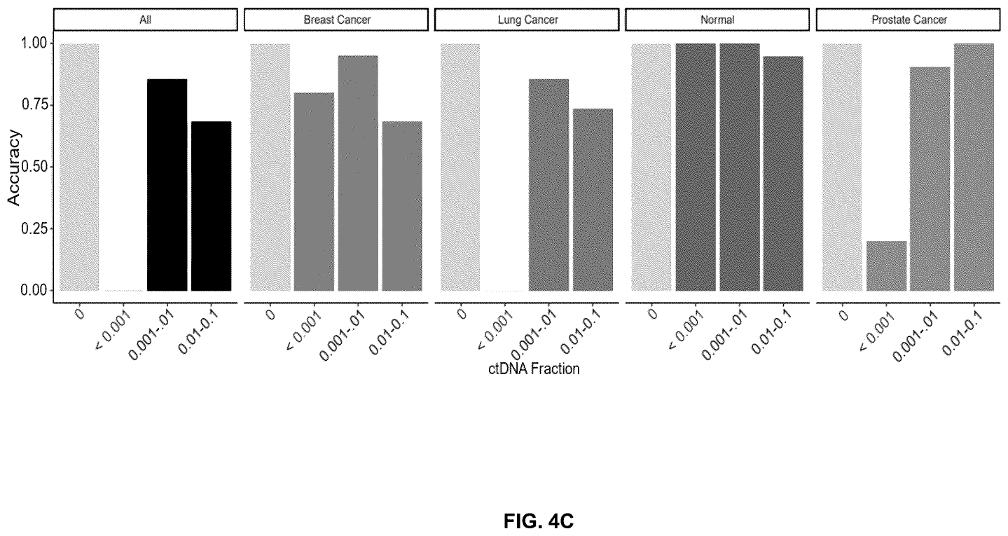

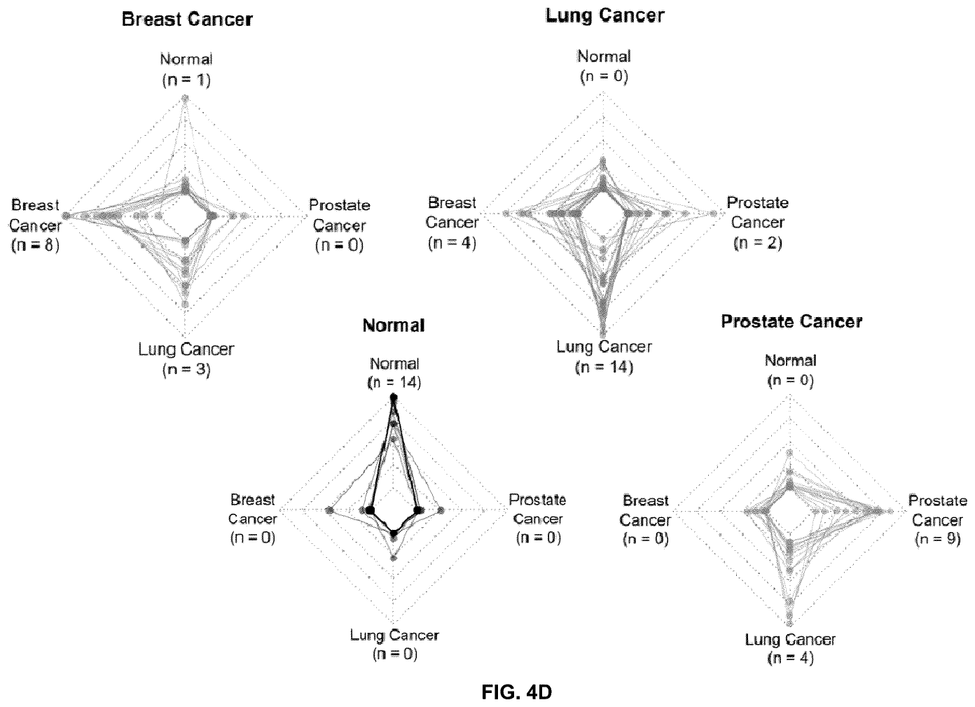

Repressed genes contain high nucleosome occupancy at their TSS, leading to a more uniform distribution of fragment reads at 167 bp^{14, 16, 43-46.} In contrast, actively expressed genes have more open chromatin at their TSS, allowing the cfDNA originating from this region to be cleaved in a more random manner, leading to a more diverse distribution of DNA fragment sizes^{14, 16, 43-46}. These changes can be detected out to 2000 bp from the TSS, which overlaps most first coding exons^{7, 14, 47}. When we compared the fragment coverage around the TSS and first coding exon in highly expressed vs. lowly expressed genes from deep WGS in a separate cohort⁴⁸, we found that the lower coverage observed at the TSS of highly expressed genes extended well into the first coding exon, indicating that fragmentation profiles in the first coding exon are linked to gene expression (FIGS. 4A-4D). This is important because the majority of targeted cancer gene panels, including the GRAIL and UW panels, do not include the TSS in most cases and instead start at the first coding exon of targeted genes.

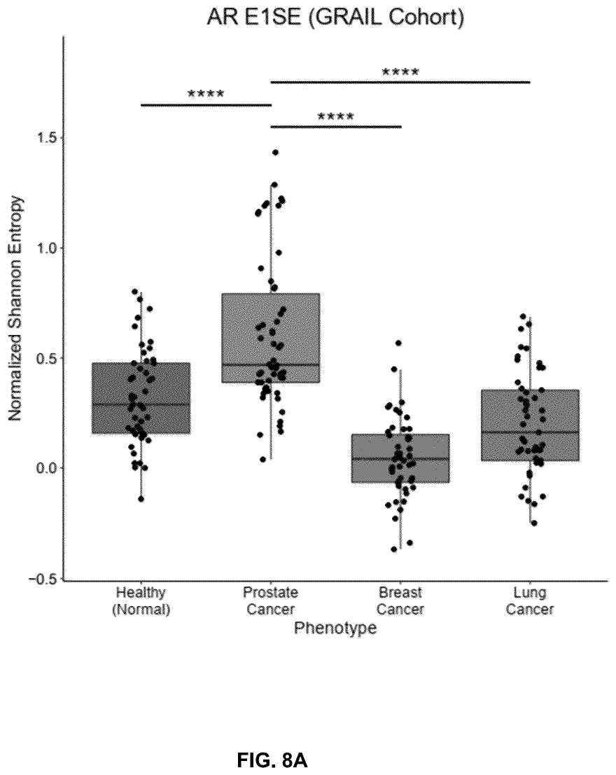

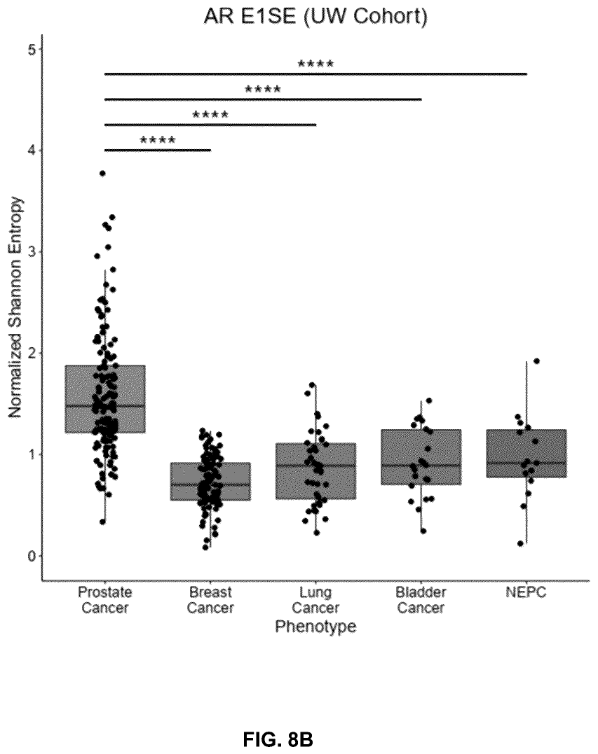

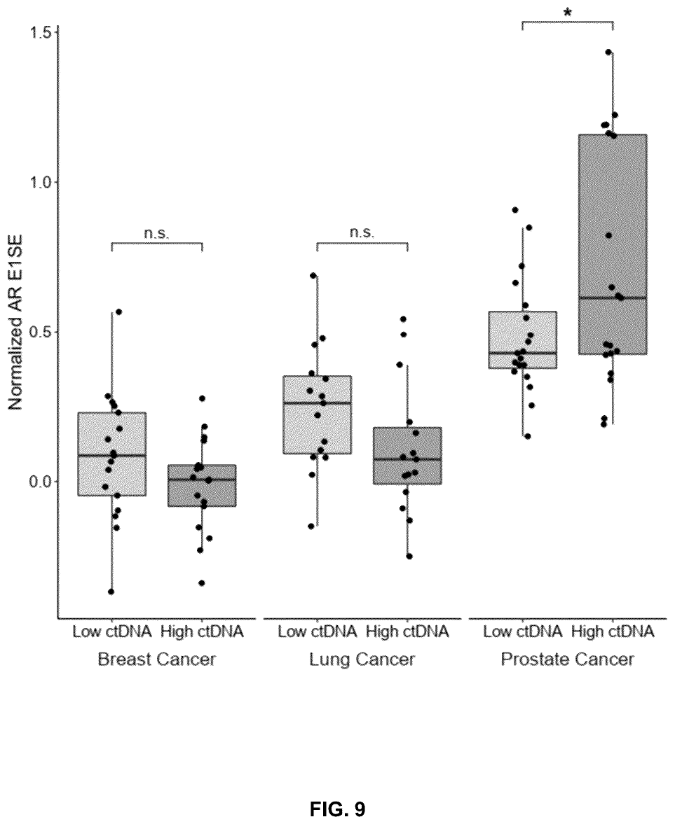

To assess the diversity of fragment sizes at the first coding exon of each gene, Shannon entropies were calculated for each individual gene in the respective sequencing panels for each patient using the distribution of fragment sizes overlapping the first coding exon. We defined this metric as Exon 1 Shannon Entropy (E1SE). To visualize the relationship between E1SE and fragment size distribution, we plotted the fragment distributions of all analyzed genes from highest to lowest E1SE within individual samples from each cohort and noted that, as expected, high E1SE genes were depleted in fragments around the mode of 167 bp with an increased proportion of fragments at lower (<120 bp) and higher (>200 bp) sizes (FIGS. 2C, 2D; individual representative sample shown for each cohort). Conversely, low E1SE genes displayed a higher proportion of fragments at the mono-nucleosome peak (167 bp), suggesting a more closed chromatin structure at exon 1 of those genes. We additionally noted that the E1SE of the androgen receptor gene (AR) was significantly higher in prostate cancer samples compared to all other cancer types and normal samples in both the GRAIL and UW cohorts (FIGS. 8A, 8B). Further, AR E1SE was observed to be higher in high ctDNA fraction prostate cancer samples, but not lung cancer or breast cancer samples, suggesting that the high AR E1SE originates from tumor-derived cfDNA (FIG. 9). This example highlights how differences in E1SE levels could help distinguish between tumor types and subtypes.

Copy number alterations are common in cancer and can affect the number of reads mapping to each gene, which could potentially bias the measurement of fragment size diversity via E1SE. However, we did not observe a clear relationship between copy number and E1SE (FIG. 2E). E1SE did start to trend up at very high copy numbers, though this should be interpreted with caution as there were only a small number of high copy number genes across our samples. Another possible influence on E1SE is the total number of observations used in its calculation, which corresponds in our application to the number of fragments analyzed per exon. Variation in depth of sequencing at each exon can occur through variations in targeted probe pull-down efficiency and other technical factors. To isolate this effect from copy number, we analyzed the effect of the number of fragments per exon on E1SE only in copy number neutral regions. The total number of reads mapped to an exon did not affect E1SE above a count of ˜100 (FIG. 2F). GC content has also been shown to potentially bias cfDNA sequencing and various studies have corrected for this bias when performing fragmentomics analyses through shallow whole genome sequencing^{17, 20, 23}. However, we did not find a significant correlation between exon 1 GC content and E1SE in either cohort (FIGS. 2G, 2H), possibly because these panels target a much smaller proportion of the genome and are comprised primarily of coding DNA. Thus, we sought to assess the potential utility of E1SE in classifying and subtyping tumors using targeted panel fragmentomics, while simultaneously allowing for standard ctDNA somatic alteration identification.

### E1SE Fragmentomics Distinguishes Tumor Subtypes

First, we examined if the E1SE fragmentation patterns could be used to reliably classify different cancer types in our institutional cohort and panel. The UW cohort contained 320 samples from patients with metastatic disease from six different tumor types: breast cancer (N=100), bladder cancer (N=22), lung cancer (N=39), and prostate cancer (N=144). In addition, we had samples from patients with metastatic neuroendocrine prostate cancer (N=15, NEPC), a molecularly and clinically distinct subtype of prostate cancer.

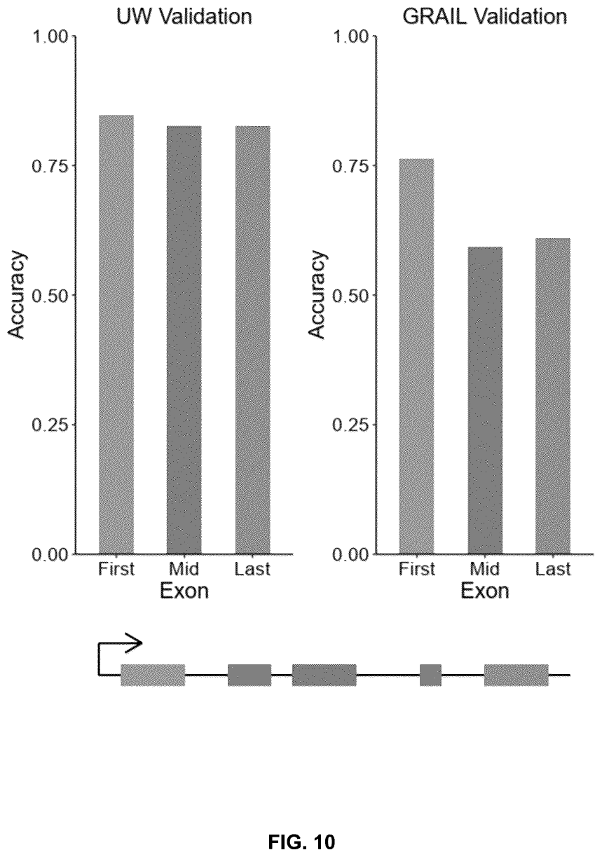

Fragmentomic differences are subtle, and many studies use machine learning approaches to assess fragmentomic biomarkers. We used elastic-net regression to train a multi-class classifier to distinguish the different tumor types in the UW cohort, which was split into 70% training and 30% independent validation. In the training cohort, we utilized 10-fold cross validation to assess performance and compared this to the independent validation. We found that in the training cohort, the E1SE model was able to distinguish the different tumor types with an overall accuracy of 82.1% on cross-validation. The performance was similar in the independent validation cohort, with an overall accuracy of 86.6% (FIG. 3A). We additionally tested the performance of the model using the middle and last coding exon of each gene and found that accuracy was highest when using the first coding exon (FIG. 10). When we examined the ROC curves for each tumor type, the AUCs for all tumor types were ≥0.89 (bladder cancer=0.98, breast cancer=0.98, lung cancer=0.89, prostate cancer=0.99, NEPC=1.00, FIG. 3B) indicating that E1SE is able to distinguish between tumor types and subtypes. These results were achieved despite a median ctDNA fraction of only 0.06. Prediction accuracy remained high across ctDNA fractions, though numbers are small in some subgroups (FIG. 3C). We additionally analyzed the prediction scores for each sample within each cancer type to determine if incorrect predictions within a cancer type were biased toward a certain cancer. In all cancer types, the majority of samples had prediction scores matching the diagnosed cancer type for that patient (FIG. 3D).

E1SE Fragmentomics Distinguishes Tumor Types and Tumor vs. Normal in Low ctDNA Fraction Samples

Given the multiplicity of targeted cfDNA sequencing platforms currently in clinical and research use that can differ quite substantially in targeted genes and depth of sequencing, we sought to test whether our approach was reproducible, robust, and independent of the specific targeted sequencing panel used. Due to differences in panel construction, an independent model would be needed for each platform of interest. We therefore performed a similar approach in the GRAIL panel and cohort, which contained 198 samples from patients with lung cancer (N=49), breast cancer (N=48), prostate cancer (N=54), as well as patients without cancer (N=47)²⁹. Approximately 347 of the genes overlap between the GRAIL and UW targeted sequencing panels. Because of the different panel designs, model training was performed again using the GRAIL cohort and panel. The median ctDNA fraction in the GRAIL cohort was 0.076 and the depth of sequencing was much higher than in our institutional cohort allowing an order of magnitude greater resolution of very low ctDNA fraction samples. Therefore, we sought to investigate the sensitivity of E1SE in distinguishing tumor types and normal samples at low ctDNA fractions. To assess this, we split the GRAIL cohort into 70% training and 30% validation based on ctDNA fractions, where the validation cohort consisted of the samples with the lowest ctDNA fractions, all <0.0481, and the training cohort contains all remaining samples.

We found that in the training cohort, the E1SE model was able to distinguish the different tumor types with an overall accuracy of 80.6% on cross-validation. Remarkably, in the independent validation, even at these low ctDNA fractions, the E1SE model had an overall accuracy of 76.3% (FIG. 4A). As with the UW cohort, we additionally tested model performance using the middle and last coding exon of each gene and found that accuracy was highest when using the first coding exon. (FIG. 10). When we examined the ROC curves for each tumor type, the AUCs were all ≥0.83 (breast cancer=0.90, lung cancer=0.83, prostate cancer=0.91, tumor vs. normal=0.99, FIG. 4B). Prediction accuracy was high in ctDNA fractions down to 0.001, with an accuracy of 85.7% in samples with ctDNA fractions from 0.001 to 0.01 (FIG. 4C). Unsurprisingly, accuracy was 0% in predicting tumor type in ctDNA fractions <0.001, thus identifying the lower limit of distinguishing different tumor types with this approach. Notably, when considering the three tumor types grouped together into a single “cancer” category, the accuracy of distinguishing cancer samples from normal samples was 100% in samples with ctDNA fraction <0.001, with the lowest ctDNA fraction being 0.0003. When we analyzed the prediction scores for each cancer type, as with the UW cohort, the majority of samples were correctly predicted as their true cancer type (FIG. 4D).

### Assessing Performance as a Function of Sequencing Depth

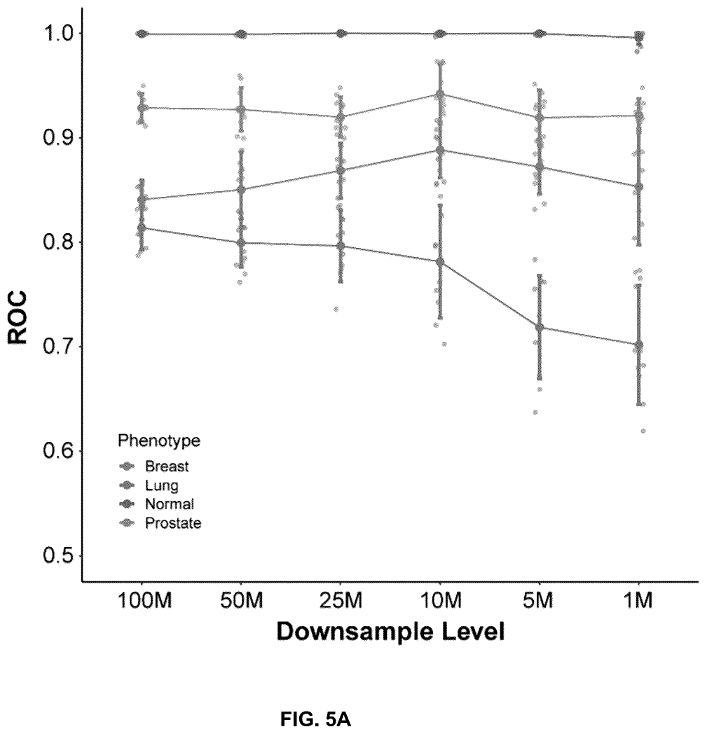

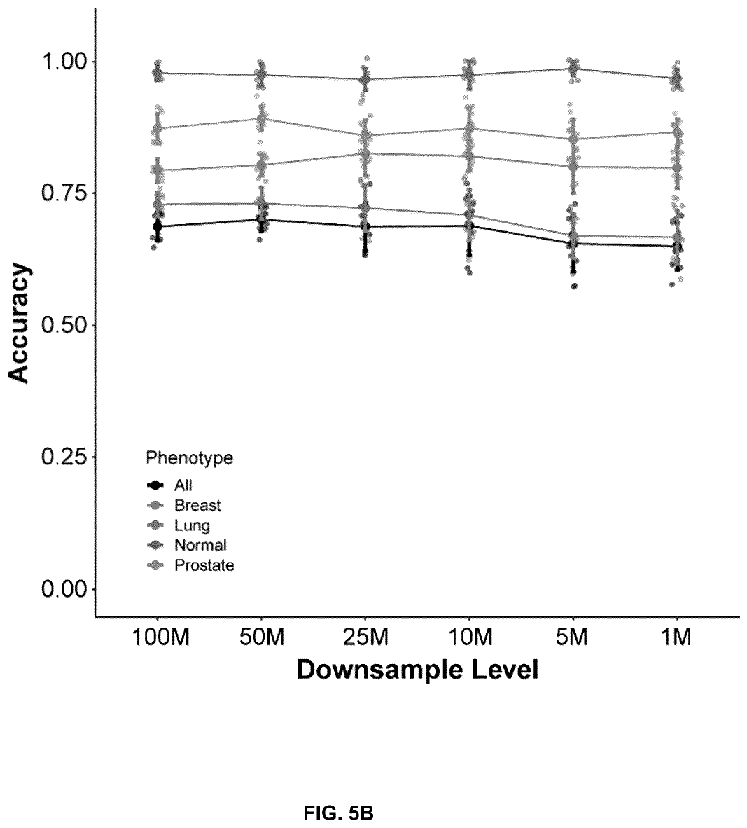

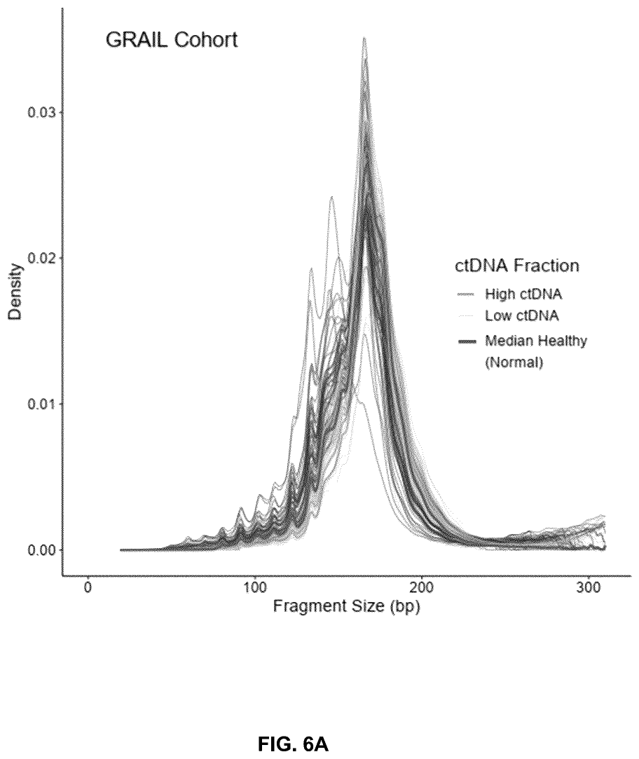

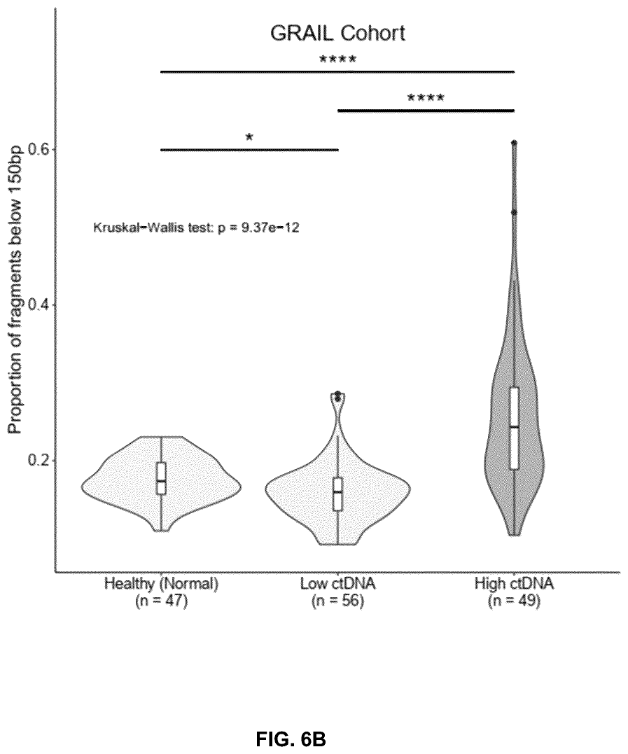

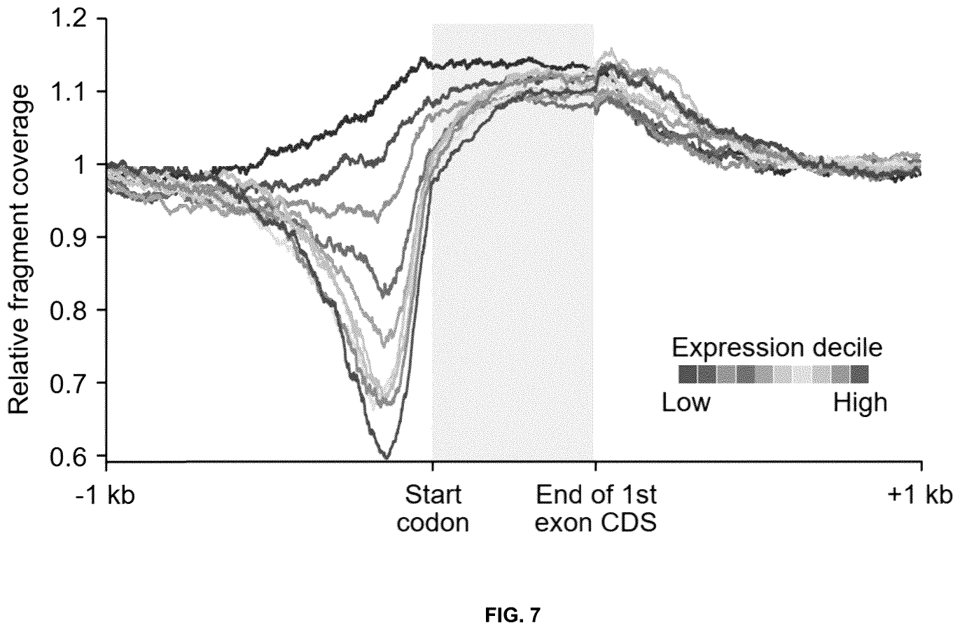

Since the cost of NGS is not trivial, we wanted to evaluate how performance of the E1SE fragmentomics model varied as a function of depth of sequencing. To do this, we performed down-sampling of GRAIL cohort after the de-duplication step as this assessed the effect of unique read depth on model performance. Due to the increased depth of sequencing from the GRAIL data, we were able to down-sample all samples to 100, 50, 25, 10, 5, and 1 million de-duplicated reads which correspond to sequencing depths of roughly 15000×, 7500×, 3750×, 1500×, 750×, and 150× respectively for a 2 Mb panel. After down-sampling, E1SE were calculated as described above. This down-sampling process was repeated ten times at each level to account for variability, and the resulting E1SE tables were used for model training, with assessment being performed in the independent validation cohort as above. Interestingly, we found that reduced sequencing had only a modest impact on model performance, with AUCs between 100 million and 10 million reads remaining stable for breast (0.841 vs 0.888), prostate (0.929 vs 0.942), lung (0.814 vs. 0.781), and tumor vs. normal (1.00 vs 0.996) (FIG. 5A). Predicting tumor vs. normal is particularly robust, with the mean AUC remaining close to 1 when down-sampled to 1 M reads (AUC=0.996). Similarly, down-sampling was found to have limited effect on the accuracy of the model, both overall and within cancer types down to 1 million reads (FIG. 5B). These results indicate that high levels of depth are not required for tumor type prediction using fragmentomics approaches within targeted panels and allows for its application to sequencing depths used in standard variant calling.

### Discussion

Fragmentomic patterns of cfDNA are non-uniform and may reflect transcriptional and epigenetic changes from their cell of origin. A major challenge with current fragmentomic approaches is the requirement for WGS, which cannot be cost-effectively used to identify somatic alterations and thus is not the current standard for clinical assays. Herein, we describe the first fragmentomic approach that can use targeted cancer gene cfDNA panels to accurately classify tumor vs. normal as well as tumor types and subtypes, which performs in the same range as commercial WGS fragmentomics approaches^{17,18}. This approach remains accurate at distinguishing different tumor types and subtypes down to a ctDNA fraction of 0.001. At this ctDNA fraction, the GRAIL assay only has a sensitivity for detecting variants of 65-75%²⁸. The ability to distinguish prostate cancer adenocarcinoma from NEPC suggests that fragmentomics on targeted panels can also be useful in identifying clinically relevant biological subtypes for other cancers. Remarkably, this approach is nearly perfect at distinguishing tumor vs. normal samples even in samples with ctDNA fractions ranging from 0.001 to 0.0003. Sensitivity at such low ctDNA fractions suggests potential clinical applications such as multi-cancer early detection (MCED) and minimal residual disease (MRD) detection.

The applicability of fragmentomics to targeted ctDNA panels represents a tremendous practical advancement to the field. A single assay could provide multiple layers of information depending on ctDNA fraction. Tumor type from fragmentomics can be identified reliably down to 0.1% ctDNA with high depth of sequencing, lower than many assays can even reliably detect somatic alterations^{28, 52, 53}. Below that, tumor vs. normal can still be identified using fragmentomic approaches. Since ctDNA fraction is unknown prior to sequencing, a single unified assay provides the maximum data regardless, and is also cost effective. In addition, a single targeted panel cfDNA sequencing assay allows for maximal use of a plasma sample, as splitting a sample for multiple assays can decrease the sensitivity of each, especially at very low ctDNA quantities. Of note, while ctDNA fraction is a useful metric for these analyses, it is not always possible to obtain due to the lack of germline sequencing, which is required for accurate ctDNA fraction estimation. An advantage of our fragmentomics approach is that it does not require germline sequencing.

In conclusion, fragmentomics of targeted ctDNA panels is not only feasible, but can accurately distinguish tumor site of origin, tumor subtypes, and tumor vs. normal even in low ctDNA samples. A single assay combining fragmentomics and somatic alteration detection provides tremendous performance, logistical, and cost benefits compared to separate assays for each. This approach merits incorporation into all existing and future targeted ctDNA studies.

Additional considerations are provided in Helzer et al. 2023 (Helzer K T, Sharifi M N, Sperger J M, Shi Y, Annala M, Bootsma M L, Reese S R, Taylor A, Kaufmann K R, Krause H K, Schehr J L, Sethakorn N, Kosoff D, Kyriakopoulos C, Burkard M E, Rydzewski N R, Yu M, Harari P M, Bassetti M, Blitzer G, Floberg J, Sjöström M, Quigley D A, Dehm S M, Armstrong A J, Beltran H, Mckay R R, Feng F Y, O'Regan R, Wisinski K B, Emamekhoo H, Wyatt A W, Lang J M, Zhao S G. Fragmentomic analysis of circulating tumor DNA-targeted cancer panels. Ann Oncol. 2023 September;34(9):813-825), which is incorporated by reference in its entirety and forms a part of the present disclosure.

### REFERENCES

1 Diaz L A, Jr., Bardelli A. Liquid biopsies: genotyping circulating tumor DNA. J Clin Oncol 2014; 32 (6): 579-586.
2 Chen M, Zhao H. Next-generation sequencing in liquid biopsy: cancer screening and early detection. Hum Genomics 2019; 13 (1): 34.
3 Yao W, Mei C, Nan X et al. Evaluation and comparison of in vitro degradation kinetics of DNA in serum, urine and saliva: A qualitative study. Gene 2016; 590 (1): 142-148.
4 Watanabe T, Takada S, Mizuta R. Cell-free DNA in blood circulation is generated by DNase1L3 and caspase-activated DNase. Biochem Biophys Res Commun 2019; 516 (3): 790-795.
5 Fan H C, Blumenfeld Y J, Chitkara U et al. Noninvasive diagnosis of fetal aneuploidy by shotgun sequencing DNA from maternal blood. Proc Natl Acad Sci U S A 2008; 105 (42): 16266-16271.
6 Lo Y M, Chan K C, Sun H et al. Maternal plasma DNA sequencing reveals the genome-wide genetic and mutational profile of the fetus. Sci Transl Med 2010; 2 (61): 61ra91.
7 Snyder M W, Kircher M, Hill A J et al. Cell-free DNA Comprises an In Vivo Nucleosome Footprint that Informs Its Tissues-Of-Origin. Cell 2016; 164 (1-2): 57-68.
8 Sanchez C, Roch B, Mazard T et al. Circulating nuclear DNA structural features, origins, and complete size profile revealed by fragmentomics. JCI Insight 2021; 6 (7).
9 Ulz P, Thallinger G G, Auer M et al. Inferring expressed genes by whole-genome sequencing of plasma DNA. Nat Genet 2016; 48 (10): 1273-1278.
10 Rao S, Han A L, Zukowski A et al. Transcription factor-nucleosome dynamics from plasma cfDNA identifies ER-driven states in breast cancer. Sci Adv 2022; 8 (34): eabm4358.
11 Luger K, Mäder A W, Richmond R K et al. Crystal structure of the nucleosome core particle at 2.8 A resolution. Nature 1997; 389 (6648): 251-260.
12 Ivanov M, Baranova A, Butler T et al. Non-random fragmentation patterns in circulating cell-free DNA reflect epigenetic regulation. BMC Genomics 2015; 16 Suppl 13 (Suppl 13): S1.
13 Ramachandran S, Ahmad K, Henikoff S. Transcription and Remodeling Produce Asymmetrically Unwrapped Nucleosomal Intermediates. Mol Cell 2017; 68 (6): 1038-1053.e1034.
14 Esfahani M S, Hamilton E G, Mehrmohamadi M et al. Inferring gene expression from cell-free DNA fragmentation profiles. Nat Biotechnol 2022; 40 (4): 585-597.
15 Herberts C, Annala M, Sipola J et al. Deep whole-genome ctDNA chronology of treatment-resistant prostate cancer. Nature 2022; 608 (7921): 199-208.
16 Zhu G, Guo Y A, Ho D et al. Tissue-specific cell-free DNA degradation quantifies circulating tumor DNA burden. Nat Commun 2021; 12 (1): 2229.
17 Cristiano S, Leal A, Phallen J et al. Genome-wide cell-free DNA fragmentation in patients with cancer. Nature 2019; 570 (7761): 385-389.
18 Mathios D, Johansen J S, Cristiano S et al. Detection and characterization of lung cancer using cell-free DNA fragmentomes. Nat Commun 2021; 12 (1): 5060.
19 Sun K, Jiang P, Cheng S H et al. Orientation-aware plasma cell-free DNA fragmentation analysis in open chromatin regions informs tissue of origin. Genome Res 2019; 29 (3): 418-427.
20 Peneder P, Stütz A M, Surdez D et al. Multimodal analysis of cell-free DNA whole-genome sequencing for pediatric cancers with low mutational burden. Nat Commun 2021; 12 (1): 3230.
21 Mouliere F, Chandrananda D, Piskorz A M et al. Enhanced detection of circulating tumor DNA by fragment size analysis. Sci Transl Med 2018; 10 (466).
22 Ulz P, Perakis S, Zhou Q et al. Inference of transcription factor binding from cell-free DNA enables tumor subtype prediction and early detection. Nat Commun 2019; 10 (1): 4666.
23 Doebley A L, Ko M, Liao H et al. A framework for clinical cancer subtyping from nucleosome profiling of cell-free DNA. Nat Commun 2022; 13 (1): 7475.
24 Jiang P, Sun K, Peng W et al. Plasma DNA End-Motif Profiling as a Fragmentomic Marker in Cancer, Pregnancy, and Transplantation. Cancer Discov 2020; 10 (5): 664-673.
25 Underhill H R, Kitzman J O, Hellwig S et al. Fragment Length of Circulating Tumor DNA. PLOS Genet 2016; 12 (7): e1006162.
26 Jiang P, Chan C W, Chan K C et al. Lengthening and shortening of plasma DNA in hepatocellular carcinoma patients. Proc Natl Acad Sci U S A 2015; 112 (11): E1317-1325.
27 Liu Y. At the dawn: cell-free DNA fragmentomics and gene regulation. Br J Cancer 2022; 126 (3): 379-390.
28 Herberts C, Wyatt A W. Technical and biological constraints on ctDNA-based genotyping. Trends Cancer 2021; 7 (11): 995-1009.
29 Razavi P, Li BT, Brown D N et al. High-intensity sequencing reveals the sources of plasma circulating cell-free DNA variants. Nat Med 2019; 25 (12): 1928-1937.
30 Li H, Durbin R. Fast and accurate short read alignment with Burrows-Wheeler transform. Bioinformatics 2009; 25 (14): 1754-1760.
31 Quinlan A R, Hall I M. BEDTools: a flexible suite of utilities for comparing genomic features. Bioinformatics 2010; 26 (6): 841-842.
32 Lai Z, Markovets A, Ahdesmaki M et al. VarDict: a novel and versatile variant caller for next-generation sequencing in cancer research. Nucleic Acids Res 2016; 44 (11): e108.
33 Bick A G, Weinstock J S, Nandakumar S K et al. Inherited causes of clonal haematopoiesis in 97,691 whole genomes. Nature 2020; 586 (7831): 763-768.
34 Talevich E, Shain A H, Botton T et al. CNVkit: Genome-Wide Copy Number Detection and Visualization from Targeted DNA Sequencing. PLOS Comput Biol 2016; 12 (4): e1004873.
35 Vandekerkhove G, Lavoie J M, Annala M et al. Plasma ctDNA is a tumor tissue surrogate and enables clinical-genomic stratification of metastatic bladder cancer. Nat Commun 2021; 12 (1): 184.
36 Wyatt A W, Annala M, Aggarwal R et al. Concordance of Circulating Tumor DNA and Matched Metastatic Tissue Biopsy in Prostate Cancer. J Natl Cancer Inst 2017; 109 (12).
37 Annala M, Taavitsainen S, Khalaf D J et al. Evolution of Castration-Resistant Prostate Cancer in ctDNA during Sequential Androgen Receptor Pathway Inhibition. Clin Cancer Res 2021; 27 (16): 4610-4623.
38 Annala M, Vandekerkhove G, Khalaf D et al. Circulating Tumor DNA Genomics Correlate with Resistance to Abiraterone and Enzalutamide in Prostate Cancer. Cancer Discov 2018; 8 (4): 444-457.
39 Vandekerkhove G, Struss W J, Annala M et al. Circulating Tumor DNA Abundance and Potential Utility in De Novo Metastatic Prostate Cancer. Eur Urol 2019; 75 (4): 667-675.
40 Vandekerkhove G, Todenhöfer T, Annala M et al. Circulating Tumor DNA Reveals

Clinically Actionable Somatic Genome of Metastatic Bladder Cancer. Clin Cancer Res 2017; 23 (21): 6487-6497.
41 Mizuno K, Sumiyoshi T, Okegawa T et al. Clinical Impact of Detecting Low-Frequency Variants in Cell-Free DNA on Treatment of Castration-Resistant Prostate Cancer. Clin Cancer Res 2021; 27 (22): 6164-6173.
42 Markus H, Chandrananda D, Moore E et al. Refined characterization of circulating tumor DNA through biological feature integration. Sci Rep 2022; 12 (1): 1928.
43 Hesson L B, Sloane M A, Wong J W et al. Altered promoter nucleosome positioning is an early event in gene silencing. Epigenetics 2014; 9 (10): 1422-1430.
44 Jiang C, Pugh B F. Nucleosome positioning and gene regulation: advances through genomics. Nat Rev Genet 2009; 10 (3): 161-172.
45 Lee C K, Shibata Y, Rao B et al. Evidence for nucleosome depletion at active regulatory regions genome-wide. Nat Genet 2004; 36 (8): 900-905.
46 Lai W K M, Pugh B F. Understanding nucleosome dynamics and their links to gene expression and DNA replication. Nat Rev Mol Cell Biol 2017; 18 (9): 548-562.
47 Davuluri R V, Grosse I, Zhang M Q. Computational identification of promoters and first exons in the human genome. Nat Genet 2001; 29 (4): 412-417.
48 Herberts C, Annala M, Sipola J et al. Deep whole-genome ctDNA chronology of treatment-resistant prostate cancer. Nature 2022; 608 (7921): 199-208.
49 Bieberstein NI, Carrillo Oesterreich F, Straube K et al. First exon length controls active chromatin signatures and transcription. Cell Rep 2012; 2 (1): 62-68.
50 Brenet F, Moh M, Funk P et al. DNA methylation of the first exon is tightly linked to transcriptional silencing. PLOS One 2011; 6 (1): e14524.
51 Fiszbein A, Krick K S, Begg B E et al. Exon-Mediated Activation of Transcription Starts. Cell 2019; 179 (7): 1551-1565.e1517.
52 Keller L, Belloum Y, Wikman H et al. Clinical relevance of blood-based ctDNA analysis: mutation detection and beyond. Br J Cancer 2021; 124 (2): 345-358.
53 Dang D K, Park B H. Circulating tumor DNA: current challenges for clinical utility. J Clin Invest 2022; 132 (12).

### Renal Cell Carcinoma

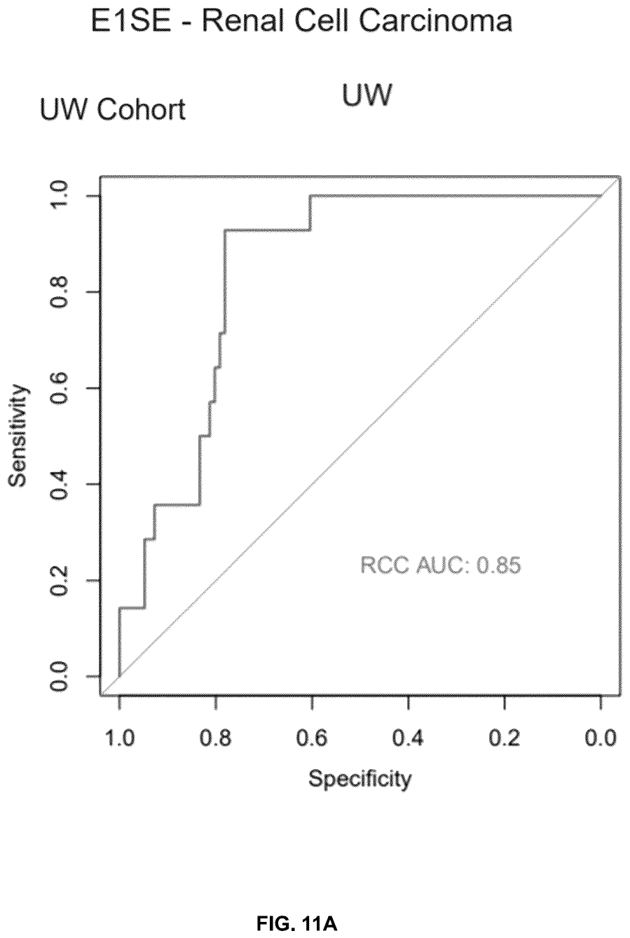

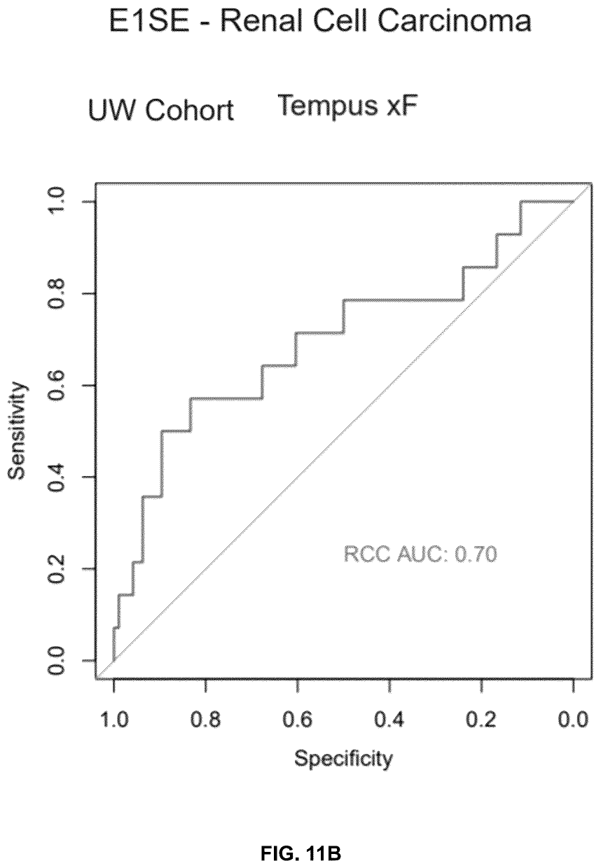

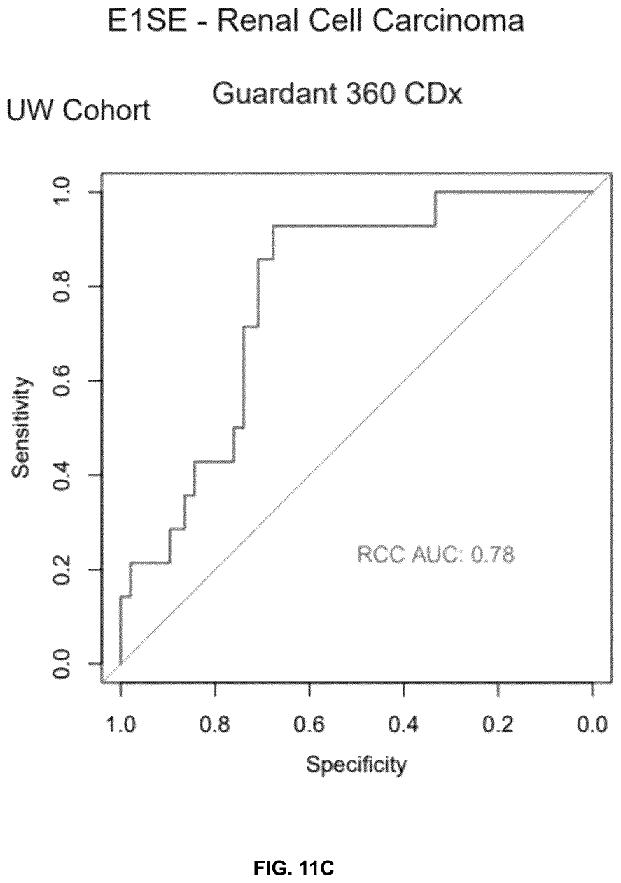

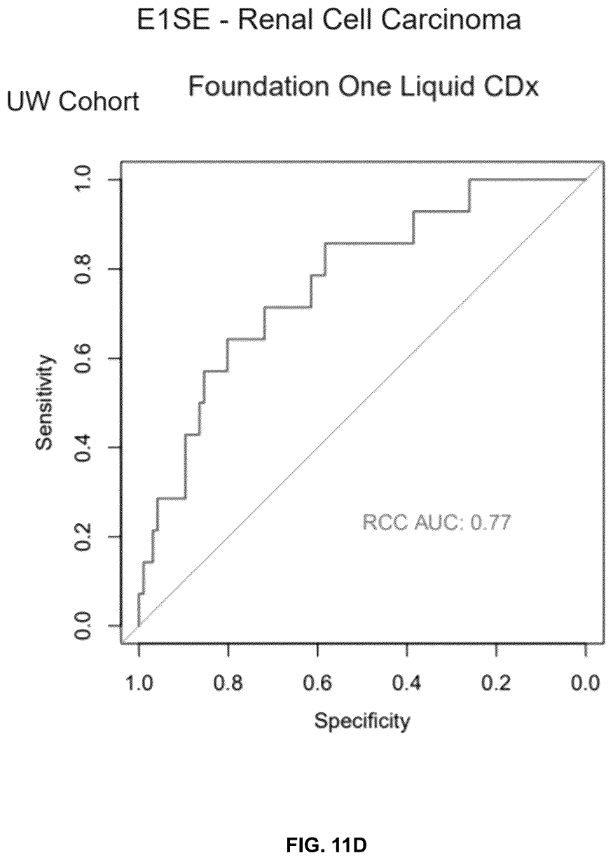

E1SE was tested for its ability to distinguish renal cell carcinoma (RCC) from other cancer types (FIGS. 11A-11D). The UW cohort contained 44 RCC samples and 320 non-RCC samples which were split 70/30 into training and validation cohort, respectively. Validation ROC AUC (which will be referred to as just AUC below) for RCC using all genes in the UW panel was 0.85. Validation AUC for RCC using common genes between the UW panel and the Tempus XF panel was 0.70. Validation AUC for RCC using common genes between the UW panel and the Guardant 360 CDx panel was 0.78. Validation AUC for RCC using common genes between the UW panel and the Foundation One Liquid CDx panel was 0.77.

### Breast Cancer Subtypes

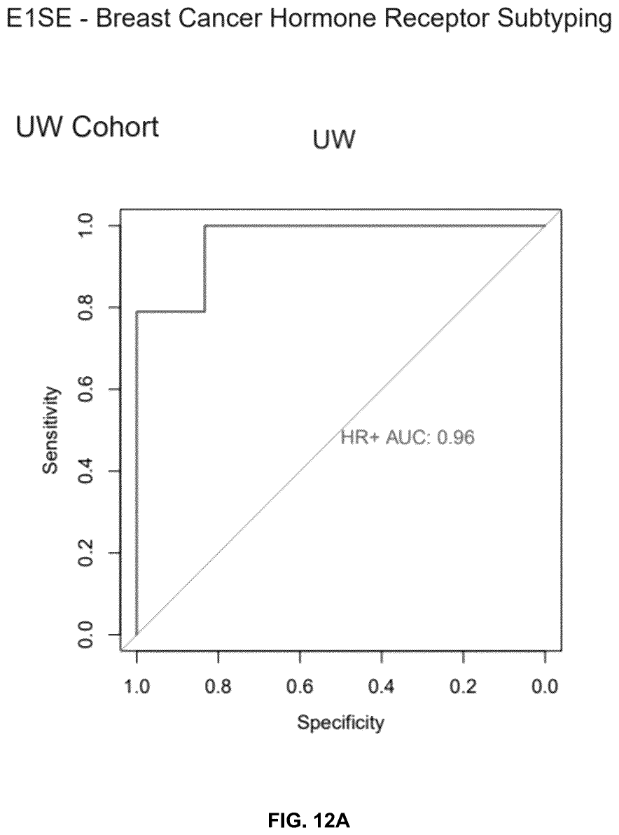

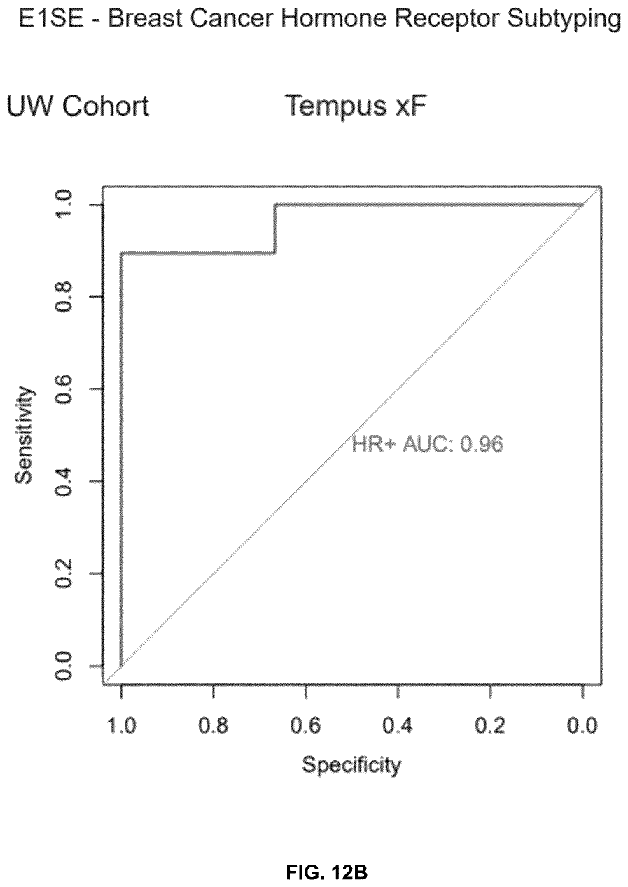

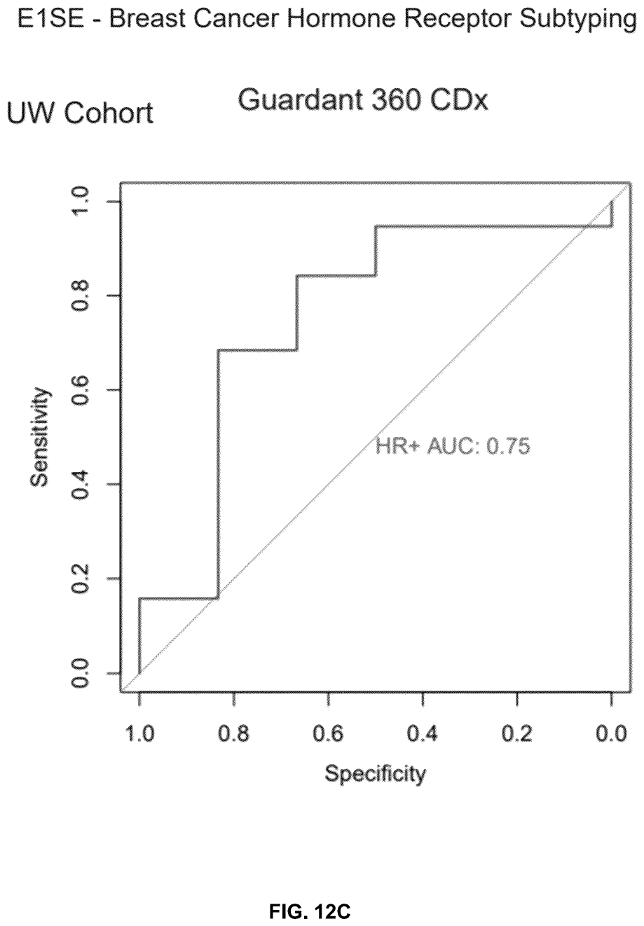

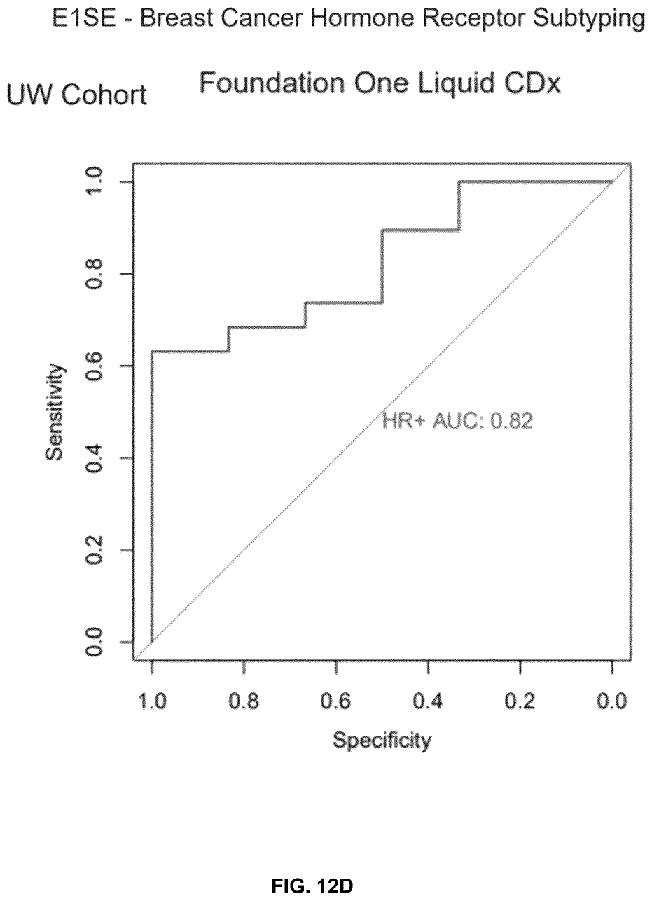

E1SE was tested for its ability to distinguish hormone receptor positive (HR+) breast cancer from triple negative breast cancer (TNBC) (FIGS. 12A-12D). The UW cohort contained 81 samples with HR subtype information, which was split 70/30 into training and validation cohort, respectively. Validation AUC for HR subtyping using all genes in the UW panel was 0.96. Validation AUC for HR subtying using common genes between the UW panel and the Tempus xF panel was 0.96. Validation AUC for HR subtying using common genes between the UW panel and the Guardant 360 CDx panel was 0.75. Validation AUC for HR subtying using common genes between the UW panel and the Foundation One Liquid CDx panel was 0.82.

### Exon 1 Shannon Entropy Using Overlapping Tempus/Guardant/Foundation Gene Lists

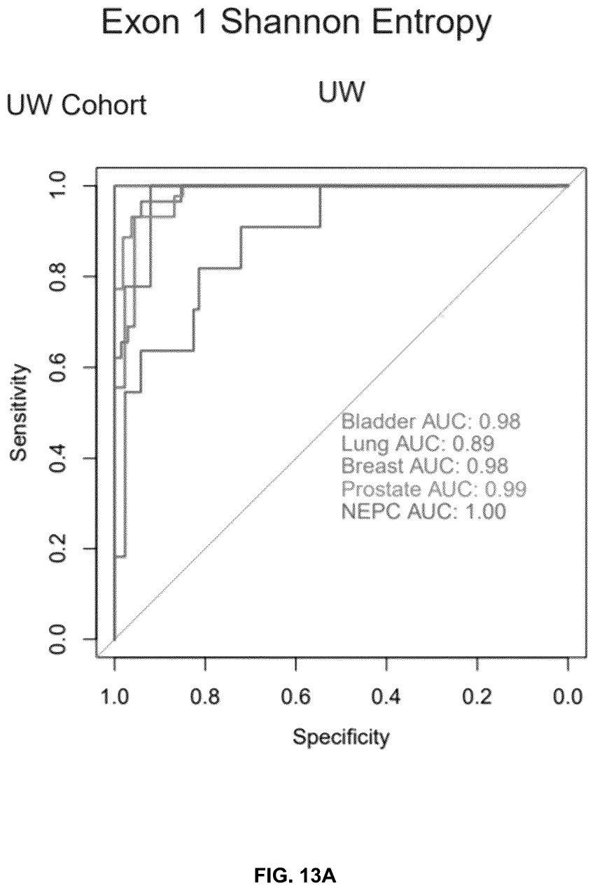

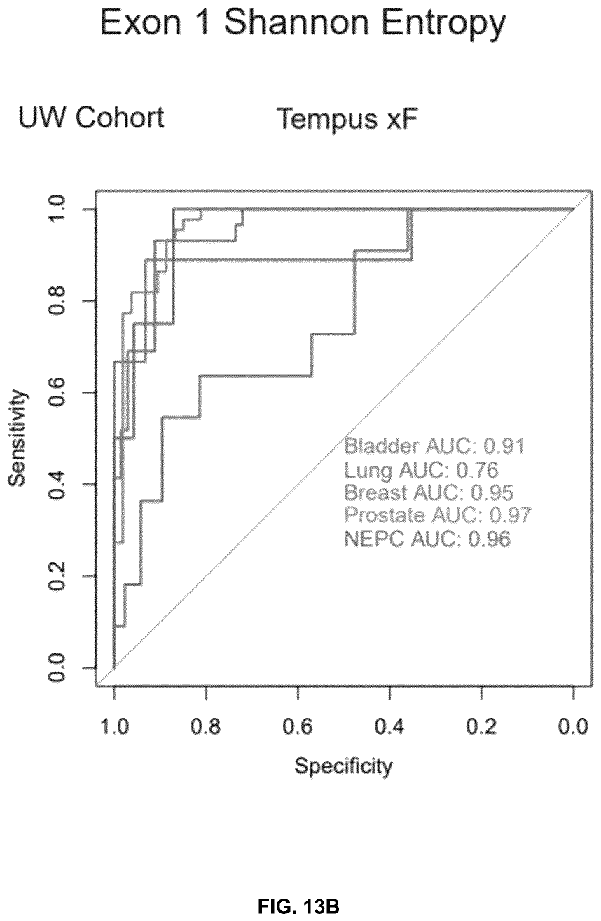

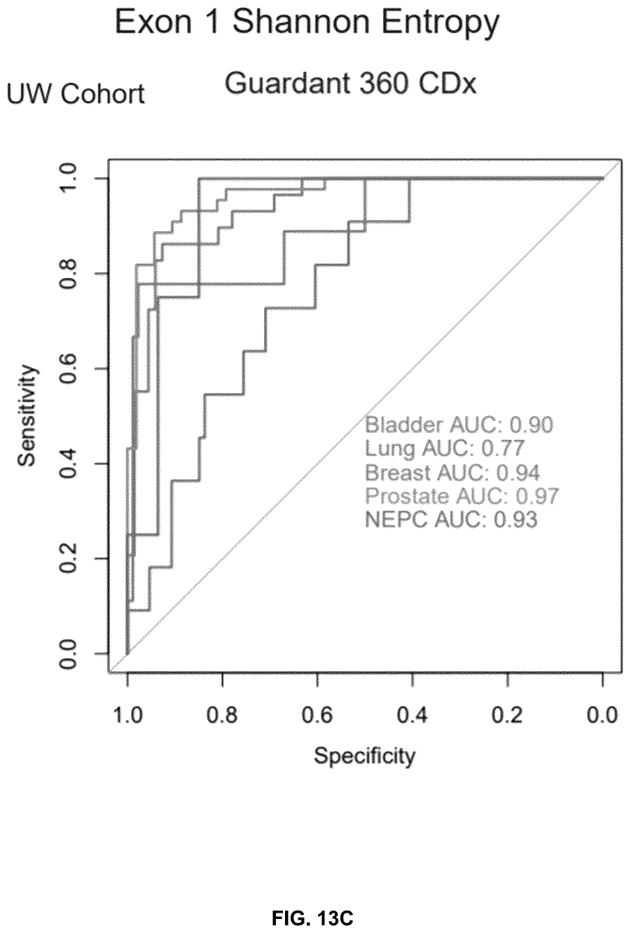

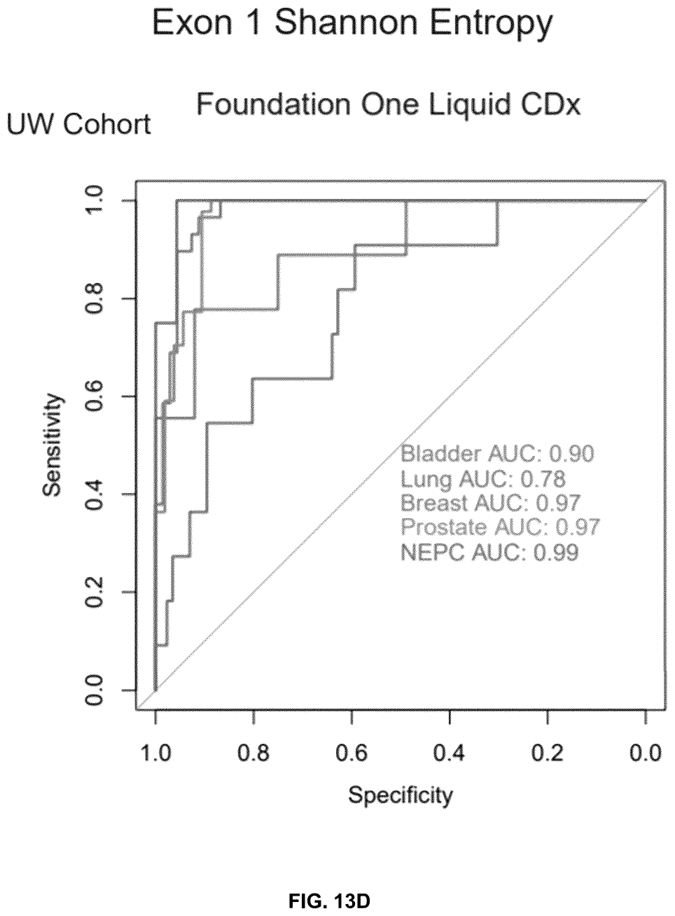

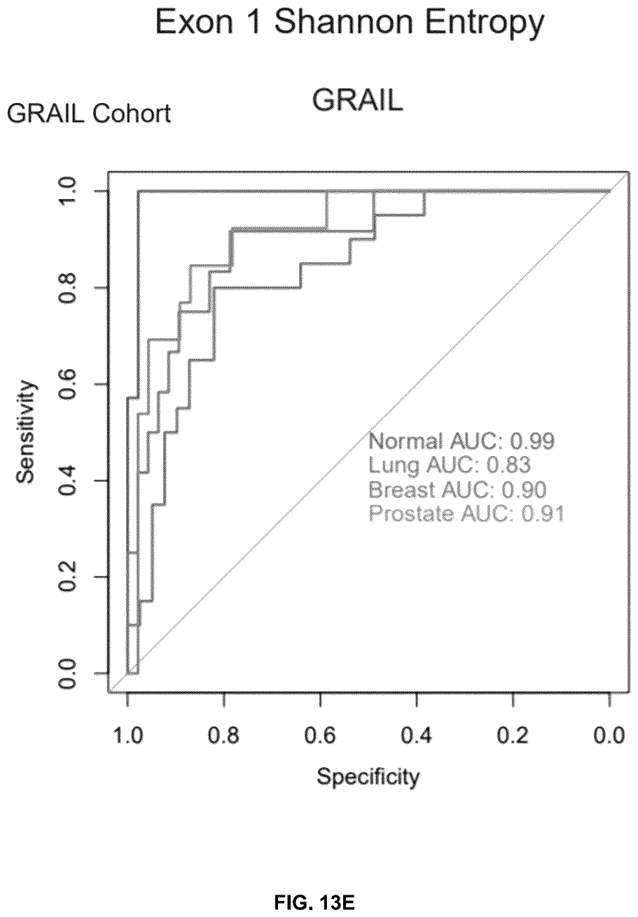

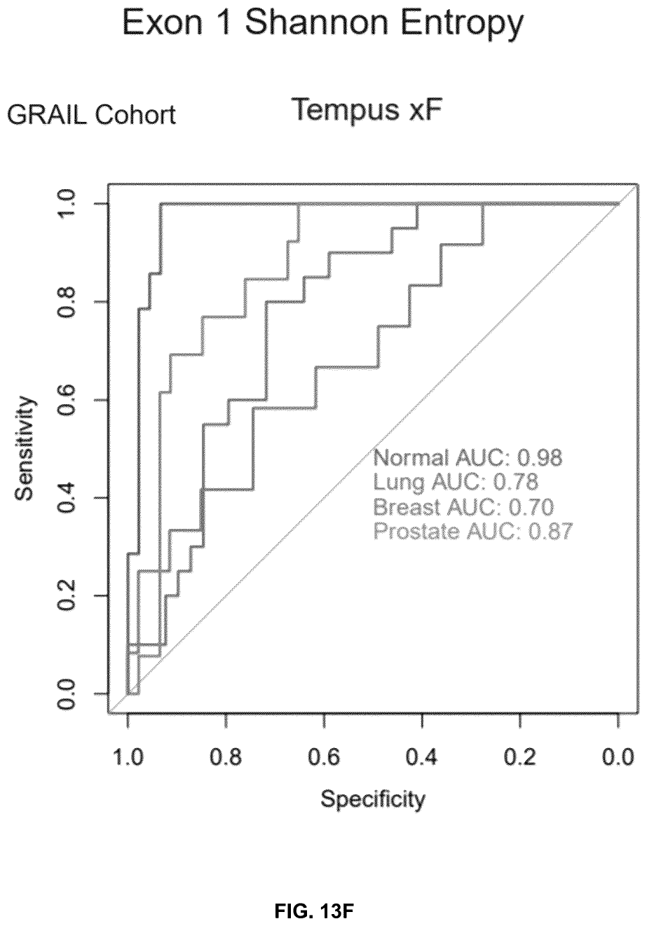

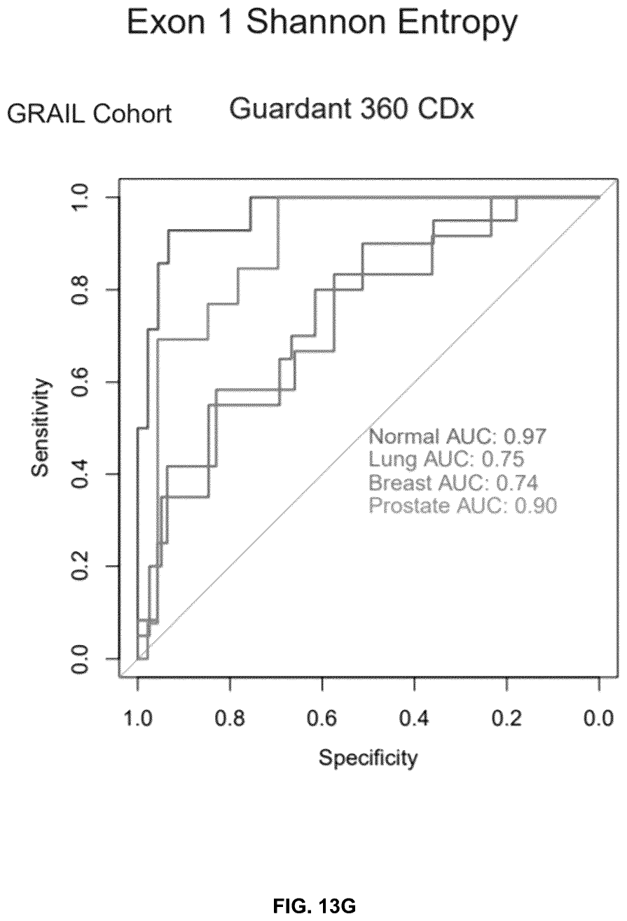

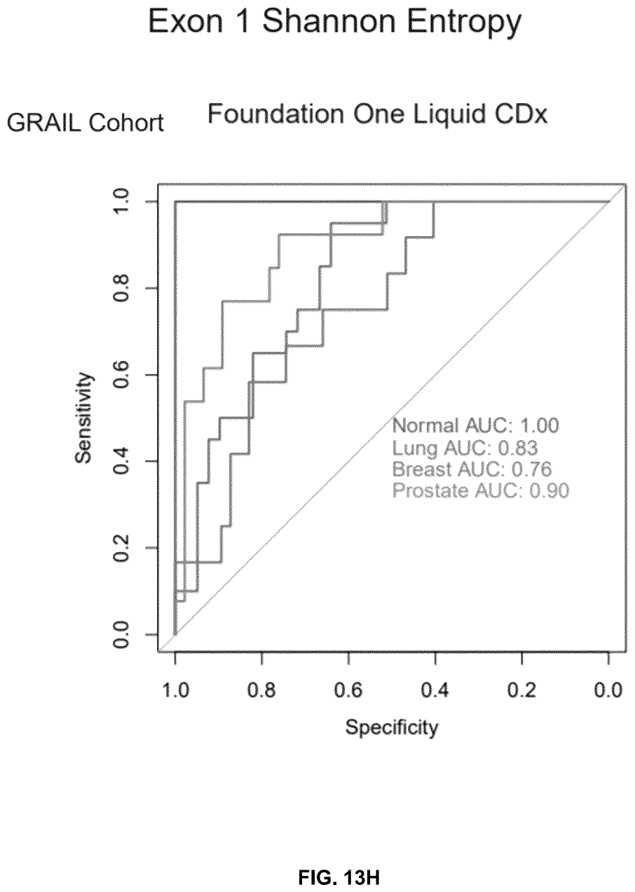

Using E1SE as above (FIGS. 13A-13H), validation AUC in the UW cohort using common genes between the UW panel and the Tempus xF panel was 0.91 for bladder cancer, 0.95 for breast cancer, 0.76 for lung cancer, 0.96 for NEPC, and 0.97 for prostate cancer. Validation AUC in the UW cohort using common genes between the UW panel and the Guardant 360 CDx panel was 0.9 for bladder cancer, 0.94 for breast cancer, 0.77 for lung cancer, 0.93 for NEPC, and 0.97 for prostate cancer. Validation AUC in the UW cohort using common genes between the UW panel and the Foundation One Liquid CDx panel was 0.9 for bladder cancer, 0.97 for breast cancer, 0.78 for lung cancer, 0.99 for NEPC, and 0.97 for prostate cancer.

Validation AUC in the GRAIL cohort using common genes between the GRAIL panel and the Tempus XF panel was 0.7 for breast cancer, 0.78 for lung cancer, 0.87 for prostate cancer, and 0.98 for cancer vs normal. Validation AUC in the GRAIL cohort using common genes between the GRAIL panel and the Guardant 360 CDx panel was 0.74 for breast cancer, 0.75 for lung cancer, 0.9 for prostate cancer, and 0.97 for cancer vs normal. Validation AUC in the GRAIL cohort using common genes between the GRAIL panel and the Foundation One Liquid CDx panel was 0.76 for breast cancer, 0.83 for lung cancer, 0.9 for prostate cancer, and 1.00 for cancer vs normal.

### Exon 1 Depth of Sequencing

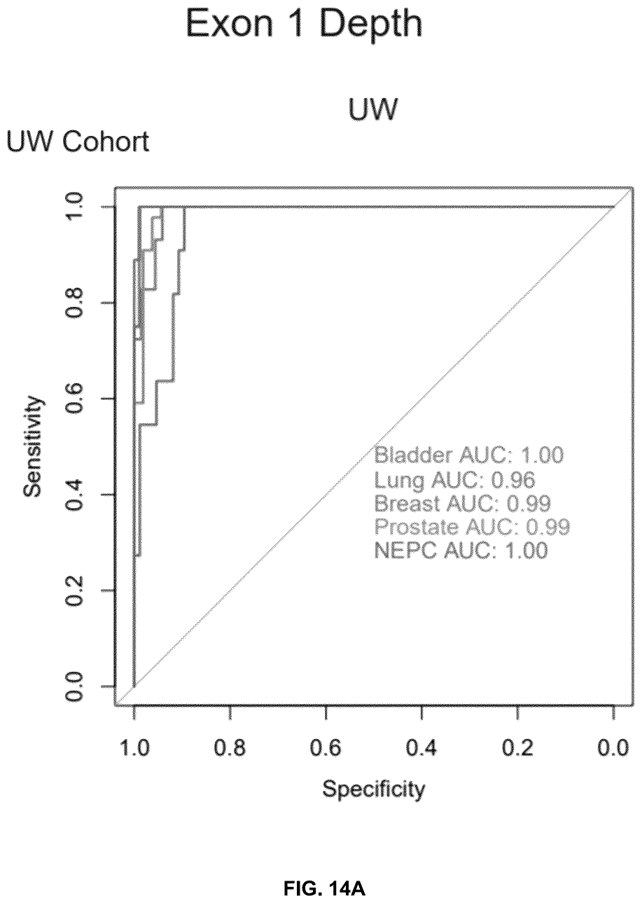

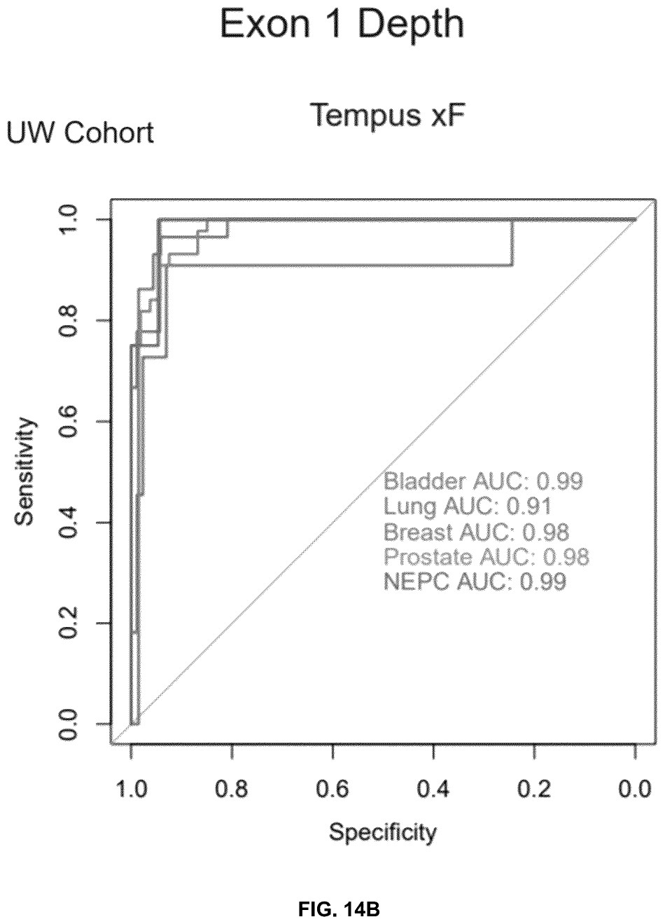

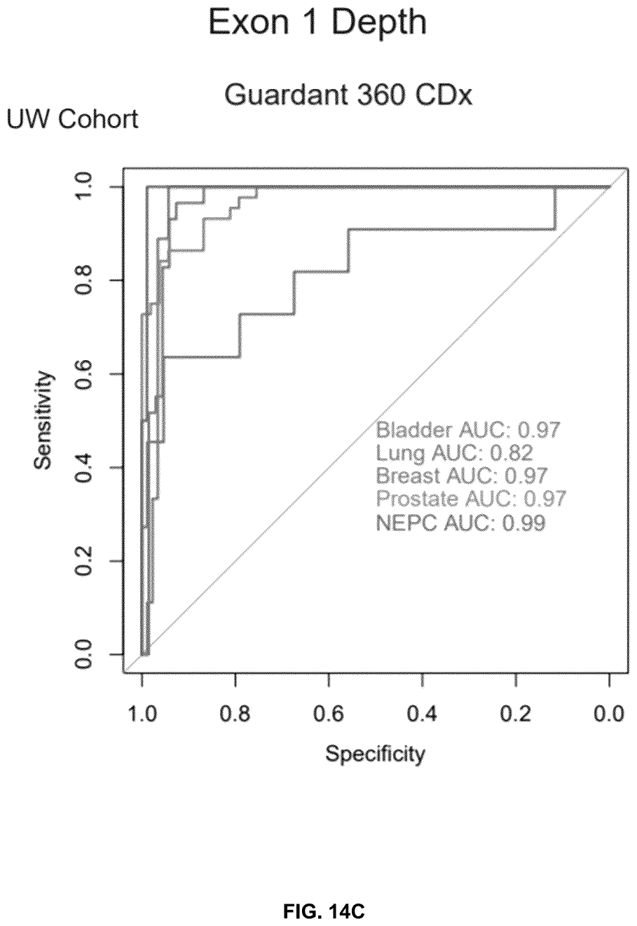

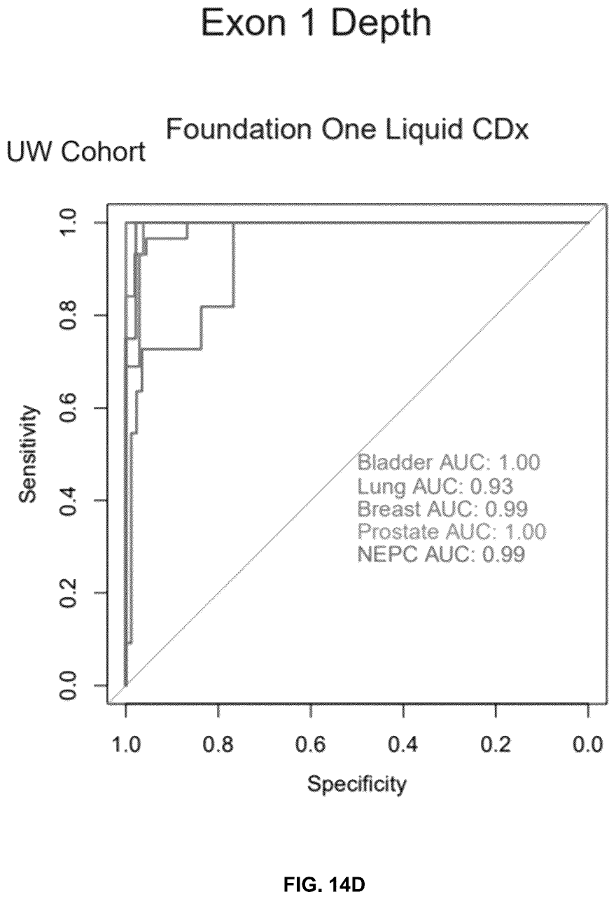

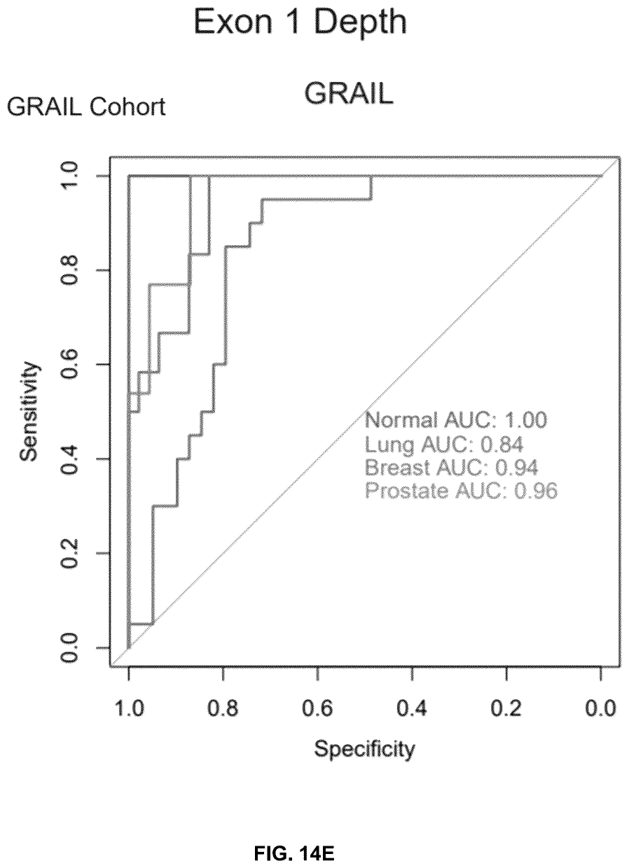

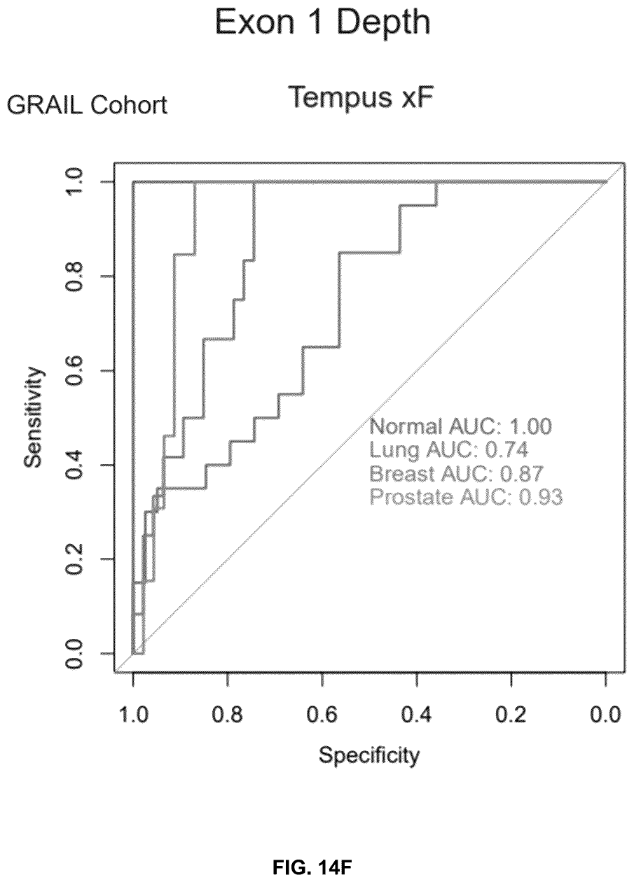

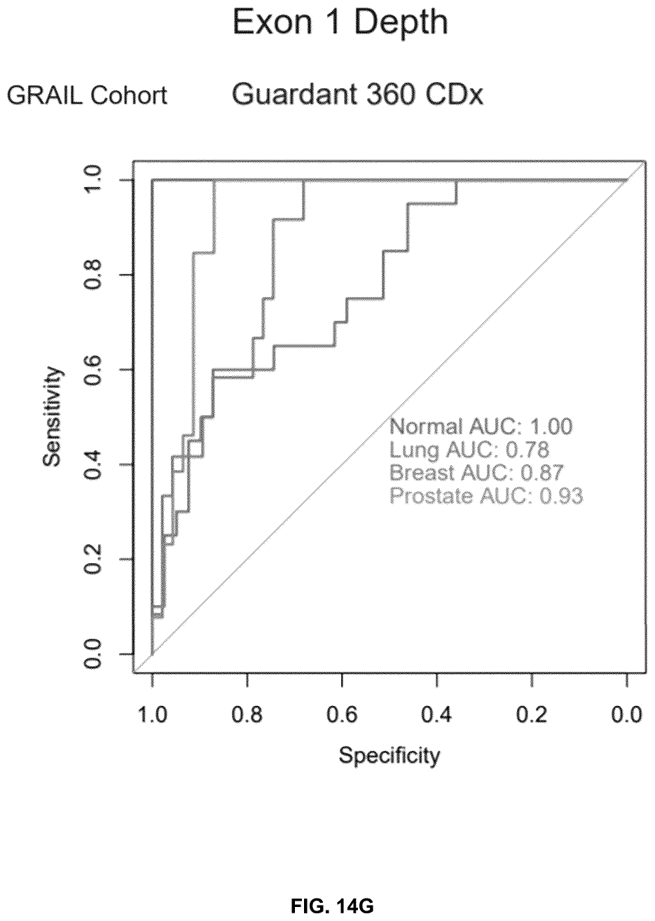

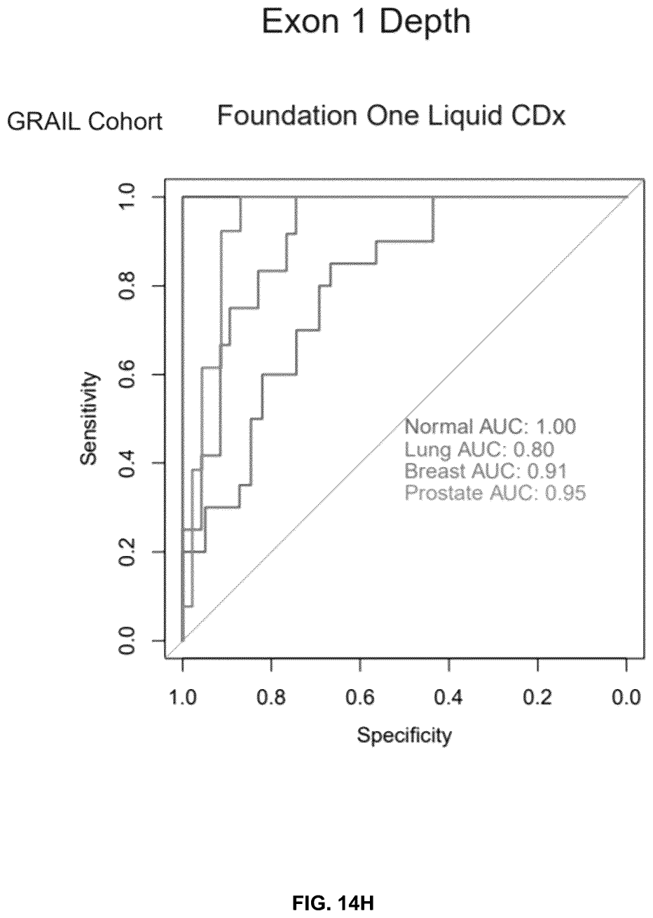

Depth of sequencing was calculated by counting the number of fragments overlapping with each individual exon across all genes in each respective panel. A minimum of 1 bp overlap was required to count as an overlap. Counts for each exon were then normalized by dividing by the size of the exon in base pairs and then dividing by the total reads in the sample. This depth metric in the first coding exons of each gene in each respective panel was used for model training and validation as in the E1SE model (FIGS. 14A-14H).

Validation AUC in the UW cohort using genes in the UW panel was 1.00 for bladder cancer, 0.99 for breast cancer, 0.96 for lung cancer, 1.00 for NEPC, and 0.99 for prostate cancer. Validation AUC in the UW cohort using common genes between the UW panel and the Tempus xF panel was 0.99 for bladder cancer, 0.98 for breast cancer, 0.91 for lung cancer, 0.99 for NEPC, and 0.98 for prostate cancer. Validation AUC in the UW cohort using common genes between the UW panel and the Guardant 360 CDx panel was 0.97 for bladder cancer, 0.97 for breast cancer, 0.82 for lung cancer, 0.99 for NEPC, and 0.97 for prostate cancer. Validation AUC in the UW cohort using common genes between the UW panel and the Foundation One Liquid CDx panel was 1.00 for bladder cancer, 0.99 for breast cancer, 0.93 for lung cancer, 0.99 for NEPC, and 1.00 for prostate cancer.

Validation AUC in the GRAIL cohort using genes in the GRAIL panel was 0.96 for breast cancer, 0.92 for lung cancer, 0.99 for prostate cancer, and 1.00 for cancer vs normal. Validation AUC in the GRAIL cohort using common genes between the GRAIL panel and the Tempus xF panel was 0.87 for breast cancer, 0.80 for lung cancer, 0.89 for prostate cancer, and 1.00 for cancer vs normal. Validation AUC in the GRAIL cohort using common genes between the GRAIL panel and the Guardant 360 CDx panel was 0.87 for breast cancer, 0.68 for lung cancer, 0.89 for prostate cancer, and 1.00 for cancer vs normal. Validation AUC in the GRAIL cohort using common genes between the GRAIL panel and the Foundation One Liquid CDx panel was 0.93 for breast cancer, 0.86 for lung cancer, 0.99 for prostate cancer, and 1.00 for cancer vs normal.

### Full Gene Depth of Sequencing

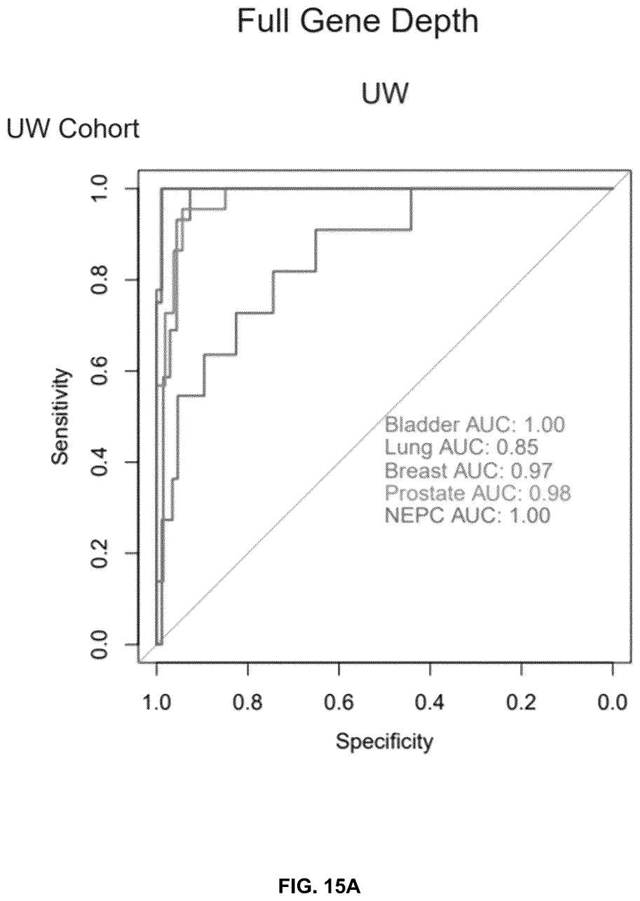

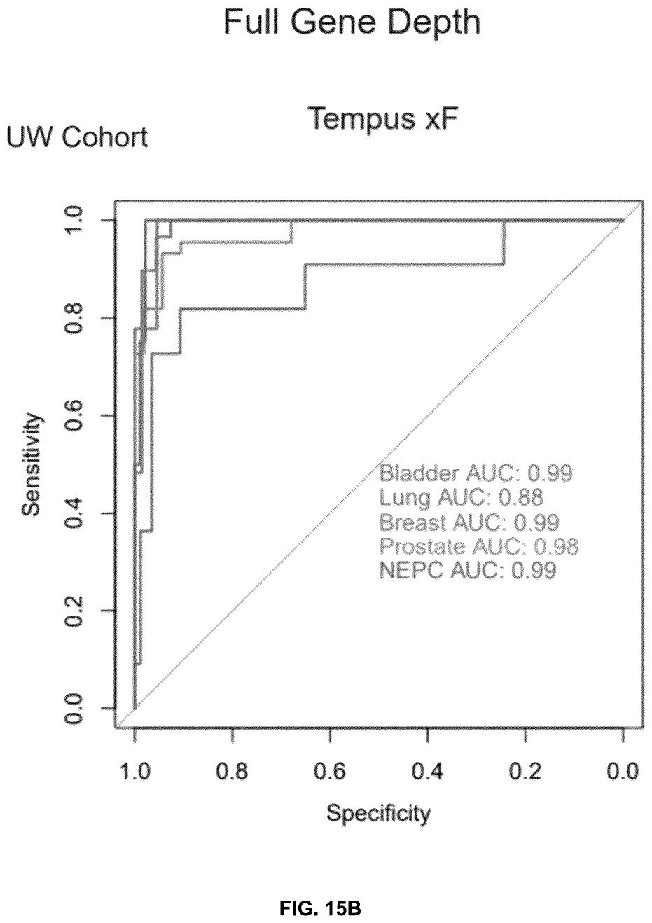

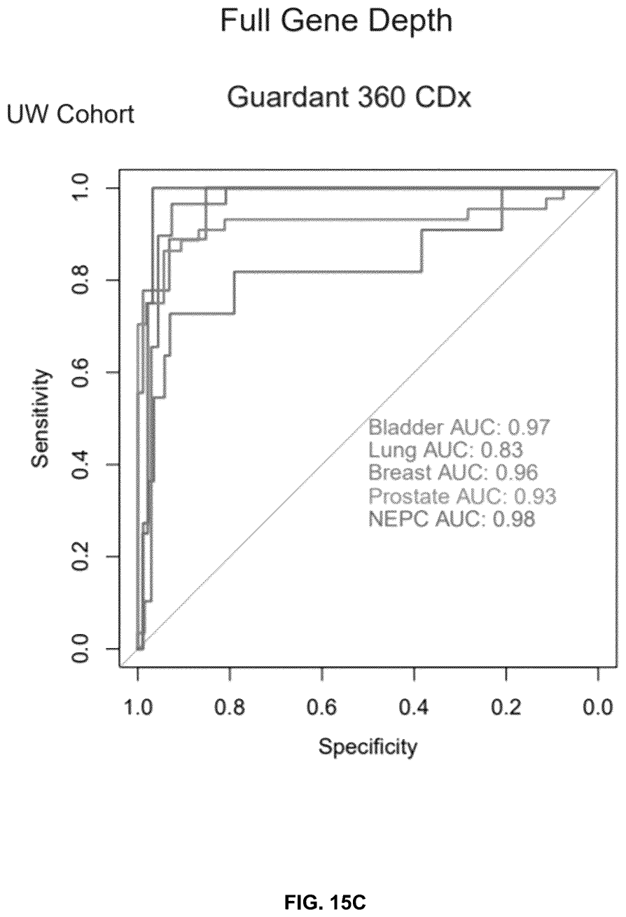

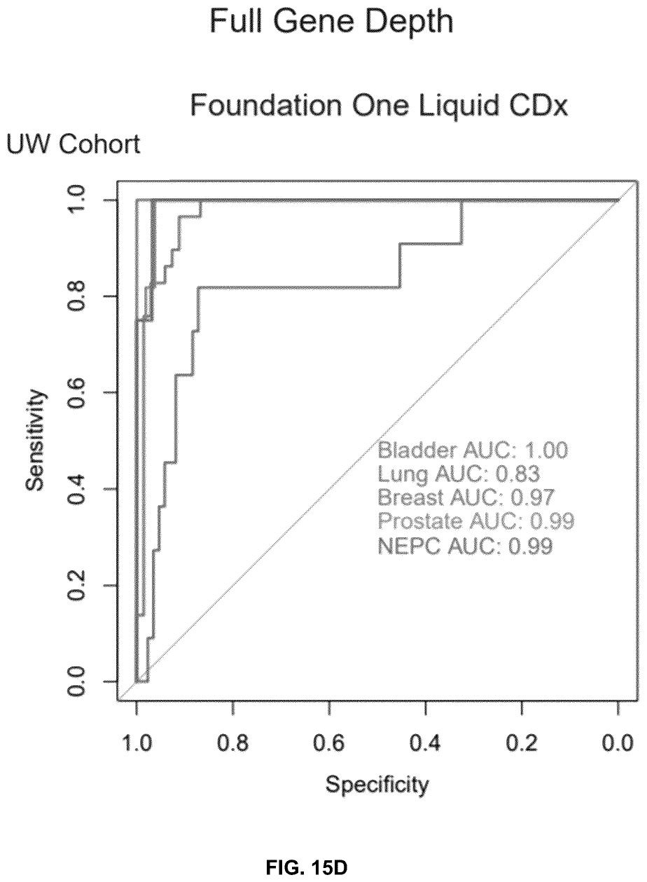

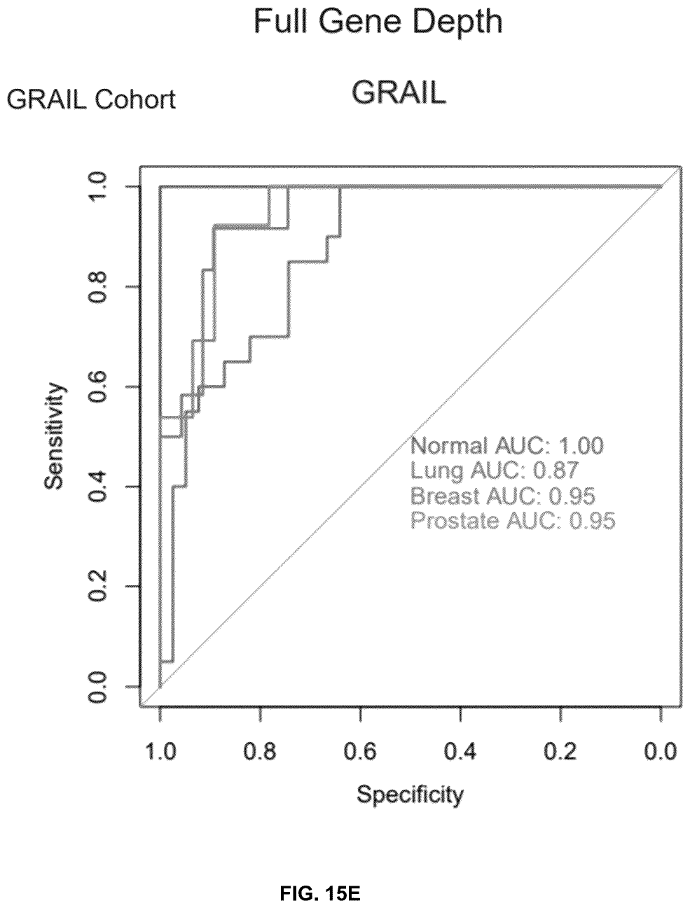

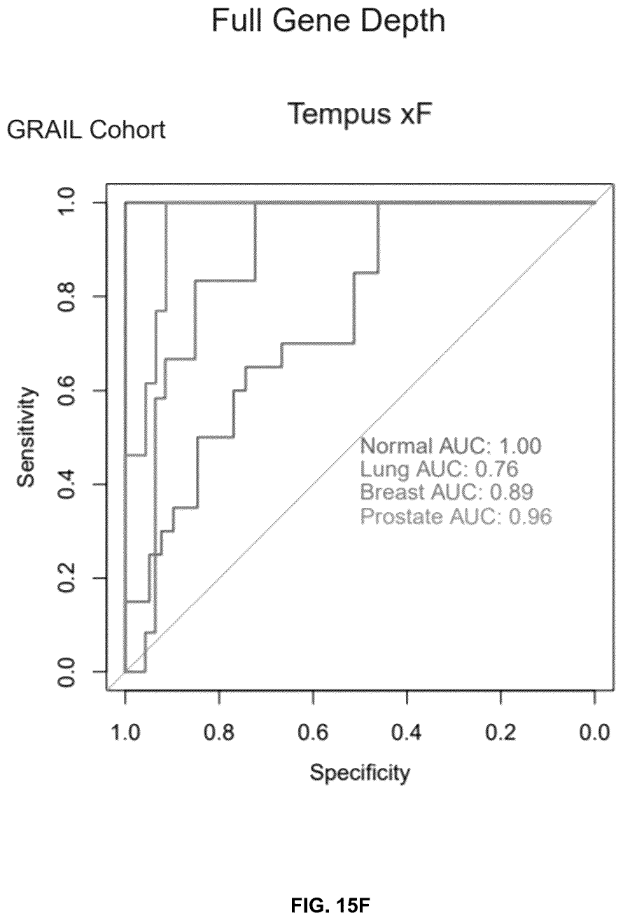

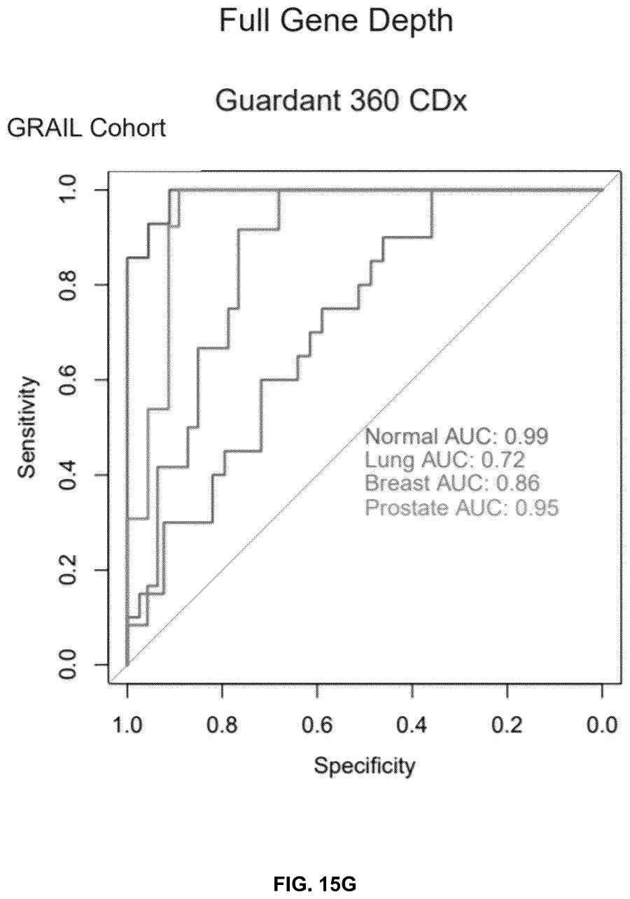

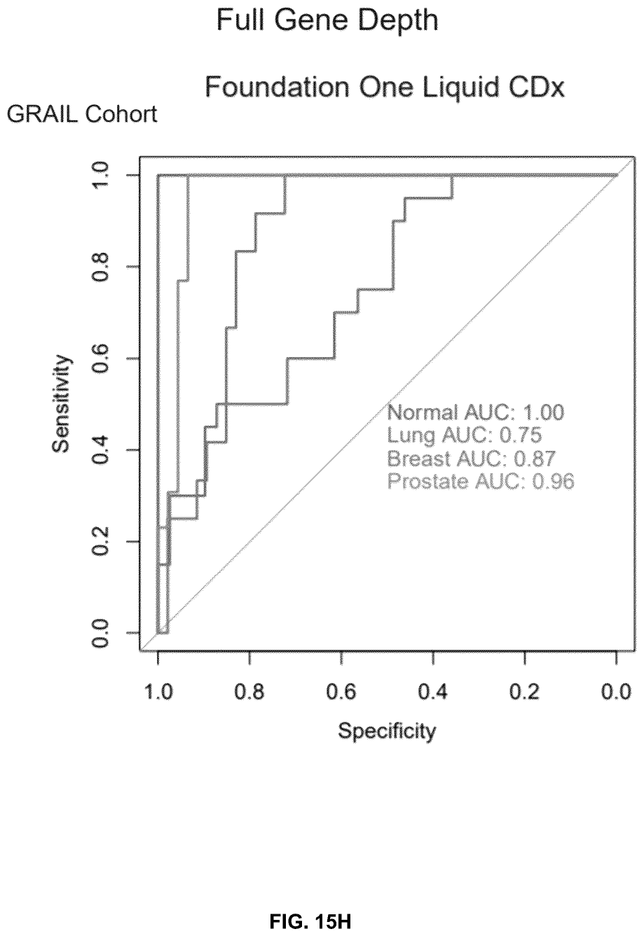

Full gene depth of sequencing was calculated by counting the number of fragments overlapping with any exon for each gene in each respective panel. A minimum of 1 bp overlap was required to count as an overlap. Counts for each gene were then normalized by dividing by the size of the sum of the gene's exons in base pairs and then dividing by the total reads in the sample. This depth metric for each gene in each respective panel was used for model training and validation as in the E1SE model (FIGS. 15A-15H).

Validation AUC in the UW cohort using genes in the UW panel was 1.00 for bladder cancer, 0.97 for breast cancer, 0.85 for lung cancer, 1.00 for NEPC, and 0.98 for prostate cancer. Validation AUC in the UW cohort using common genes between the UW panel and the Tempus xF panel was 0.99 for bladder cancer, 0.99 for breast cancer, 0.88 for lung cancer, 0.99 for NEPC, and 0.98 for prostate cancer. Validation AUC in the UW cohort using common genes between the UW panel and the Guardant 360 CDx panel was 0.97 for bladder cancer, 0.96 for breast cancer, 0.83 for lung cancer, 0.98 for NEPC, and 0.93 for prostate cancer. Validation AUC in the UW cohort using common genes between the UW panel and the Foundation One Liquid CDx panel was 1.00 for bladder cancer, 0.97 for breast cancer, 0.83 for lung cancer, 0.99 for NEPC, and 0.99 for prostate cancer.

Validation AUC in the GRAIL cohort using genes in the GRAIL panel was 0.95 for breast cancer, 0.87 for lung cancer, 0.95 for prostate cancer, and 1.00 for cancer vs normal. Validation AUC in the GRAIL cohort using common genes between the GRAIL panel and the Tempus xF panel was 0.89 for breast cancer, 0.76 for lung cancer, 0.96 for prostate cancer, and 1.00 for cancer vs normal. Validation AUC in the GRAIL cohort using common genes between the GRAIL panel and the Guardant 360 CDx panel was 0.86 for breast cancer, 0.72 for lung cancer, 0.95 for prostate cancer, and 0.99 for cancer vs normal. Validation AUC in the GRAIL cohort using common genes between the GRAIL panel and the Foundation One Liquid CDx panel was 0.87 for breast cancer, 0.75 for lung cancer, 0.96 for prostate cancer, and 1.00 for cancer vs normal.

### Exon 1 Motif Diversity

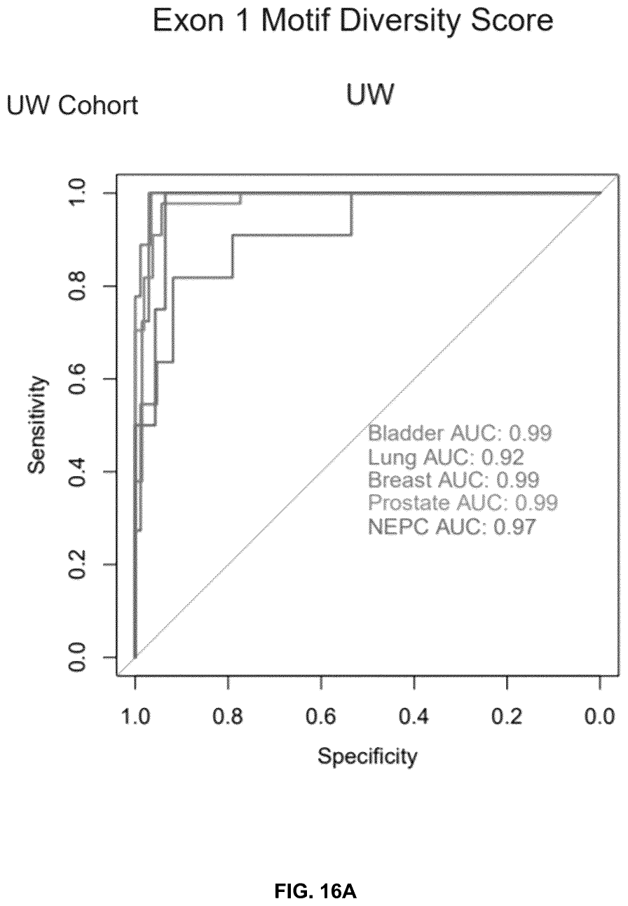

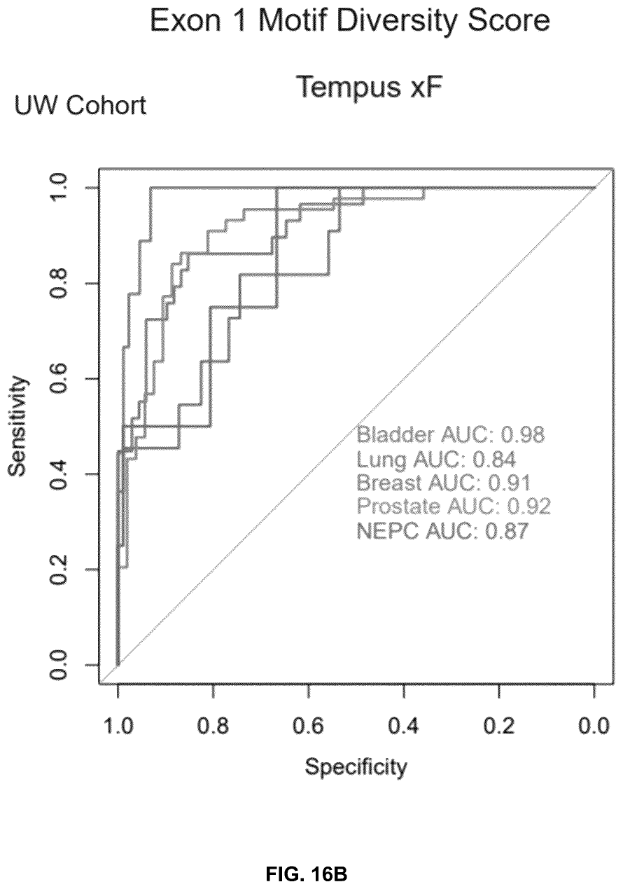

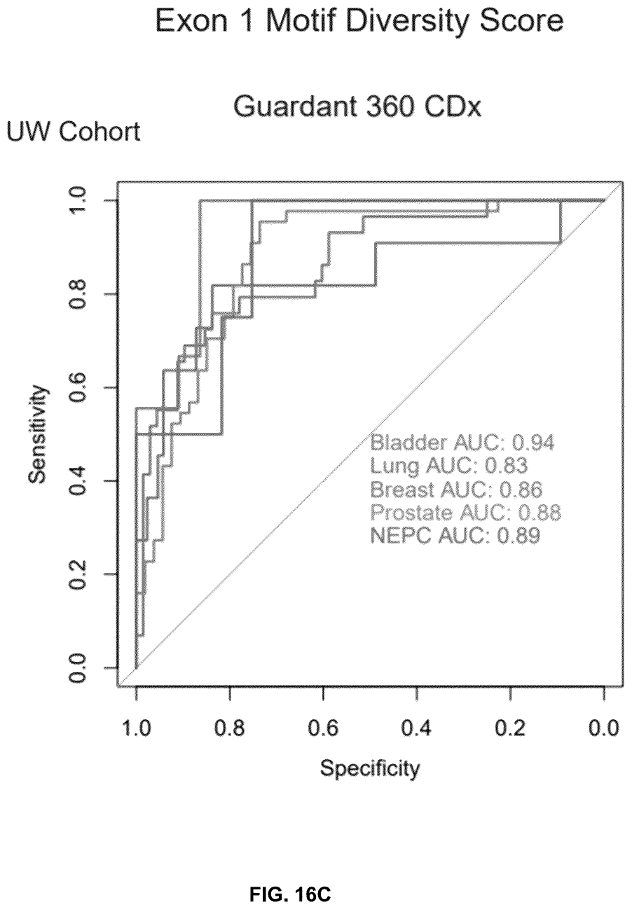

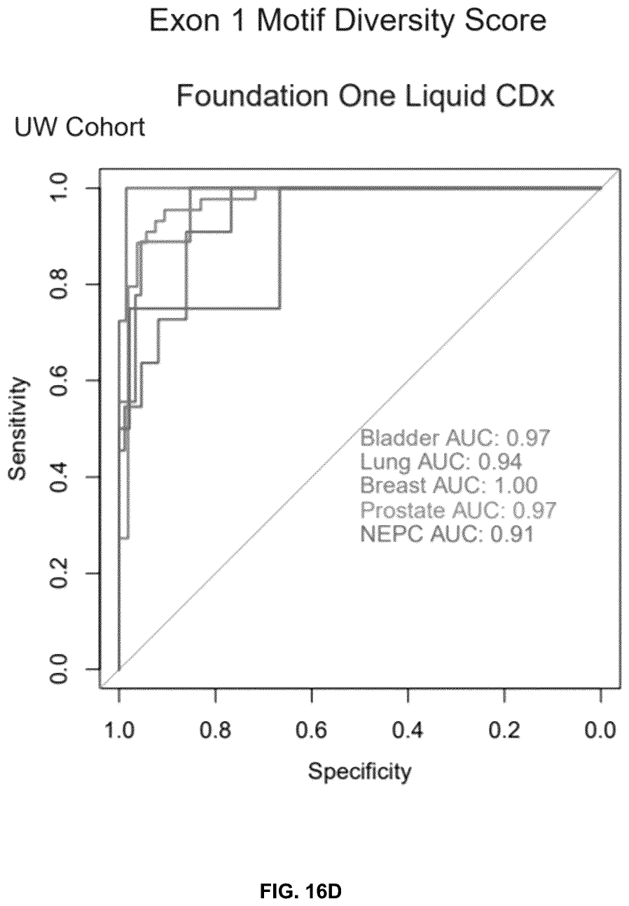

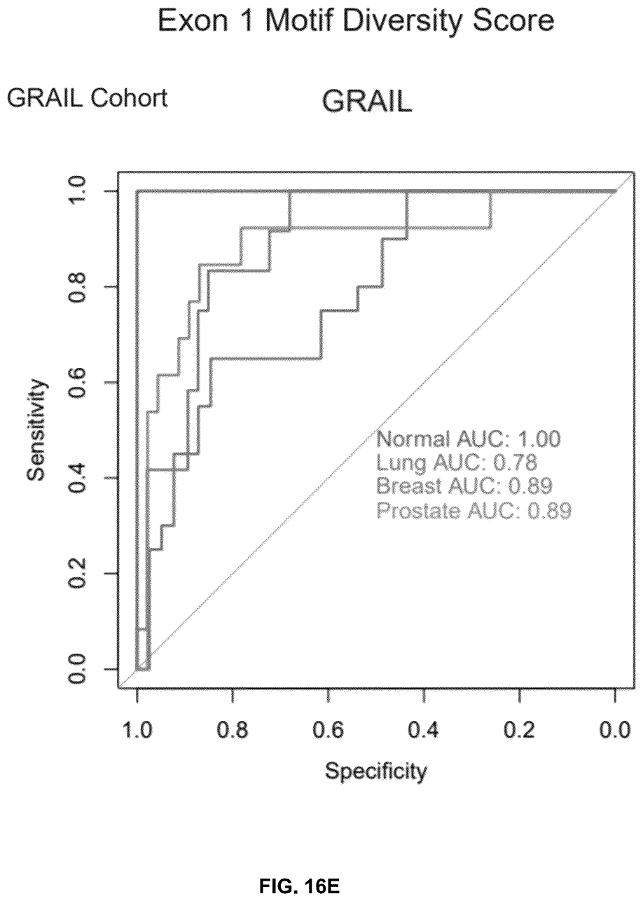

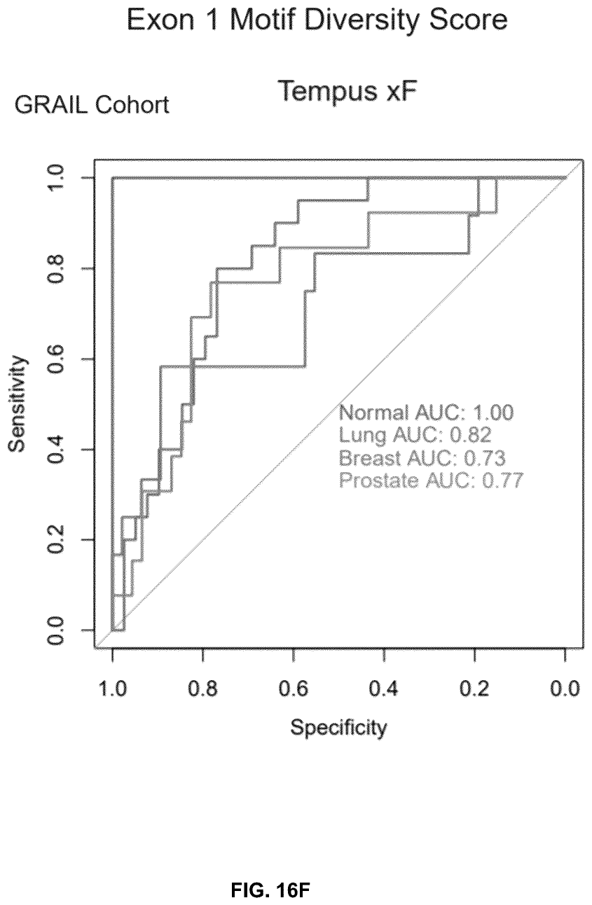

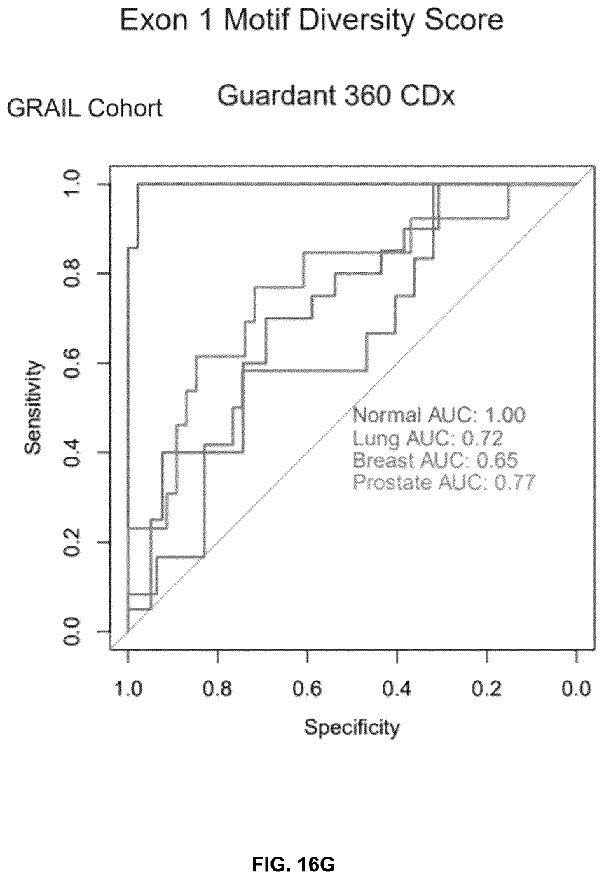

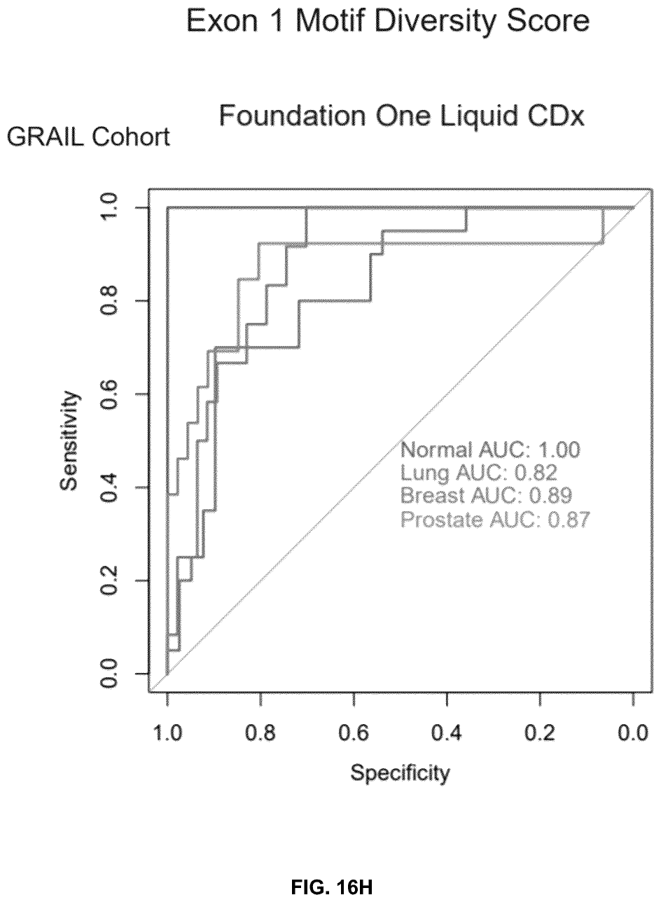

For each exon in each gene in each respective panel, the motif diversity score (MDS) was calculated for the set of fragments overlapping each exon. A minimum of 1 bp overlap was required to count as an overlap. MDS was calculated as reported previously²⁴. The MDS metric at the first coding exon of all genes in the respective panels was used for model training and validation as in the E1SE model (FIGS. 16A-16H).

Validation AUC in the UW cohort using genes in the UW panel was 0.99 for bladder cancer, 0.99 for breast cancer, 0.92 for lung cancer, 0.97 for NEPC, and 0.99 for prostate cancer. Validation AUC in the UW cohort using common genes between the UW panel and the Tempus xF panel was 0.98 for bladder cancer, 0.91 for breast cancer, 0.84 for lung cancer, 0.87 for NEPC, and 0.92 for prostate cancer. Validation AUC in the UW cohort using common genes between the UW panel and the Guardant 360 CDx panel was 0.94 for bladder cancer, 0.86 for breast cancer, 0.83 for lung cancer, 0.89 for NEPC, and 0.88 for prostate cancer. Validation AUC in the UW cohort using common genes between the UW panel and the Foundation One Liquid CDx panel was 0.97 for bladder cancer, 1.00 for breast cancer, 0.94 for lung cancer, 0.91 for NEPC, and 0.97 for prostate cancer.

Validation AUC in the GRAIL cohort using genes in the GRAIL panel was 0.89 for breast cancer, 0.78 for lung cancer, 0.89 for prostate cancer, and 1.00 for cancer vs normal. Validation AUC in the GRAIL cohort using common genes between the GRAIL panel and the Tempus xF panel was 0.73 for breast cancer, 0.82 for lung cancer, 0.77 for prostate cancer, and 1.00 for cancer vs normal. Validation AUC in the GRAIL cohort using common genes between the GRAIL panel and the Guardant 360 CDx panel was 0.65 for breast cancer, 0.72 for lung cancer, 0.77 for prostate cancer, and 1.00 for cancer vs normal. Validation AUC in the GRAIL cohort using common genes between the GRAIL panel and the Foundation One Liquid CDx panel was 0.89 for breast cancer, 0.82 for lung cancer, 0.87 for prostate cancer, and 1.00 for cancer vs normal.

### Exon 1 Fragment Size Bins

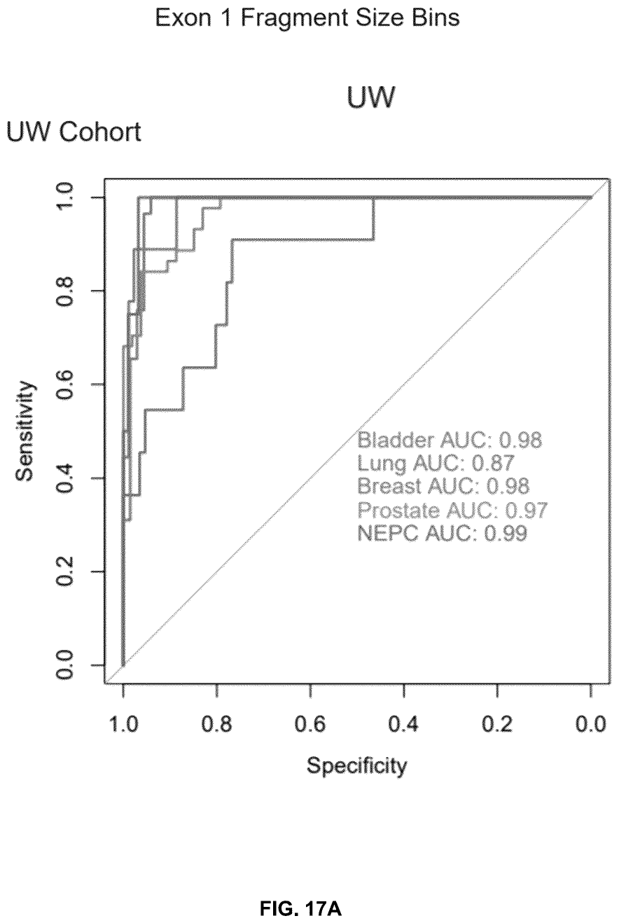

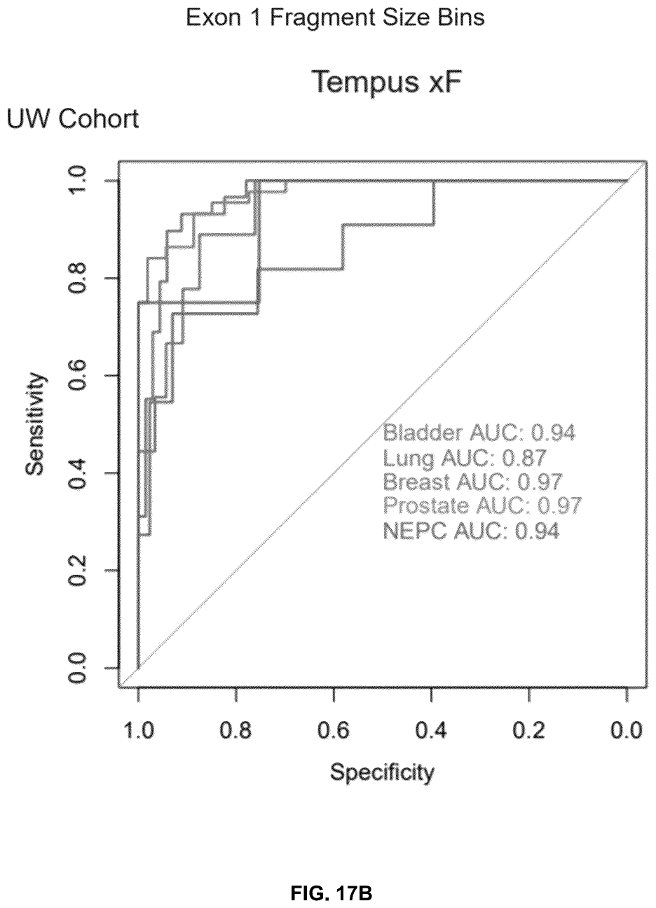

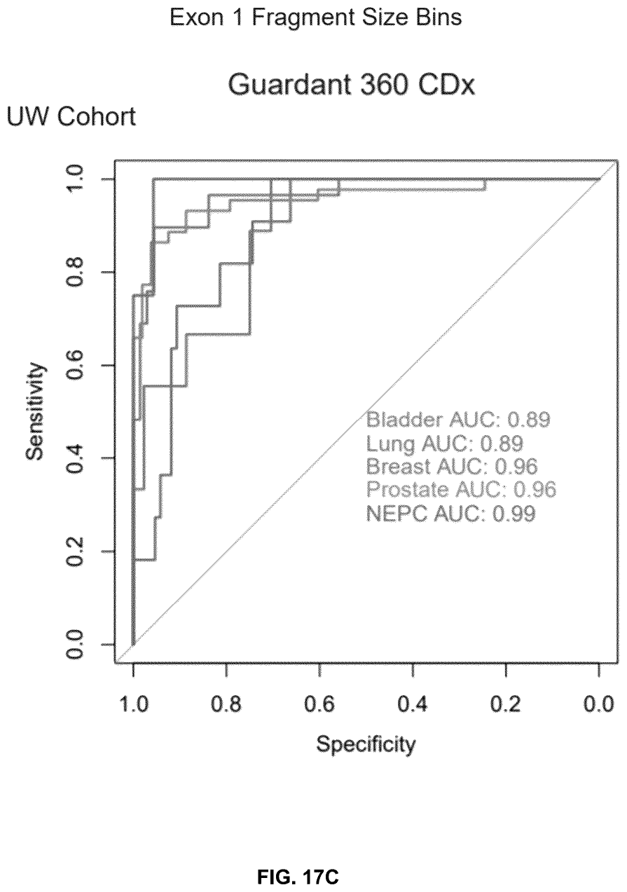

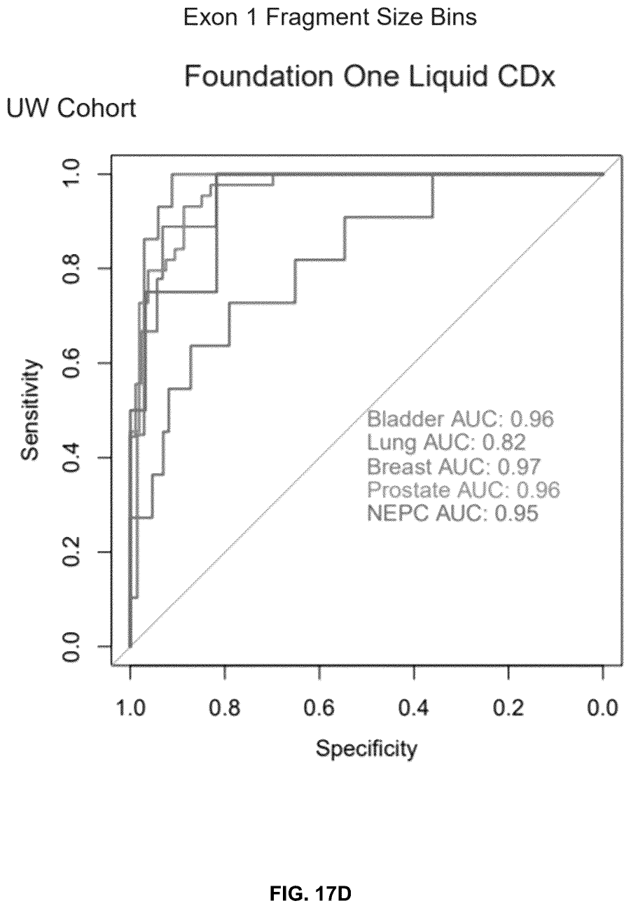

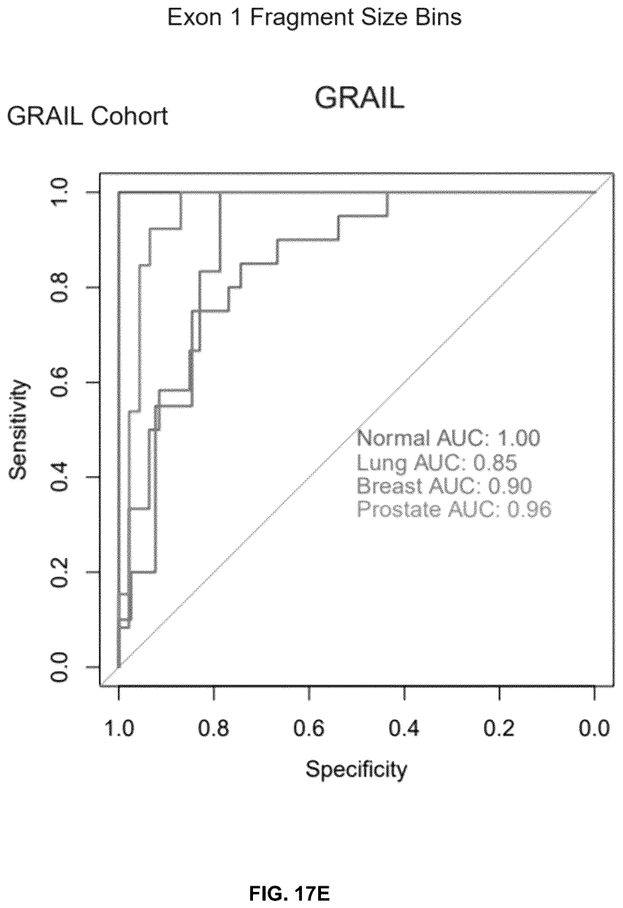

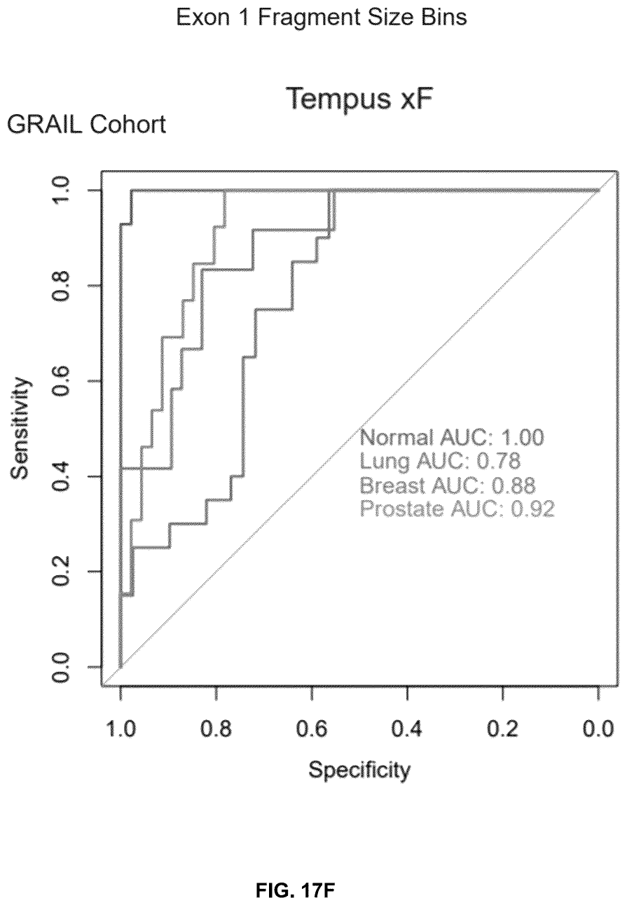

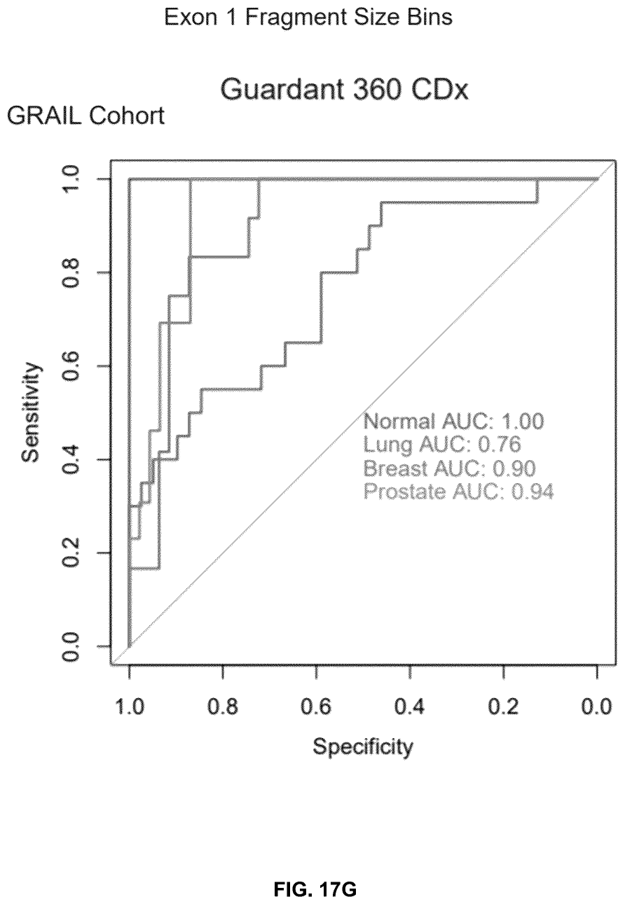

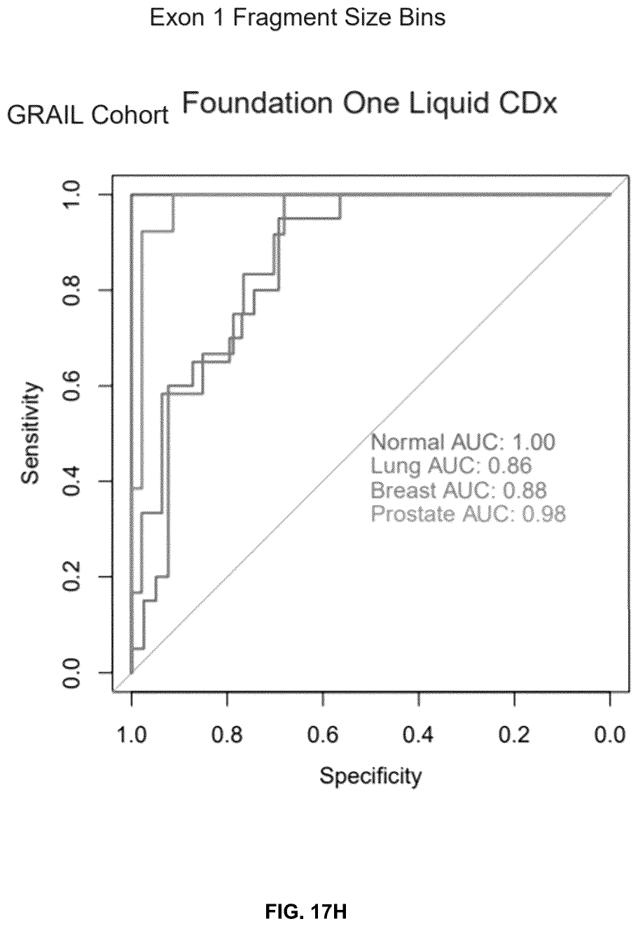

For each exon in each gene in each respective panel, fragments overlapping each exon were extracted and then binned by fragment size. A minimum of 1 bp overlap was required to count as an overlap. The fragment size bins were 0-100 bp, 101-150 bp, 151-200 bp, 201-250 bp, 251-300 bp, and greater than 300 bp. The proportion of fragments falling into each of these bins for each exon was calculated by dividing the number of fragments in each bin by the total number of fragments overlapping the respective exon. Each exon is represented by six fragment size bins. The fragment bins for the first coding exon of all genes in the respective panel was used for model training and validation as in the E1SE model (FIGS. 17A-17H).

Validation AUC in the UW cohort using genes in the UW panel was 0.98 for bladder cancer, 0.98 for breast cancer, 0.87 for lung cancer, 0.99 for NEPC, and 0.97 for prostate cancer. Validation AUC in the UW cohort using common genes between the UW panel and the Tempus xF panel was 0.94 for bladder cancer, 0.97 for breast cancer, 0.87 for lung cancer, 0.94 for NEPC, and 0.97 for prostate cancer. Validation AUC in the UW cohort using common genes between the UW panel and the Guardant 360 CDx panel was 0.89 for bladder cancer, 0.96 for breast cancer, 0.89 for lung cancer, 0.99 for NEPC, and 0.96 for prostate cancer. Validation AUC in the UW cohort using common genes between the UW panel and the Foundation One Liquid CDx panel was 0.96 for bladder cancer, 0.97 for breast cancer, 0.82 for lung cancer, 0.95 for NEPC, and 0.96 for prostate cancer.

Validation AUC in the GRAIL cohort using genes in the GRAIL panel was 0.90 for breast cancer, 0.85 for lung cancer, 0.96 for prostate cancer, and 1.00 for cancer vs normal. Validation AUC in the GRAIL cohort using common genes between the GRAIL panel and the Tempus xF panel was 0.88 for breast cancer, 0.78 for lung cancer, 0.92 for prostate cancer, and 1.00 for cancer vs normal. Validation AUC in the GRAIL cohort using common genes between the GRAIL panel and the Guardant 360 CDx panel was 0.90 for breast cancer, 0.76 for lung cancer, 0.94 for prostate cancer, and 1.00 for cancer vs normal. Validation AUC in the GRAIL cohort using common genes between the GRAIL panel and the Foundation One Liquid CDx panel was 0.88 for breast cancer, 0.86 for lung cancer, 0.98 for prostate cancer, and 1.00 for cancer vs normal.

### Exon 1 Small Fragment Proportions

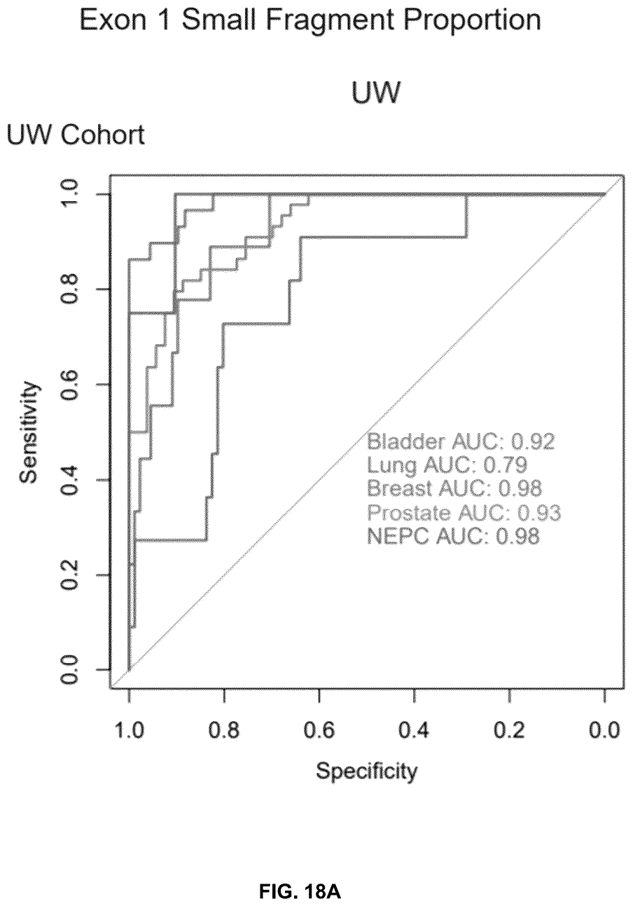

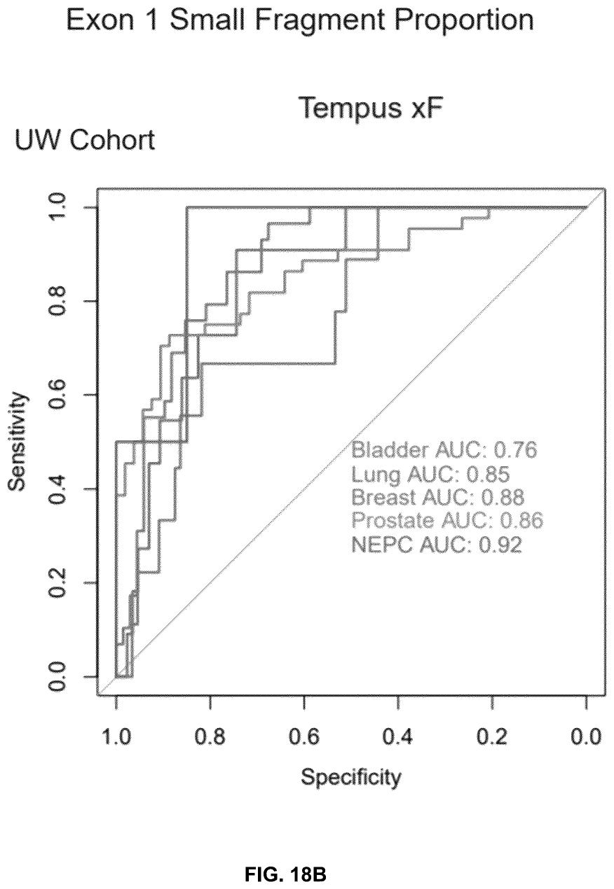

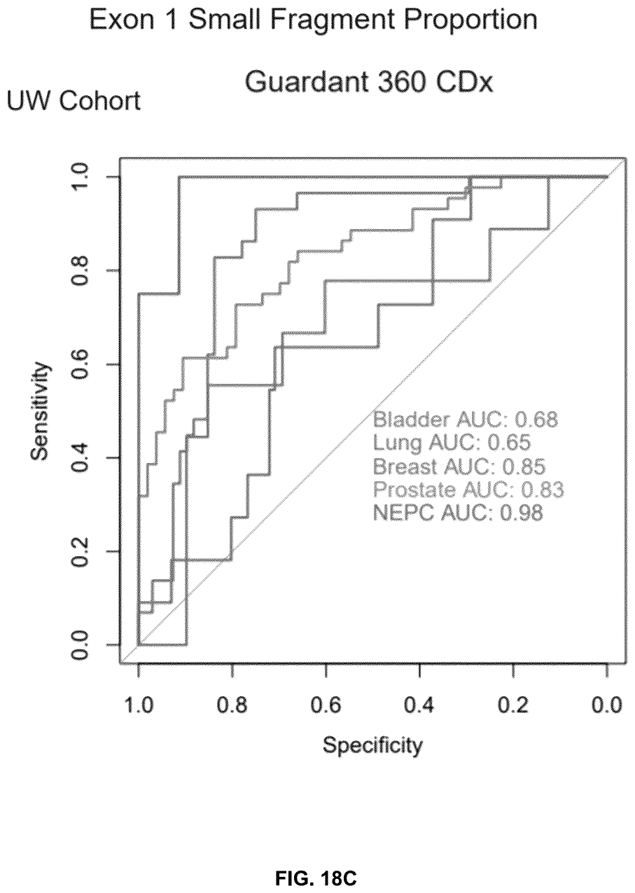

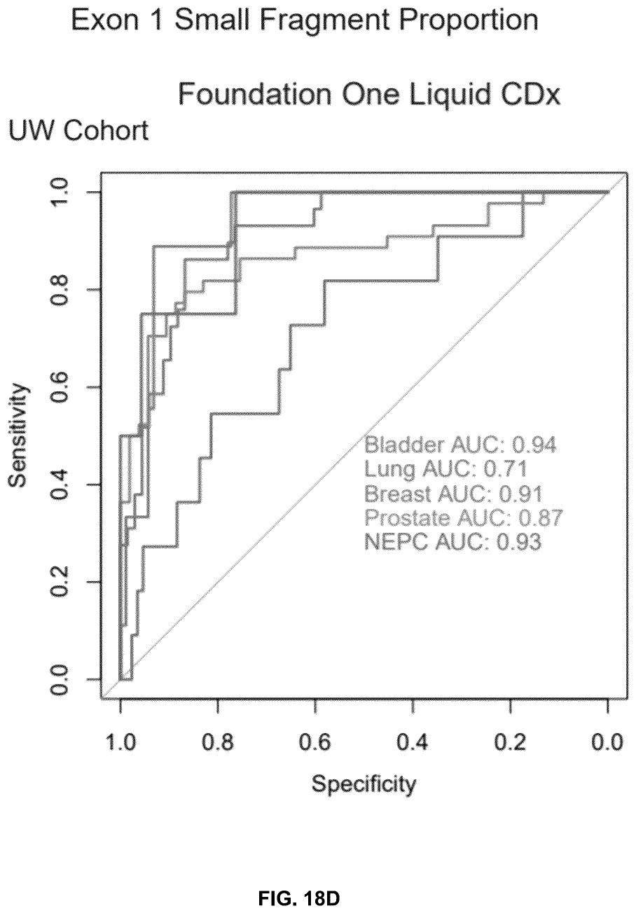

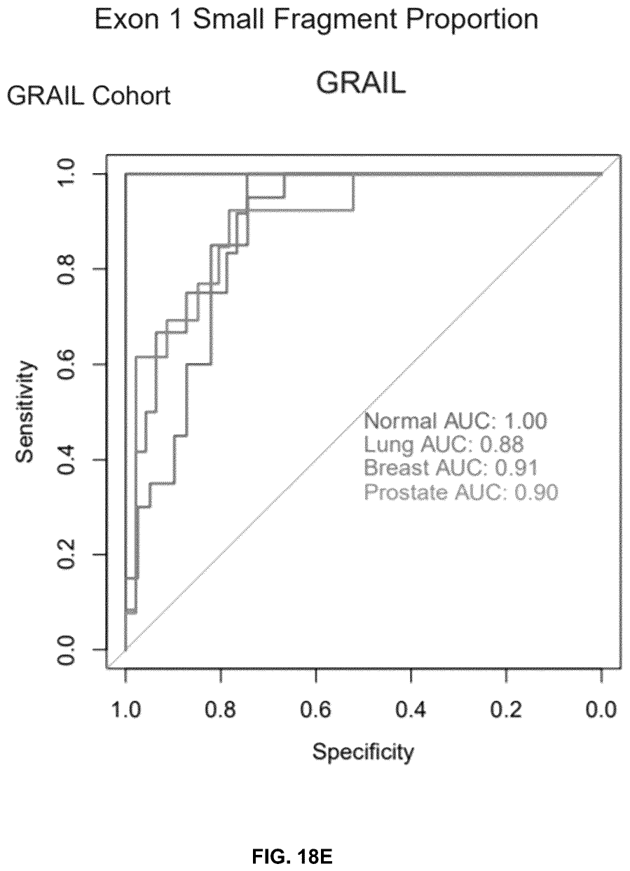

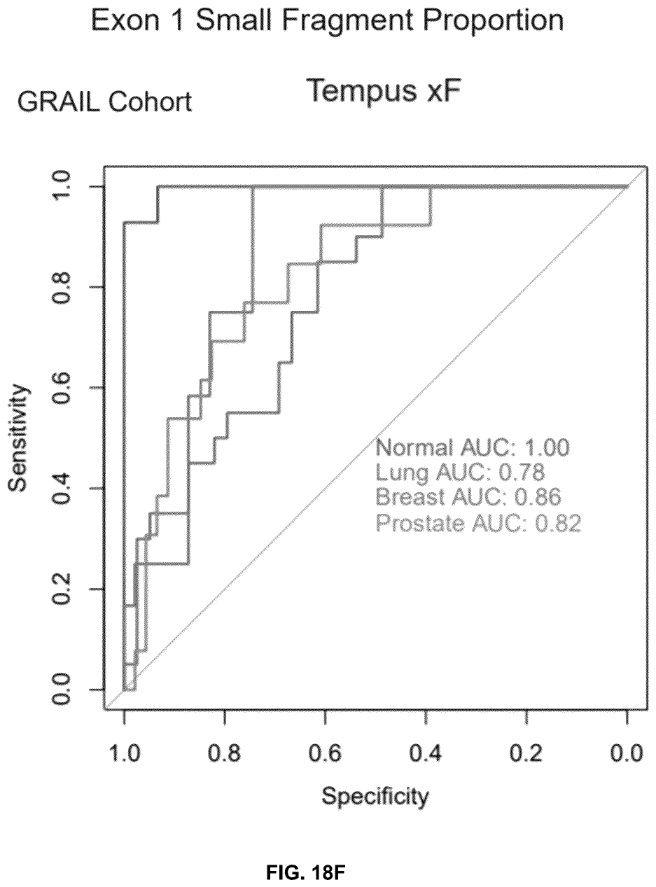

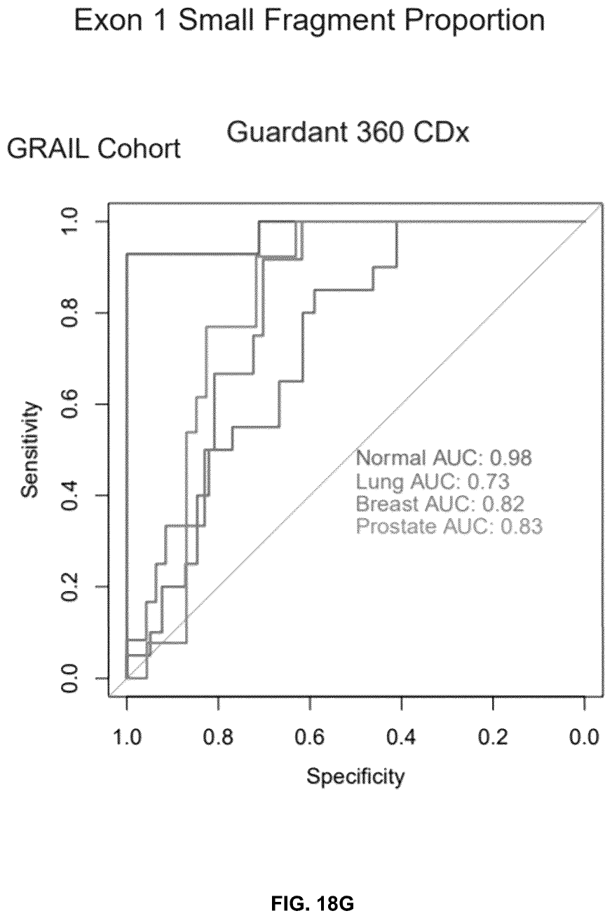

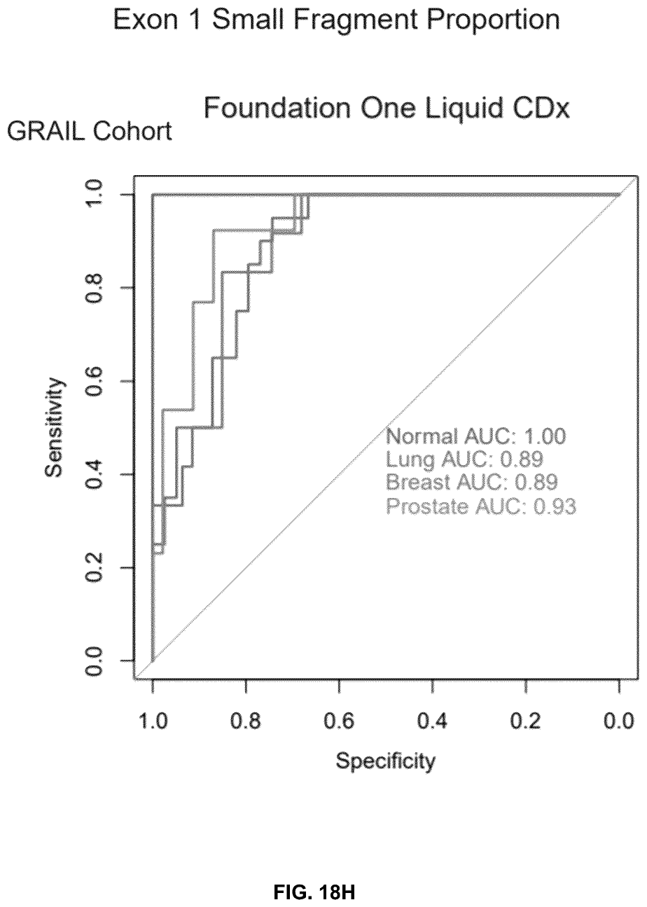

For each exon in each gene in each respective panel, fragments overlapping each exon were extracted and then the proportion of fragments less than or equal to 150 bp was calculated for each individual exon. A minimum of 1 bp overlap was required to count as an overlap. The proportion of small fragments for the first coding exon of all genes in the respective panel was used for model training and validation as in the E1SE model (FIGS. 18A-18H).

Validation AUC in the UW cohort using genes in the UW panel was 0.92 for bladder cancer, 0.98 for breast cancer, 0.79 for lung cancer, 0.98 for NEPC, and 0.93 for prostate cancer. Validation AUC in the UW cohort using common genes between the UW panel and the Tempus xF panel was 0.76 for bladder cancer, 0.88 for breast cancer, 0.85 for lung cancer, 0.92 for NEPC, and 0.86 for prostate cancer. Validation AUC in the UW cohort using common genes between the UW panel and the Guardant 360 CDx panel was 0.68 for bladder cancer, 0.85 for breast cancer, 0.65 for lung cancer, 0.98 for NEPC, and 0.83 for prostate cancer. Validation AUC in the UW cohort using common genes between the UW panel and the Foundation One Liquid CDx panel was 0.94 for bladder cancer, 0.91 for breast cancer, 0.71 for lung cancer, 0.93 for NEPC, and 0.87 for prostate cancer.

Validation AUC in the GRAIL cohort using genes in the GRAIL panel was 0.91 for breast cancer, 0.88 for lung cancer, 0.90 for prostate cancer, and 1.00 for cancer vs normal. Validation AUC in the GRAIL cohort using common genes between the GRAIL panel and the Tempus xF panel was 0.86 for breast cancer, 0.78 for lung cancer, 0.82 for prostate cancer, and 1.00 for cancer vs normal. Validation AUC in the GRAIL cohort using common genes between the GRAIL panel and the Guardant 360 CDx panel was 0.82 for breast cancer, 0.73 for lung cancer, 0.83 for prostate cancer, and 0.98 for cancer vs normal. Validation AUC in the GRAIL cohort using common genes between the GRAIL panel and the Foundation One Liquid CDx panel was 0.89 for breast cancer, 0.89 for lung cancer, 0.93 for prostate cancer, and 1.00 for cancer vs normal.

### All Exons Shannon Entropy

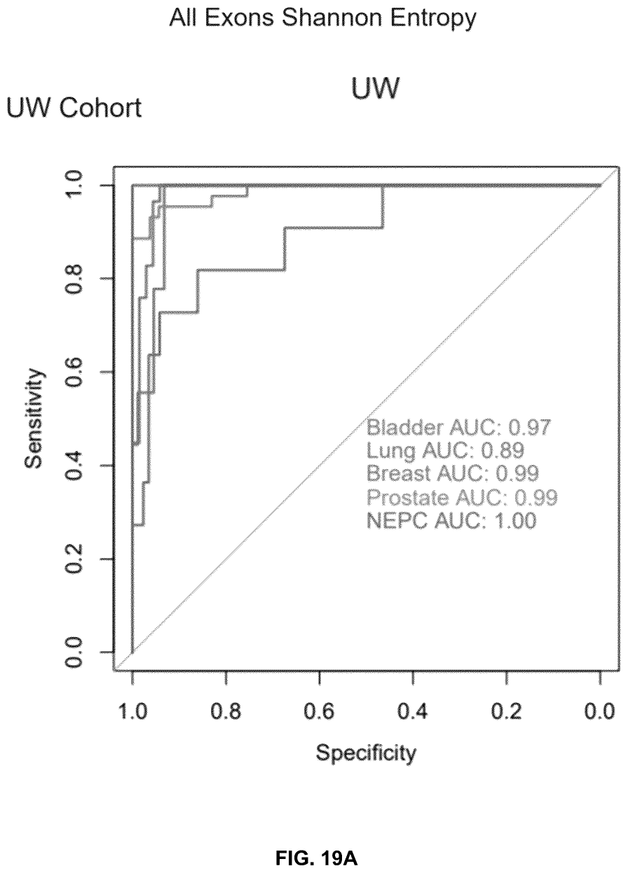

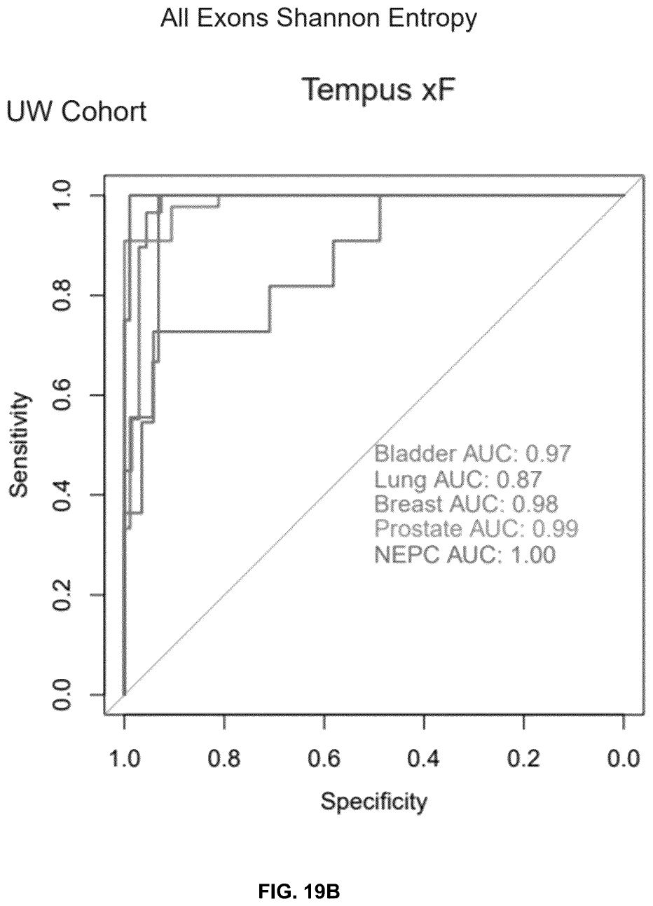

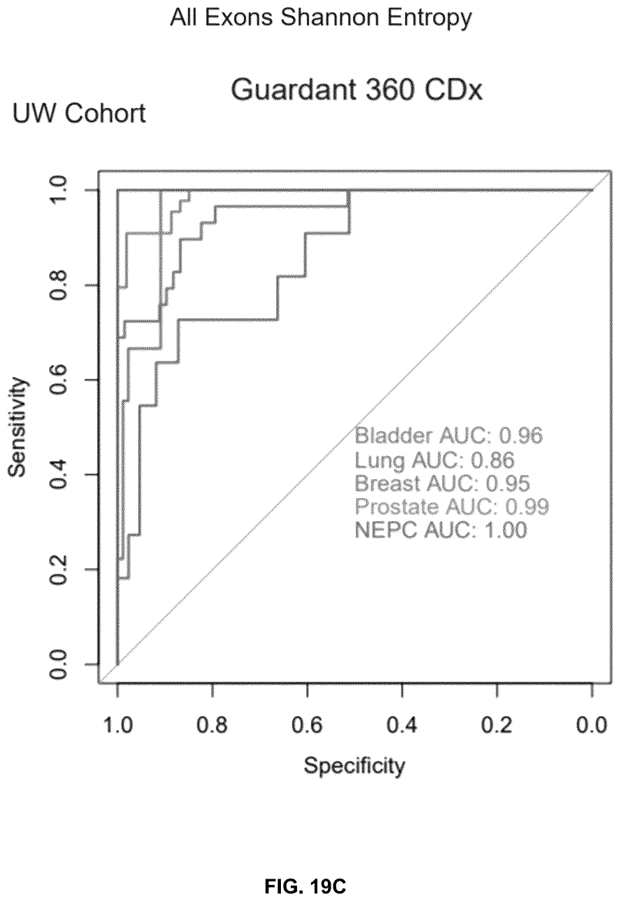

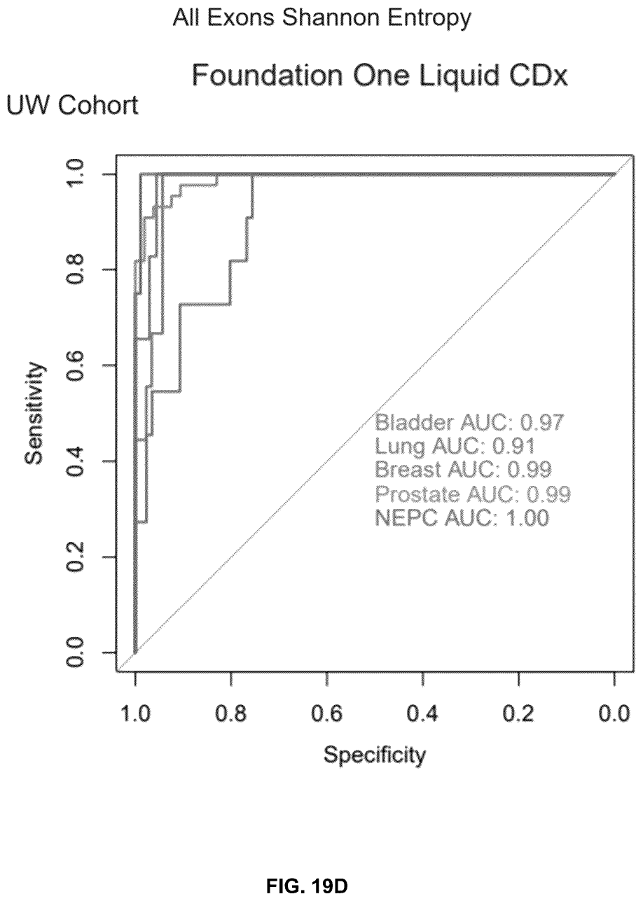

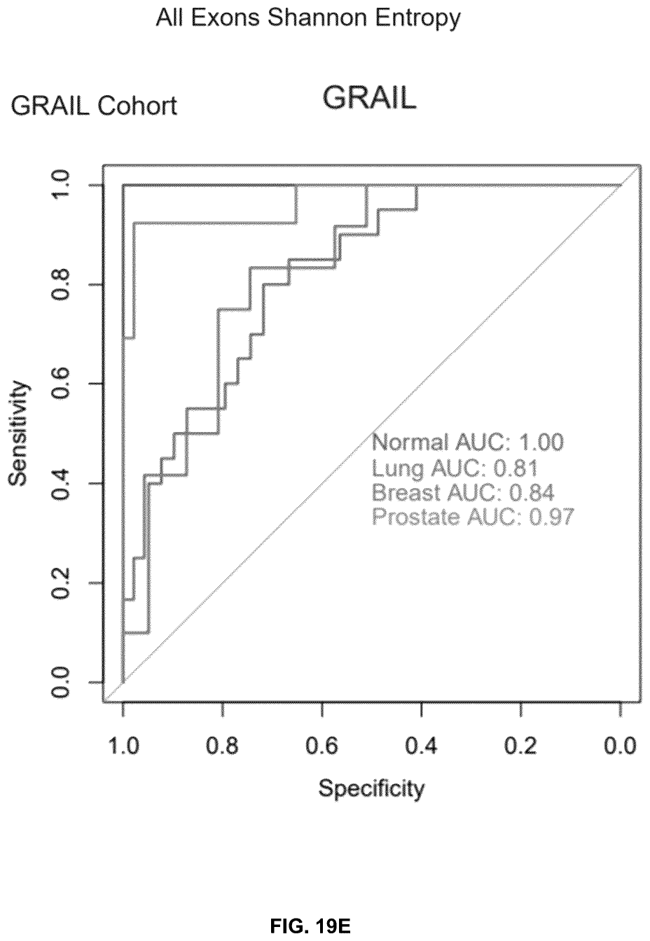

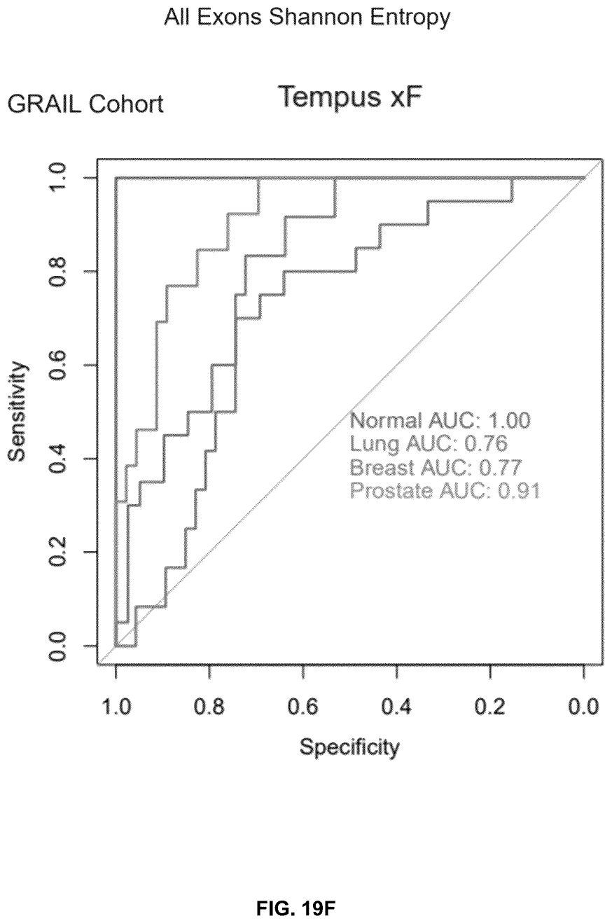

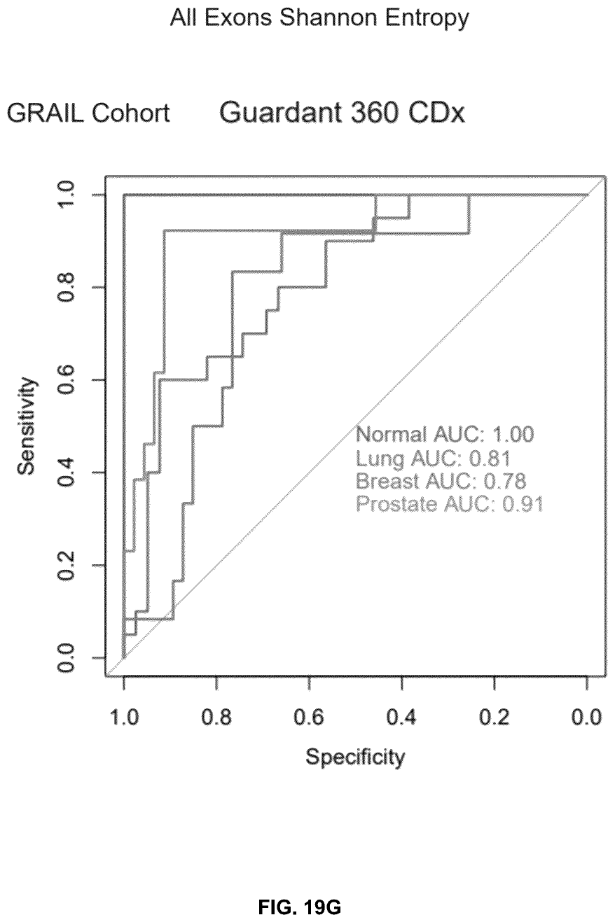

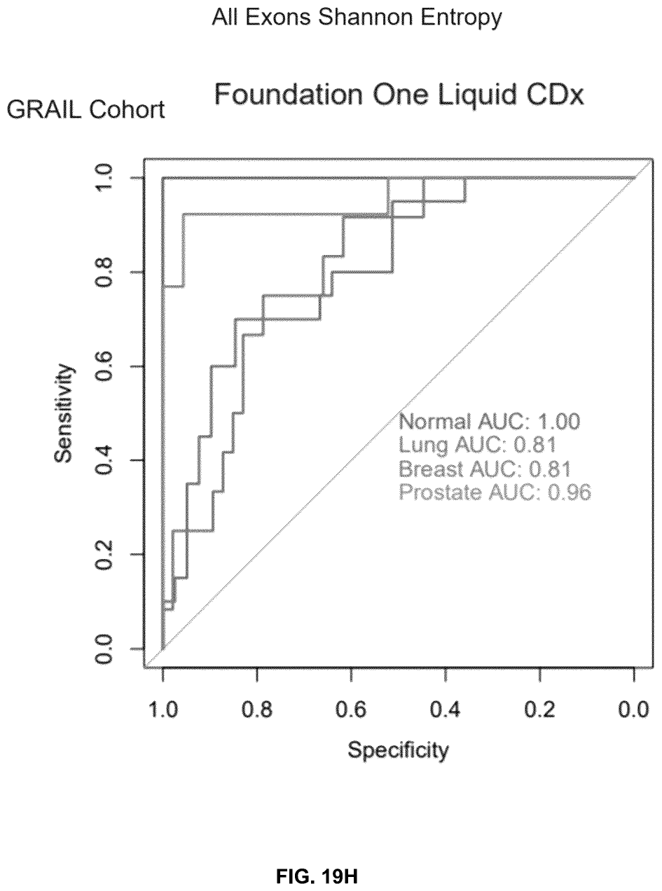

Shannon entropy (SE) was calculated as described above for all exons for all genes in each respective gene panel. SE for all exons for all genes were used as features for model training and validation (FIGS. 19A-19H).

Validation AUC in the UW cohort using genes in the UW panel was 0.97 for bladder cancer, 0.99 for breast cancer, 0.89 for lung cancer, 1.00 for NEPC, and 0.99 for prostate cancer. Validation AUC in the UW cohort using common genes between the UW panel and the Tempus xF panel was 0.97 for bladder cancer, 0.98 for breast cancer, 0.87 for lung cancer, 1.00 for NEPC, and 0.99 for prostate cancer. Validation AUC in the UW cohort using common genes between the UW panel and the Guardant 360 CDx panel was 0.96 for bladder cancer, 0.95 for breast cancer, 0.86 for lung cancer, 1.00 for NEPC, and 0.99 for prostate cancer. Validation AUC in the UW cohort using common genes between the UW panel and the Foundation One Liquid CDx panel was 0.97 for bladder cancer, 0.99 for breast cancer, 0.91 for lung cancer, 1.00 for NEPC, and 0.99 for prostate cancer.

Validation AUC in the GRAIL cohort using genes in the GRAIL panel was 0.84 for breast cancer, 0.81 for lung cancer, 0.97 for prostate cancer, and 1.00 for cancer vs normal. Validation AUC in the GRAIL cohort using common genes between the GRAIL panel and the Tempus xF panel was 0.77 for breast cancer, 0.76 for lung cancer, 0.91 for prostate cancer, and 1.00 for cancer vs normal. Validation AUC in the GRAIL cohort using common genes between the GRAIL panel and the Guardant 360 CDx panel was 0.78 for breast cancer, 0.81 for lung cancer, 0.91 for prostate cancer, and 1.00 for cancer vs normal. Validation AUC in the GRAIL cohort using common genes between the GRAIL panel and the Foundation One Liquid CDx panel was 0.81 for breast cancer, 0.81 for lung cancer, 0.96 for prostate cancer, and 1.00 for cancer vs normal.

### All Exons Depth

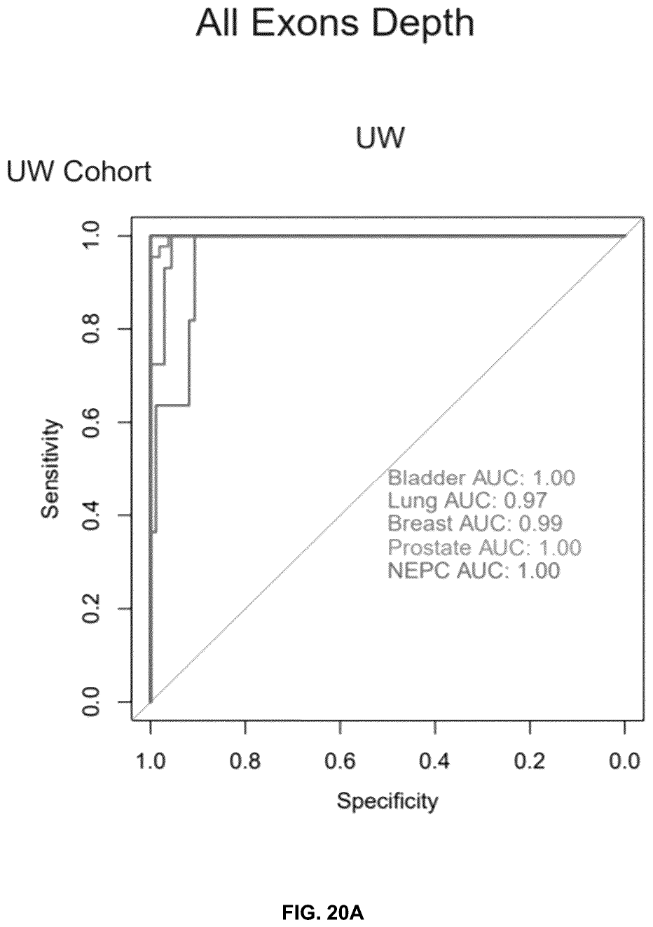

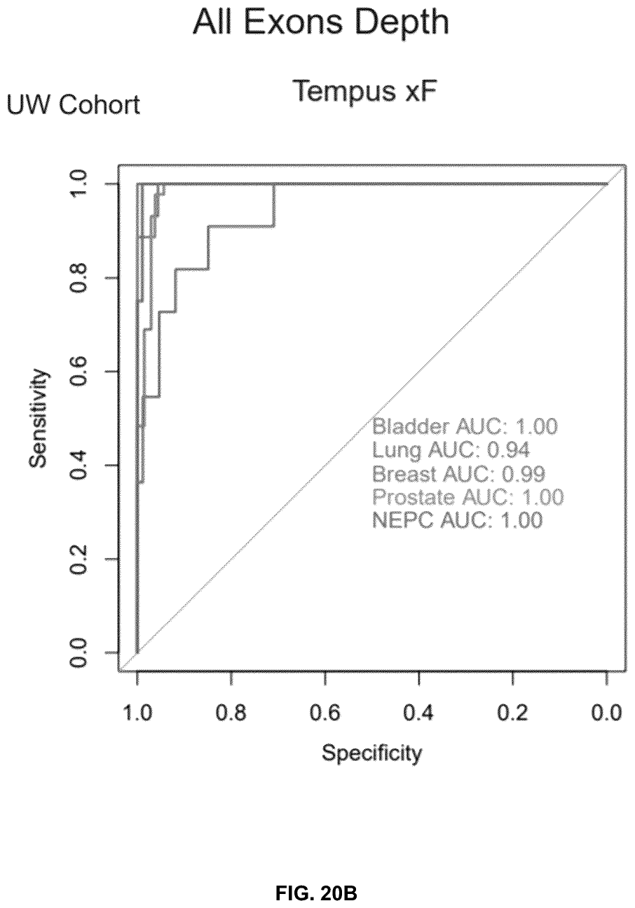

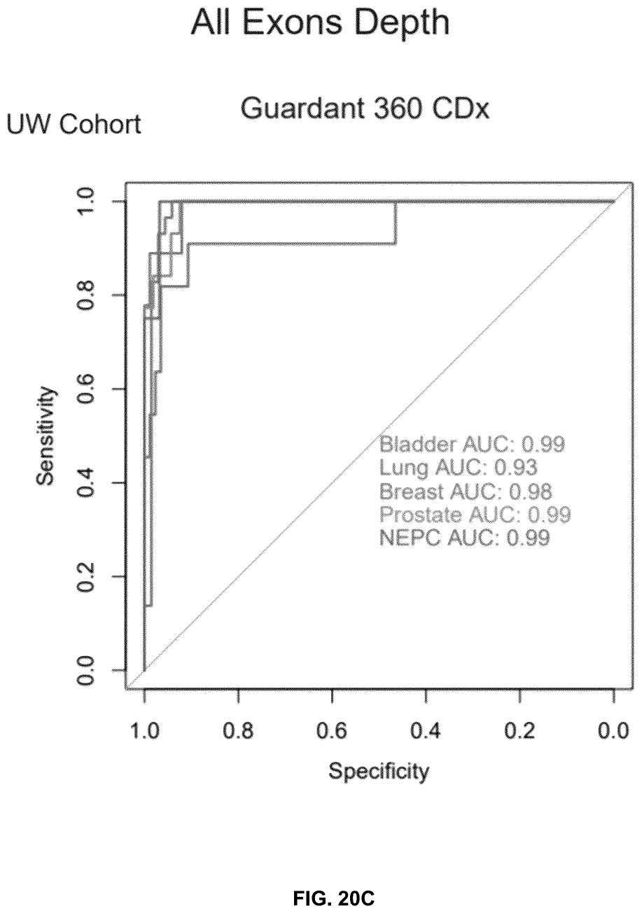

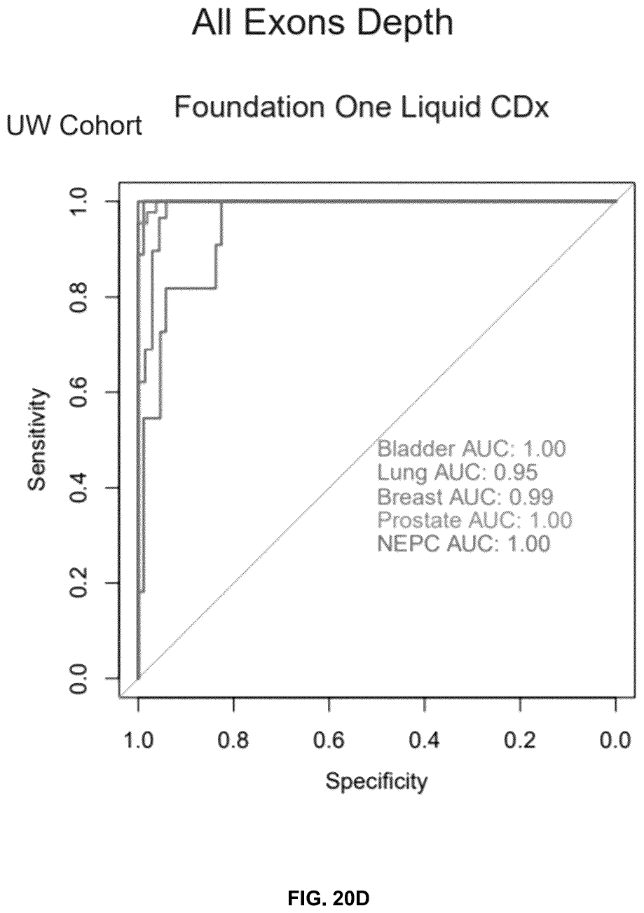

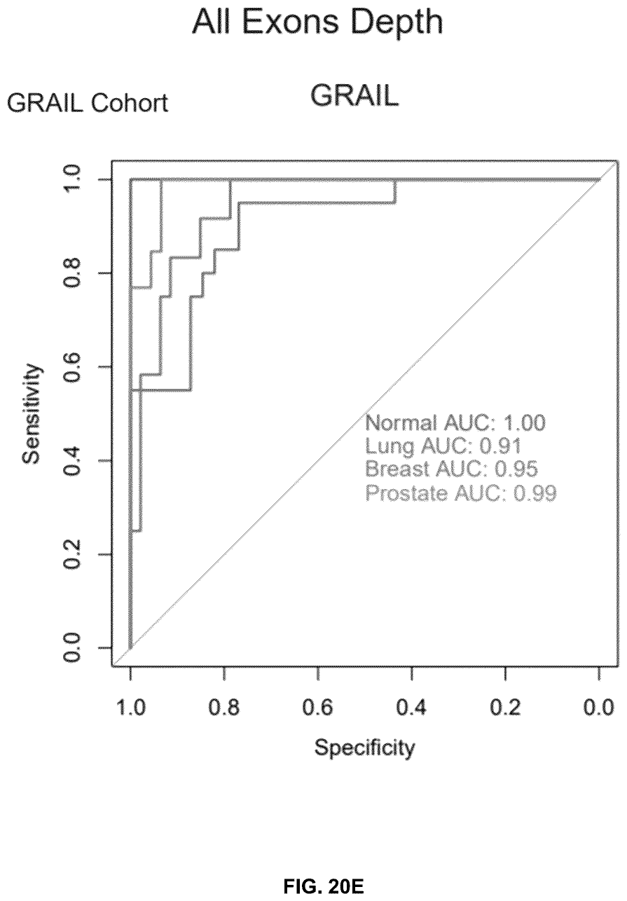

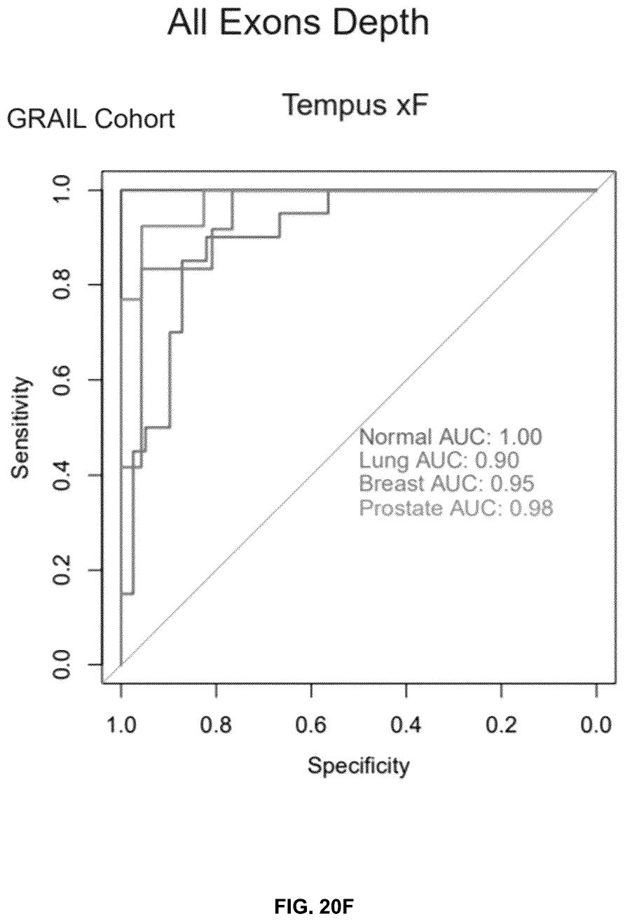

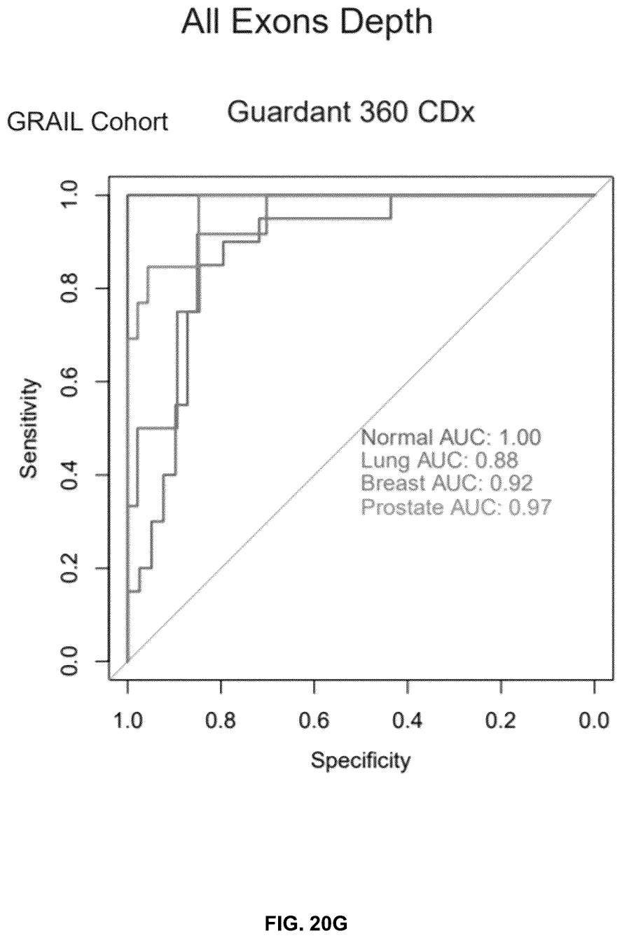

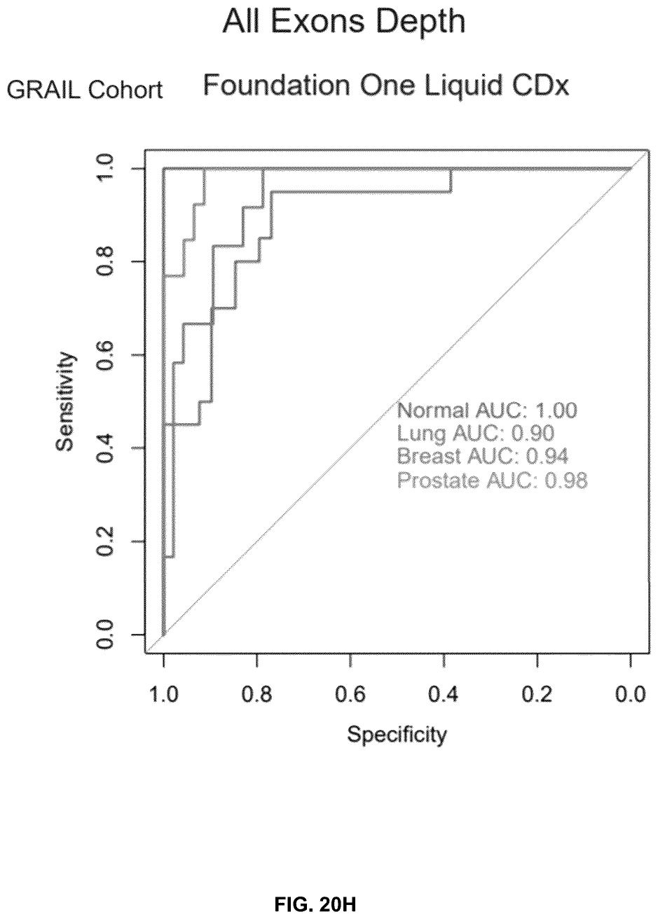

Depth of sequencing was calculated by counting the number of fragments overlapping with each individual exon acorss all genes in each respective panel. A minimum of 1 bp overlap was required to count as an overlap. Counts for each exon were then normalized by dividing by the size of the exon in base pairs and then dividing by the total reads in the sample. This depth metric in all coding exons of each gene in each respective panel was used for model training and validation (FIGS. 20A-20H).

Validation AUC in the UW cohort using genes in the UW panel was 1.00 for bladder cancer, 0.99 for breast cancer, 0.97 for lung cancer, 1.00 for NEPC, and 1.00 for prostate cancer. Validation AUC in the UW cohort using common genes between the UW panel and the Tempus xF panel was 1.00 for bladder cancer, 0.99 for breast cancer, 0.94 for lung cancer, 1.00 for NEPC, and 1.00 for prostate cancer. Validation AUC in the UW cohort using common genes between the UW panel and the Guardant 360 CDx panel was 0.99 for bladder cancer, 0.98 for breast cancer, 0.93 for lung cancer, 0.99 for NEPC, and 0.99 for prostate cancer. Validation AUC in the UW cohort using common genes between the UW panel and the Foundation One Liquid CDx panel was 1.00 for bladder cancer, 0.99 for breast cancer, 0.95 for lung cancer, 1.00 for NEPC, and 1.00 for prostate cancer.

Validation AUC in the GRAIL cohort using genes in the GRAIL panel was 0.95 for breast cancer, 0.91 for lung cancer, 0.99 for prostate cancer, and 1.00 for cancer vs normal. Validation AUC in the GRAIL cohort using common genes between the GRAIL panel and the Tempus xF panel was 0.95 for breast cancer, 0.90 for lung cancer, 0.98 for prostate cancer, and 1.00 for cancer vs normal. Validation AUC in the GRAIL cohort using common genes between the GRAIL panel and the Guardant 360 CDx panel was 0.92 for breast cancer, 0.88 for lung cancer, 0.97 for prostate cancer, and 1.00 for cancer vs normal. Validation AUC in the GRAIL cohort using common genes between the GRAIL panel and the Foundation One Liquid CDx panel was 0.94 for breast cancer, 0.90 for lung cancer, 0.98 for prostate cancer, and 1.00 for cancer vs normal.

### All Exons Motif Diversity Score

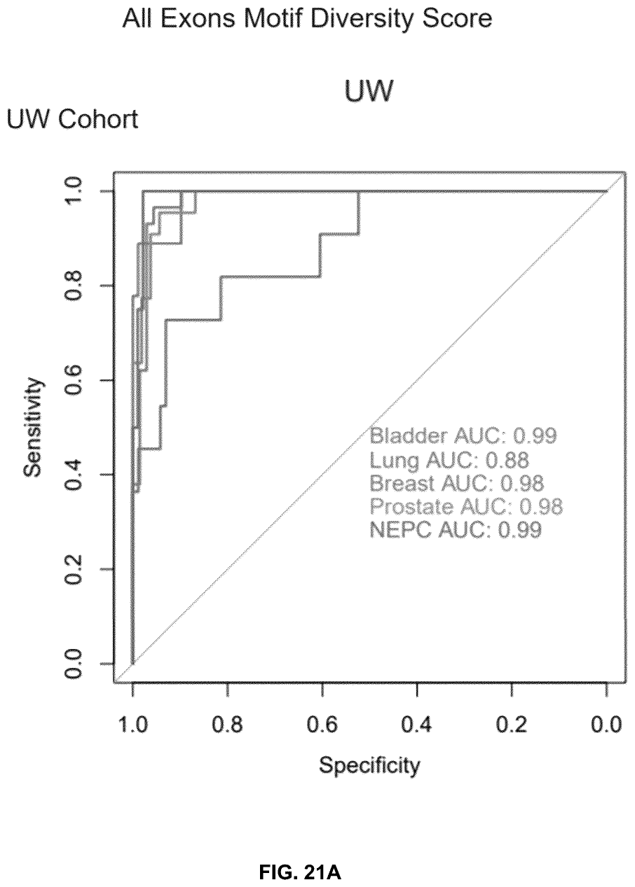

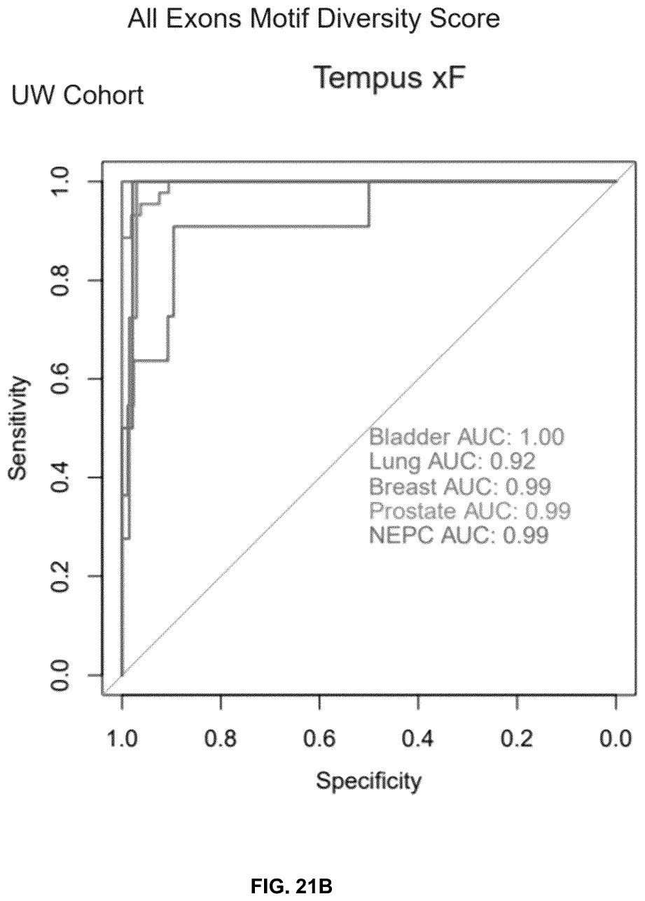

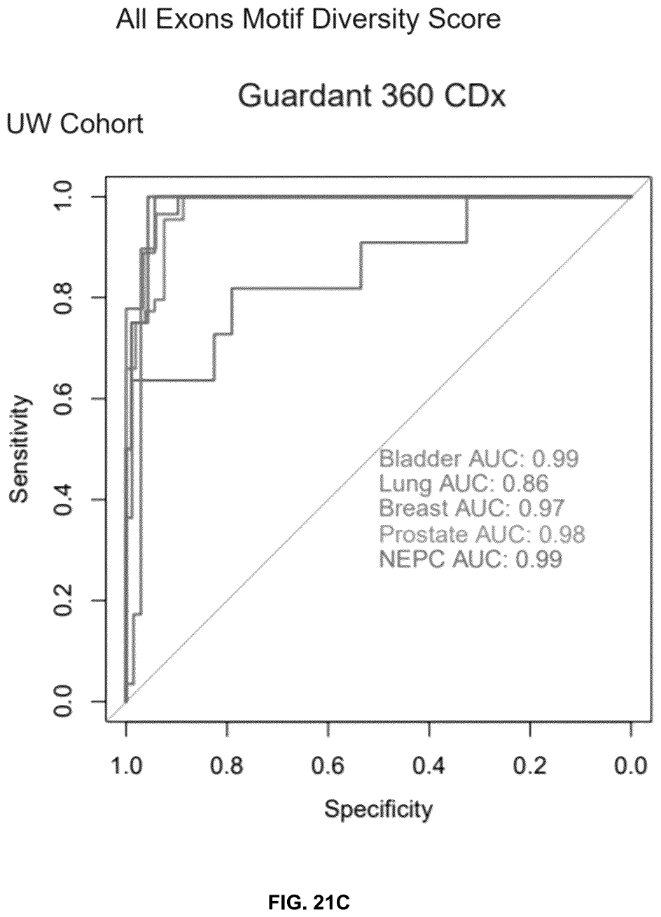

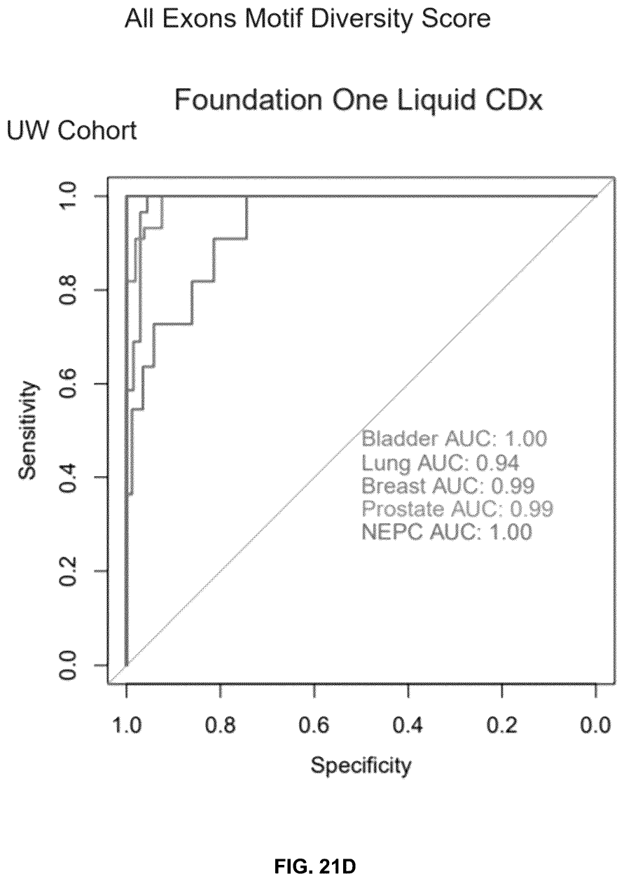

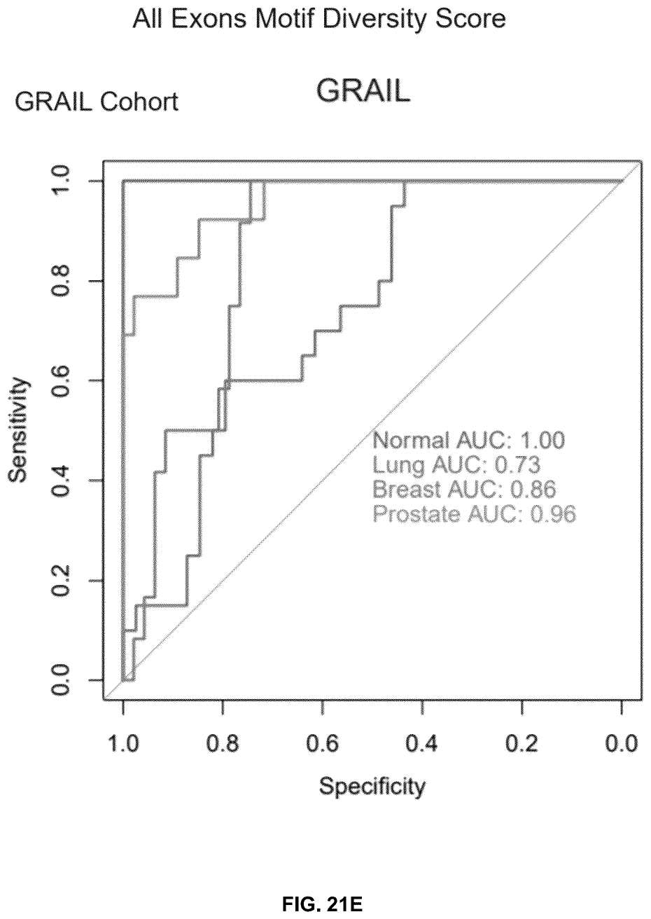

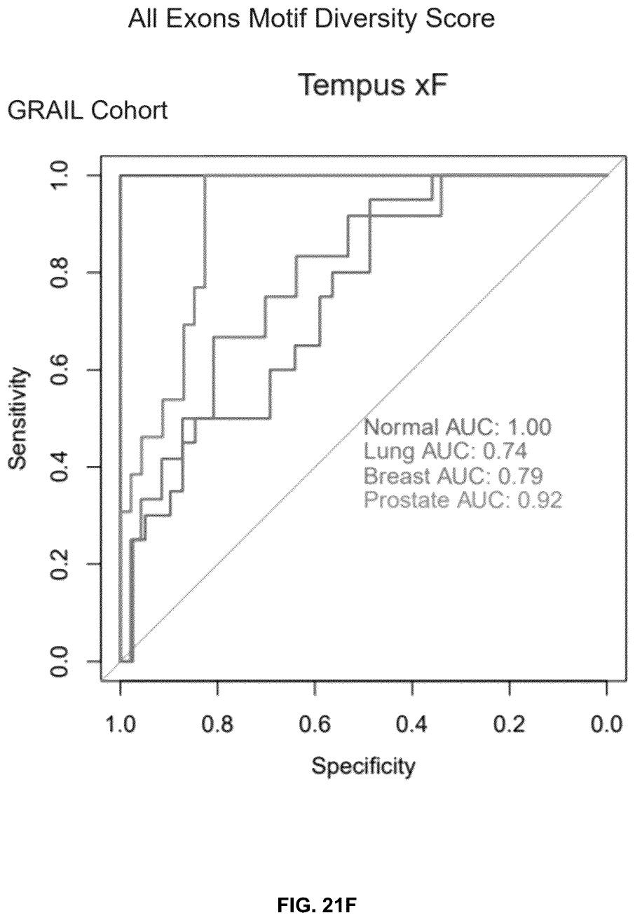

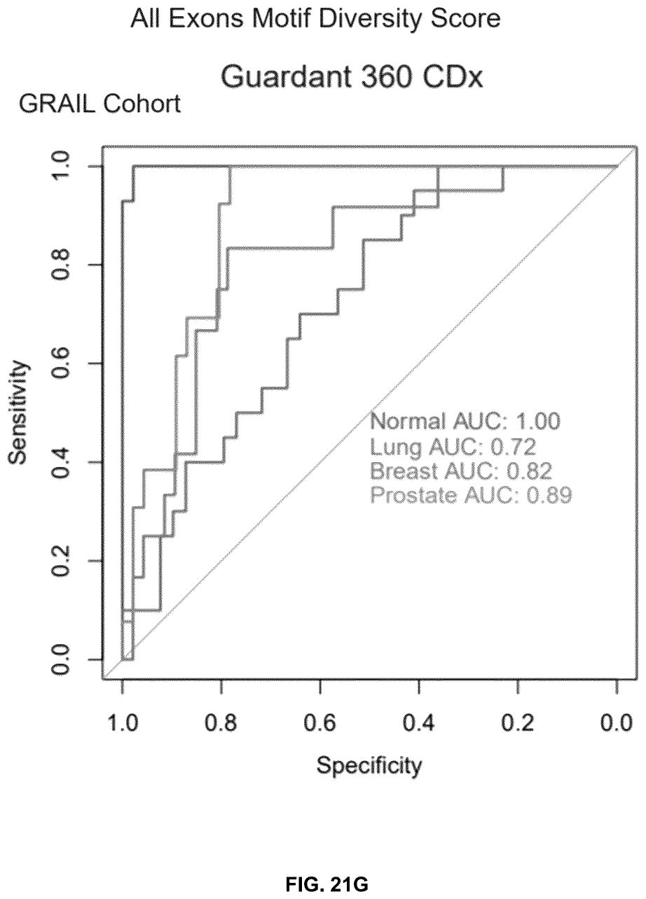

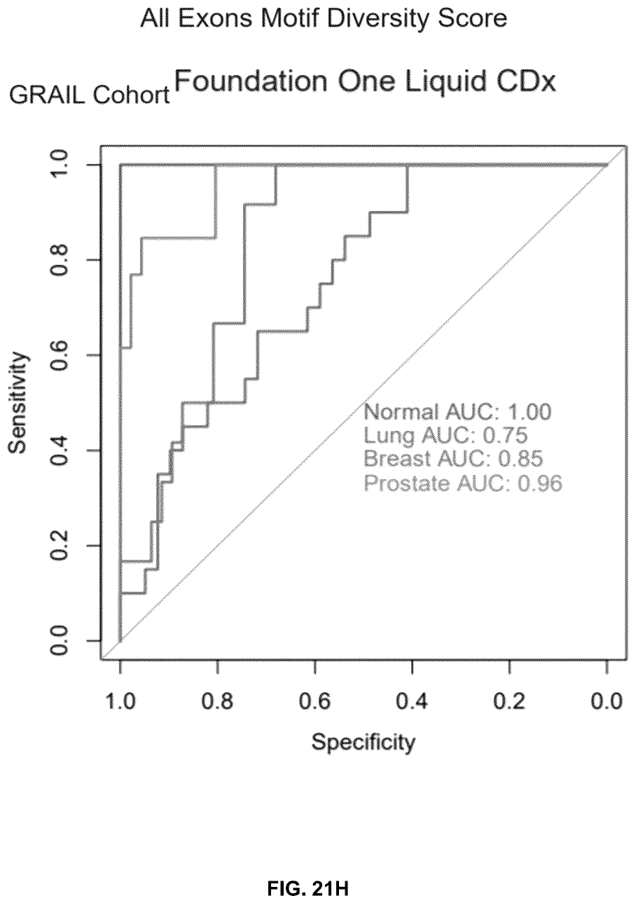

For each exon in each gene in each respective panel, the motif diversity score (MDS) was calculated for the set of fragments overlapping each exon. A minimum of 1 bp overlap was required to count as an overlap. MDS was calculated as reported previously24. The MDS metric at all coding exon of all genes in the respective panels was used for model training and validation as in the E1SE model (FIGS. 21A-21H).

Validation AUC in the UW cohort using genes in the UW panel was 0.99 for bladder cancer, 0.98 for breast cancer, 0.88 for lung cancer, 0.99 for NEPC, and 0.98 for prostate cancer. Validation AUC in the UW cohort using common genes between the UW panel and the Tempus xF panel was 1.00 for bladder cancer, 0.99 for breast cancer, 0.92 for lung cancer, 0.99 for NEPC, and 0.99 for prostate cancer. Validation AUC in the UW cohort using common genes between the UW panel and the Guardant 360 CDx panel was 0.99 for bladder cancer, 0.97 for breast cancer, 0.86 for lung cancer, 0.99 for NEPC, and 0.98 for prostate cancer. Validation AUC in the UW cohort using common genes between the UW panel and the Foundation One Liquid CDx panel was 1.00 for bladder cancer, 0.99 for breast cancer, 0.94 for lung cancer, 1.00 for NEPC, and 0.99 for prostate cancer.

Validation AUC in the GRAIL cohort using genes in the GRAIL panel was 0.86 for breast cancer, 0.73 for lung cancer, 0.96 for prostate cancer, and 1.00 for cancer vs normal. Validation AUC in the GRAIL cohort using common genes between the GRAIL panel and the Tempus xF panel was 0.79 for breast cancer, 0.74 for lung cancer, 0.92 for prostate cancer, and 1.00 for cancer vs normal. Validation AUC in the GRAIL cohort using common genes between the GRAIL panel and the Guardant 360 CDx panel was 0.82 for breast cancer, 0.72 for lung cancer, 0.89 for prostate cancer, and 1.00 for cancer vs normal. Validation AUC in the GRAIL cohort using common genes between the GRAIL panel and the Foundation One Liquid CDx panel was 0.85 for breast cancer, 0.75 for lung cancer, 0.96 for prostate cancer, and 1.00 for cancer vs normal.

### All Exons Small Fragment Proportions

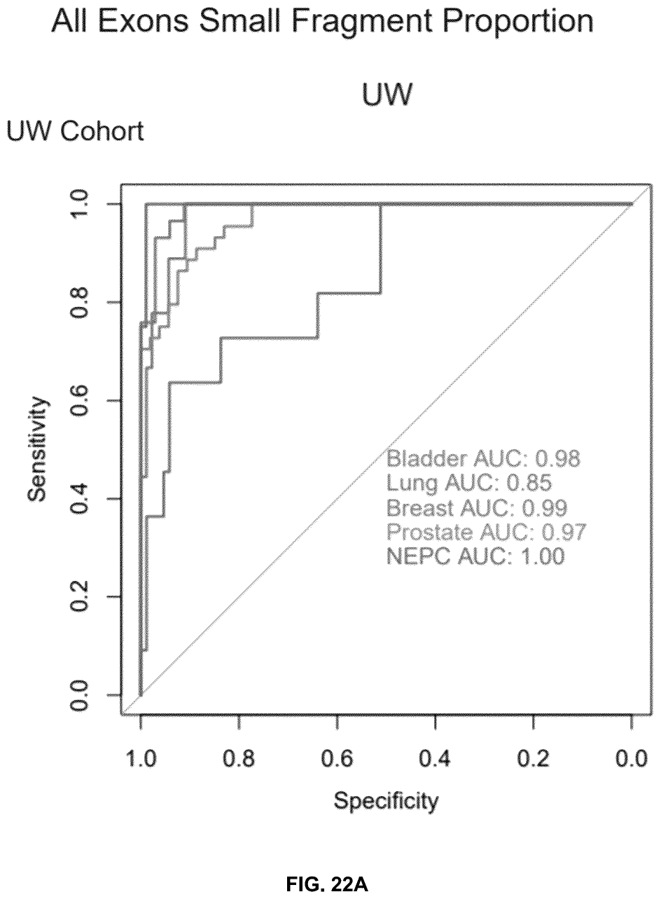

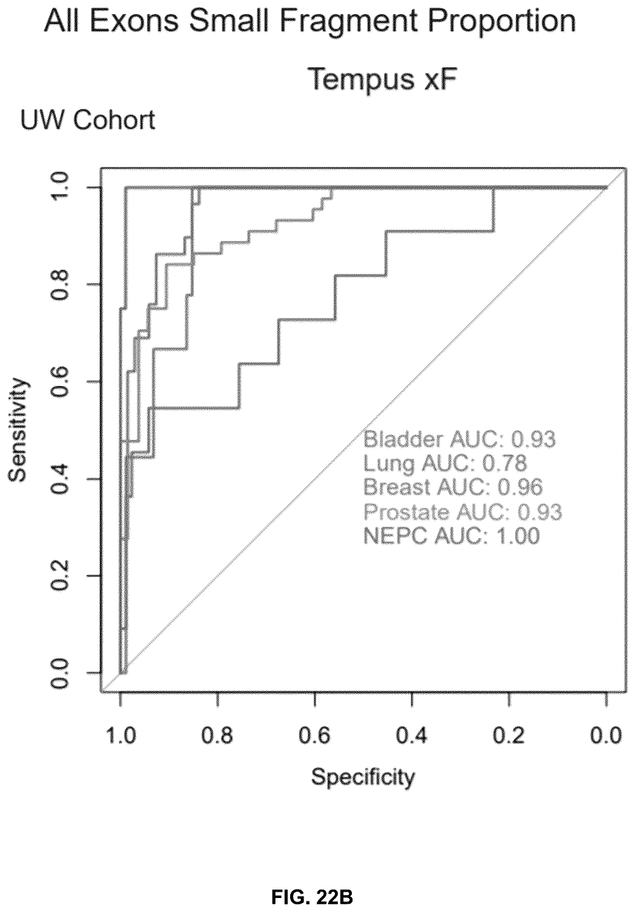

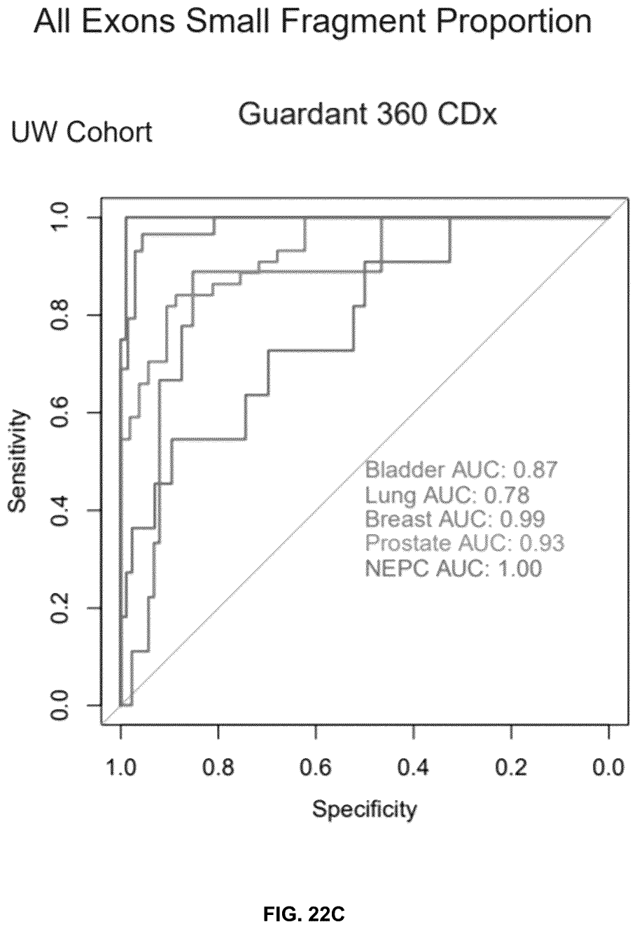

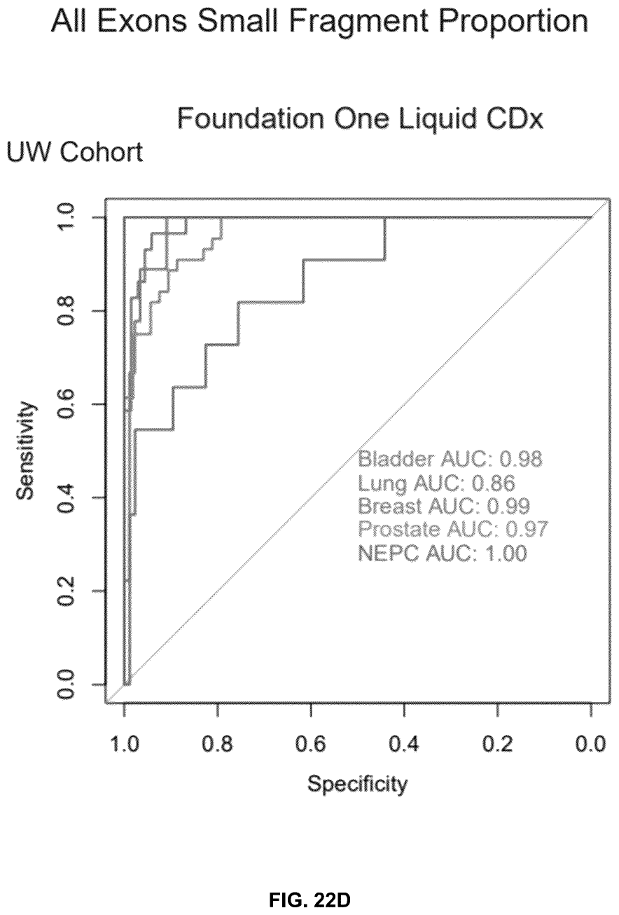

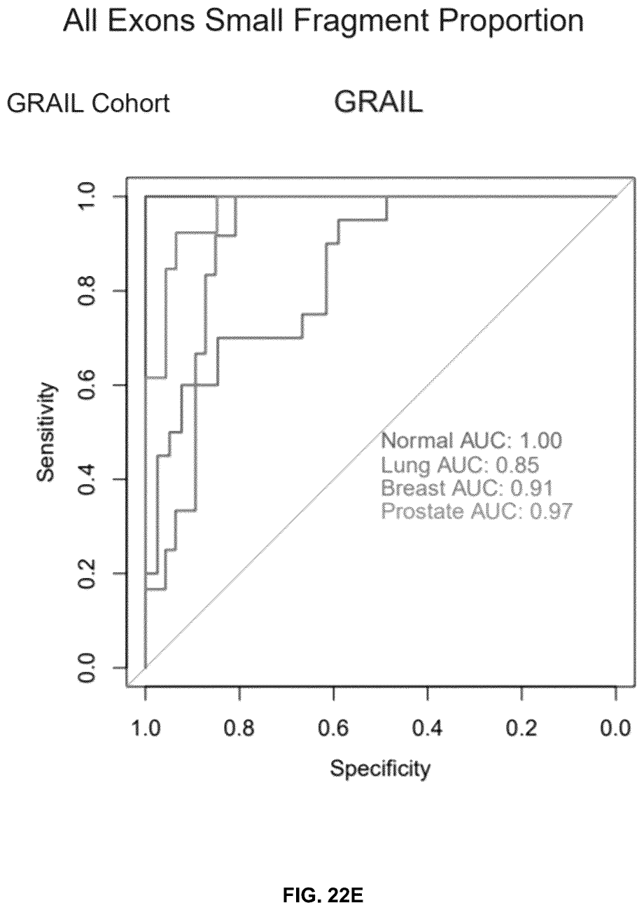

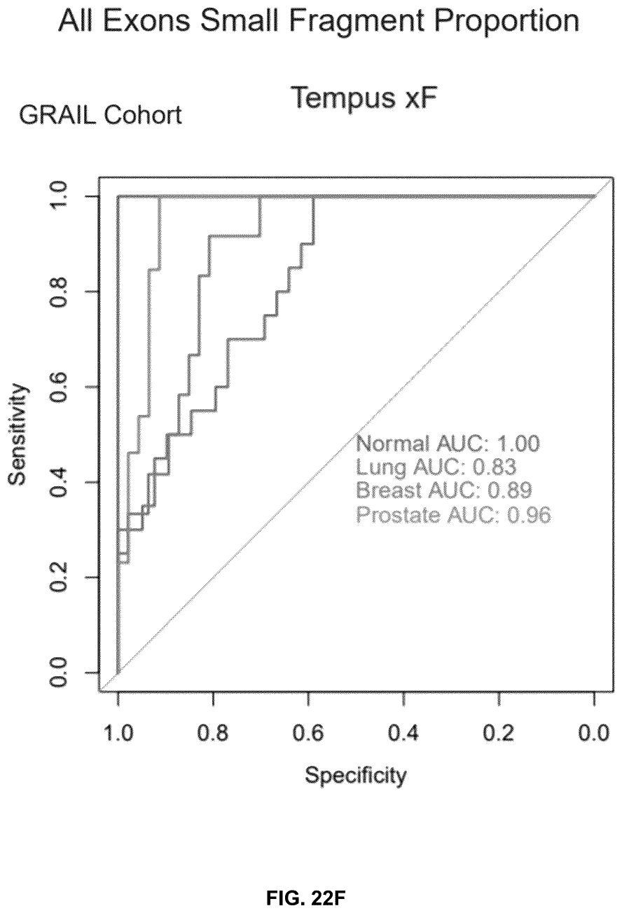

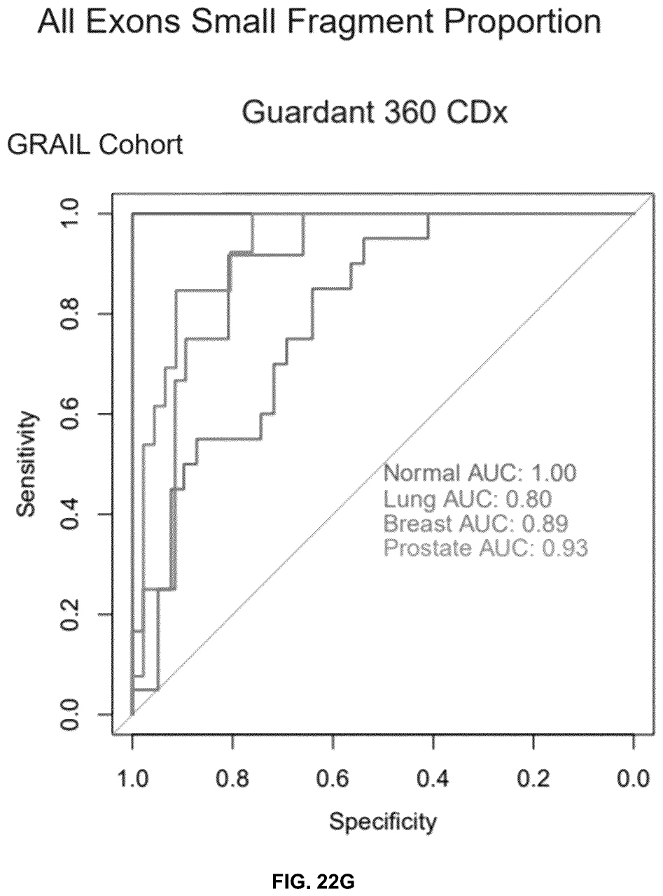

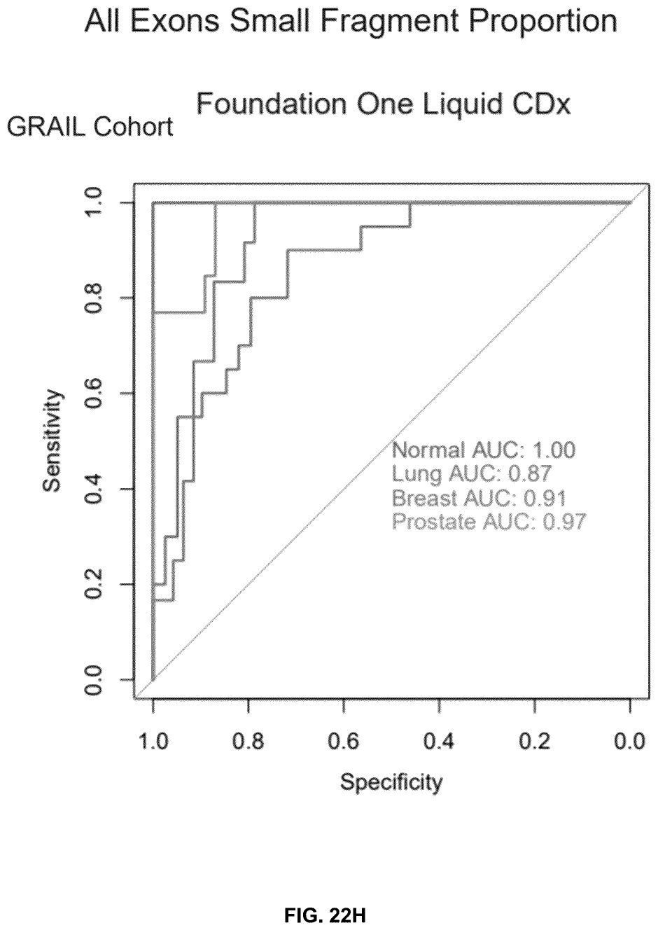

For each exon in each gene in each respective panel, fragments overlapping each exon were extracted and then the proportion of fragments less than or equal to 150 bp was calculated for each individual exon. A minimum of 1 bp overlap was required to count as an overlap. The proportion of small fragments for all coding exons of all genes in each respective panel was used for model training and validation as in the E1SE model (FIGS. 22A-22H).

Validation AUC in the UW cohort using genes in the UW panel was 0.98 for bladder cancer, 0.99 for breast cancer, 0.85 for lung cancer, 1.00 for NEPC, and 0.97 for prostate cancer. Validation AUC in the UW cohort using common genes between the UW panel and the Tempus xF panel was 0.93 for bladder cancer, 0.96 for breast cancer, 0.78 for lung cancer, 1.00 for NEPC, and 0.93 for prostate cancer. Validation AUC in the UW cohort using common genes between the UW panel and the Guardant 360 CDx panel was 0.87 for bladder cancer, 0.99 for breast cancer, 0.78 for lung cancer, 1.00 for NEPC, and 0.93 for prostate cancer. Validation AUC in the UW cohort using common genes between the UW panel and the Foundation One Liquid CDx panel was 0.98 for bladder cancer, 0.99 for breast cancer, 0.86 for lung cancer, 1.00 for NEPC, and 0.97 for prostate cancer.

Validation AUC in the GRAIL cohort using genes in the GRAIL panel was 0.91 for breast cancer, 0.85 for lung cancer, 0.97 for prostate cancer, and 1.00 for cancer vs normal. Validation AUC in the GRAIL cohort using common genes between the GRAIL panel and the Tempus xF panel was 0.89 for breast cancer, 0.83 for lung cancer, 0.96 for prostate cancer, and 1.00 for cancer vs normal. Validation AUC in the GRAIL cohort using common genes between the GRAIL panel and the Guardant 360 CDx panel was 0.89 for breast cancer, 0.80 for lung cancer, 0.93 for prostate cancer, and 1.00 for cancer vs normal. Validation AUC in the GRAIL cohort using common genes between the GRAIL panel and the Foundation One Liquid CDx panel was 0.91 for breast cancer, 0.87 for lung cancer, 0.97 for prostate cancer, and 1.00 for cancer vs normal.

### Combination Strategies—E1Se+E1depth

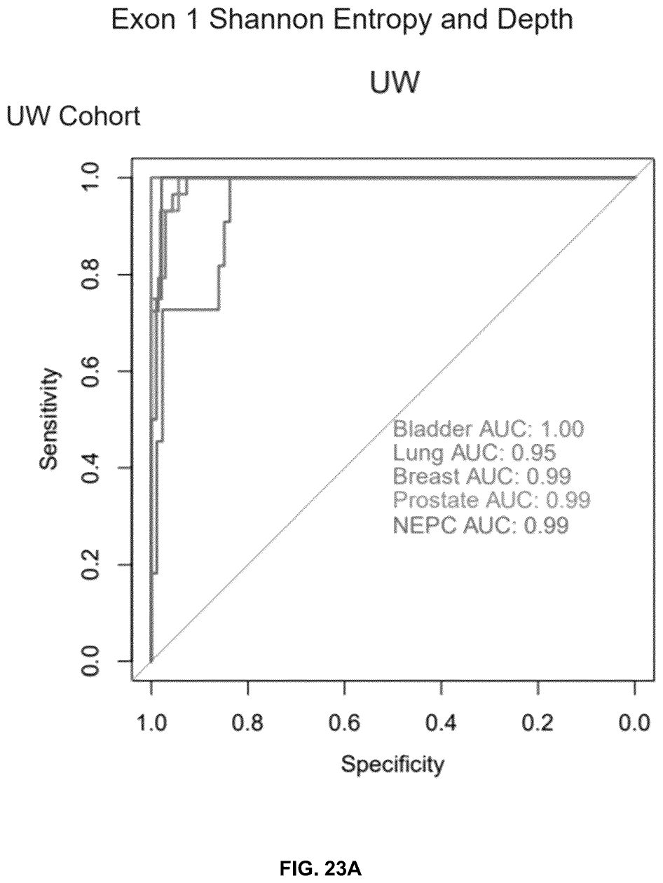

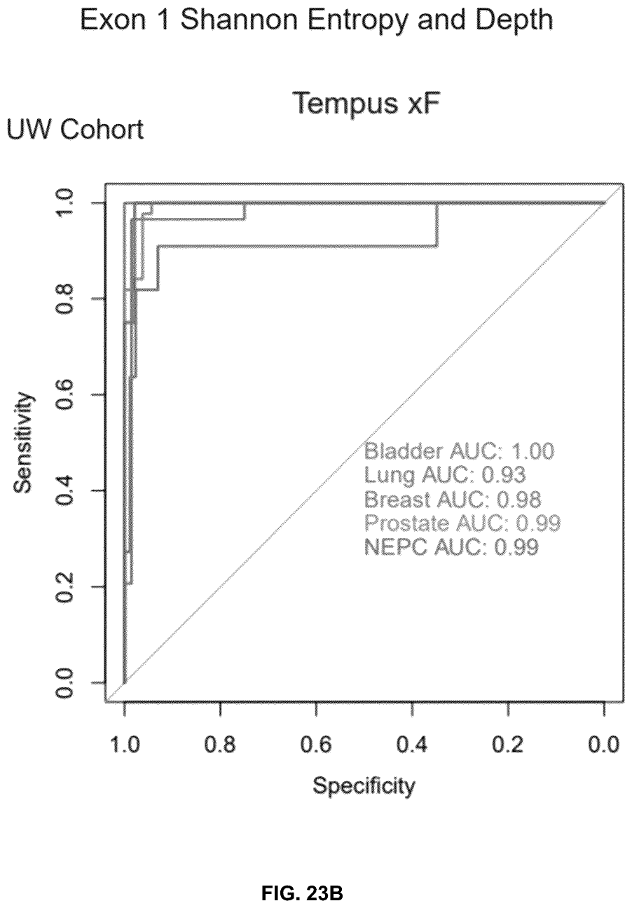

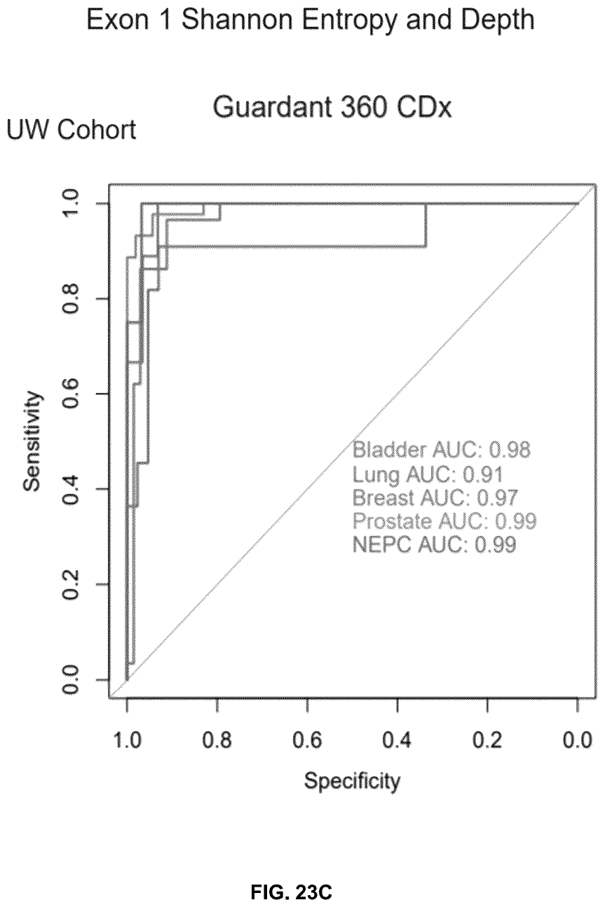

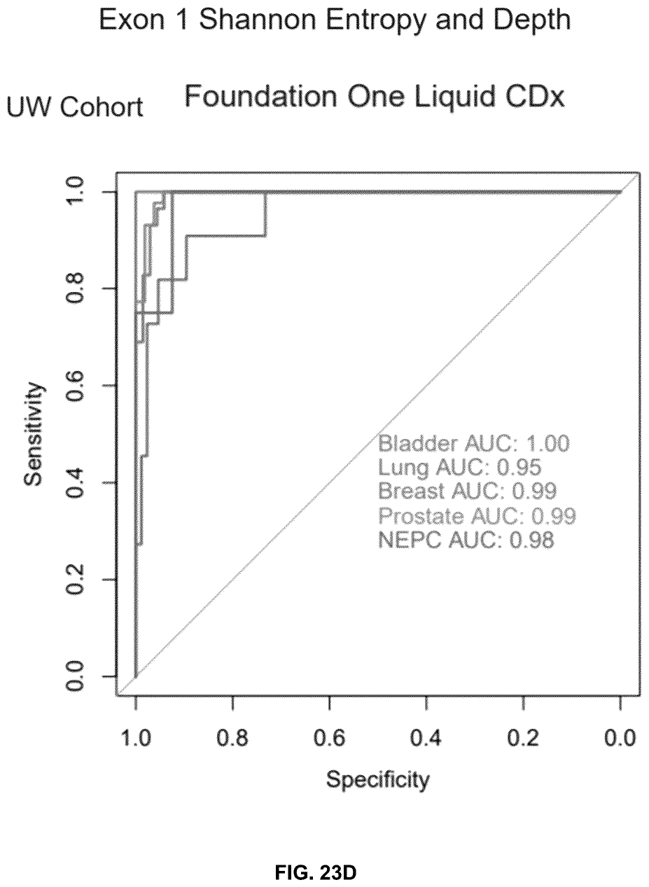

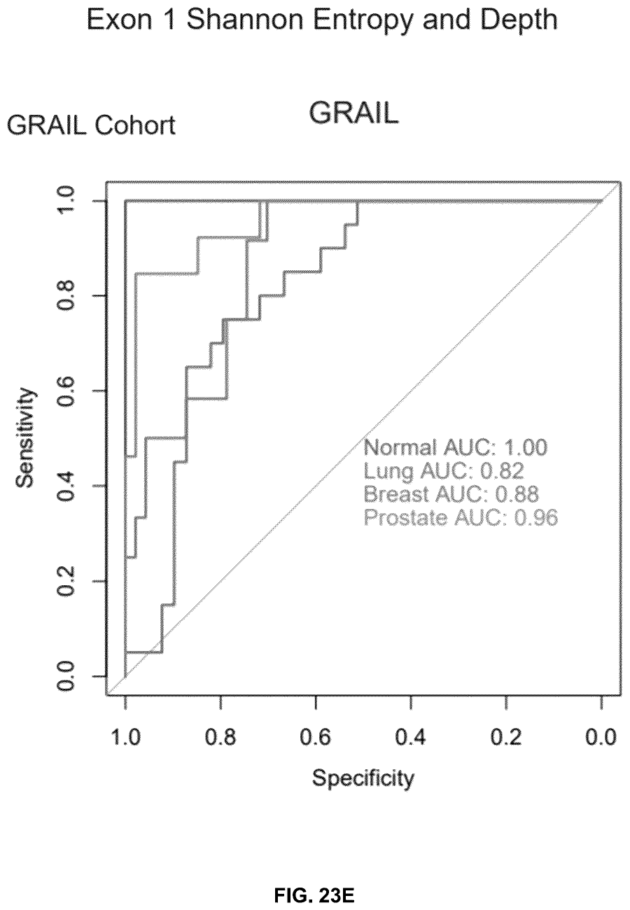

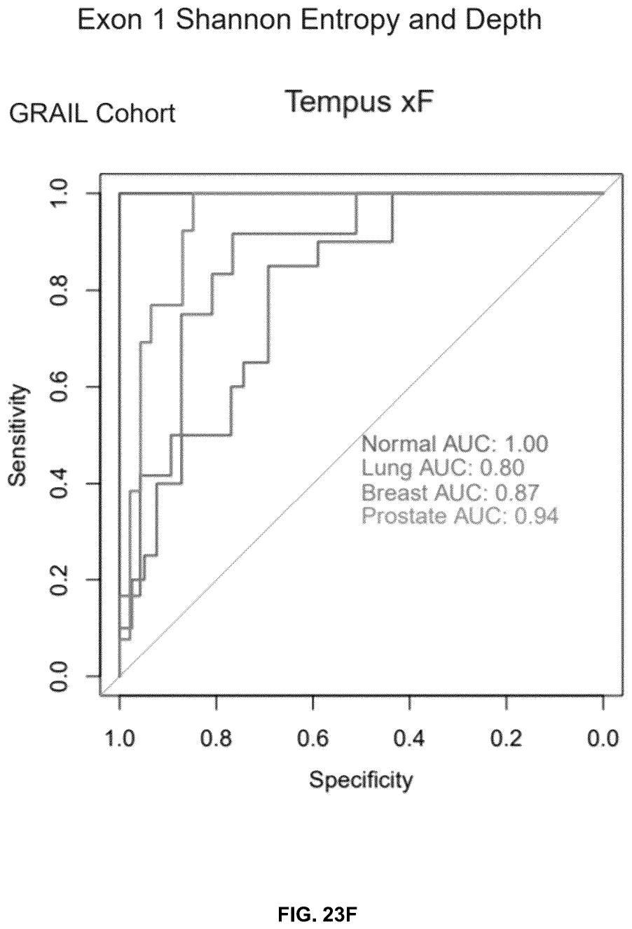

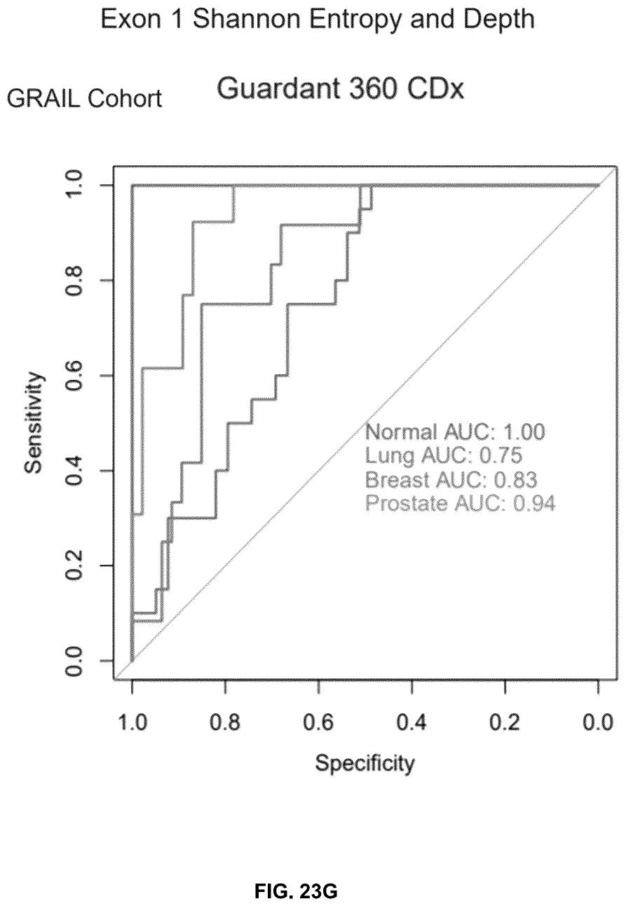

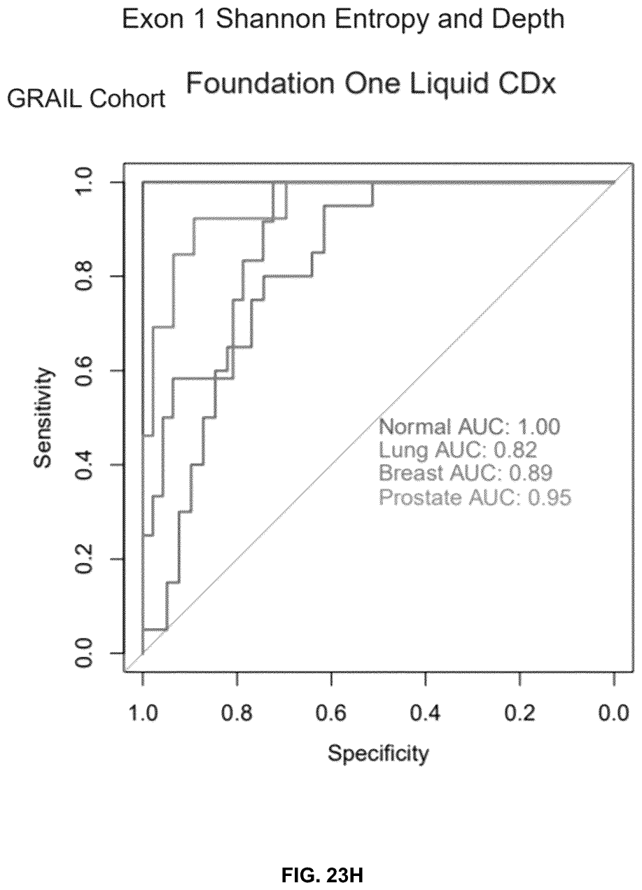

The features from E1SE and E1depth were combined into one feature table which was then used for model training and validation (FIGS. 23A-23H).

Validation AUC in the UW cohort using genes in the UW panel was 1.00 for bladder cancer, 0.99 for breast cancer, 0.95 for lung cancer, 0.99 for NEPC, and 0.99 for prostate cancer. Validation AUC in the UW cohort using common genes between the UW panel and the Tempus xF panel was 1.00 for bladder cancer, 0.98 for breast cancer, 0.93 for lung cancer, 0.99 for NEPC, and 0.99 for prostate cancer. Validation AUC in the UW cohort using common genes between the UW panel and the Guardant 360 CDx panel was 0.98 for bladder cancer, 0.97 for breast cancer, 0.91 for lung cancer, 0.99 for NEPC, and 0.99 for prostate cancer. Validation AUC in the UW cohort using common genes between the UW panel and the Foundation One Liquid CDx panel was 1.00 for bladder cancer, 0.99 for breast cancer, 0.95 for lung cancer, 0.98 for NEPC, and 0.99 for prostate cancer.

Validation AUC in the GRAIL cohort using genes in the GRAIL panel was 0.88 for breast cancer, 0.82 for lung cancer, 0.96 for prostate cancer, and 1.00 for cancer vs normal. Validation AUC in the GRAIL cohort using common genes between the GRAIL panel and the Tempus xF panel was 0.87 for breast cancer, 0.80 for lung cancer, 0.94 for prostate cancer, and 1.00 for cancer vs normal. Validation AUC in the GRAIL cohort using common genes between the GRAIL panel and the Guardant 360 CDx panel was 0.83 for breast cancer, 0.75 for lung cancer, 0.94 for prostate cancer, and 1.00 for cancer vs normal. Validation AUC in the GRAIL cohort using common genes between the GRAIL panel and the Foundation One Liquid CDx panel was 0.89 for breast cancer, 0.82 for lung cancer, 0.95 for prostate cancer, and 1.00 for cancer vs normal.

### Combination Strategies—All Exons Shannon Entropy and Depth

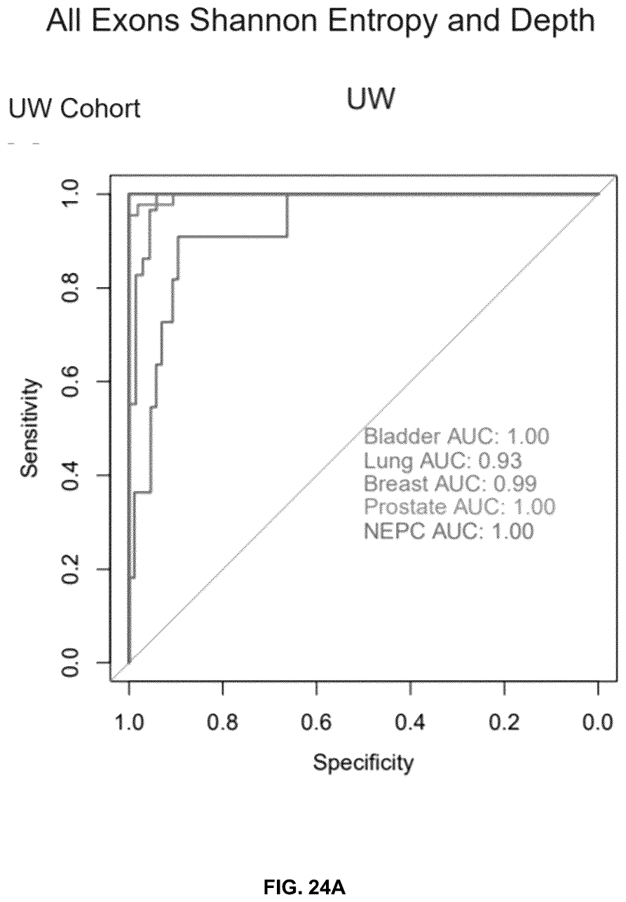

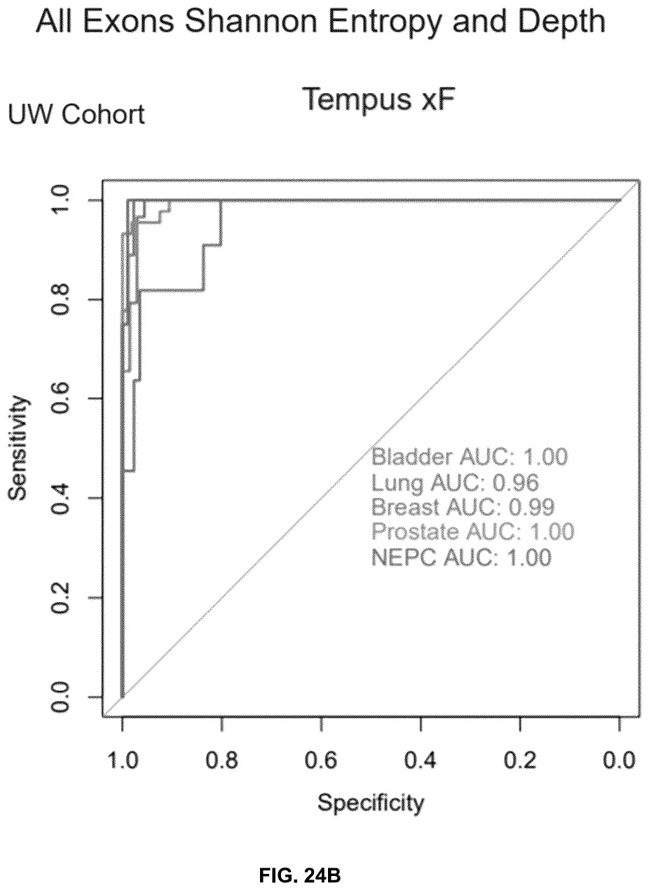

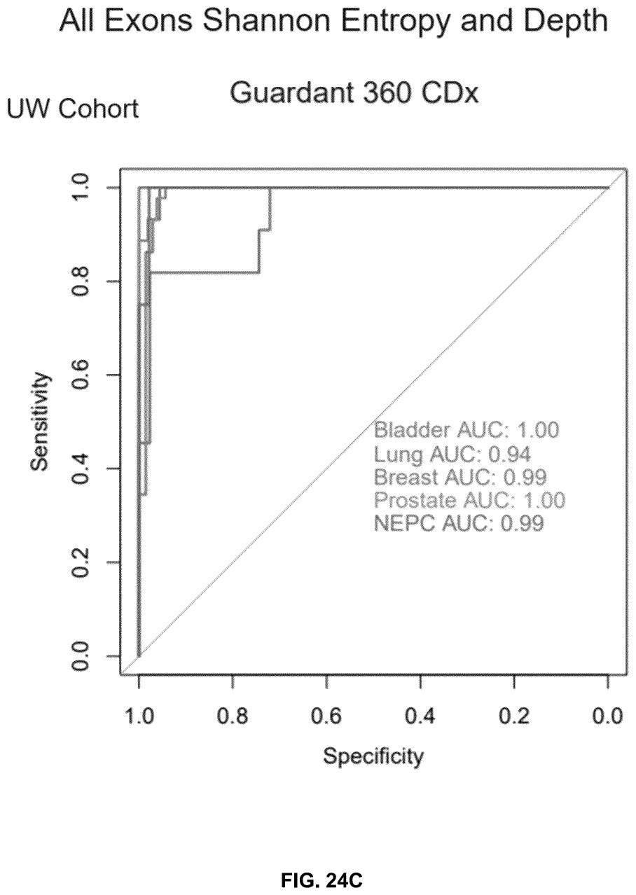

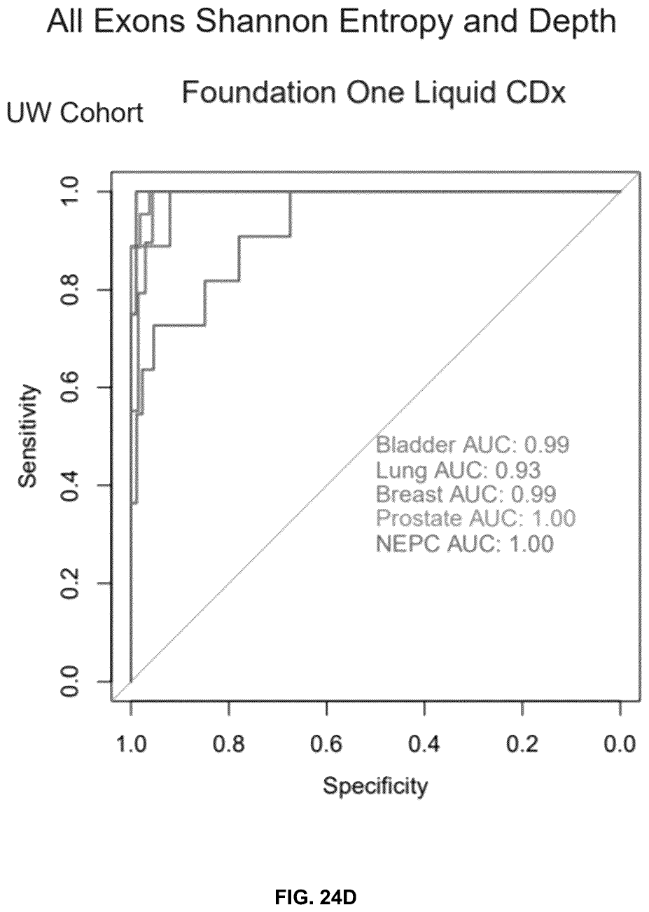

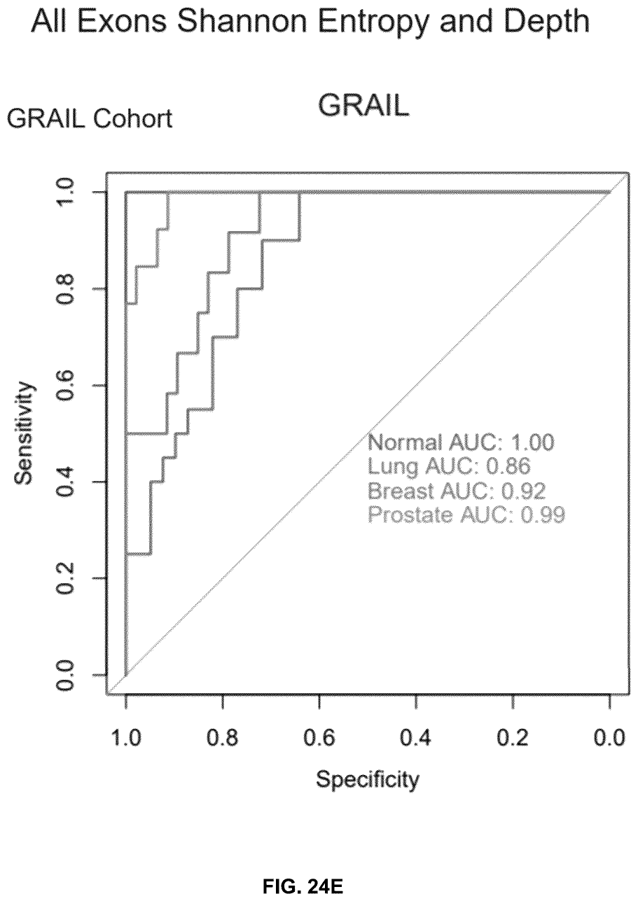

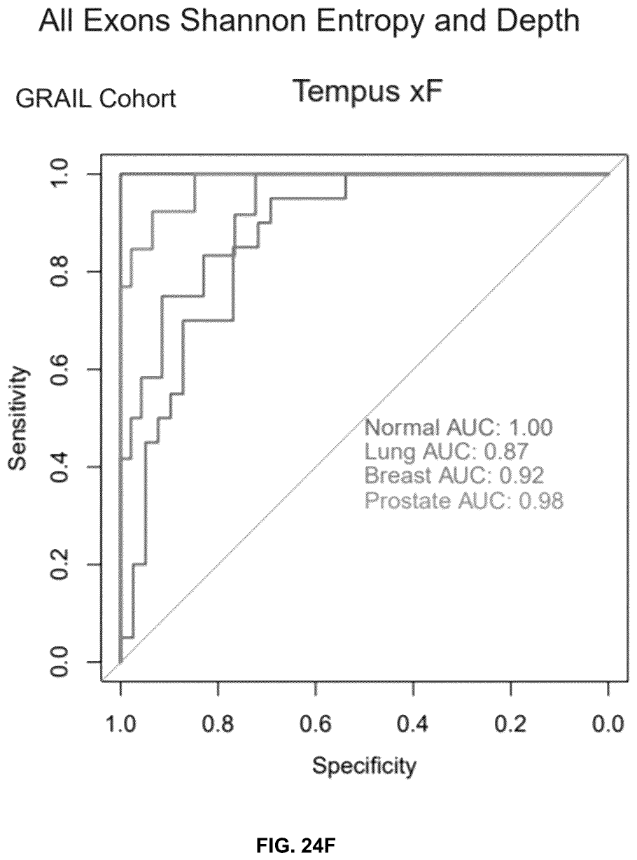

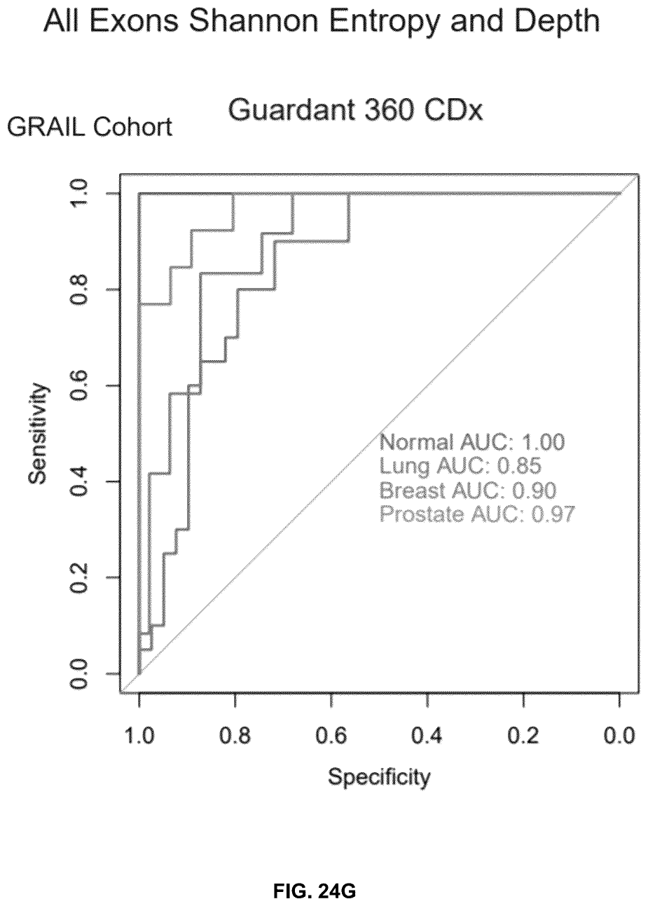

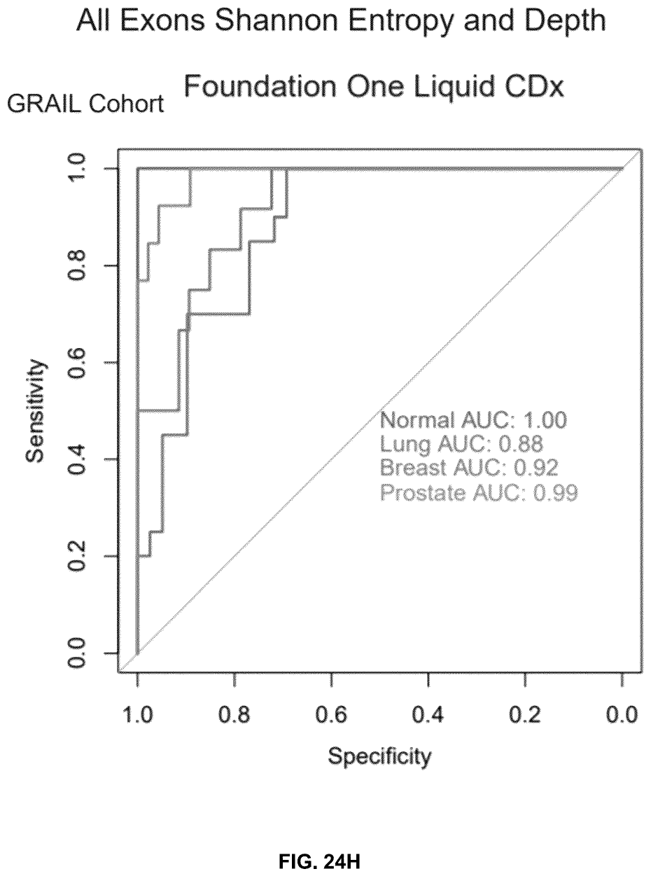

The Shannon Entropy and Depth for each exon for each gene in each respective gene panel were combined into one feature table which was then used for model training and validation. In instances where the number of features was greater than 15000, the features were limited to the top 15000 feature with the highest variance across samples (FIGS. 24A-24H).

Validation AUC in the UW cohort using genes in the UW panel was 1.00 for bladder cancer, 0.99 for breast cancer, 0.93 for lung cancer, 1.00 for NEPC, and 1.00 for prostate cancer. Validation AUC in the UW cohort using common genes between the UW panel and the Tempus xF panel was 1.00 for bladder cancer, 0.99 for breast cancer, 0.96 for lung cancer, 1.00 for NEPC, and 1.00 for prostate cancer. Validation AUC in the UW cohort using common genes between the UW panel and the Guardant 360 CDx panel was 1.00 for bladder cancer, 0.99 for breast cancer, 0.94 for lung cancer, 0.99 for NEPC, and 1.00 for prostate cancer. Validation AUC in the UW cohort using common genes between the UW panel and the Foundation One Liquid CDx panel was 0.99 for bladder cancer, 0.99 for breast cancer, 0.93 for lung cancer, 1.00 for NEPC, and 1.00 for prostate cancer.

Validation AUC in the GRAIL cohort using genes in the GRAIL panel was 0.92 for breast cancer, 0.86 for lung cancer, 0.99 for prostate cancer, and 1.00 for cancer vs normal. Validation AUC in the GRAIL cohort using common genes between the GRAIL panel and the Tempus xF panel was 0.92 for breast cancer, 0.87 for lung cancer, 0.98 for prostate cancer, and 1.00 for cancer vs normal. Validation AUC in the GRAIL cohort using common genes between the GRAIL panel and the Guardant 360 CDx panel was 0.90 for breast cancer, 0.85 for lung cancer, 0.97 for prostate cancer, and 1.00 for cancer vs normal. Validation AUC in the GRAIL cohort using common genes between the GRAIL panel and the Foundation One Liquid CDx panel was 0.92 for breast cancer, 0.88 for lung cancer, 0.99 for prostate cancer, and 1.00 for cancer vs normal.

### Predicting Ctdna Fraction Using Exon 1 Shannon Entropy

Cancer samples in both the UW and GRAIL cohorts were separated into two categories based on their ctDNA fraction. Samples with a ctDNA fraction less than 0.05 were categorized as “low” and samples with a ctDNA fraction greater than or equal to 0.05 were categorized as “high”.

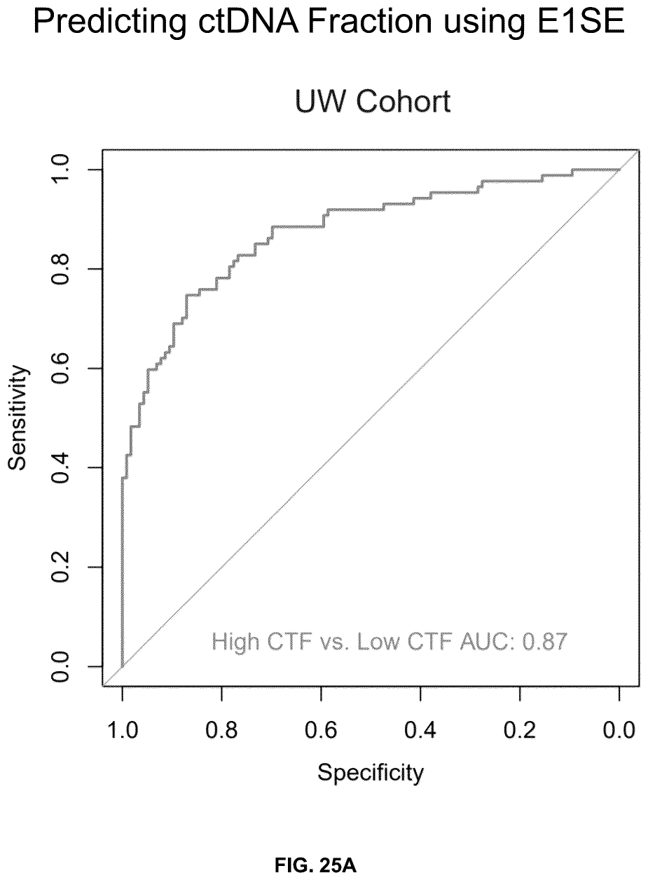

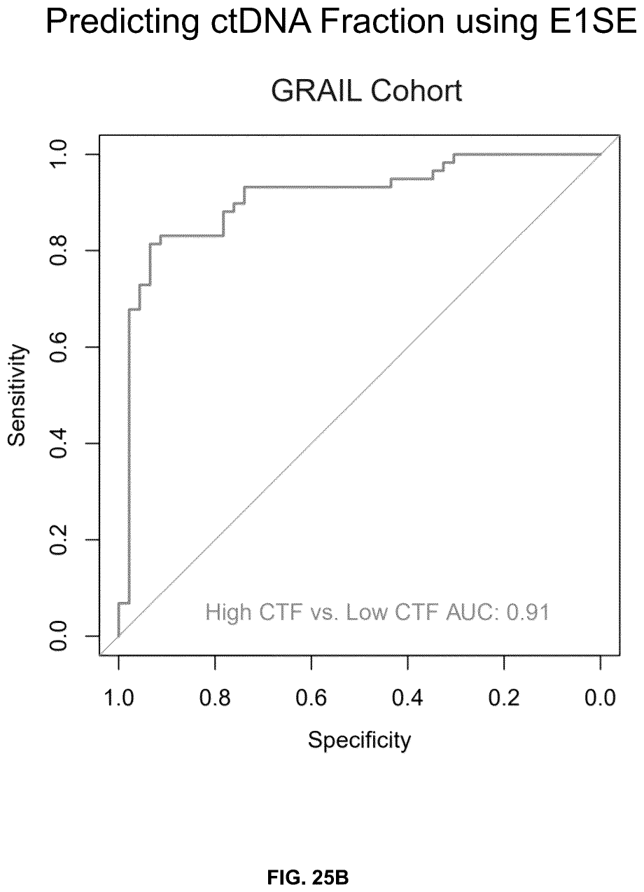

The E1SE metric from each gene in each respective gene panel was then used to predict high or low ctDNA fraction using a 10-fold cross validation approach in each cohort separately (FIGS. 25A-25B). The AUC for predicting high vs. low ctDNA fraction in the UW cohort was 0.87 and the AUC for predicting high vs. low ctDNA fraction in the GRAIL cohort was 0.91.

### Shannon Entropy of Reads Overlapping Trascription Factor Binding Sites

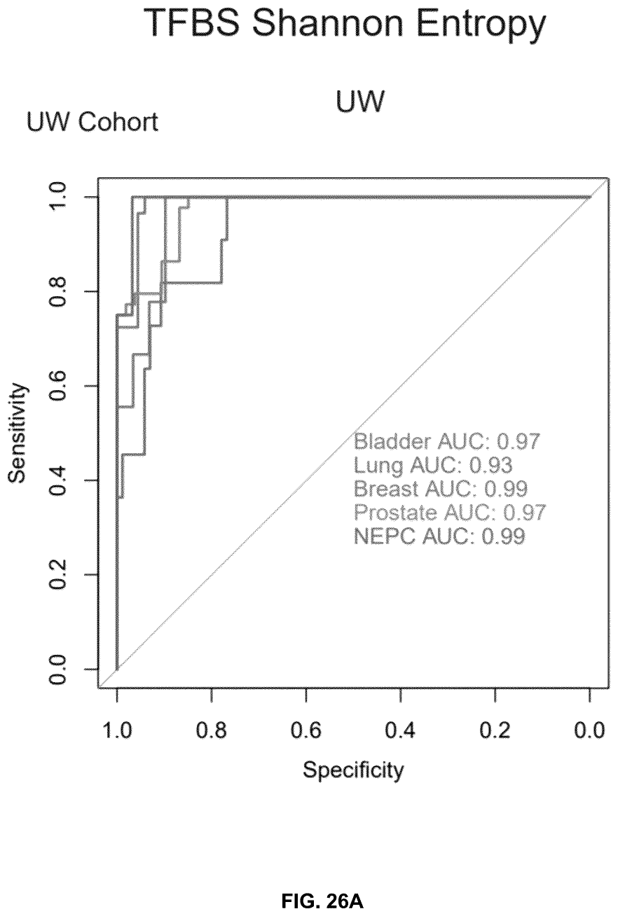

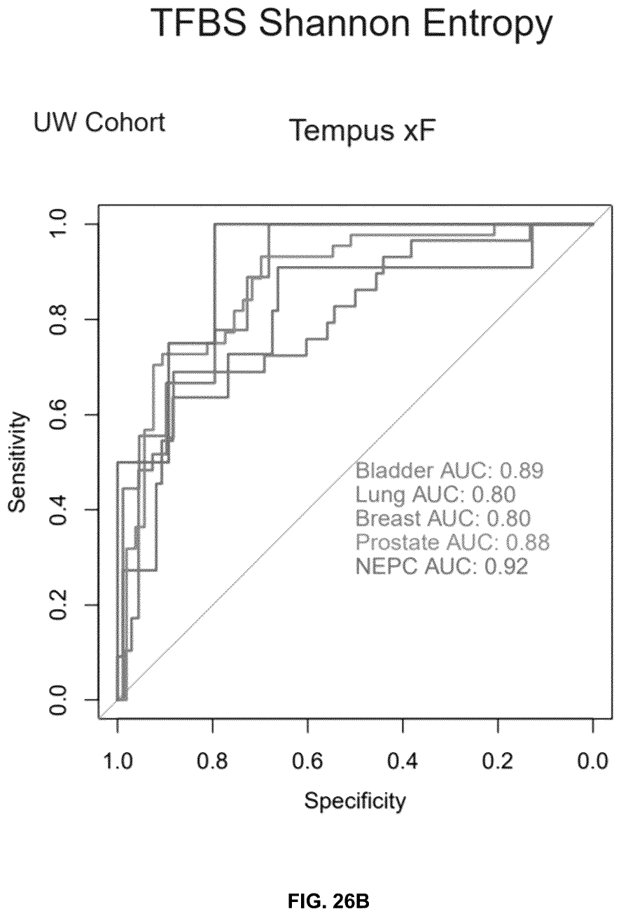

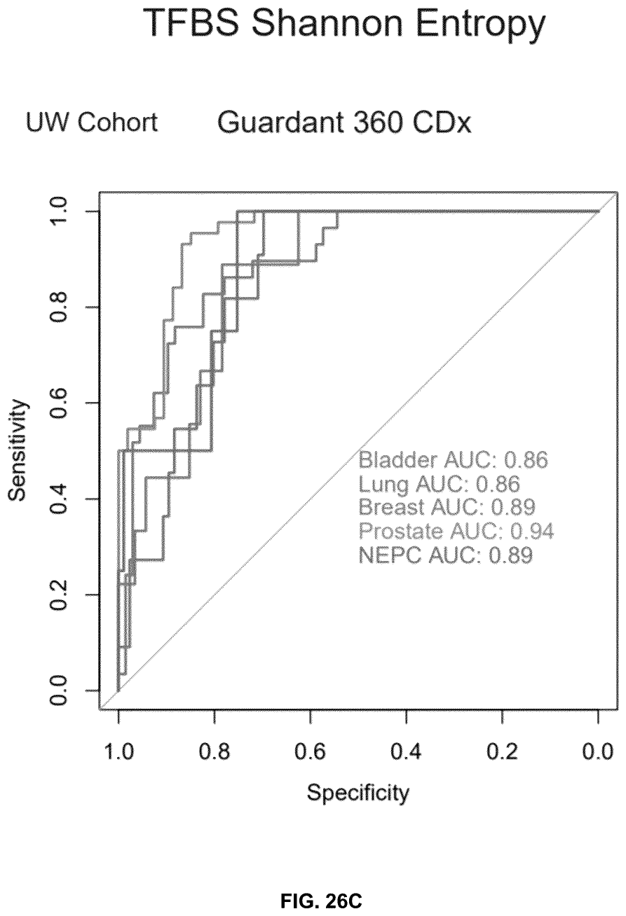

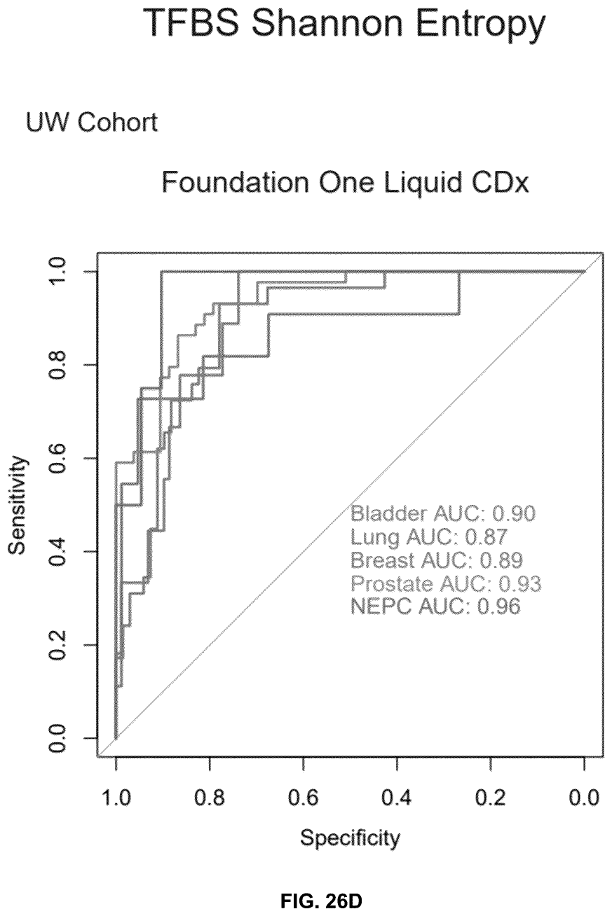

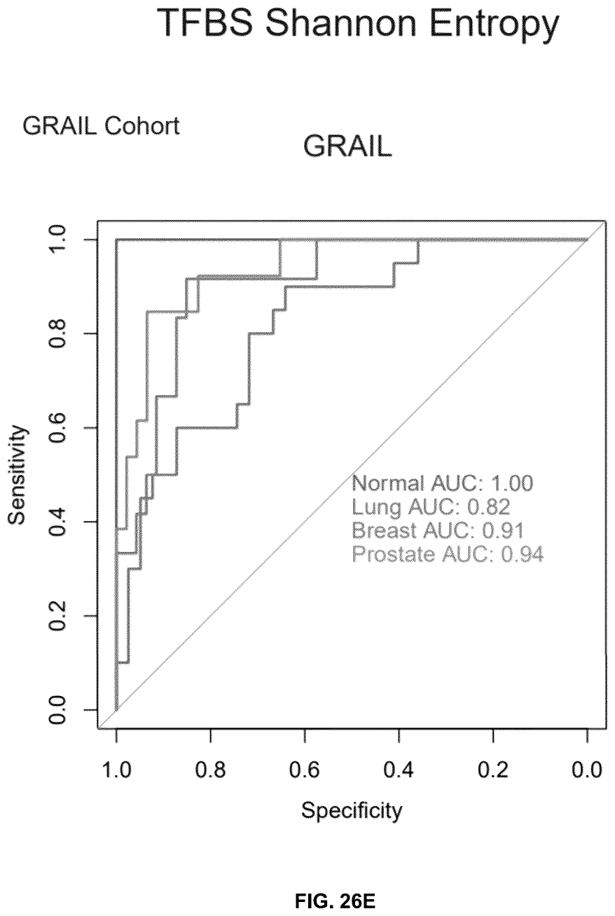

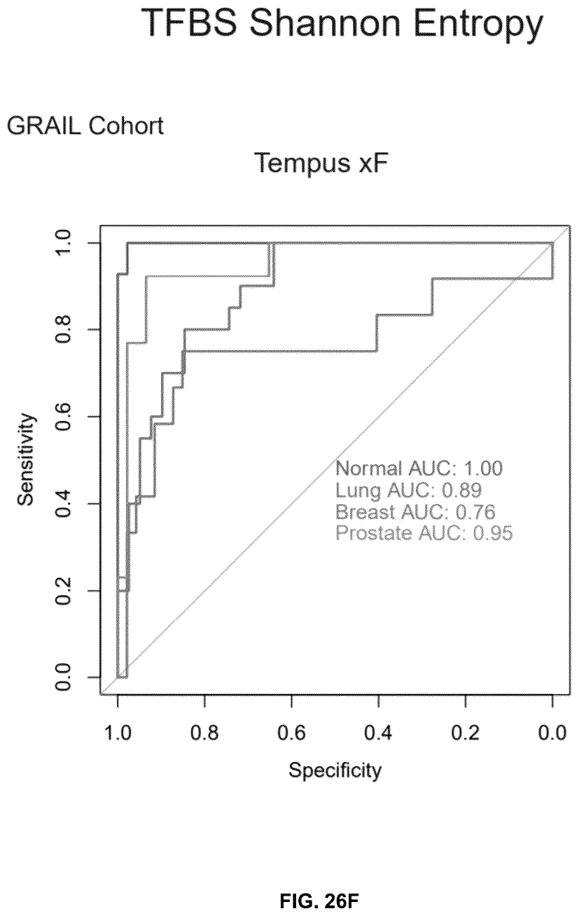

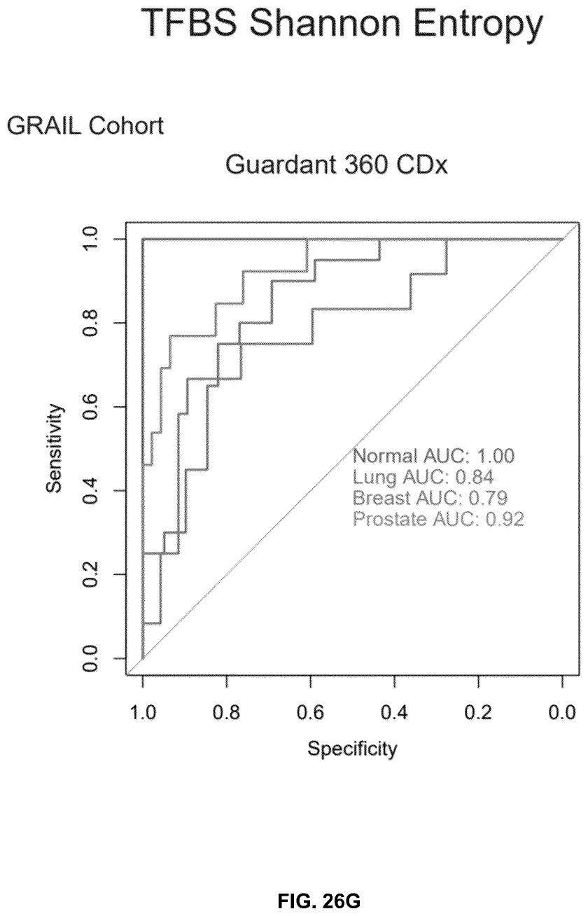

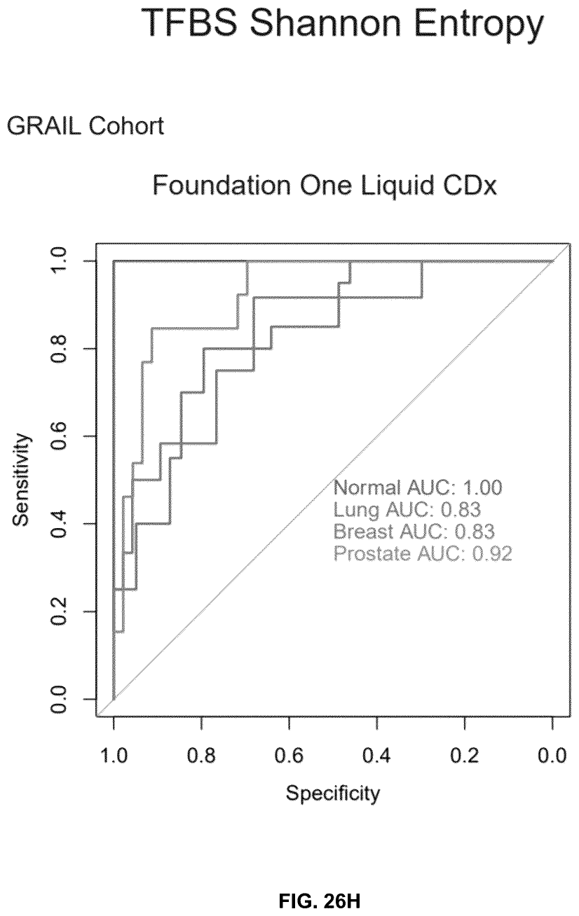

Consensus transcription factor binding sites (TFBS) were obtained from the Gene Transcription Regulation Database (GTRD). The transcription factors analyzed were ADA2, ADCYAP1, ADNP, AEBP2, AFF1, AFF4, AGO1, AGO2, AHR, AHRR, ALKBH3, ALX4, ALYREF, AMH, APC, APOBEC3B, AR, ARHGAP35, ARID1A, ARID1B, ARID2, ARID3A, ARID3B, ARID4B, ARID5B, ARNT, ARNT2, ARNTL, ARRB1, ASCL1, ASCL2, ASF1A, ASHIL, ASH2L, ASXL2, ATF1, ATF2, ATF3, ATF4, ATF5, ATF6, ATF7, ATF7IP, ATM, ATOHI, ATRX, AUTS2, BACHI, BACH2, BAHD1, BANP, BAP1, BARHL1, BARX1, BARX2, BATF, BATF2, BATF3, BBX, BCHE, BCLI1A, BCL11B, BCL3, BCL6, BCL6B, BCLAF1, BCOR, BDP1, BHLHE40, BHLHE41, BICRA, BMII, BPTF, BRCA1, BRD1, BRD2, BRD3, BRD4, BRD7, BRD9, BRF1, BRF2, BRPF3, CARMI, CASP8AP2, CASZI, CAT, CAVIN1, CBFA2T2, CBFA2T3, CBFB, CBX1, CBX2, CBX3, CBX5, CBX6, CBX7, CBX8, CC2D1A, CCAR2, CCND2, CCNT2, CDC5L, CDC73, CDK12, CDK2, CDK7, CDK8, CDK9, CDKN1B, CDX1, CDX2, CEBPA, CEBPB, CEBPD, CEBPE, CEBPG, CEBPZ, CENPA, CENPT, CGAS, CHAF1A, CHAFIB, CHAMP1, CHD1, CHD2, CHD4, CHD7, CHD8, CHTOP, CIC, CIITA, CLOCK, c-myc, CNOT3, COBLL1, COIL, COPS2, CPSF3, CREB1, CREB3, CREB3L1, CREB3L2, CREB3L4, CREBBP, CREBL2, CREM, CRY1, CSHL1, CSNK2A1, CTBP1, CTBP2, CTCF, CTCFL, CTNNB1, CTR9, CUL4A, CUX1, CXXC1, CXXC4, DACHI, DAXX, DBP, DCP1A, DDIT3, DDX11, DDX20, DDX21, DDX5, DEAF1, DEK, DICER1, DIDO1, DKK1, DLX1, DLX2, DLX4, DLX6, DMAP1, DMC1, DMRT1, DNMT1, DNMT3A, DNMT3B, DOT1L, DPF2, DPPA3, DRAP1, DROSHA, DTL, DUX4, DYRK1A, E2F1, E2F2, E2F3, E2F4, E2F5, E2F6, E2F7, E2F8, E4F1, EBF1, EBF3, EBNAIBP2, EBP, EED, EGFR, EGR1, EGR2, EGR3, EHF, EHMT2, ELF1, ELF2, ELF3, ELF4, ELF5, ELK1, ELK3, ELK4, ELL2, EMSY, EMX1, EOMES, EP300, EP400, EPAS1, EPC1, ERCC2, ERCC3, ERCC6, ERCC8, ERF, ERG, ESCO2, ESR1, ESR2, ESRRA, ETS1, ETS2, ETV1, ETV2, ETV4, ETV5, ETV6, ETV7, EVX1, EWSR1, EZH2, F10, F2RL1, FAM208A, FANCD2, FEV, FEZF1, FGFR1, FIP1L1, FLI1, FOS, FOSB, FOSL1, FOSL2, FOXA1, FOXA2, FOXA3, FOXC1, FOXD2, FOXD3, FOXEI, FOXF1, FOXF2, FOXG1, FOXHI, FOXJ2, FOXK1, FOXK2, FOXMI, FOXN3, FOXO1, FOXO3, FOXO4, FOXP1, FOXP2, FOXP3, FOXQ1, FOXR2, FUS, FXR1, FXR2, GABPA, GABPBI, GATA1, GATA2, GATA3, GATA4, GATA6, GATAD1, GATAD2A, GATAD2B, GFII, GFIIB, GLI1, GLI2, GLI3, GLI4, GLIS1, GLIS2, GLIS3, GMEBI, GMEB2, GREB1, GRHL1, GRHL2, GRHL3, GTF2A2, GTF2B, GTF2E2, GTF2F1, GTF3A, GTF3C2, GTF3C5, GUCY1B3, GZF1, H2AFZ, HAND1, HAND2, HBP1, HBZ, HCFC1, HDAC1, HDAC2, HDAC3, HDAC4, HDAC6, HDAC8, HDGF, HDGFL3, HES1, HES2, HES4, HES5, HES7, HESX1, HEXIMI, HEY1, HEY2, HEYL, HHEX, HIC1, HIC2, HIF1A, HIF3A, HINFP, HIRA, HISTIHIT, HIVEP1, HIVEP3, HJURP, HLF, HMBOX1, HMCES, HMG20A, HMG20B, HMGA1, HMGA2, HMGB1, HMGB2, HMGN3, HMGXB4, HNF1A, HNFIB, HNF4A, HNF4G, HNRNPHI, HNRNPK, HNRNPL, HNRNPLL, HNRNPUL1, HOMEZ, HOXA1, HOXA10, HOXA13, HOXA2, HOXA4, HOXA5, HOXA6, HOXA7, HOXA9, HOXB13, HOXB4, HOXB5, HOXB6, HOXB7, HOXB8, HOXC11, HOXC13, HOXC5, HOXC6, HOXC8, HOXC9, HOXD1, HOXD11, HOXD4, HOXD9, HSD17B8, HSF1, HSF2, HSF4, ID1, ID2, ID3, ID4, IGF1R, IGLV5-37, IKZF1, IKZF2, IKZF3, IKZF5, ILF3, ILK, ING2, ING5, INO80, INSM2, INSR, INTS11, INTS12, INTS13, INTS3, IRF1, IRF2, IRF3, IRF4, IRF5, IRF8, IRF9, IRX2, IRX3, IRX5, ISL1, ISL2, IVNSIABP, JARID2, JDP2, JMJD6, JUN, JUNB, JUND, KAT2A, KAT2B, KAT5, KAT7, KAT8, KDM1A, KDMIB, KDM2B, KDM3A, KDM3B, KDM4A, KDM4B, KDM4C, KDM5A, KDM5B, KDM5C, KDM5D, KDM6A, KDM6B, KDM7A, KLF1, KLF10, KLF11, KLF12, KLF13, KLF14, KLF15, KLF16, KLF17, KLF3, KLF4, KLF5, KLF6, KLF7, KLF8, KLF9, KMT2A, KMT2B, KMT2C, KMT2D, L3 MBTL2, L3 MBTL4, LAMB3, LAMTOR5, LARP7, LCORL, LDB1, LEF1, LEO1, LHX2, LHX3, LHX4, LHX5, LHX6, LHX9, LMNA, LMNB1, LMNB2, LMO1, LMTK3, LMXIB, LYL1, MAF, MAFB, MAFF, MAFG, MAFK, MAML1, MAPILC3B, MAP2K1, MAPK14, MAPK3, MAX, MAZ, MBD1, MBD2, MBD3, MBD3L2, MBD4, MBL2, MBTD1, MBTPS2, MCM2, MCM3, MCM5, MCM7, MCRS1, MDM2, MEI, ME3, MECOM, MECP2, MED12, MED26, MEF2A, MEF2B, MEF2C, MEF2D, MEIS1, MEIS2, MEN1, MEOX2, METTL 14, METTL3, MGA, MIER1, MIER2, MIER3, MITF, MIXL1, MLLT1, MLLT3, MLX, MLXIP, MNT, MNX1, MORC2, MPHOSPH8, MSC, MSX1, MSX2, MTA1, MTA2, MTA3, MTHFD1, MTOR, MUC22, MXD1, MXD3, MXD4, MXII, MYB, MYBL1, MYBL2, MYC, MYCN, MYF5, MYF6, MYH11, MYNN, MYOCD, MYOD1, MYOG, MYRF, MZF1, NAB2, NANOG, NBN, NCAPH2, NCOA1, NCOA2, NCOA3, NCOA4, NCOA6, NCOR1, NCOR2, NELFA, NELFE, NEUROD1, NEUROD2, NEUROG2, NEUROG3, NFAT5, NFATC1, NFATC3, NFATC4, NFE2, NFE2L1, NFE2L2, NFE2L3, NF1A, NFIB, NFIC, NFIL3, NFKB1, NFKB2, NFKB1A, NFKBIZ, NFRKB, NFXL1, NFYA, NFYB, NFYC, NHLHI, NIPBL, NKX2-1, NKX2-2, NKX2-3, NKX2-5, NKX2-8, NKX3-1, NKX6-1, NME2, NONO, NOTCH1, NOTCH3, NPAT, NROBI, NRID1, NRIH2, NR1H3, NRIH4, NR112, NR2C1, NR2C2, NR2E3, NR2F1, NR2F2, NR2F6, NR3C1, NR3C2, NR4A1, NR4A2, NR5A1, NR5A2, NRF1, NSD2, NUFIP1, NUP153, NUP98, NXF1, OGG1, ONECUT2, OR2M7, ORC1, ORC2, OSR2, OTX1, OTX2, OVOL2, OVOL3, p65, PADI2, PAF1, PALB2, PARP1, PATZ1, PAX2, PAX3, PAX5, PAX6, PAX7, PAX8, PBRM1, PBX1, PBX2, PBX3, PBX4, PBXIP1, PCBP1, PCBP2, PCF11, PCGF1, PCGF2, PCGF5, PCGF6, PDX1, PER1, PEX2, PGBD5, PGR, PHB2, PHF2, PHF20, PHF21A, PHF5A, PHF6, PHF8, PHOX2B, PIAS1, PIAS4, PITX1, PITX3, PKNOX1, PLAG1, PLRG1, PML, POU2F1, POU2F2, POU3F2, POUSF1, PPARA, PPARD, PPARG, PPARGC1A, PRDM1, PRDM10, PRDM11, PRDM12, PRDM14, PRDM2, PRDM4, PRDM5, PRDM6, PRDM9, PRKCQ, PRKDC, PRMT1, PRMT5, PROX1, PRPF4, PSMB5, PTBP1, PTEN, PTPRA, PTTG1, PYGO2, RAD21, RAD51, RAG1, RAG2, RARA, RARB, RARG, RAX2, RB1, RBAK, RBBP5, RBFOX2, RBL1, RBL2, RBM14, RBM15, RBM17, RBM22, RBM25, RBM34, RBM39, RBPJ, RCOR1, RCOR2, REL, RELA, RELB, REPIN1, RERE, REST, RFX1, RFX2, RFX3, RFX5, RFX7, RFXANK, RING1, RLF, RNF2, RNGTT, RORA, RPA1, RPA2, RUNX1, RUNX1T1, RUNX2, RUNX3, RUVBL1, RUVBL2, RXRA, RXRB, RYBP, SAFB, SAFB2, SALL1, SALL2, SALL3, SALL4, SAP130, SAP30, SATBI, SCML2, SCRT1, SCRT2, SETBP1, SETD1A, SETD7, SETDB1, SETX, SFMBT1, SFPQ, SIGMAR1, SIN3A, SIN3B, SIPA1, SIRT1, SIRT3, SIRT6, SIX1, SIX2, SIX4, SIX5, SK1, SKIL, SKP2, SLC30A9, SMAD1, SMAD2, SMAD3, SMAD4, SMAD5, SMARCA1, SMARCA2, SMARCA4, SMARCA5, SMARCBI, SMARCC1, SMARCC2, SMARCE1, SMC1A, SMC3, SMCHD1, SMN1, SNAII, SNAI2, SNAPC2, SNAPC4, SNIP1, SNRNP70, SOD1, SON, SOX10, SOX11, SOX13, SOX15, SOX17, SOX2, SOX3, SOX4, SOX5, SOX6, SOX9, SP1, SP140, SP2, SP3, SP4, SP5, SP7, SPDEF, SPII, SPIB, SQSTMI, SRC, SRCAP, SREBF1, SREBF2, SRF, SRPK1, SRPK2, SRSF3, SRSF4, SRSF7, SRSF9, SS18, SS18/SSX1, SSRP1, SSU72, STAG1, STAG2, STAT1, STAT2, STAT3, STAT4, STAT5A, STAT5B, STAT6, STN1, SUMO1, SUMO2, SUPT16H, SUPT20H, SUPT5H, SUZ12, SVIL, SYNCRIP, T, TAF1, TAF12, TAF15, TAF3, TAF7, TAF9B, TAL1, TARDBP, TAZ, TBLIXR1, TBP, TBPL1, TBX1, TBX2, TBX21, TBX3, TBX5, TCF12, TCF3, TCF4, TCF7, TCF7L1, TCF7L2, TCFL5, TCOF1, TDRD3, TEAD1, TEAD2, TEAD3, TEAD4, TERF1, TERF2, TERT, TET1, TET2, TET3, TFAM, TFAP2A, TFAP2C, TFAP4, TFCP2, TFDP1, TFDP2, TFE3, TFEB, TGIF1, TGIF2, THAP1, THAP11, THRA, THRAP3, THRB, TLE3, TLX1, TOP1, TOP2B, top2beta, TOX4, TP53, TP53BP1, TP63, TP73, TRIM22, TRIM24, TRIM25, TRIM28, TRIP13, TRPS1, TSC22D4, TSHZ1, TUBG1, TWIST1, U2AF1, U2AF2, UBE2I, UBN1, UBP1, UBTF, USF1, USF2, USP7, VDR, VEZF1, WDHD1, WDR5, WIZ, WRN, WRNIP1, WT1, XBP1, XRCC3, XRCC4, XRCC5, XRN2, YAP1, YBX1, YBX3, YYI, YY2, ZA, ZBED1, ZBED4, ZBED5, ZBTBI, ZBTB10, ZBTB11, ZBTB12, ZBTB14, ZBTB16, ZBTB17, ZBTB18, ZBTB2, ZBTB20, ZBTB21, ZBTB24, ZBTB25, ZBTB26, ZBTB33, ZBTB39, ZBTB40, ZBTB42, ZBTB44, ZBTB48, ZBTB49, ZBTB5, ZBTB6, ZBTB7A, ZBTB7B, ZBTB8A, ZC3H11A, ZC3H8, ZEBI, ZEB2, ZFAT, ZFHX2, ZFHX3, ZFP1, ZFP28, ZFP3, ZFP36, ZFP36L1, ZFP37, ZFP42, ZFP64, ZFP69B, ZFP82, ZFP91, ZFX, ZGPAT, ZHX1, ZHX2, ZIC2, ZIK1, ZIM3, ZKSCAN1, ZKSCAN8, ZMIZI, ZMYM2, ZMYM3, ZMYND11, ZMYND8, ZNF10, ZNF101, ZNF112, ZNF114, ZNF121, ZNF132, ZNF133, ZNF134, ZNF136, ZNF138, ZNF140, ZNF143, ZNF146, ZNF148, ZNF155, ZNF157, ZNF16, ZNF165, ZNF169, ZNF174, ZNF175, ZNF18, ZNF184, ZNF189, ZNF19, ZNF195, ZNF197, ZNF2, ZNF202, ZNF205, ZNF207, ZNF211, ZNF213, ZNF214, ZNF217, ZNF22, ZNF221, ZNF223, ZNF224, ZNF23, ZNF236, ZNF239, ZNF24, ZNF248, ZNF250, ZNF257, ZNF26, ZNF260, ZNF263, ZNF264, ZNF266, ZNF274, ZNF280A, ZNF280C, ZNF280D, ZNF281, ZNF282, ZNF292, ZNF3, ZNF30, ZNF300, ZNF302, ZNF311, ZNF316, ZNF317, ZNF318, ZNF320, ZNF322, ZNF324, ZNF329, ZNF331, ZNF335, ZNF33A, ZNF34, ZNF341, ZNF35, ZNF350, ZNF354A, ZNF354B, ZNF354C, ZNF362, ZNF366, ZNF37A, ZNF382, ZNF384, ZNF391, ZNF394, ZNF395, ZNF398, ZNF404, ZNF407, ZNF41, ZNF410, ZNF416, ZNF418, ZNF419, ZNF423, ZNF426, ZNF433, ZNF436, ZNF444, ZNF445, ZNF449, ZNF454, ZNF467, ZNF473, ZNF48, ZNF486, ZNF488, ZNF490, ZNF491, ZNF493, ZNF501, ZNF502, ZNF507, ZNF510, ZNF511, ZNF512, ZNF512B, ZNF513, ZNF514, ZNF518A, ZNF521, ZNF524, ZNF528, ZNF529, ZNF530, ZNF532, ZNF544, ZNF547, ZNF548, ZNF549, ZNF554, ZNF555, ZNF558, ZNF560, ZNF561, ZNF563, ZNF571, ZNF574, ZNF577, ZNF579, ZNF580, ZNF581, ZNF582, ZNF584, ZNF585B, ZNF586, ZNF589, ZNF592, ZNF595, ZNF596, ZNF597, ZNF600, ZNF610, ZNF614, ZNF618, ZNF621, ZNF622, ZNF623, ZNF624, ZNF626, ZNF629, ZNF639, ZNF644, ZNF645, ZNF652, ZNF654, ZNF658, ZNF660, ZNF662, ZNF664, ZNF667, ZNF669, ZNF670, ZNF677, ZNF680, ZNF687, ZNF692, ZNF697, ZNF7, ZNF701, ZNF704, ZNF707, ZNF708, ZNF711, ZNF740, ZNF747, ZNF75A, ZNF76, ZNF766, ZNF768, ZNF770, ZNF774, ZNF776, ZNF777, ZNF778, ZNF781, ZNF784, ZNF785, ZNF791, ZNF792, ZNF8, ZNF816, ZNF83, ZNF830, ZNF837, ZNF84, ZNF843, ZNF85, ZNF92, ZSCAN16, ZSCAN18, ZSCAN2, ZSCAN21, ZSCAN22, ZSCAN23, ZSCAN26, ZSCAN29, ZSCAN30, ZSCAN31, ZSCAN4, ZSCANSA, ZSCAN5B, ZSCAN5C, ZSCANSDP, ZSCAN9, ZXDB, ZXDC, and ZZZ3. Within each transcription factor, the top 5,000 sites with the greatest experimental support were chosen. In this section “experimental support” is defined as the number of experiments which detected the site. cfDNA fragments were then overlapped with each set of TFBS to yield a set of reads which overlapped with each set of TFBS. Shannon entropy was calculated using the counts of the read lengths for the reads overlapping each set of TFBS for each sample to yield one feature per transcription factor. This feature table was used for model training and validation as in the ESE model (FIGS. 26A-26H).

Validation AUC in the UW cohort using genes in the UW panel was 0.97 for bladder cancer, 0.99 for breast cancer, 0.93 for lung cancer, 0.99 for NEPC, and 0.97 for prostate cancer. Validation AUC in the UW cohort using common genes between the UW panel and the Tempus xF panel was 0.89 for bladder cancer, 0.80 for breast cancer, 0.80 for lung cancer, 0.92 for NEPC, and 0.88 for prostate cancer. Validation AUC in the UW cohort using common genes between the UW panel and the Guardant 360 CDx panel was 0.86 for bladder cancer, 0.89 for breast cancer, 0.86 for lung cancer, 0.89 for NEPC, and 0.94 for prostate cancer. Validation AUC in the UW cohort using common genes between the UW panel and the Foundation One Liquid CDx panel was 0.90 for bladder cancer, 0.89 for breast cancer, 0.87 for lung cancer, 0.96 for NEPC, and 0.93 for prostate cancer.

Validation AUC in the GRAIL cohort using genes in the GRAIL panel was 0.91 for breast cancer, 0.82 for lung cancer, 0.94 for prostate cancer, and 1.00 for cancer vs normal. Validation AUC in the GRAIL cohort using common genes between the GRAIL panel and the Tempus xF panel was 0.76 for breast cancer, 0.89 for lung cancer, 0.95 for prostate cancer, and 1.00 for cancer vs normal. Validation AUC in the GRAIL cohort using common genes between the GRAIL panel and the Guardant 360 CDx panel was 0.79 for breast cancer, 0.84 for lung cancer, 0.92 for prostate cancer, and 1.00 for cancer vs normal. Validation AUC in the GRAIL cohort using common genes between the GRAIL panel and the Foundation One Liquid CDx panel was 0.83 for breast cancer, 0.83 for lung cancer, 0.92 for prostate cancer, and 1.00 for cancer vs normal.

### Shannon Entropy of Reads Overlapping Areas of Open Chromatin Defined by Atac-Seq

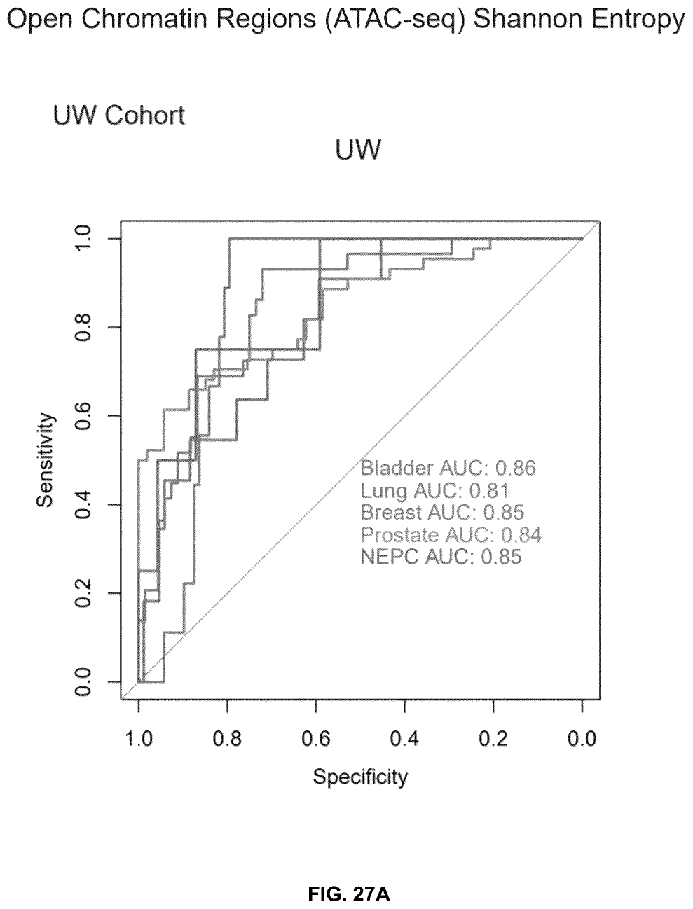

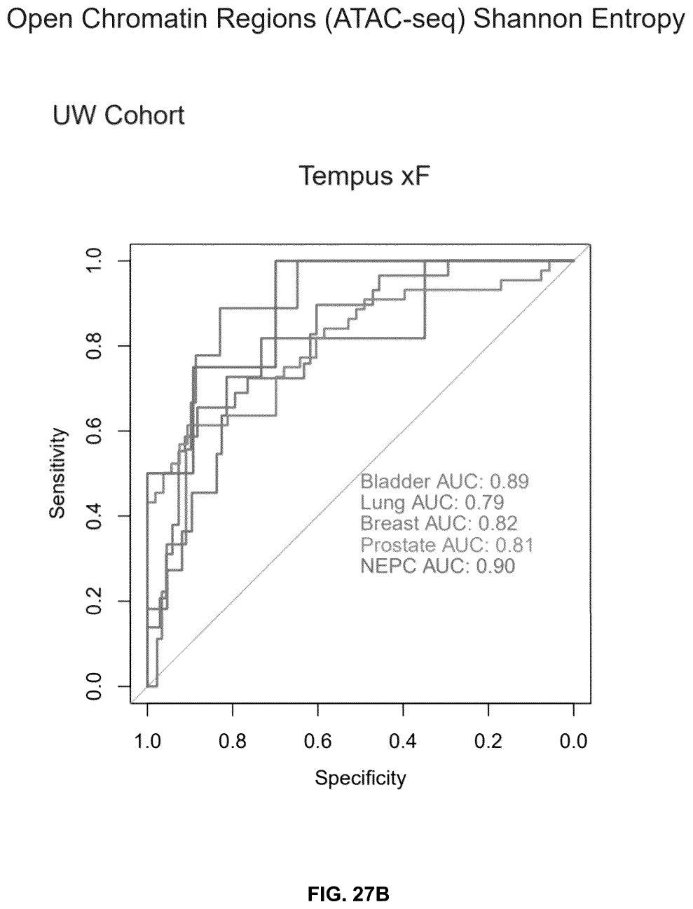

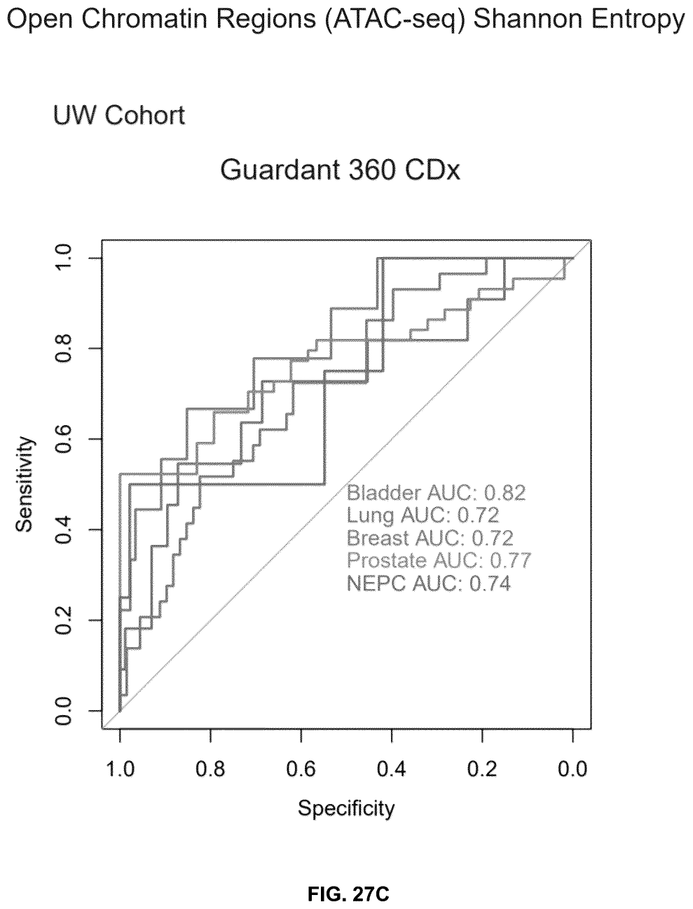

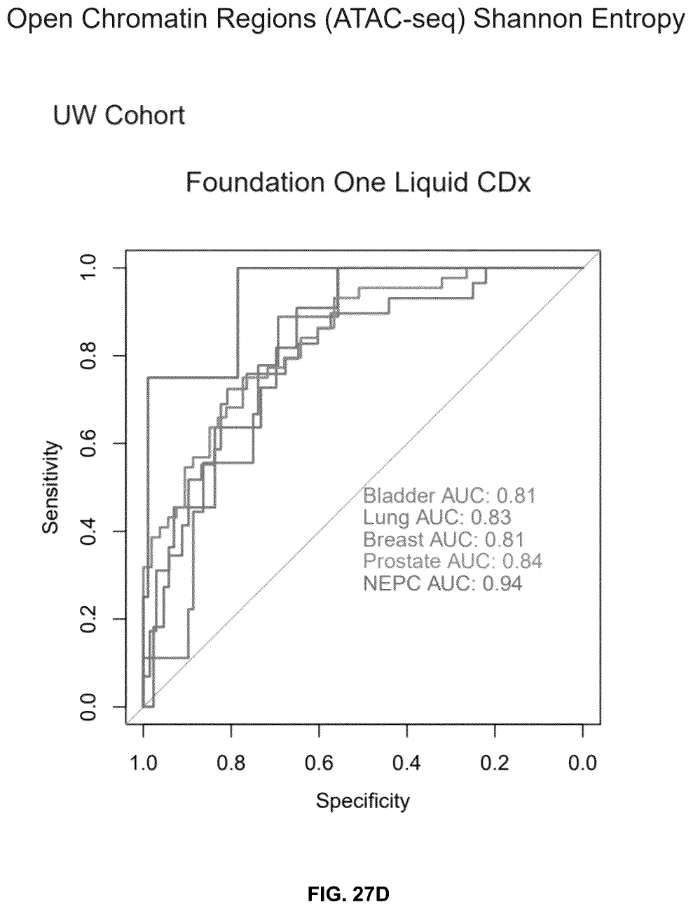

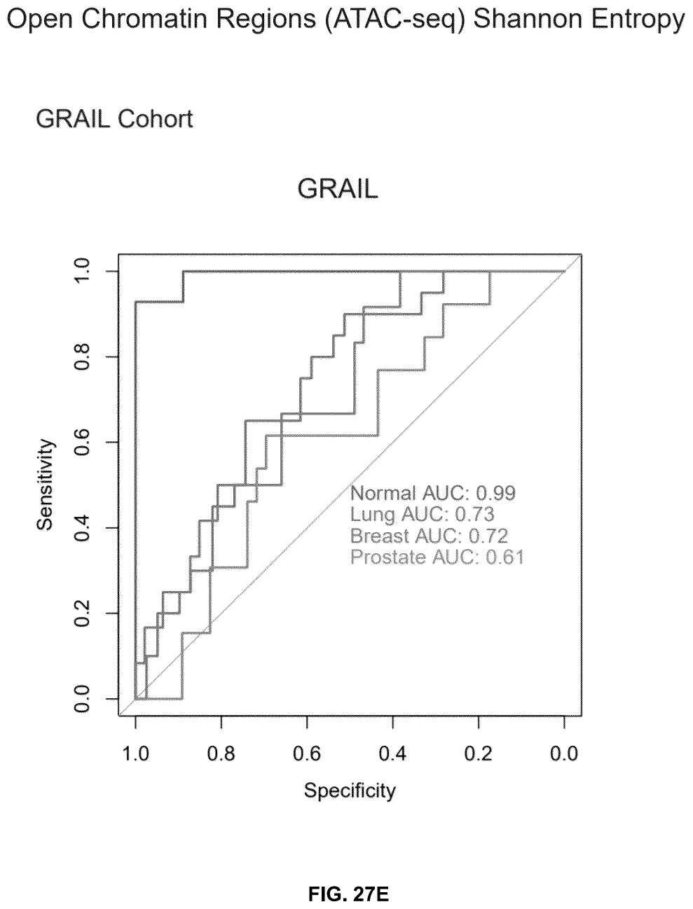

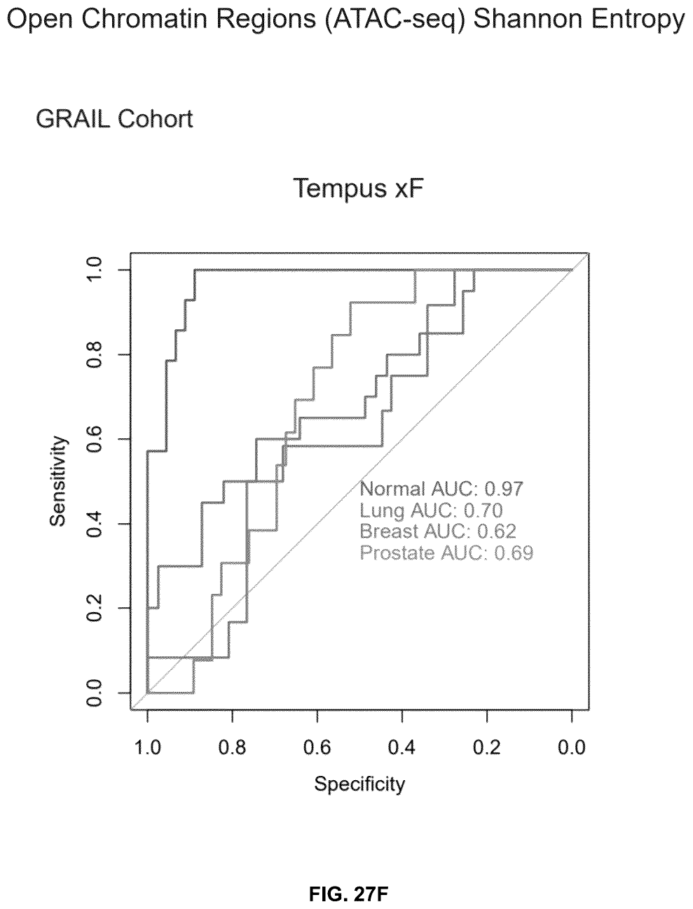

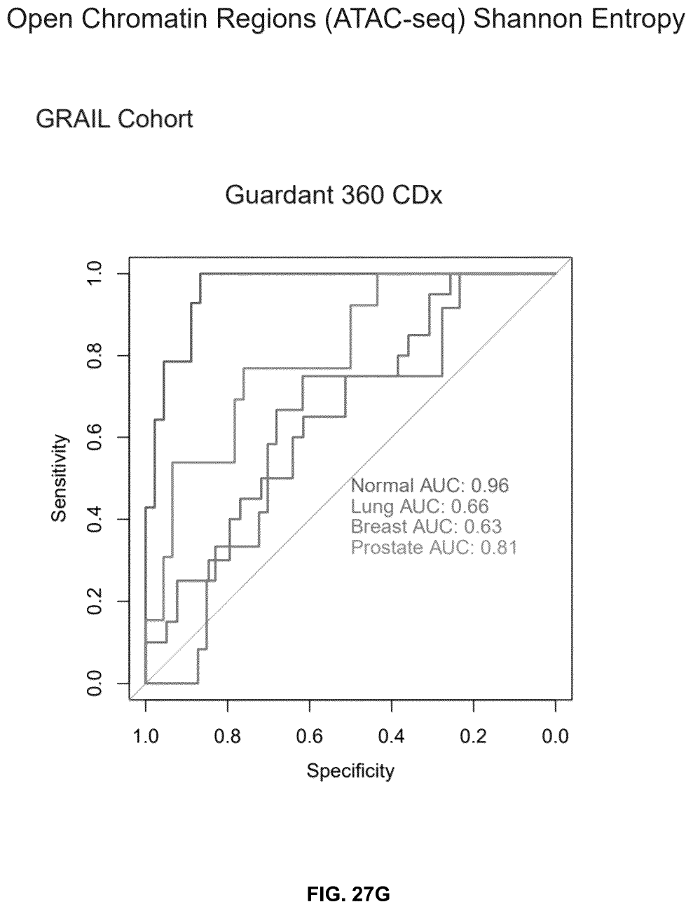

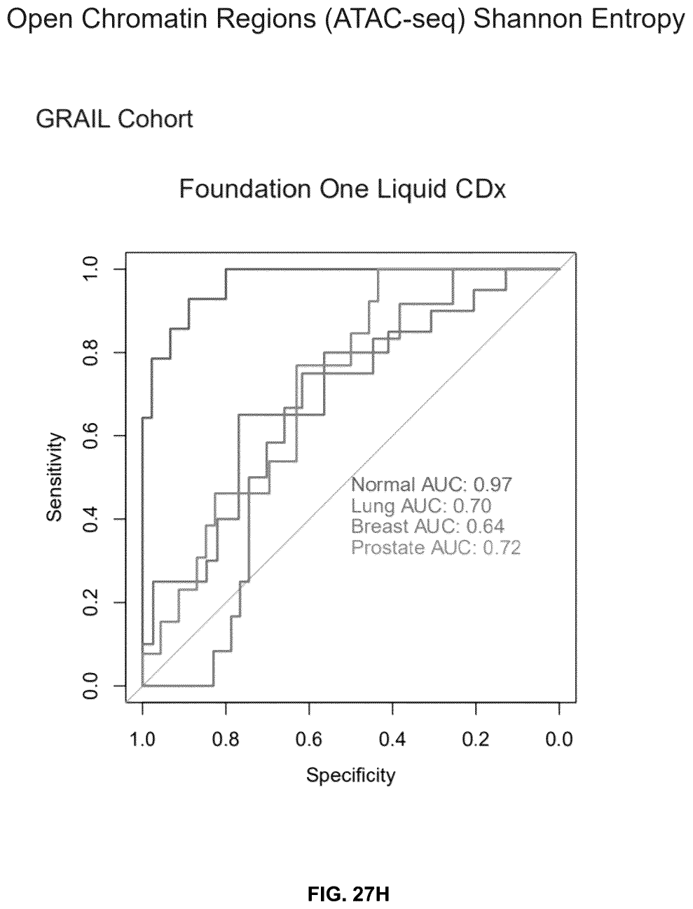

Consensus genomic regions of open chromatin as defined by the Assay for Transposase-Accessible Chromatin with sequencing (ATAC-Seq) was downloaded from The Cancer Genome Atlas (TCGA) for 23 different cancer types. The cancer types analyzed were Adrenocortical carcinoma (ACC), Bladder Urothelial Carcinoma (BLCA), Breast invasive carcinoma (BRCA), squamous cell carcinoma and endocervical adenocarcinoma (CESC), Cervical Cholangiocarcinoma (CHOL), Colon adenocarcinoma (COAD), Esophageal carcinoma (ESCA), Glioblastoma multiforme (GBM), Head and Neck squamous cell carcinoma (HNSC), Kidney renal clear cell carcinoma (KIRC), Kidney renal papillary cell carcinoma (KIRP), Low Grade Glioma (LGG), Liver hepatocellular carcinoma (LIHC), Lung adenocarcinoma (LUAD), Lung squamous cell carcinoma (LUSC), Mesothelioma (MESO), Pheochromocytoma and Paraganglioma (PCPG), Prostate adenocarcinoma (PRAD), Skin Cutaneous Melanoma (SKCM), Stomach adenocarcinoma (STAD), Testicular Germ Cell Tumors (TGCT), Thyroid carcinoma (THCA), and Uterine Corpus Endometrial Carcinoma (UCEC). cfDNA fragments were then overlapped with each set of open chromatin regions to yield a set of reads which overlapped with each set of open chromatin regions for each cancer type. Shannon entropy was calculated using the counts of the read lengths for reads overlapping each set of open chromatin regions for each sample to yield one feature per cancer type listed above. This feature table was used for model training and validation as in the E1SE model (FIGS. 27A-27H).

Validation AUC in the UW cohort using genes in the UW panel was 0.86 for bladder cancer, 0.85 for breast cancer, 0.81 for lung cancer, 0.85 for NEPC, and 0.84 for prostate cancer. Validation AUC in the UW cohort using common genes between the UW panel and the Tempus xF panel was 0.89 for bladder cancer, 0.82 for breast cancer, 0.79 for lung cancer, 0.90 for NEPC, and 0.81 for prostate cancer. Validation AUC in the UW cohort using common genes between the UW panel and the Guardant 360 CDx panel was 0.82 for bladder cancer, 0.72 for breast cancer, 0.72 for lung cancer, 0.74 for NEPC, and 0.74 for prostate cancer. Validation AUC in the UW cohort using common genes between the UW panel and the Foundation One Liquid CDx panel was 0.81 for bladder cancer, 0.81 for breast cancer, 0.83 for lung cancer, 0.94 for NEPC, and 0.84 for prostate cancer.

Validation AUC in the GRAIL cohort using genes in the GRAIL panel was 0.72 for breast cancer, 0.73 for lung cancer, 0.61 for prostate cancer, and 0.99 for cancer vs normal. Validation AUC in the GRAIL cohort using common genes between the GRAIL panel and the Tempus xF panel was 0.62 for breast cancer, 0.70 for lung cancer, 0.69 for prostate cancer, and 0.97 for cancer vs normal. Validation AUC in the GRAIL cohort using common genes between the GRAIL panel and the Guardant 360 CDx panel was 0.63 for breast cancer, 0.66 for lung cancer, 0.81 for prostate cancer, and 0.96 for cancer vs normal. Validation AUC in the GRAIL cohort using common genes between the GRAIL panel and the Foundation One Liquid CDx panel was 0.64 for breast cancer, 0.70 for lung cancer, 0.72 for prostate cancer, and 0.97 for cancer vs normal.

### Testing Model Performance Across Features

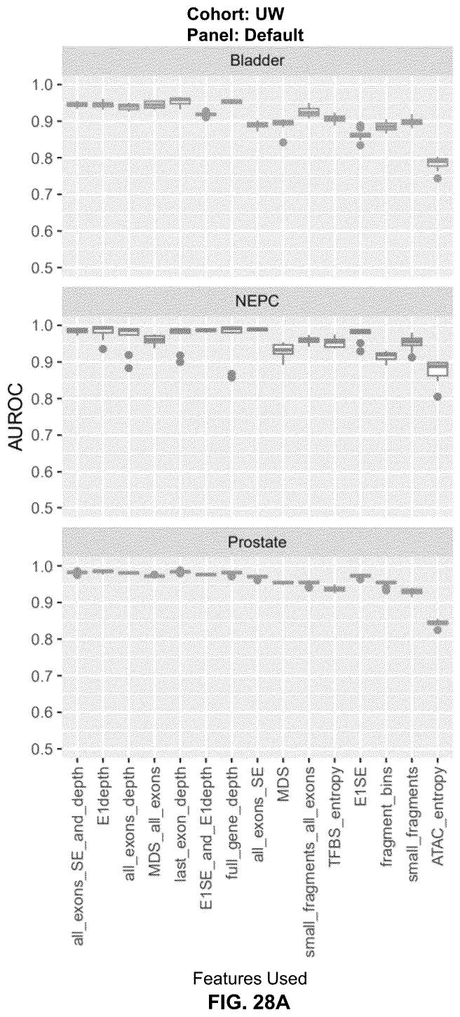

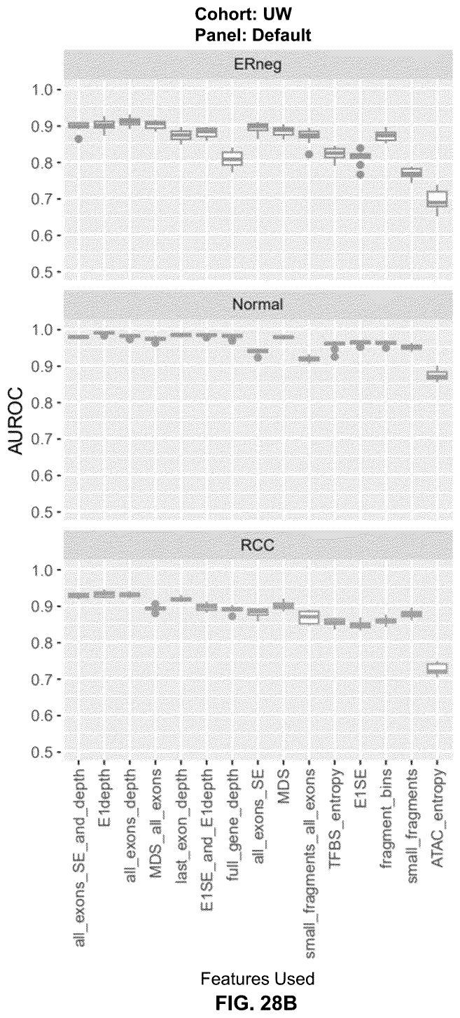

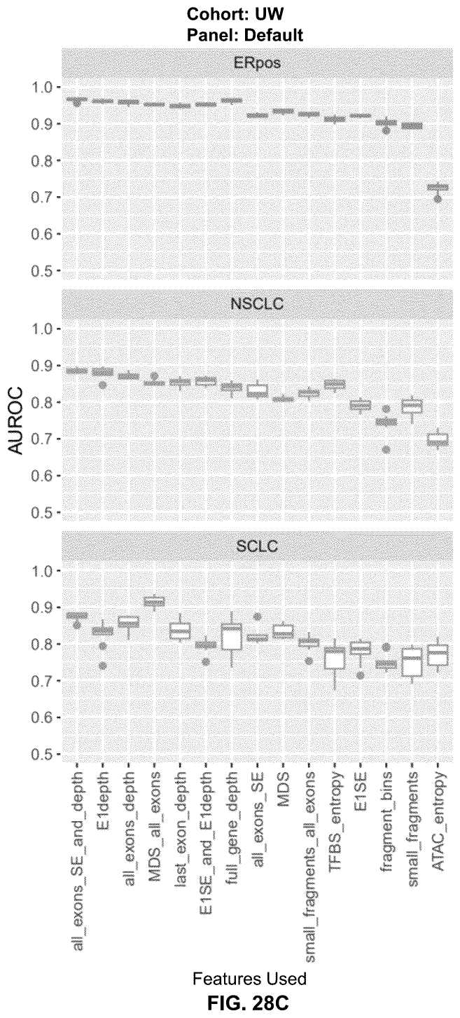

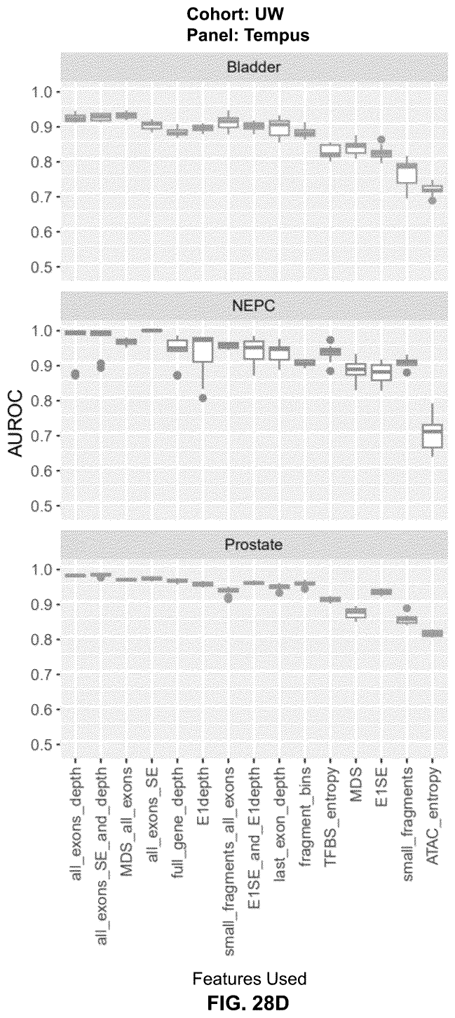

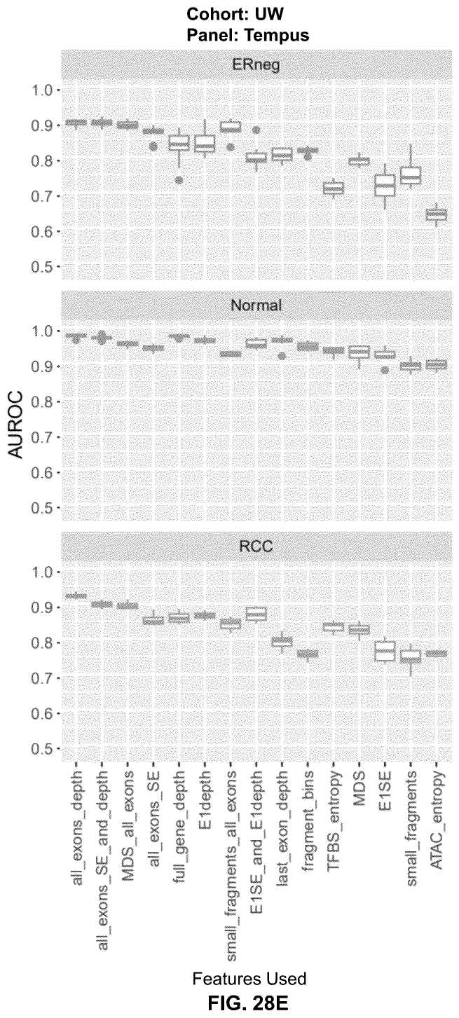

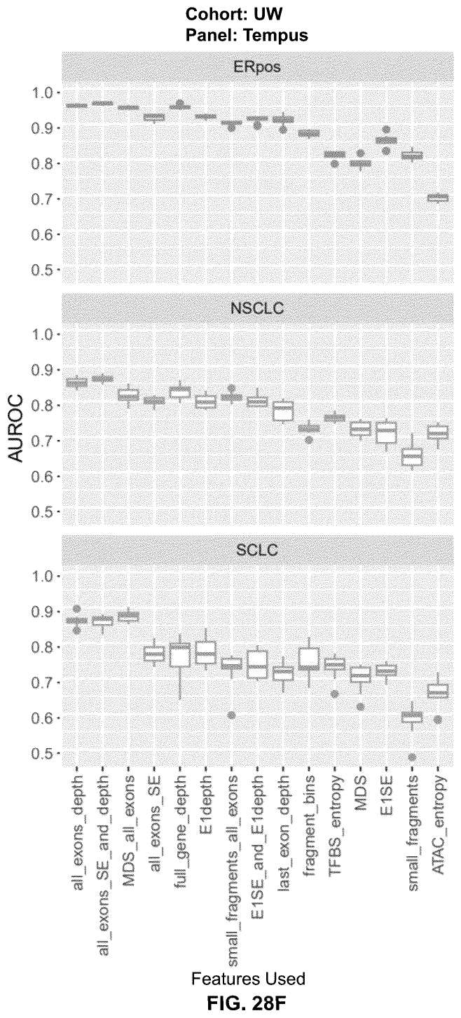

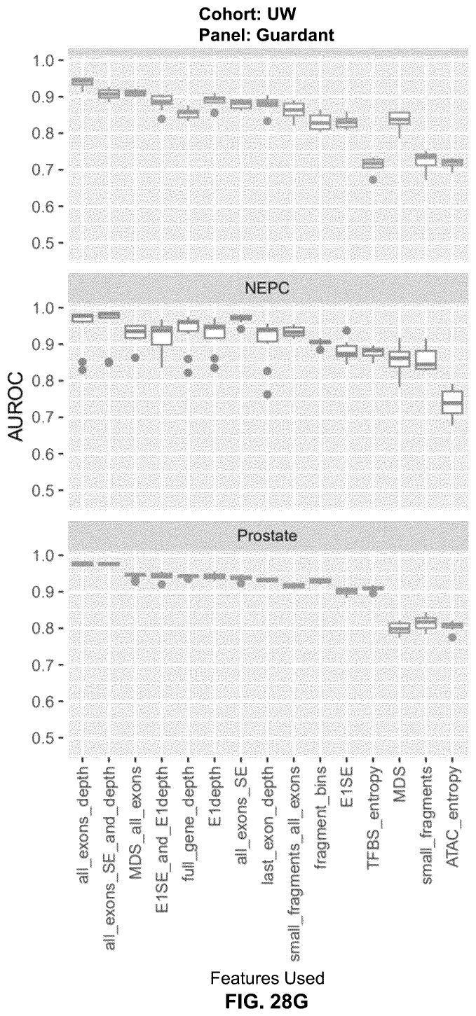

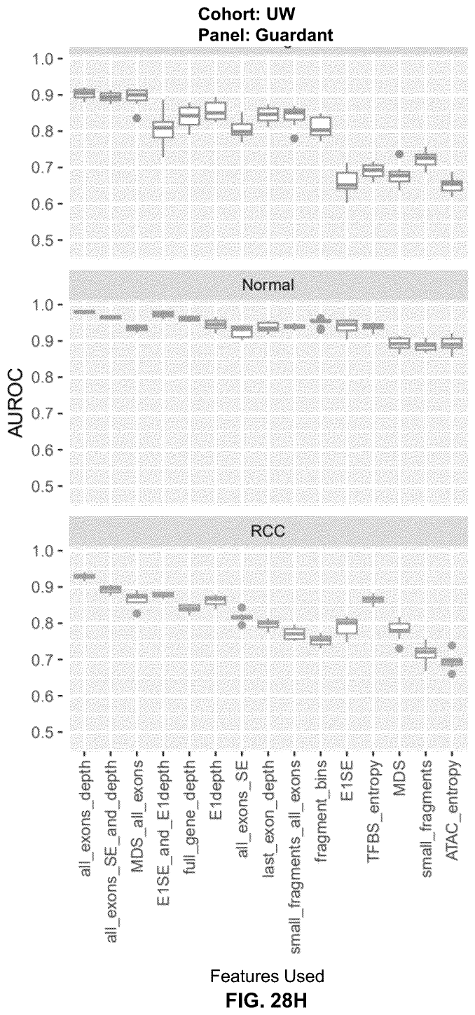

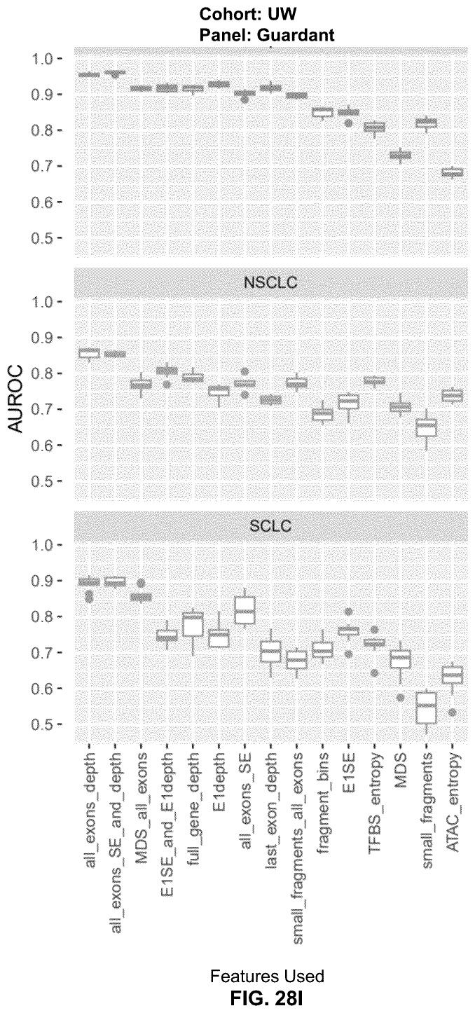

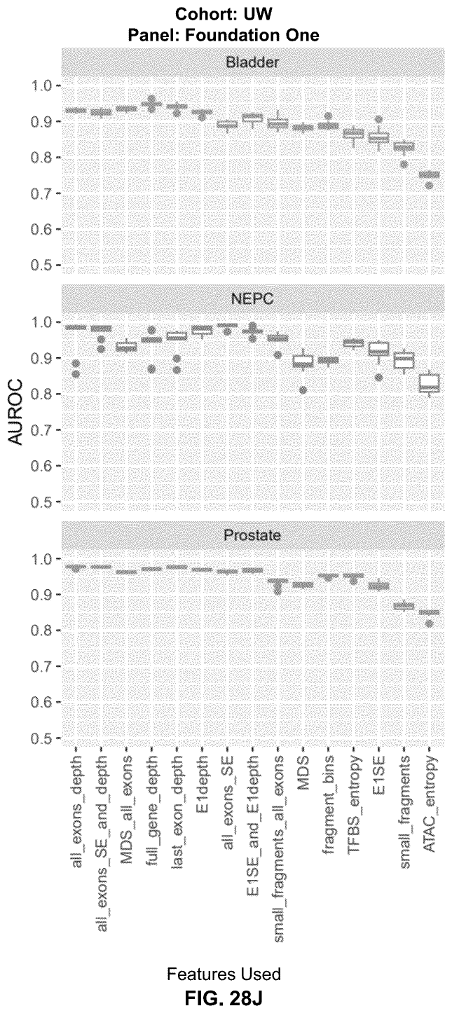

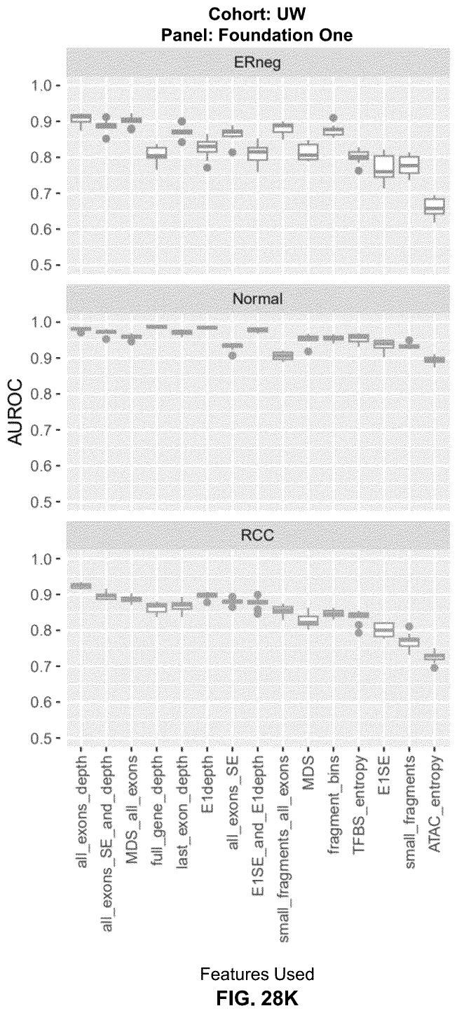

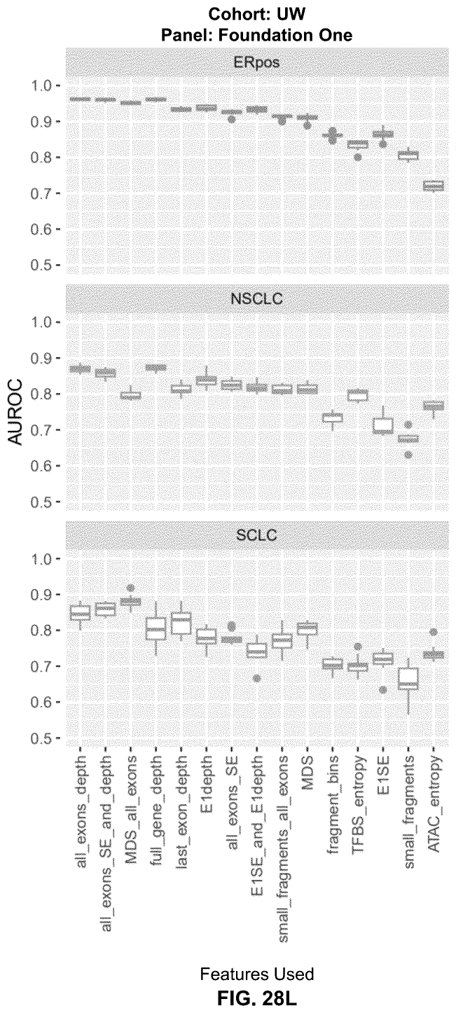

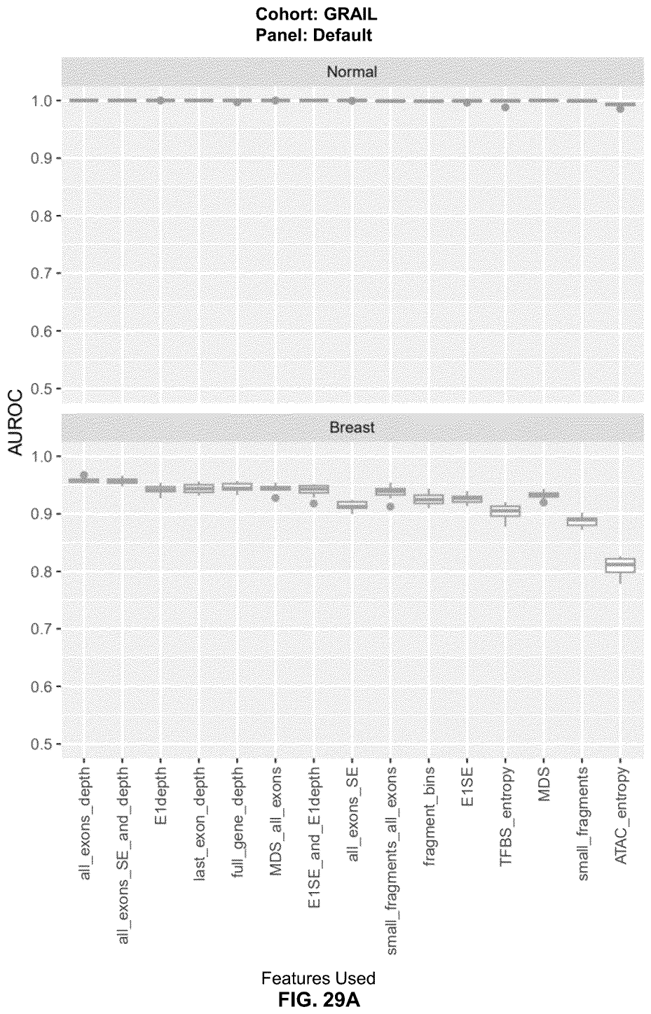

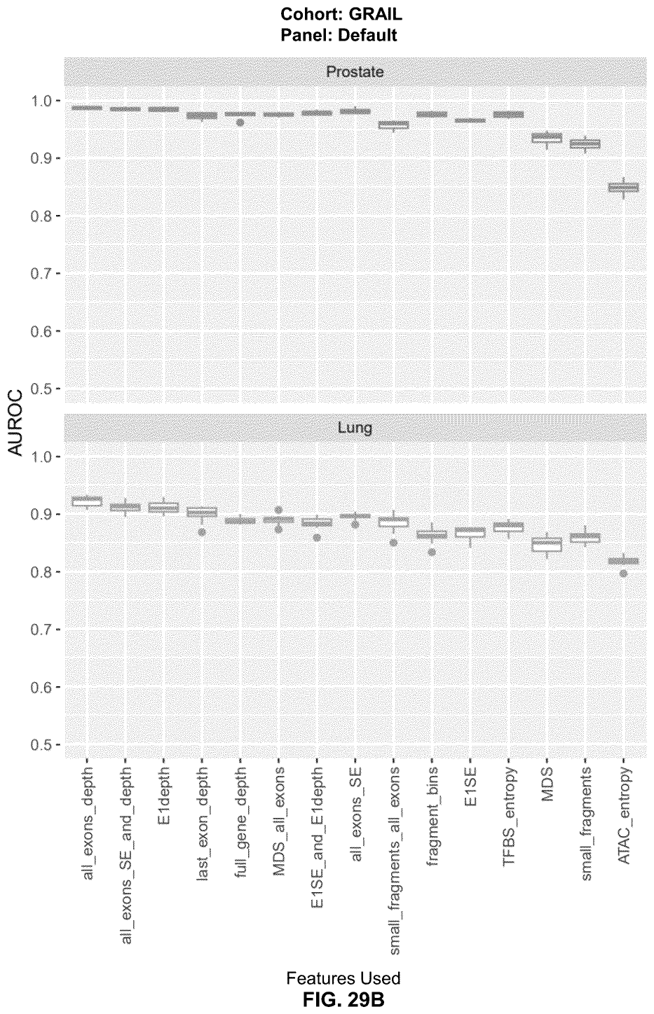

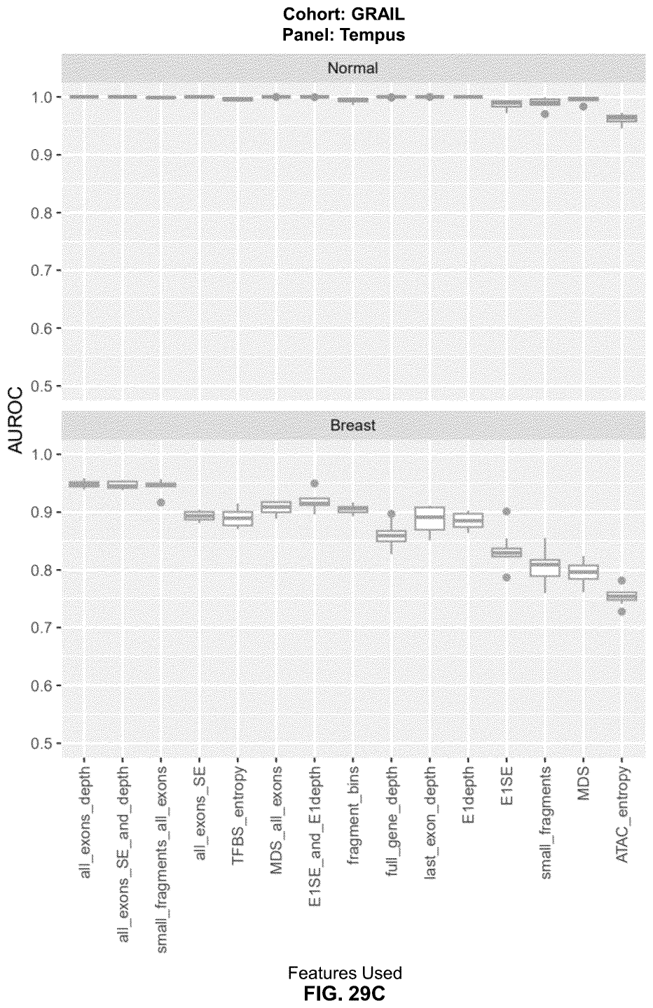

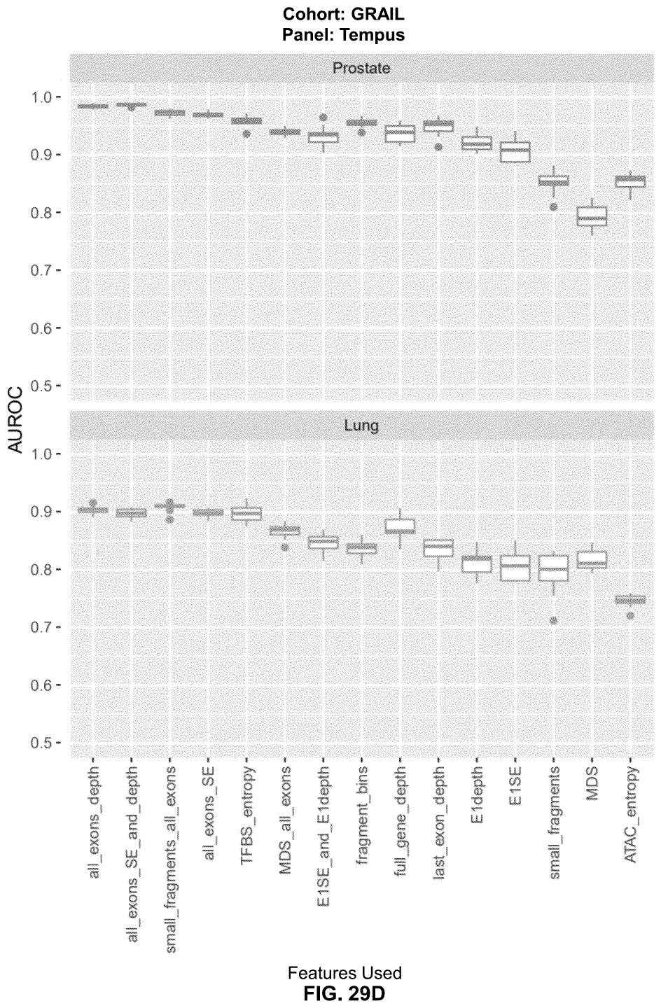

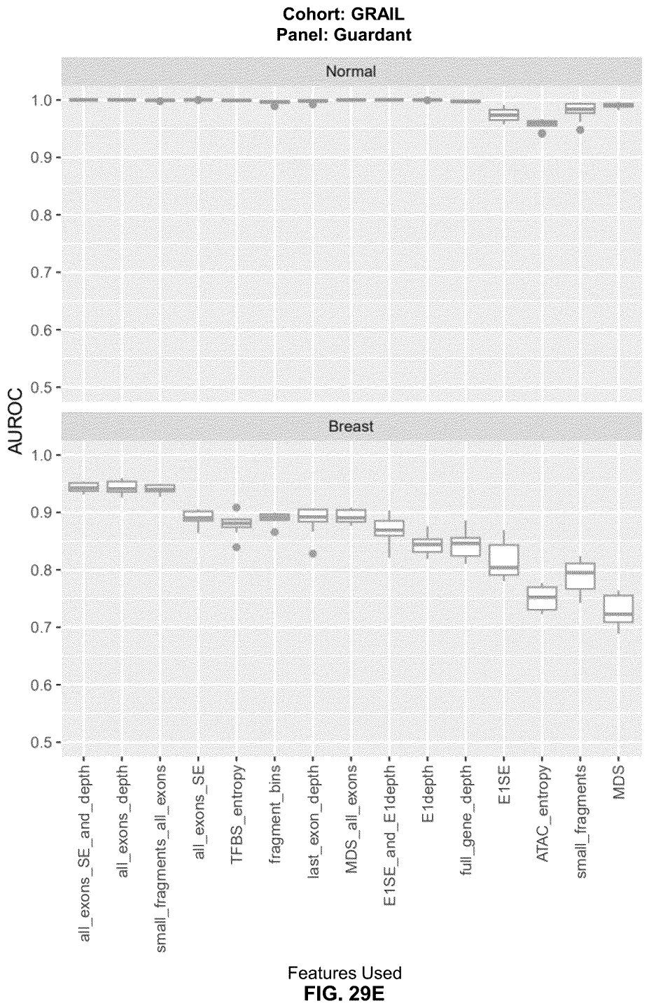

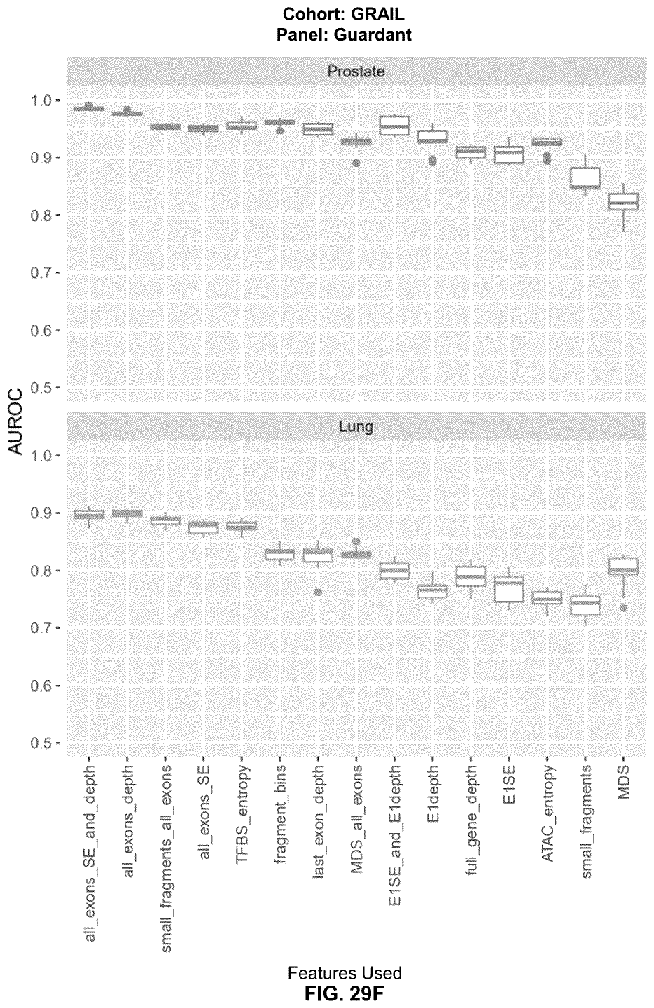

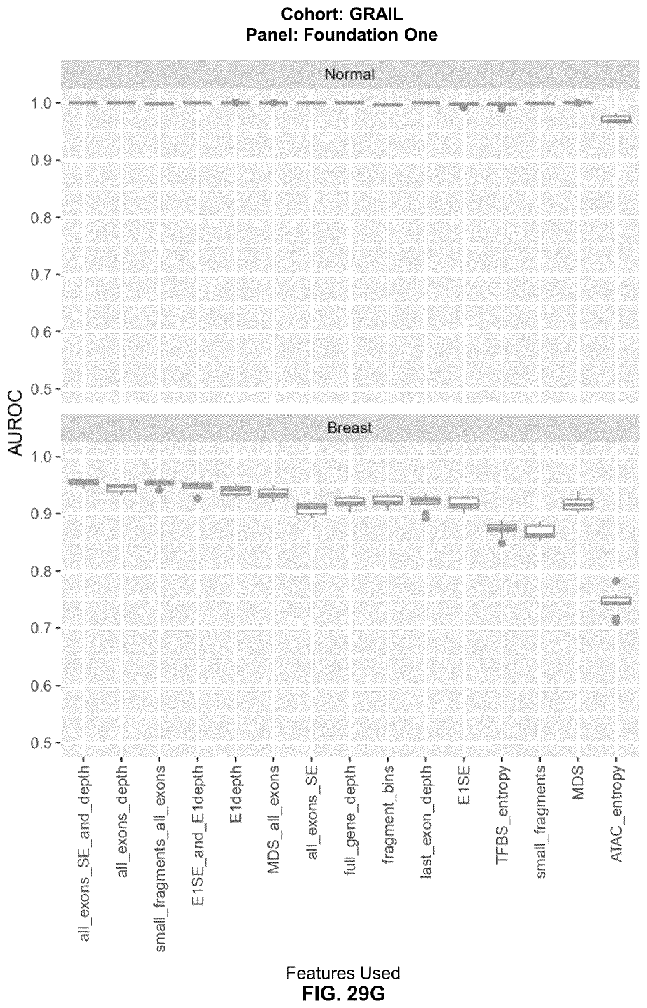

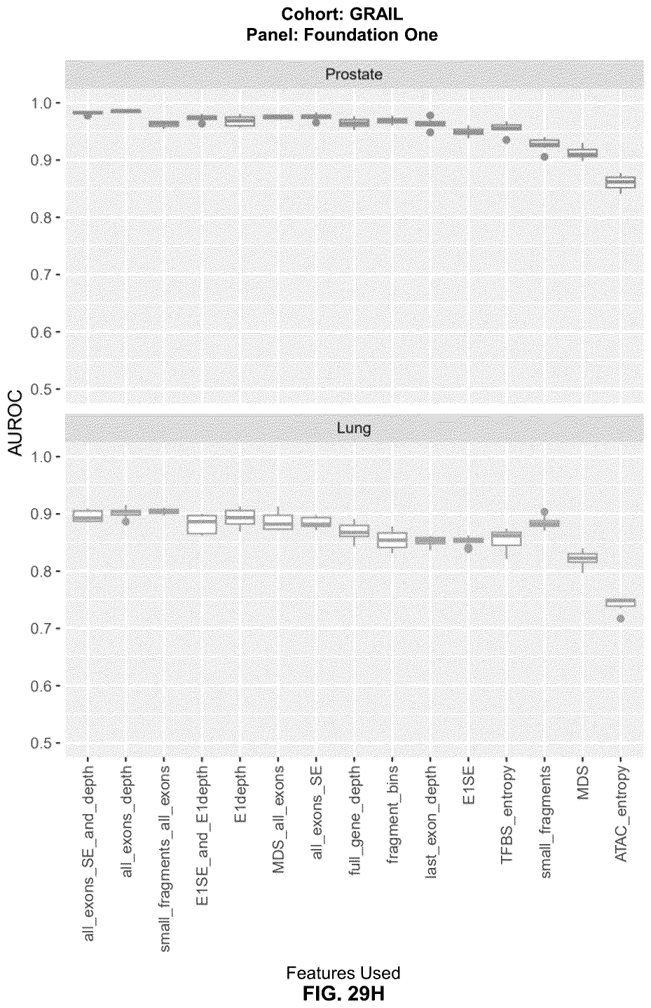

Using all metrics calculated (E1SE, exon 1 depth, E1SE and exon 1 depth, all exons Shannon entropy (SE), all exons depth, combining all exons depth and Shannon entropy, full gene depth, exon 1 MDS, all exon MDS, exon 1 small fragment proportions, all exons small fragment proportions, fragment size bins, TFBS entropy, and ATAC region entropy) samples in the UW cohort (FIGS. 28A-28L) and the GRAIL cohort (FIGS. 29A-29H) were analyzed for model performance to predict cancer type. The UW cohort comprises bladder cancer, breast cancer, lung cancer, renal cell cancer (RCC), prostate adenocarcinoma (Prostate), and neuroendocrine prostate cancer (NEPC). UW breast cancer samples were further split into ER positive (ERpos) and ER negative (ERneg) samples. UW lung cancer samples were further split into small cell lung cancer (SCLC) and non-small cell lung cancer (NSCLC). Ten replicates of the 10-fold cross-validation model were performed and AUROC was calculated to assess performance. The best performing metric in the UW cohort with the UW panel was “all exons Shannon entropy and depth” with mean AUROCs ranging from 0.872-0.985. Across all feature types, the mean AUROC ranged from 0.692-0.989 (FIG. 28A). The best performing metric in the UW cohort with the Tempus xF panel was “all exons depth” with mean AUROCs ranging from 0.852-0.975. Across all feature types, the mean AUROC ranged from 0.584-0.991 (FIG. 28B). The best performing metric in the UW cohort with the Guardant 360 CDx panel was “all exons depth” with mean AUROCs ranging from 0.856-0.978. Across all feature types, the mean AUROC ranged from 0.546-0.978 (FIG. 28C). The best performing metric in the UW cohort with the Foundation One Liquid CDx panel was “all exons depth” with mean AUROCs ranging from 0.844-0.980. Across all feature types, the mean AUROC ranged from 0.657-0.989 (FIG. 28D). The best performing metric in the GRAIL cohort with the GRAIL panel was “all exons depth” with mean AUROCs ranging from 0.922-1.000. Across all feature types, the mean AUROC ranged from 0.807-1.000 (FIG. 29A). The best performing metric in the GRAIL cohort with the Tempus XF panel was “all exons depth” with mean AUROCs ranging from 0.904-1.000. Across all feature types, the mean AUROC ranged from 0.745-1.000 (FIG. 29B). The best performing metric in the GRAIL cohort with the Guardant 360 CDx panel was “all exons SE and depth” with mean AUROCs ranging from 0.894-1.000. Across all feature types, the mean AUROC ranged from 0.728-1.000 (FIG. 29C). The best performing metric in the GRAIL cohort with the Foundation One Liquid CDx panel was “all exons SE and depth” with mean AUROCs ranging from 0.895-1.000. Across all feature types, the mean AUROC ranged from 0.743-1.000 (FIG. 29D).

### Testing Model Performance Across Features by Ctdna Fraction

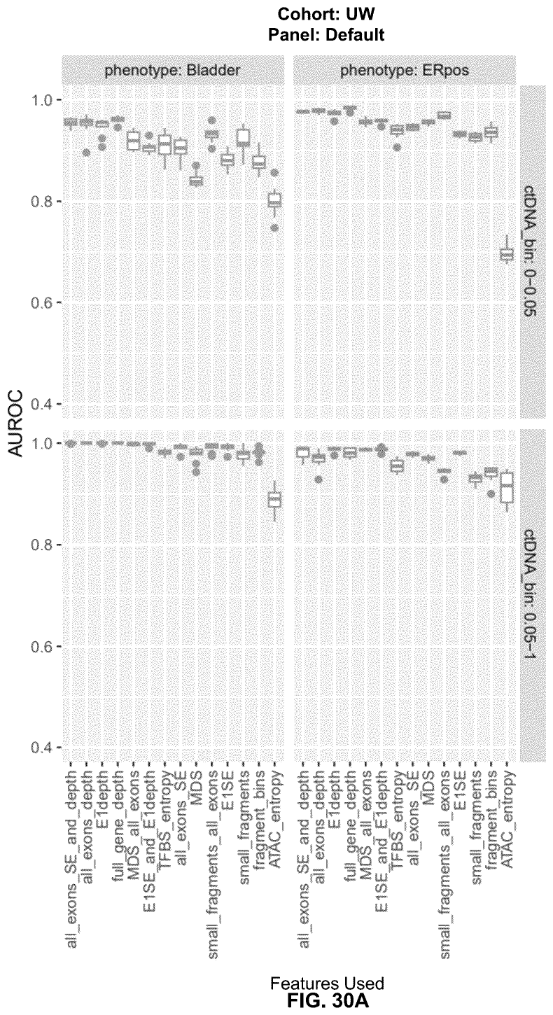

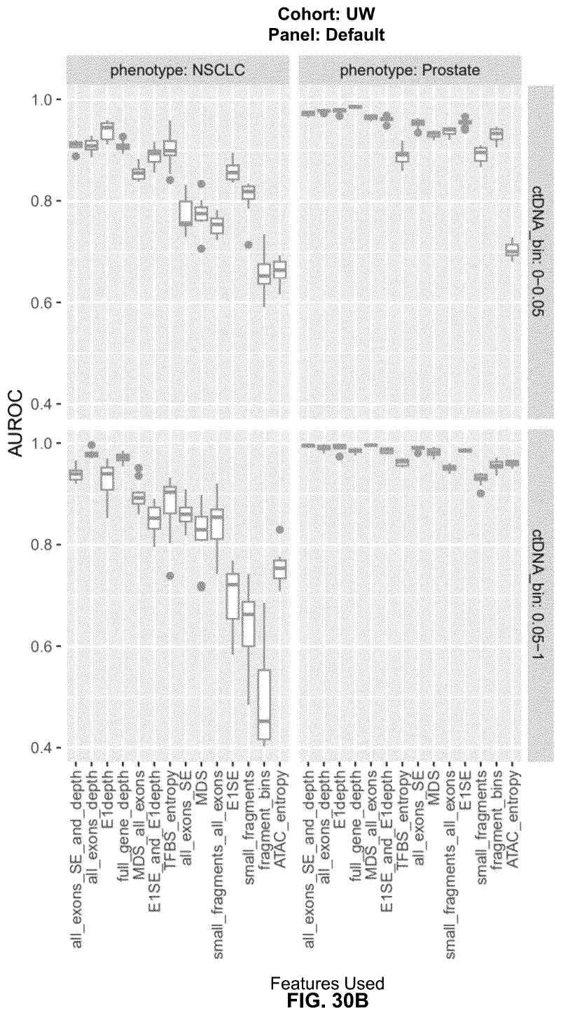

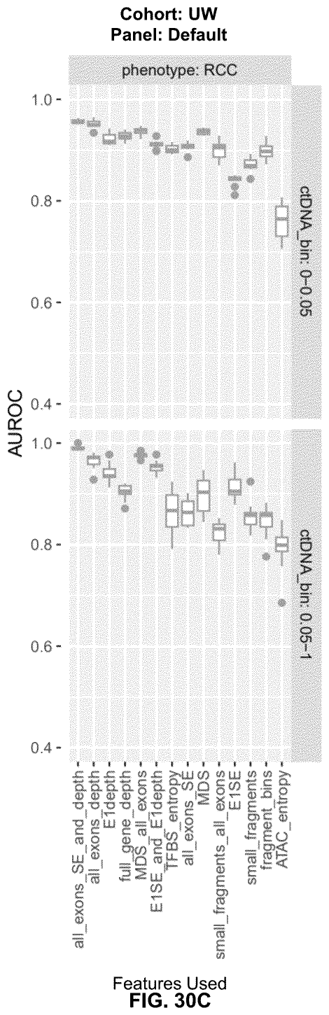

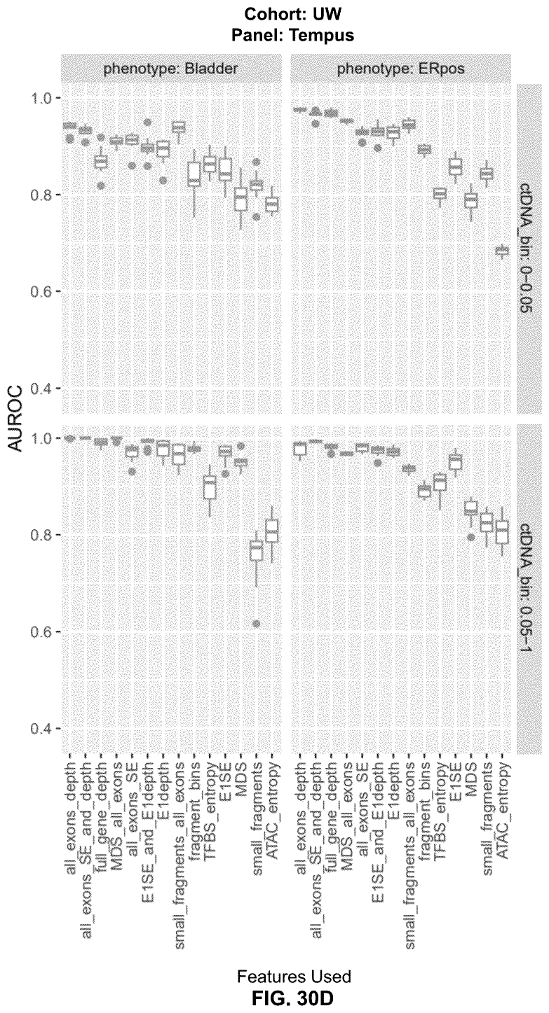

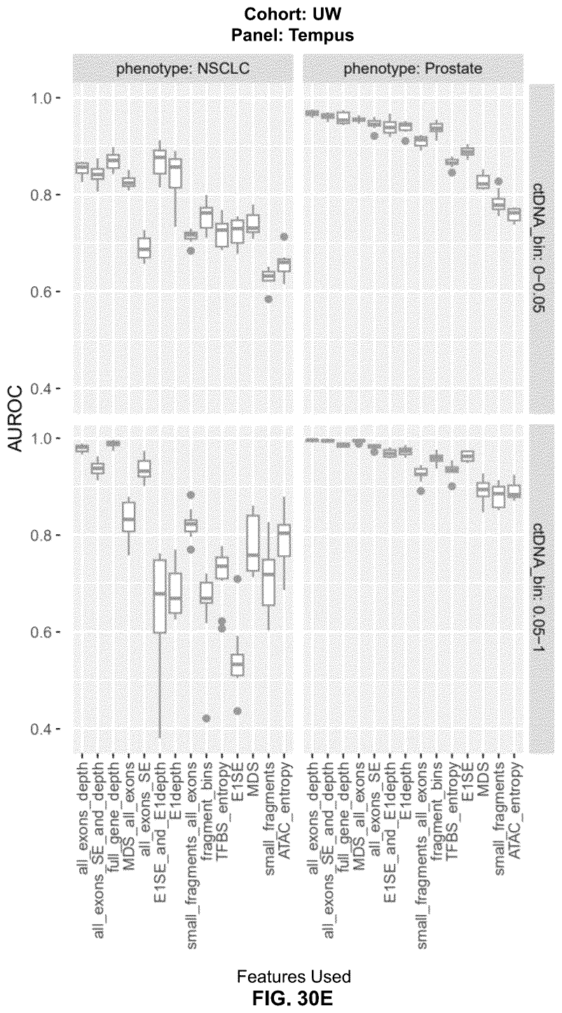

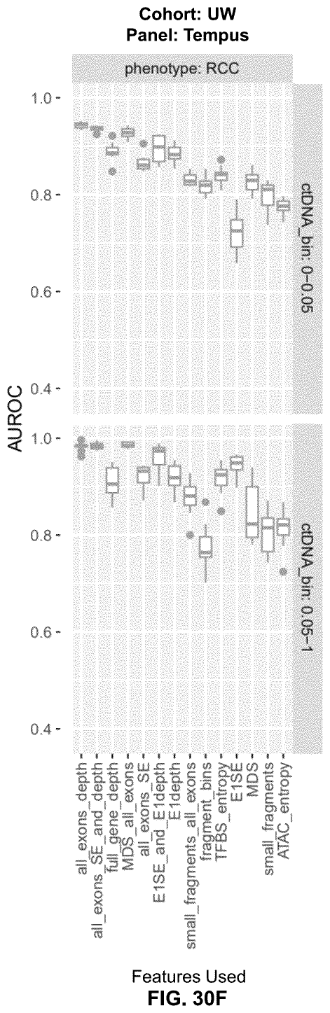

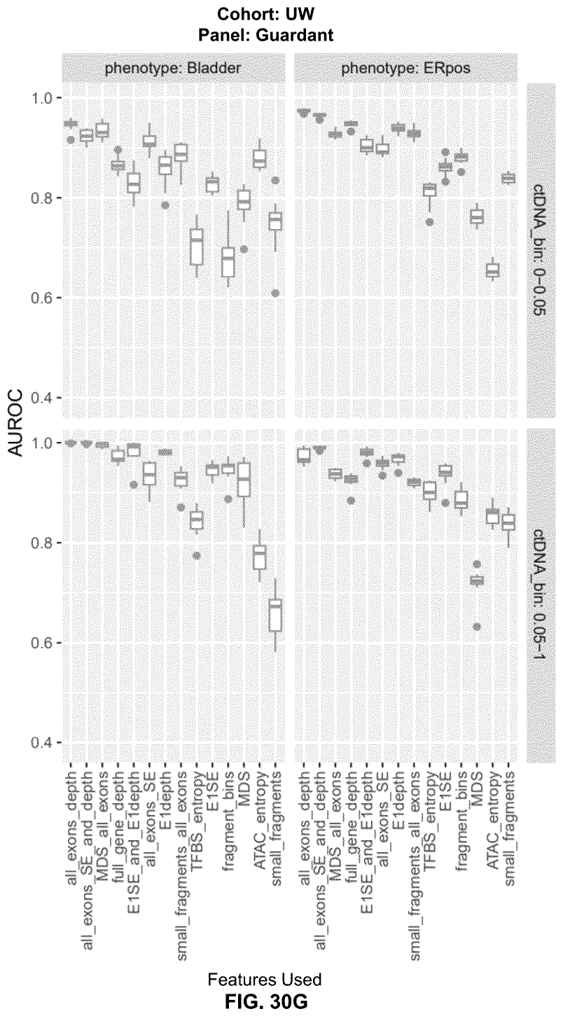

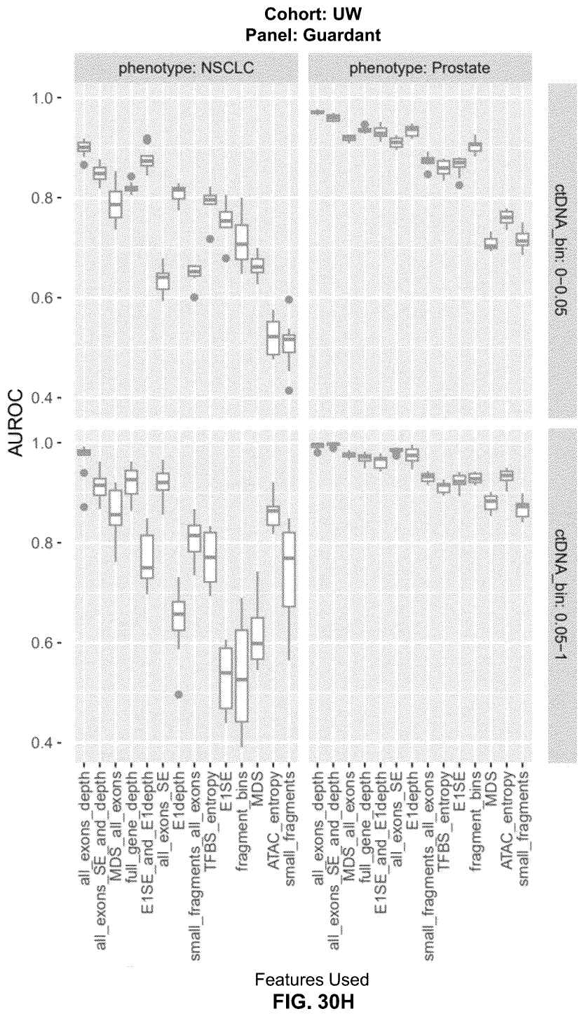

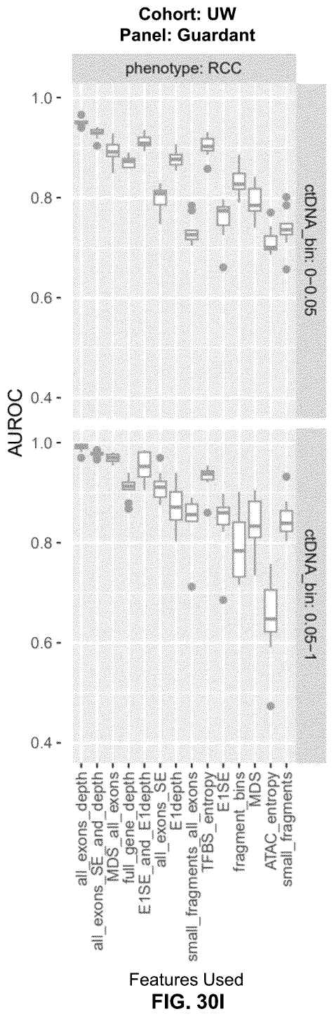

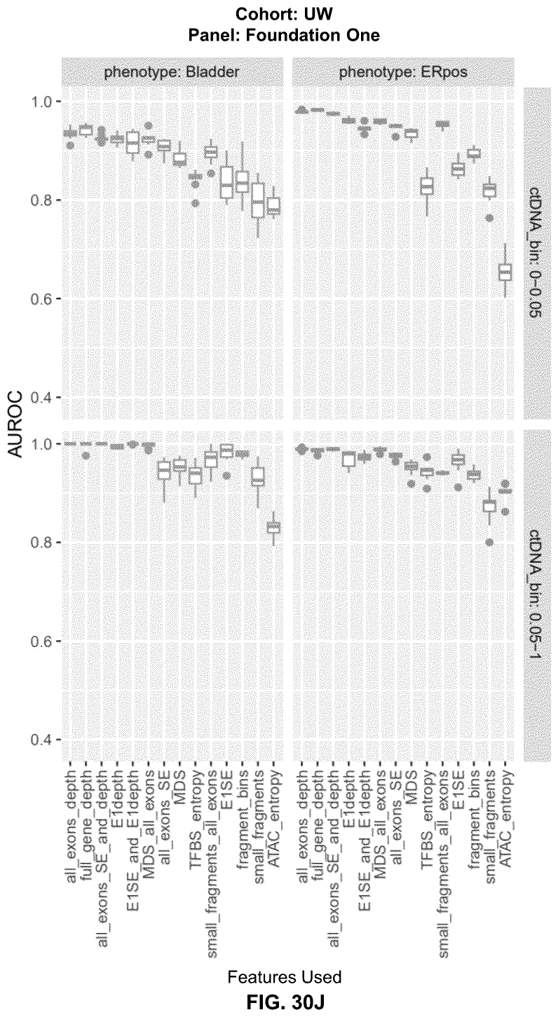

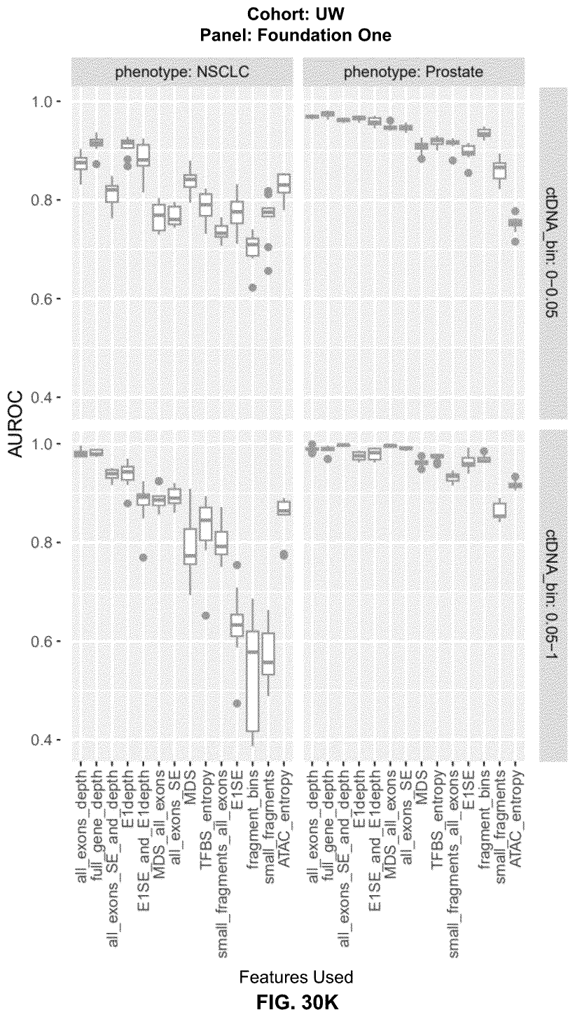

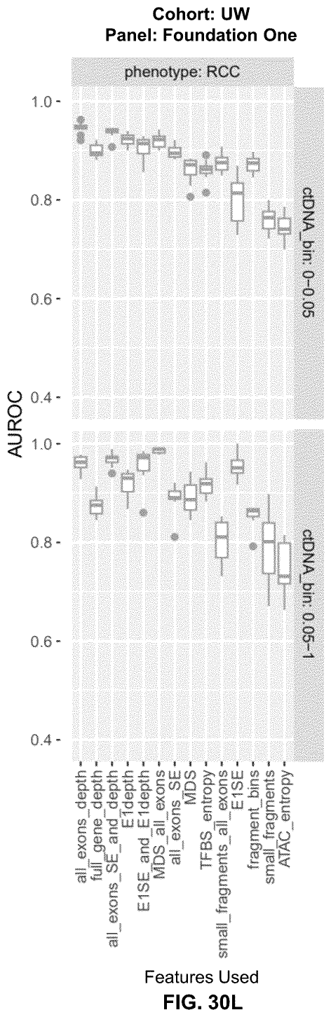

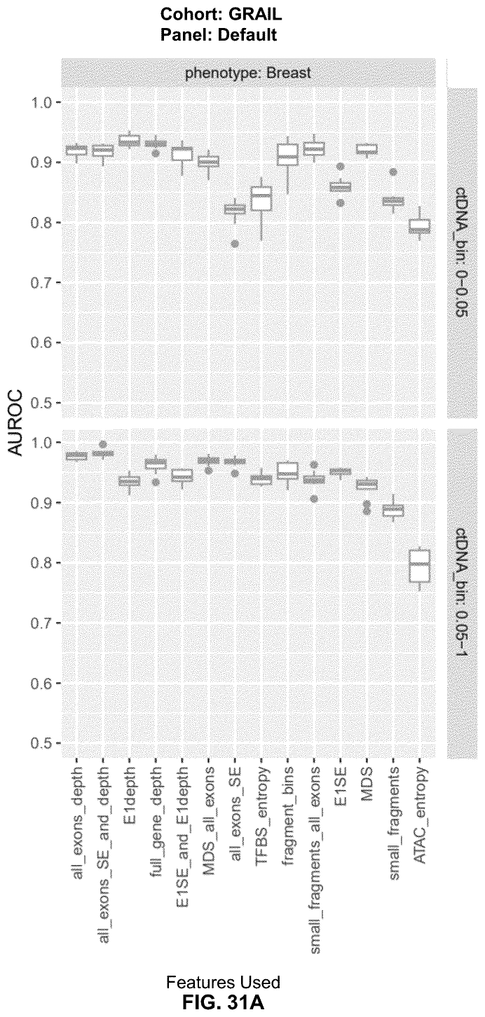

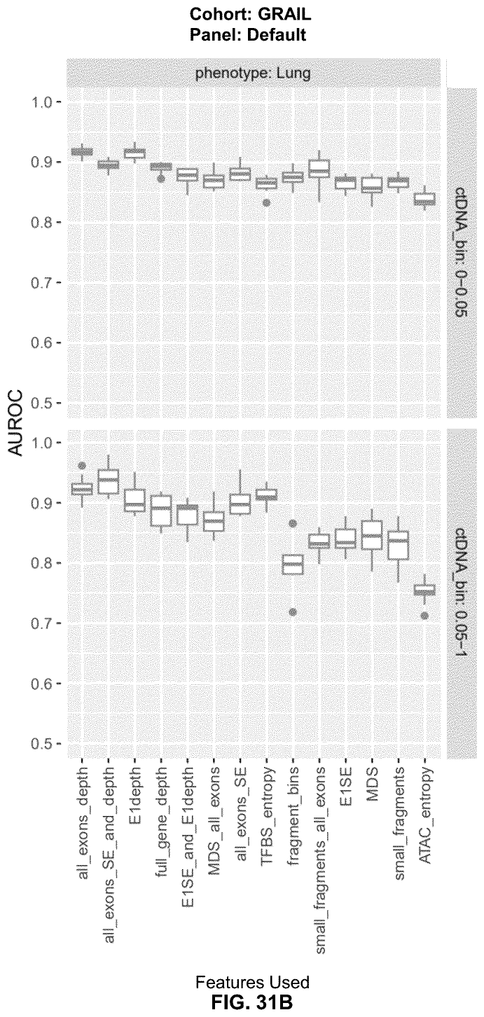

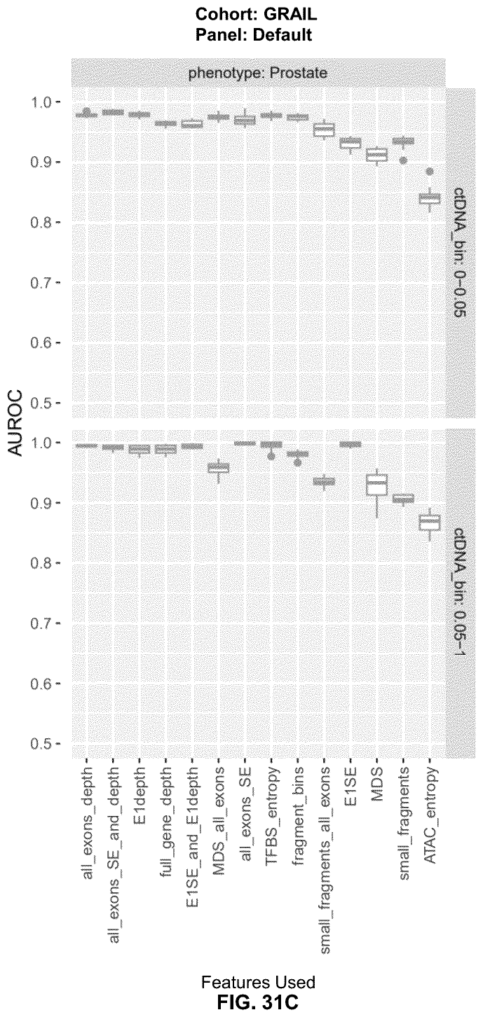

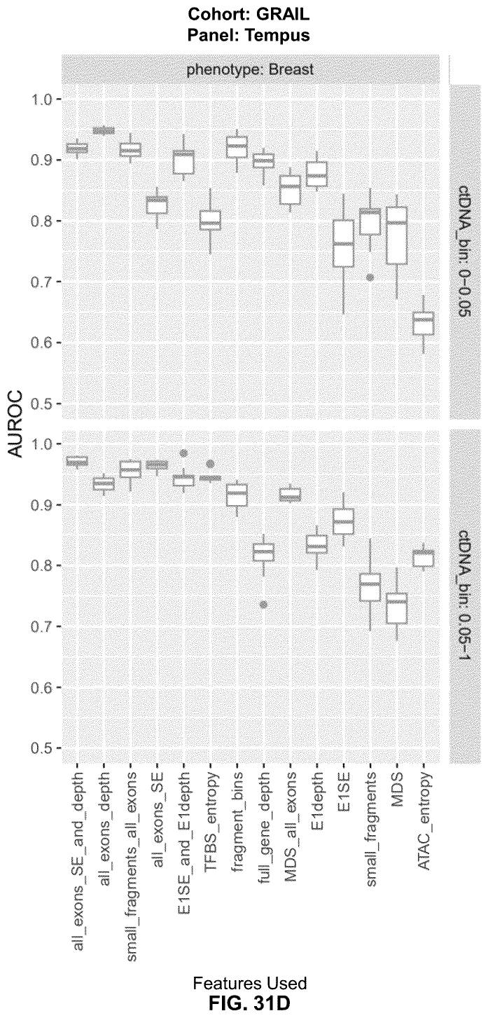

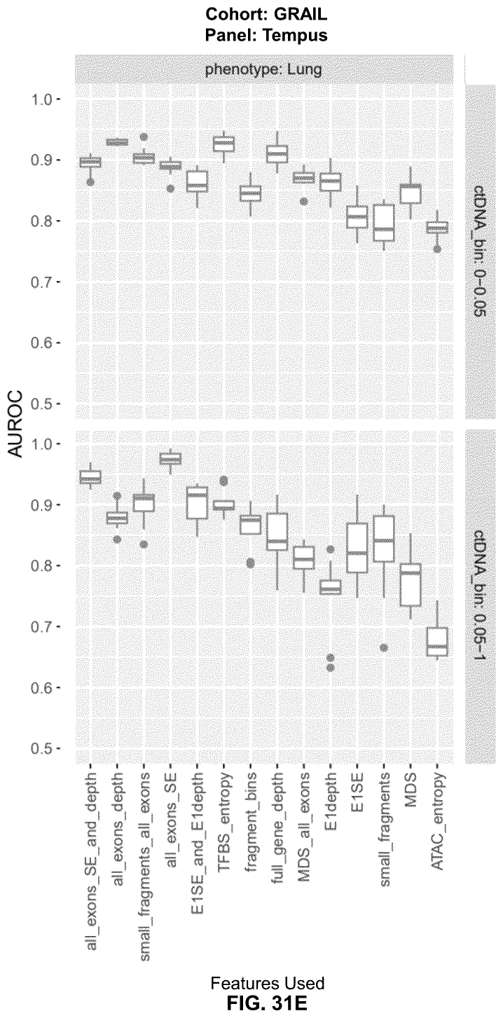

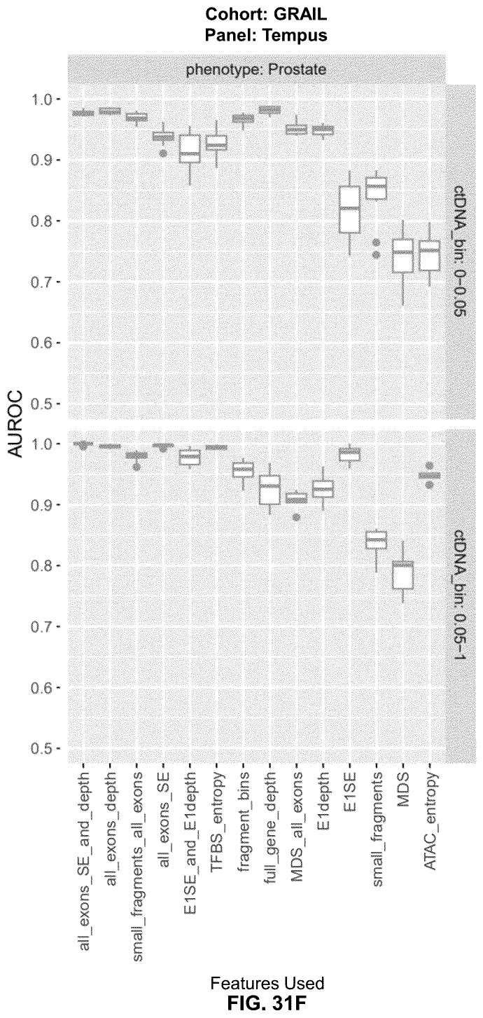

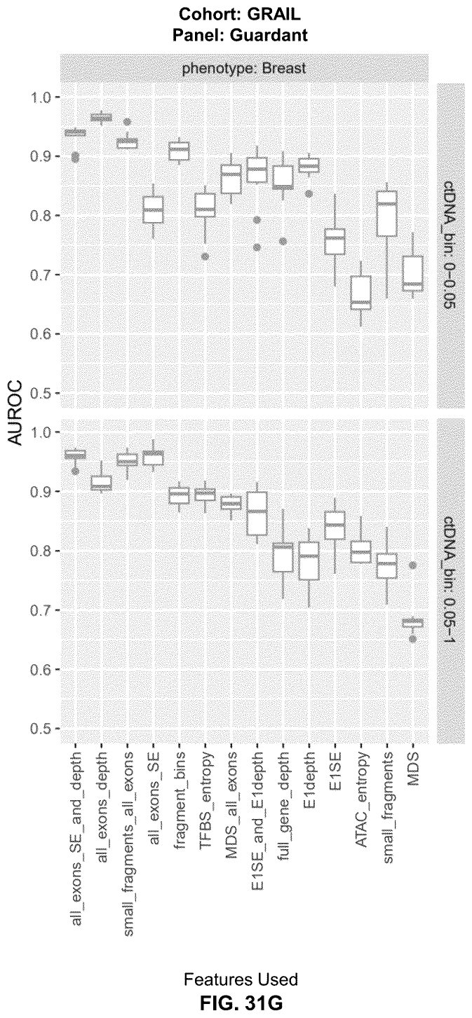

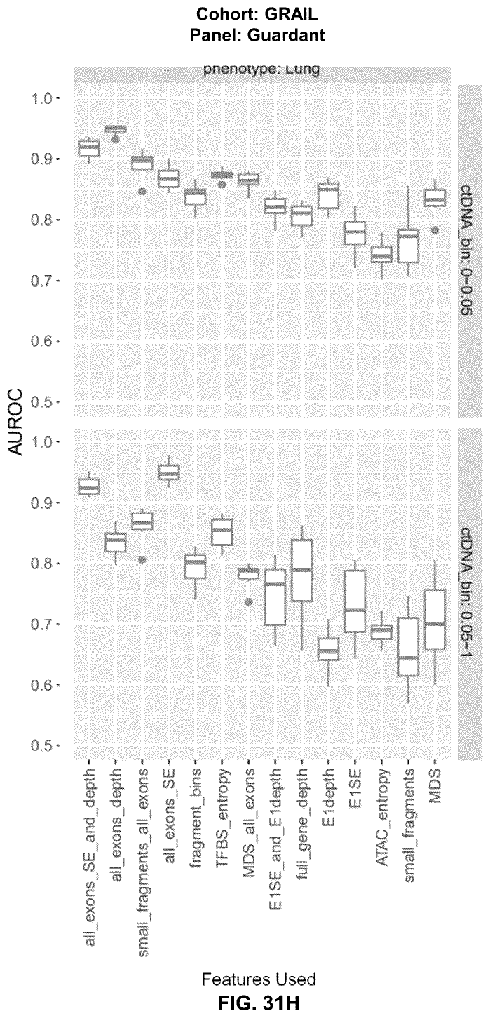

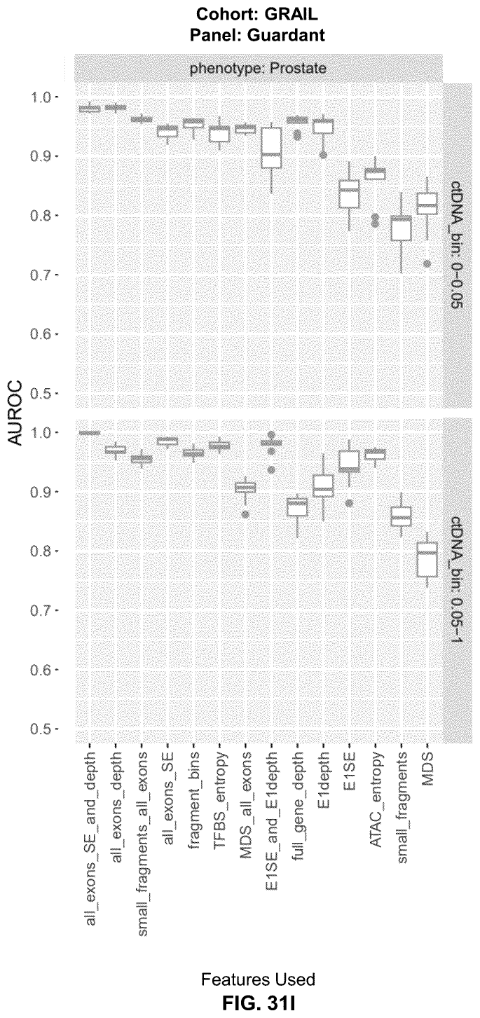

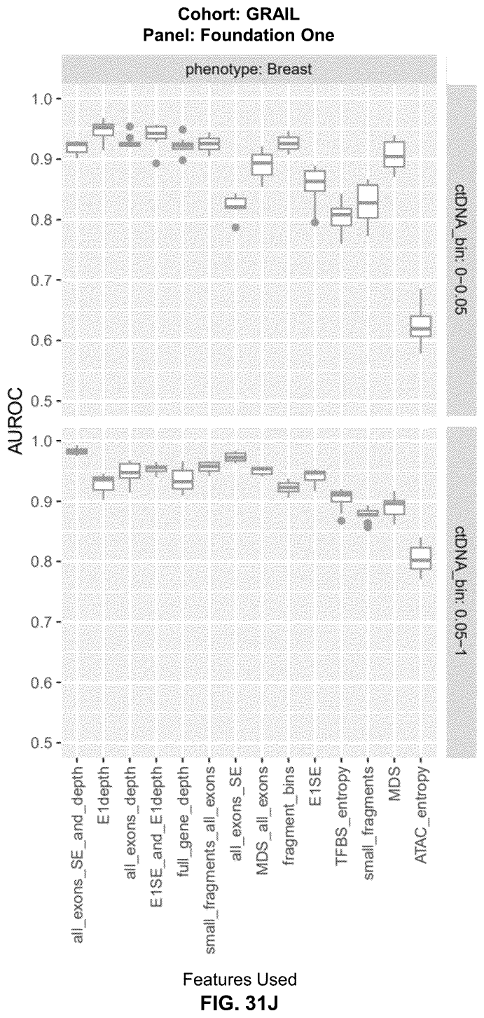

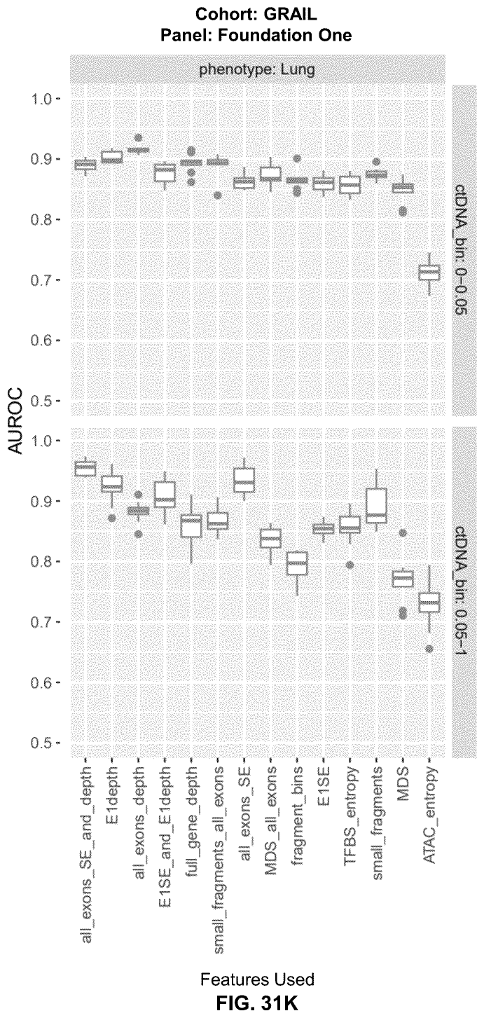

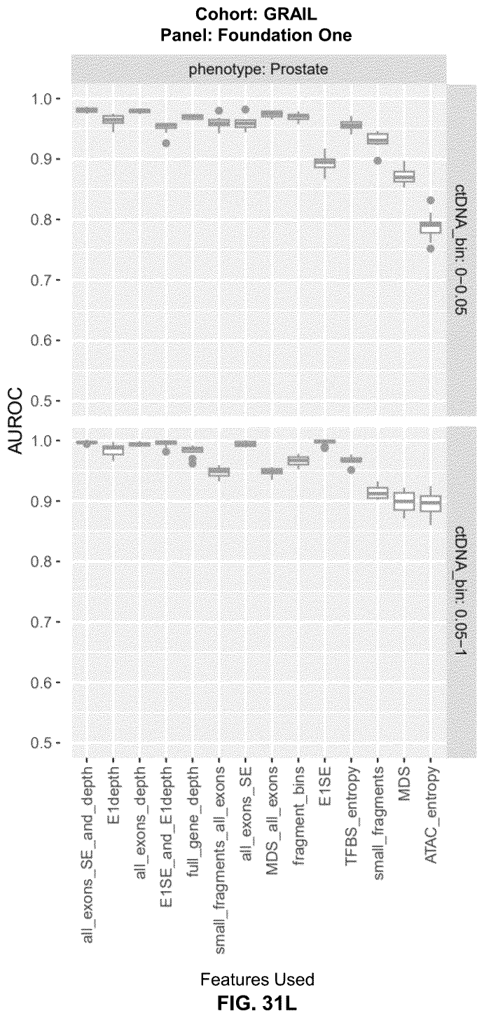

Using all metrics calculated (E1SE, exon 1 depth, E1SE and exon 1 depth, all exons Shannon entropy (SE), all exons depth, combining all exons depth and Shannon entropy, full gene depth, exon 1 MDS, all exon MDS, exon 1 small fragment proportions, all exons small fragment proportions, fragment size bins, TFBS entropy, and ATAC region entropy) samples in the UW cohort (FIGS. 30A-30L) and the GRAIL cohort (FIGS. 31A-31L) were analyzed for model performance to predict cancer type by ctDNA fraction bin. Samples were separated into “low” ctDNA fraction (0-0.05) and “high” ctDNA fraction (0.05-1). The best performing metric in the UW cohort with the UW panel was “all exons depth and SE” with mean AUROCs in the low ctDNA fraction ranging from 0.910-0.976 and mean AUROCs in the high ctDNA fraction ranging from 0.939-0.999. Across all feature types, the mean AUROC ranged from 0.494-1.000 (FIG. 30A). The best performing metric in the UW cohort with the Tempus XF panel was “all exons depth” with mean AUROCs in the low ctDNA fraction ranging from 0.853-0.974 and mean AUROCs in the high ctDNA fraction ranging from 0.978-0.999. Across all feature types, the mean AUROC ranged from 0.544-1.000 (FIG. 30B). The best performing metric in the UW cohort with the Guardant 360 CDx panel was “all exons depth” with mean AUROCs in the low ctDNA fraction ranging from 0.899-0.974 and mean AUROCs in the high ctDNA fraction ranging from 0.968-0.999. Across all feature types, the mean AUROC ranged from 0.507-0.999 (FIG. 30C). The best performing metric in the UW cohort with the Foundation One Liquid CDx panel was “all exons depth” with mean AUROCs in the low ctDNA fraction ranging from 0.873-0.978 and mean AUROCs in the high ctDNA fraction ranging from 0.959-1.000. Across all feature types, the mean AUROC ranged from 0.535-1.000 (FIG. 30D). The best performing metric in the GRAIL cohort with the GRAIL panel was “all exons depth” with mean AUROCs in the low ctDNA fraction ranging from 0.917-0.978 and mean AUROCs in the high ctDNA fraction ranging from 0.924-0.994. Across all feature types, the mean AUROC ranged from 0.751-0.998 (FIG. 31A). The best performing metric in the GRAIL cohort with the Tempus XF panel was “all exons SE and depth” with mean AUROCs in the low ctDNA fraction ranging from 0.892-0.977 and mean AUROCs in the high ctDNA fraction ranging from 0.945-0.999. Across all feature types, the mean AUROC ranged from 0.632-0.999 (FIG. 31B). The best performing metric in the GRAIL cohort with the Guardant 360 CDx panel was “all exons SE and depth” with mean AUROCs in the low ctDNA fraction ranging from 0.916-0.980 and mean AUROCs in the high ctDNA fraction ranging from 0.926-0.998. Across all feature types, the mean AUROC ranged from 0.640-0.998 (FIG. 31C). The best performing metric in the GRAIL cohort with the Foundation One Liquid CDx panel was “all exons SE and depth” with mean AUROCs in the low ctDNA fraction ranging from 0.890-0.980 and mean AUROCs in the high ctDNA fraction ranging from 0.954-0.997. Across all feature types, the mean AUROC ranged from 0.626-0.997 (FIG. 31D).

### Testing Model Performance to Predict Ctdna Fraciton Across Features

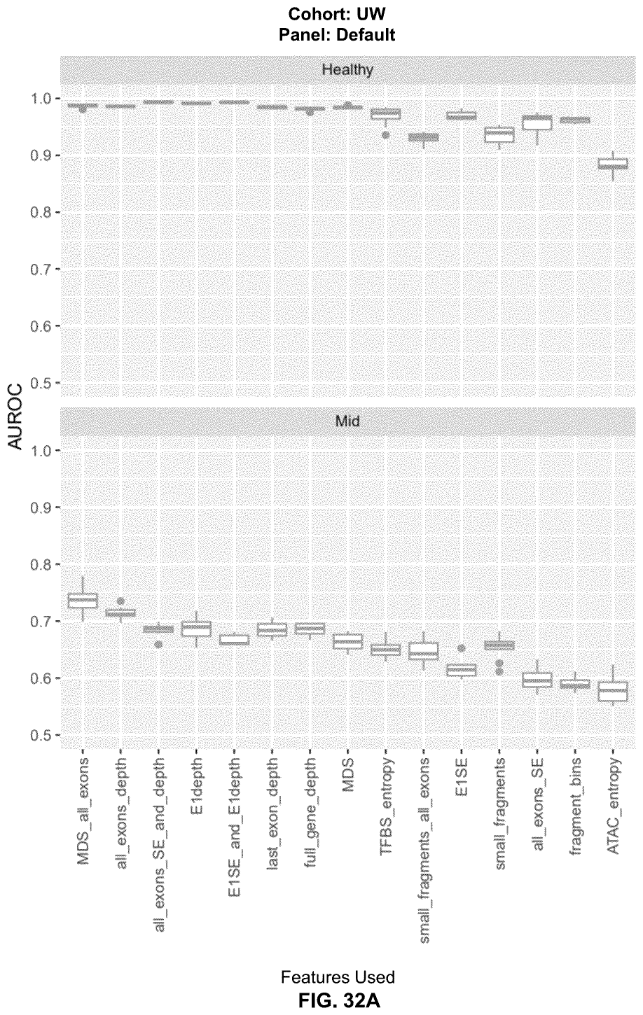

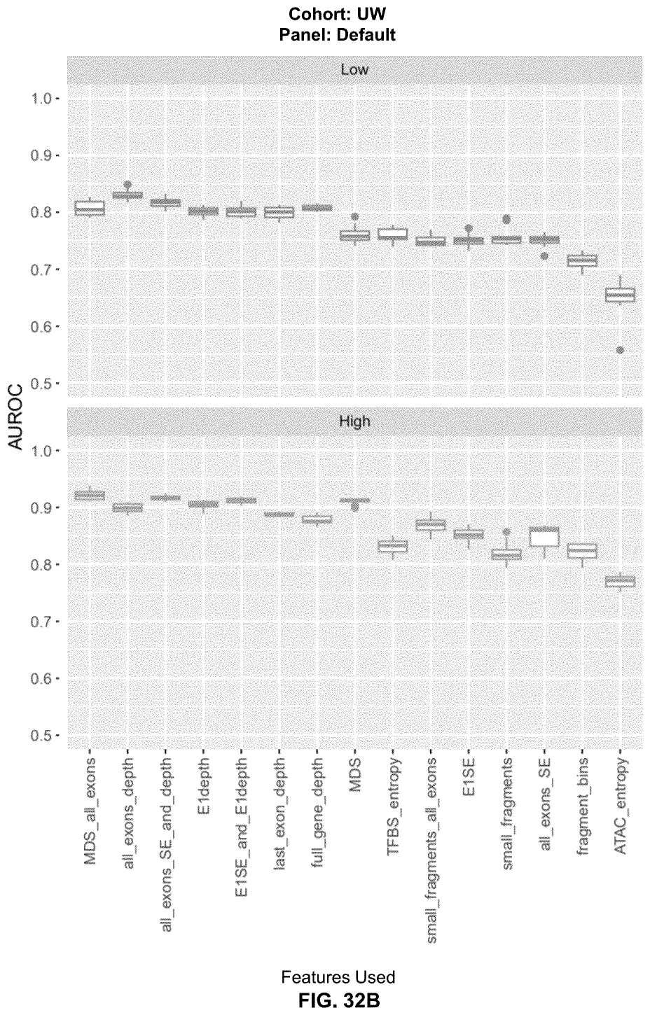

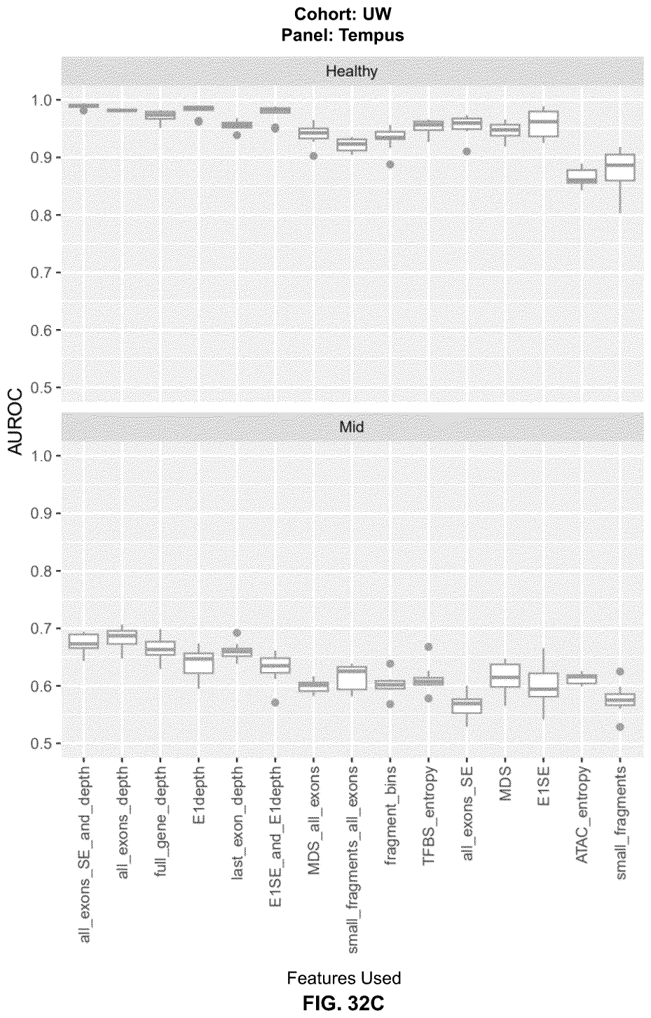

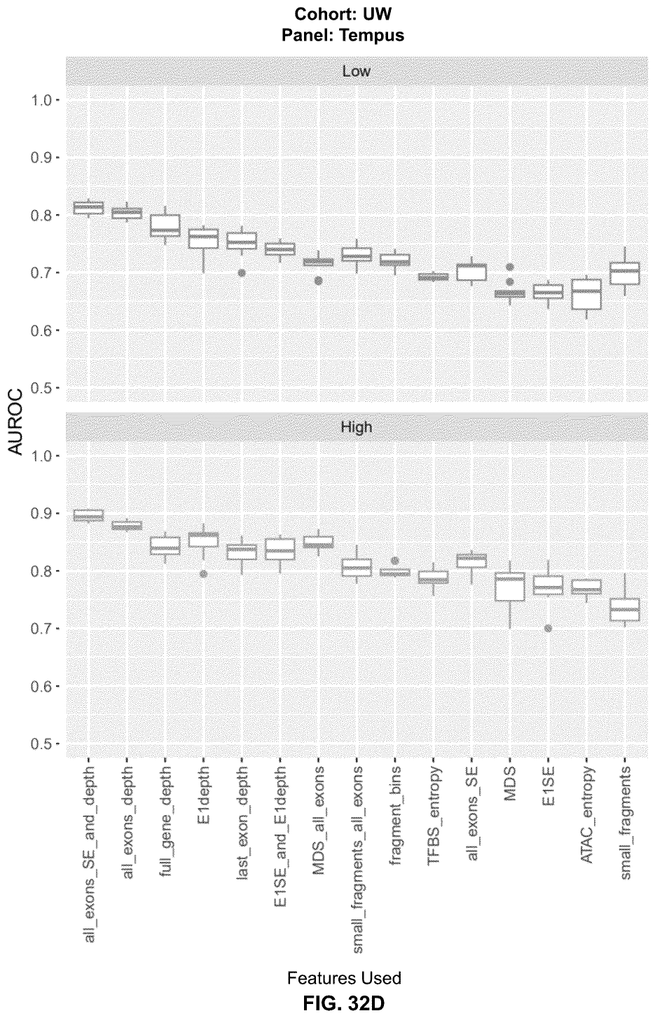

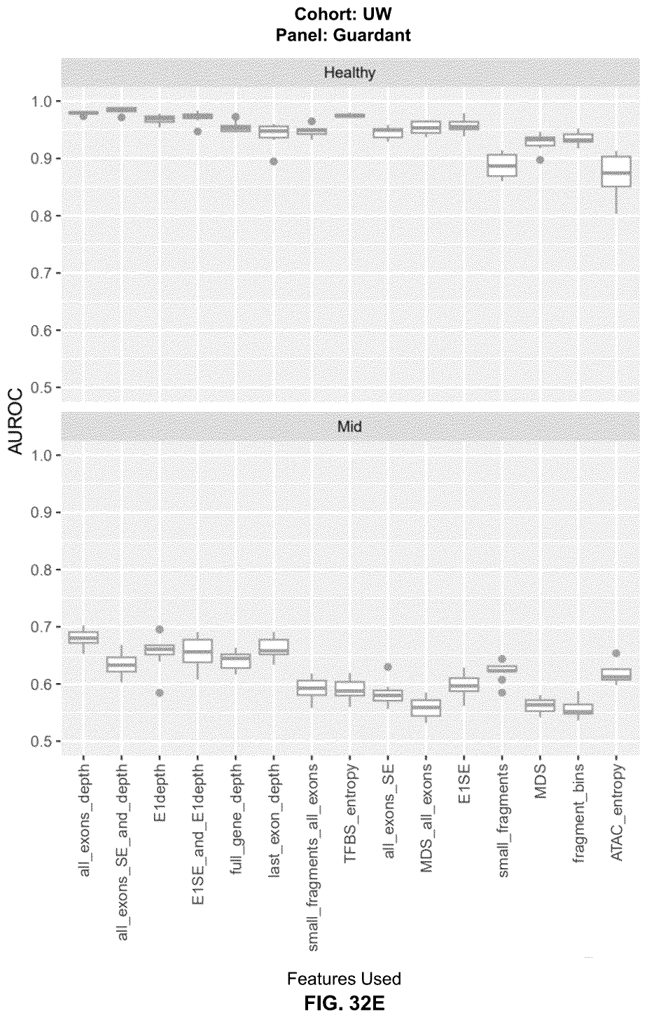

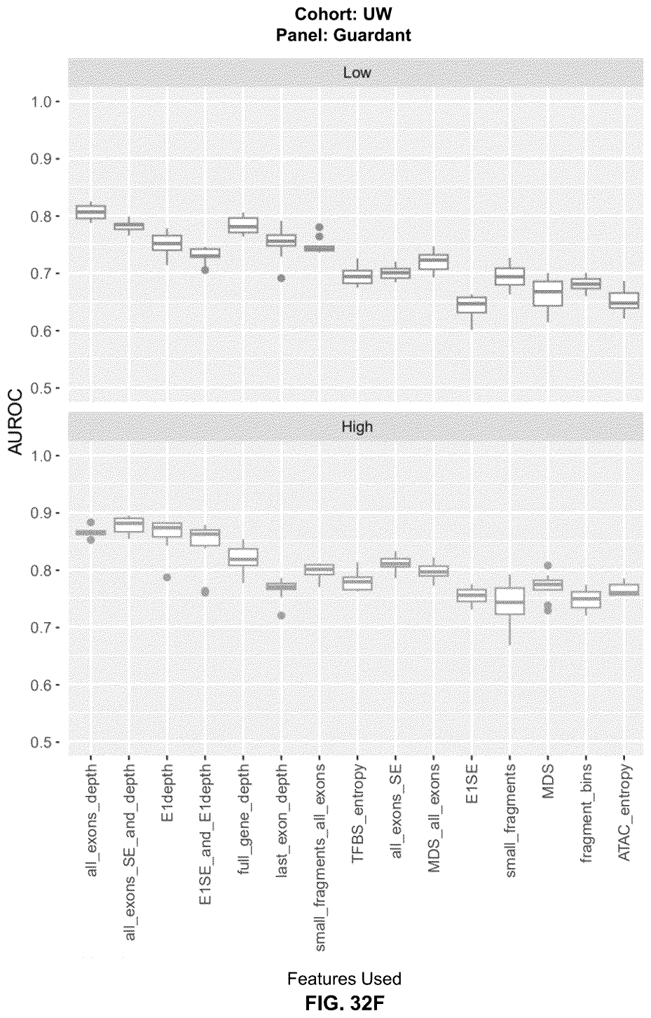

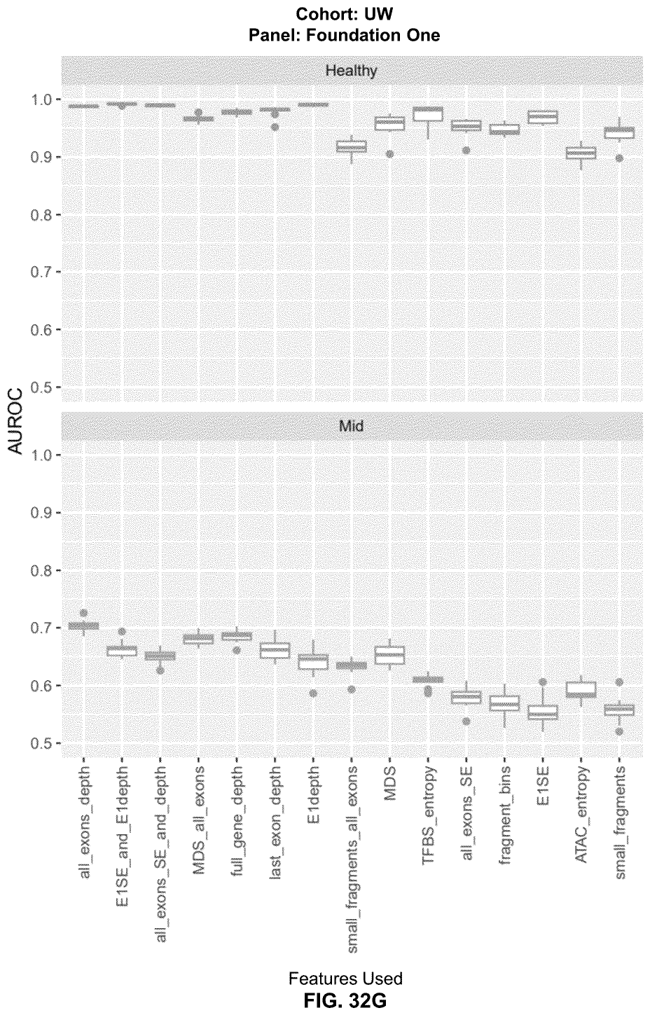

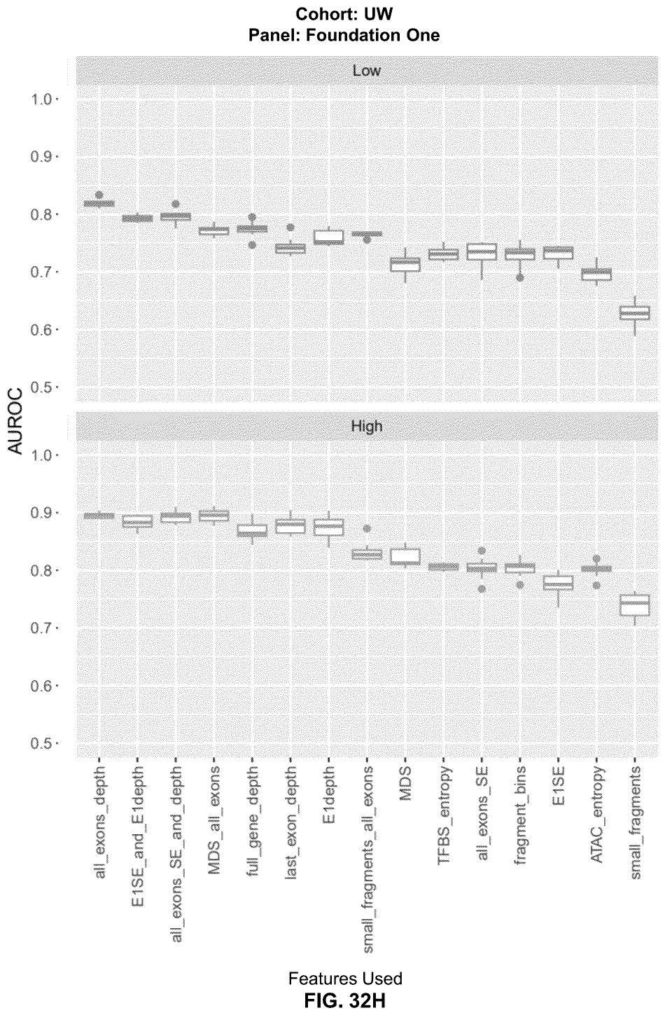

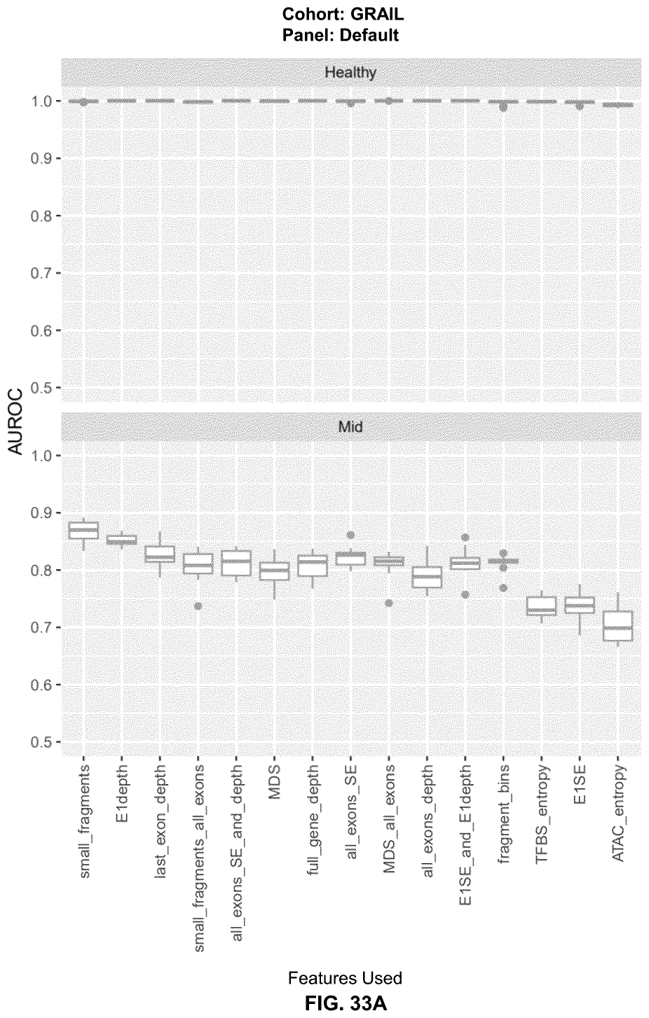

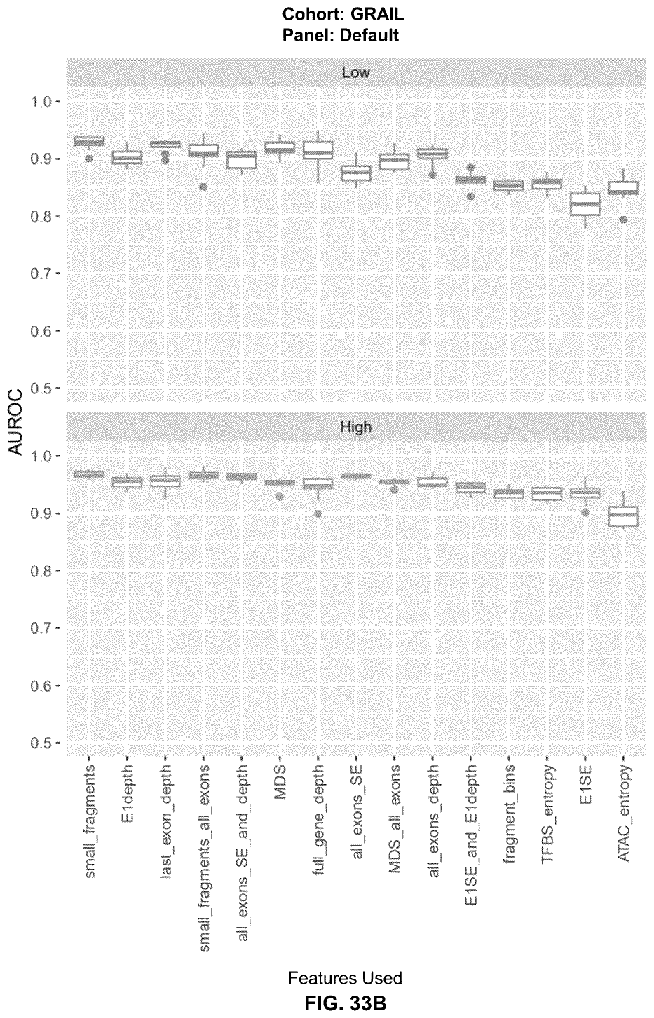

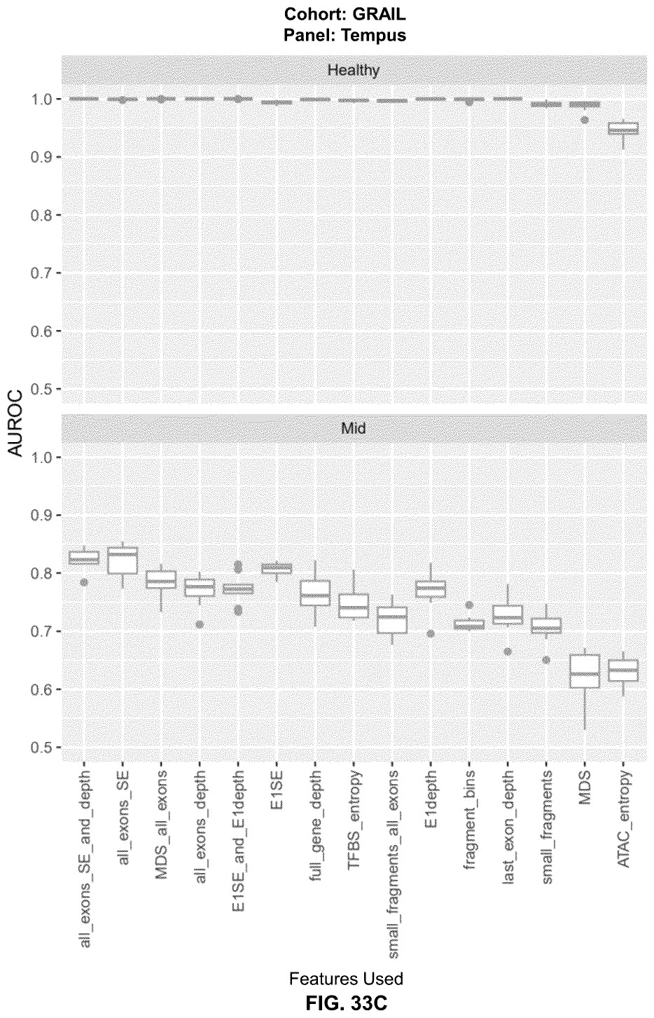

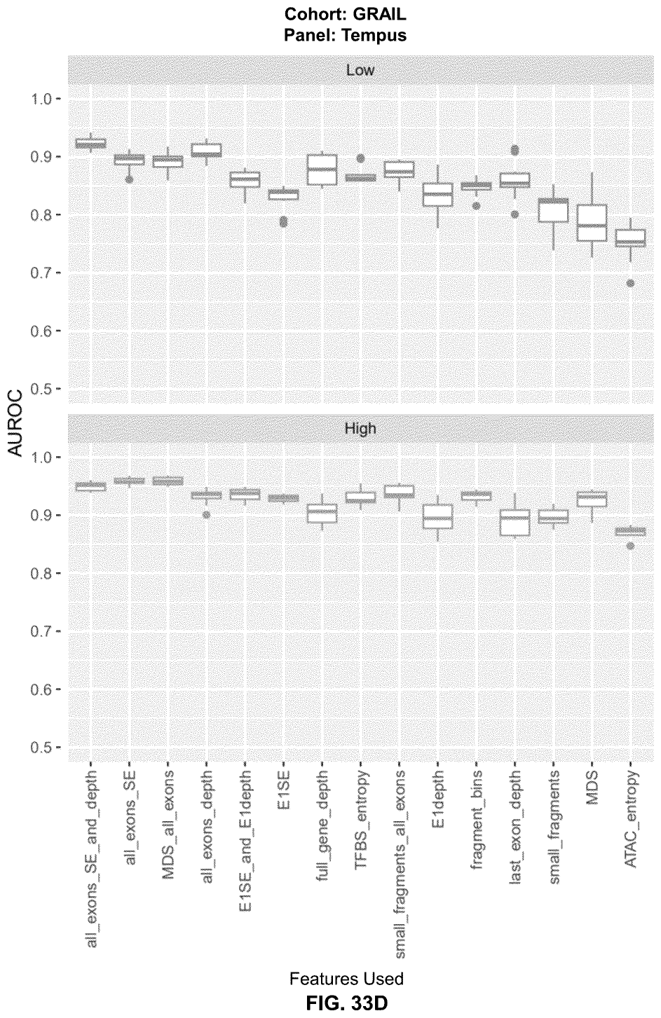

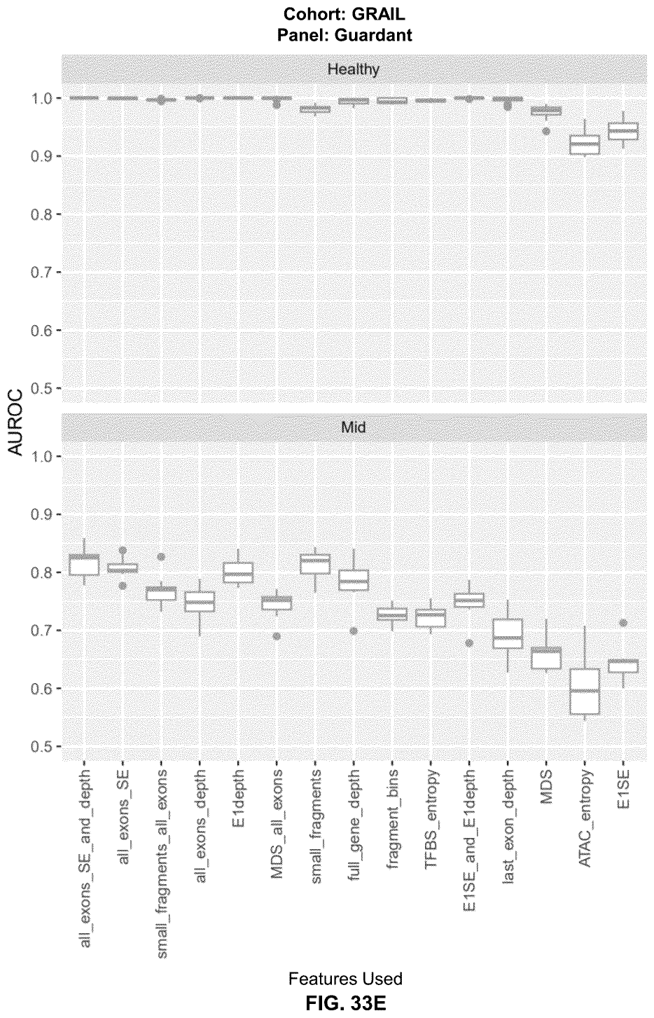

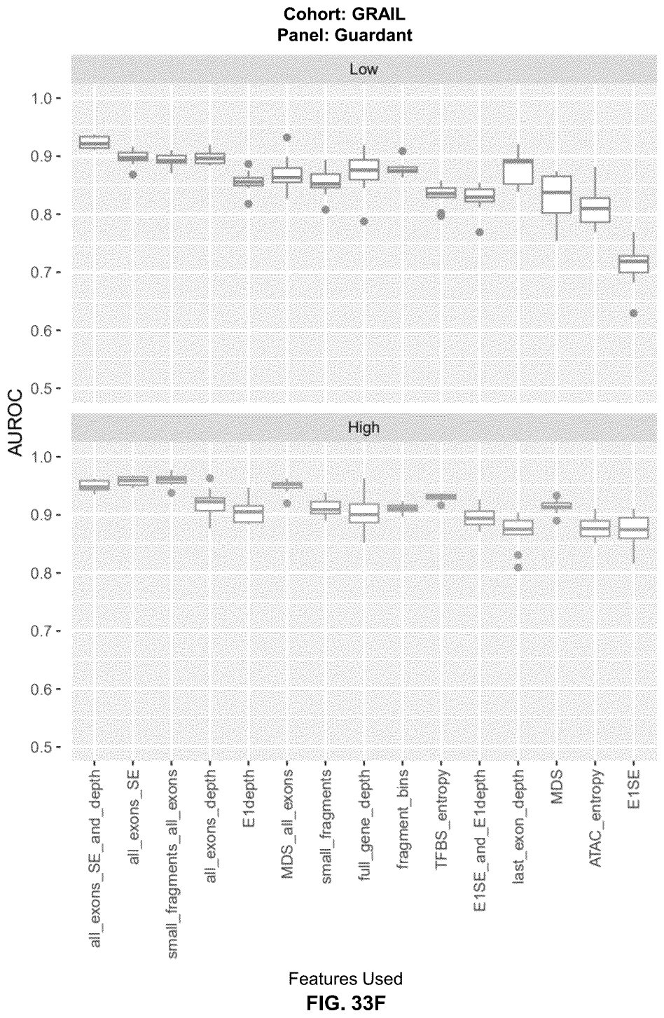

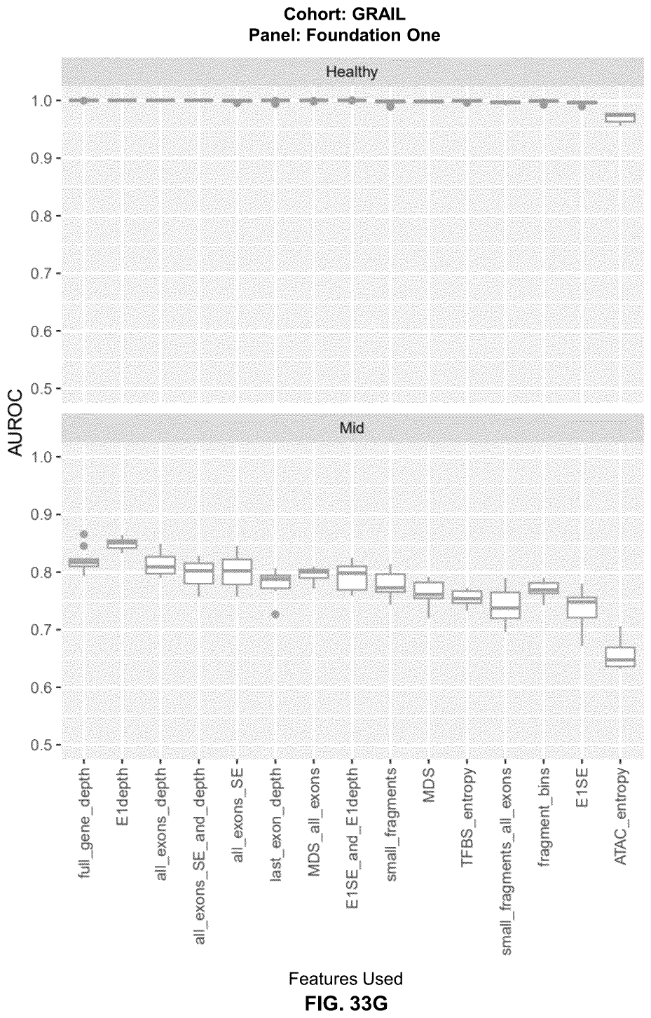

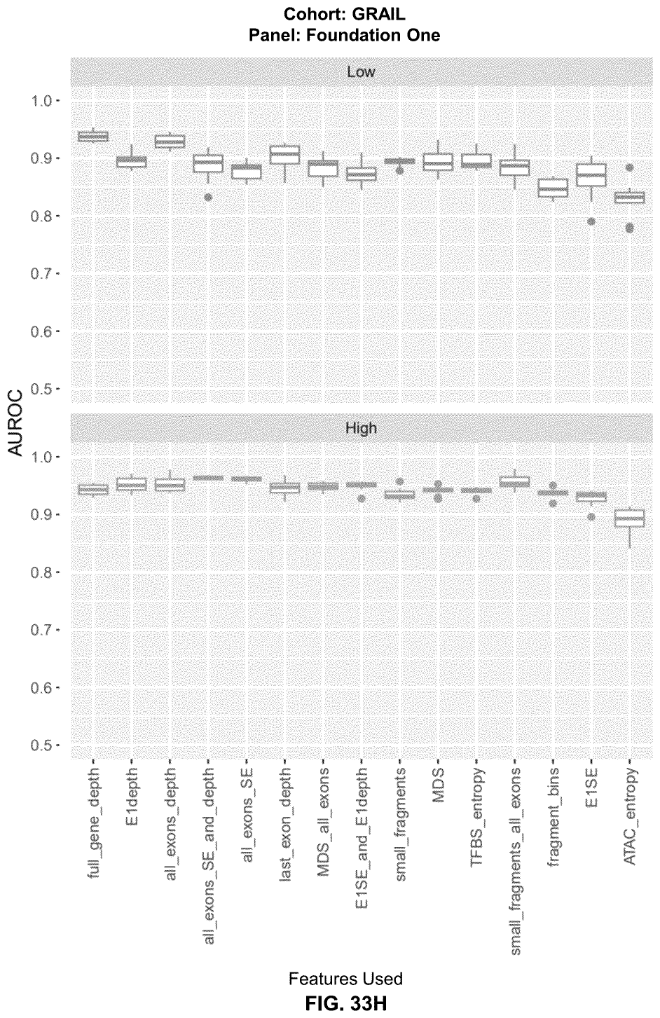

Using all metrics calculated (E1SE, exon 1 depth, E1SE and exon 1 depth, all exons Shannon entropy (SE), all exons depth, combining all exons depth and Shannon entropy, full gene depth, exon 1 MDS, all exon MDS, exon 1 small fragment proportions, all exons small fragment proportions, fragment size bins, TFBS entropy, and ATAC region entropy) samples in the UW cohort (FIGS. 32A-32H) and the GRAIL cohort (FIGS. 33A-33H) were analyzed for model performance to predict ctDNA fraction. Samples were binned into four groups of ctDNA fraction levels which were low (0-0.01 ctDNA fraction), mid (0.01-0.1 ctDNA fraction), high (0.1-1.0 ctDNA fraction) and healthy samples. The best performing metric in the UW cohort with the UW panel was “MDS all exons” with mean AUROCs for predicting ctDNA fraction ranging from 0.737-0.987. Across all feature types, the mean AUROC ranged from 0.580-0.993 (FIG. 32A). The best performing metric in the UW cohort with the Tempus XF panel was “all exons SE and depth” with mean AUROCs for predicting ctDNA fraction ranging from 0.673-0.989. Across all feature types, the mean AUROC ranged from 0.566-0.989 (FIG. 32B). The best performing metric in the UW cohort with the Guardant 360 CDx panel was “all exons depth” with mean AUROCs for predicting ctDNA fraction ranging from 0.680-0.979. Across all feature types, the mean AUROC ranged from 0.556-0.984 (FIG. 32C). The best performing metric in the UW cohort with the Foundation One Liquid CDx panel was “all exons depth” with mean AUROCs for predicting ctDNA fraction ranging from 0.702-0.987. Across all feature types, the mean AUROC ranged from 0.557-0.991 (FIG. 32D). The best performing metric in the GRAIL cohort with the GRAIL panel was “small fragment” with mean AUROCs for predicting ctDNA fraction ranging from 0.867-0.999. Across all feature types, the mean AUROC ranged from 0.705-1.000 (FIG. 33A). The best performing metric in the GRAIL cohort with the Tempus XF panel was “all exons SE and depth” with mean AUROCs for predicting ctDNA fraction ranging from 0.823-1.000. Across all feature types, the mean AUROC ranged from 0.624-1.000 (FIG. 33B). The best performing metric in the GRAIL cohort with the Guardant 360 CDx panel was “all exons SE and depth” with mean AUROCs for predicting ctDNA fraction ranging from 0.815-1.000. Across all feature types, the mean AUROC ranged from 0.604-1.000 (FIG. 33C). The best performing metric in the GRAIL cohort with the Foundation One Liquid CDx panel was “full gene depth” with mean AUROCs for predicting ctDNA fraction ranging from 0.820-0.999. Across all feature types, the mean AUROC ranged from 0.656-1.000 (FIG. 33D).

### Genes

### Tempus Gene List

Genes from the Tempus Xf sequencing panel were: AKT1, AKT2, ALK, APC, AR, ARAF, ARID1A, ATM, ATR, B2M, BAP1, BRAF, BRCA1, BRCA2, BTK, CCND1, CCND2, CCND3, CCNE1, CD274 (PD-L1), CDH1, CDK4, CDK6, CDKN2A, CTNNB1, DDR2, DPYD, EGFR, ERBB2 (HER2), ERRFI1, ESR1, EZH2, FBXW7, FGFR1, FGFR2, FGFR3, FGFR4, FLT3, FOXL2, GATA3, GNA11, GNAQ, GNAS, HNF1A, HRAS, IDH1, IDH2, JAK1, JAK2, JAK3, KDR, KEAP1, KIT, KMT2A, KRAS, MAP2K1, MAP2K2, MAPK1, MET, MLH1, MPL, MSH2, MSH3, MSH6, MTOR, MYC, MYCN, NF1, NF2, NFE2L2, NOTCH1, NPM1, NRAS, NTRK1, PALB2, PBRM1, PDCD1LG2, PDGFRA, PDGFRB, PIK3CA, PIK3R1, PMS2, PTCH1, PTEN, PTPN11, RAD51C, RAF1, RB1, RET, RHEB, RHOA, RIT1, RNF43, ROS1, SDHA, SMAD4, SMO, SPOP, STK11, TERT, TP53, TSC1, TSC2, UGT1A1, and VHL. Of the 105 genes in the Tempus Xf gene panel, 99 genes overlapped with the UW panel, and 98 genes overlapped with the GRAIL panel.

### Foundation Gene List

Genes from the Foundation One CDx sequencing panel were: ABL1, ACVR1B, AKT1, AKT2, AKT3, ALK, ALOX12B, AMERI (FAM123B), APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BRAF, BRCA1, BRCA2, BRD4, BRIP1, BTG1, BTG2, BTK, C11orf30 (EMSY), C17orf39 (GID4), CALR, CARD11, CASP8, CBFB, CBL, CCND1, CCND2, CCND3, CCNEI, CD22, CD274 (PD-L1), CD70, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSFIR, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EP300, EPHA3, EPHB1, EPHB4, ERBB2, ERBB3, ERBB4, ERCC4, ERG, ERRFI1, ESR1, EZH2, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1 (MEK1), MAP2K2 (MEK2), MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYC, MYCL (MYCL1), MYCN, MYD88, NBN, NF1, NF2, NFE2L2, NFKB1A, NKX2-1, NOTCH1, NOTCH2, NOTCH3, NPM1, NRAS, NSD3 (WHSCILI), NT5C2, NTRK1, NTRK2, NTRK3, P2RY8, PALB2, PARK2, PARP1, PARP2, PARP3, PAX5, PBRM1, PDCDI (PD-1), PDCD1LG2 (PD-L2), PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKC1, PTCH1, PTEN, PTPN11, PTPRO, QK1, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, SDHA, SDHB, SDHC, SDHD, SETD2, SF3B1, SGK1, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TET2, TGFBR2, TIPARP, TNFAIP3, TNFRSF14, TP53, TSC1, TSC2, TYRO3, U2AF1, VEGFA, VHL, WHSC1, WT1, XPO1, XRCC2, ZNF217, and ZNF703. Of the 309 genes in the Foundation One CDx gene panel, 228 genes overlapped with the UW panel, and 267 genes overlapped with the GRAIL panel.

### Guardant Gene List

Genes from the Foundation One CDx sequencing panel were: AKT1, ALK, APC, AR, ARAF, ATM, BRAF, BRCA1, BRCA2, CCND1, CDH1, CDK12, CDK4, CDK6, CDKN2A, CTNNB1, EGFR, ERBB2, ESR1, FGFR1, FGFR2, FGFR3, GATA3, GNA11, GNAQ, HNF1A, HRAS, IDH1, IDH2, KIT, KRAS, MAP2K1, MAP2K2, MET, MLH1, MTOR, MYC, NF1, NFE2L2, NRAS, NTRK1, NTRK3, PDGFRA, PIK3CA, PTEN, RAF1, RET, RHEB, ROS1, SMAD4, SMO, STK11, TERT, TSC1, and VHL. Of the 55 genes in the Guardant 360 CDx gene panel, 53 genes overlapped with the UW panel, and 54 genes overlapped with the GRAIL panel.

1. A method of detecting cancer or a particular type or subtype thereof in a subject and, optionally, treating the cancer or particular type or subtype thereof, the method comprising:
determining fragmentation patterns of classifier cell-free deoxyribonucleic acid (cfDNA) from the subject, wherein the classifier cfDNA comprises cfDNA from the subject corresponding to at least a portion of at least one exon of at least one classifier gene in a panel of one or more classifier genes; and
classifying the fragmentation patterns to identify the subject as being negative or positive for the cancer or the particular type or subtype thereof.

2. The method of claim 1, wherein the at least the portion of the at least one exon of the at least one classifier gene comprises a coding sequence of a first exon of the at least one classifier gene.

3. The method of claim 1, wherein the at least the portion of the at least one exon of the at least one classifier gene comprises one or more predefined exon regions selected from the group consisting of transcription factor binding sites, regions of open chromatin, and specific motifs.

4. The method of claim 1, wherein the classifier cfDNA excludes cfDNA from the subject corresponding to one or more exons of the at least one classifier gene other than the at least one exon.

5. The method of claim 1, wherein the classifier cfDNA corresponds to less than 2,500 Mb of a genome of the subject.

6. The method of claim 1, further comprising isolating from the subject a biological sample comprising the classifier cfDNA.

7. The method of claim 1, further comprising isolating the classifier cfDNA from at least some non-classifier cfDNA, wherein the non-classifier cfDNA is cfDNA that is not classifier cfDNA.

8. The method of claim 1, further comprising sequencing the classifier cfDNA.

9. The method of claim 8, wherein the sequencing comprises sequencing the classifier cfDNA at a deduplicated sequencing depth of at least 100×.

10. The method of claim 1, wherein the method excludes sequencing at least some non-classifier cfDNA from the subject.

11. The method of any one of claims 8-10, wherein the method sequences cfDNA corresponding to no more than 2,500 Mb of a genome of the subject.

12. The method of claim 1, wherein the determining the fragmentation patterns comprises determining a fragment size distribution of the classifier cfDNA.

13. The method of claim 1, wherein the determining the fragmentation patterns comprises determining a separate fragment size distribution of the classifier cfDNA corresponding to each classifier gene.

14. The method of claim 1, wherein each classifier gene comprises a coding region of an exon and the determining the fragmentation patterns comprises determining a separate fragment size distribution of the classifier cfDNA corresponding to the coding region of each exon.

15. The method of claim 1, wherein each classifier gene comprises a coding region of a first exon and the determining the fragmentation patterns comprises determining a separate fragment size distribution of the classifier cfDNA corresponding to the coding region of each first exon.

16. The method of claim 1, wherein each classifier gene comprises a coding region of multiple exons and the determining the fragmentation patterns comprises determining a separate fragment size distribution of the classifier cfDNA corresponding to the coding region of each of the multiple exons.

17. The method of claim 12, wherein the determining the fragmentation patterns comprises quantitating each fragment size distribution.

18. The method of claim 17, wherein the determining the fragmentation patterns comprises quantitating each fragment size distribution using size bins.

19. The method of claim 17, wherein the quantitating comprises quantitating an entropy value for each fragment size distribution.

20. The method of claim 17, wherein the quantitating comprises quantitating a number of reads (depth) for each fragment size distribution.

21. The method of claim 1, wherein the determining the fragmentation patterns comprises determining a motif diversity score.

22. The method of claim 1, wherein the determining the fragmentation patterns comprises determining the fragmentation patterns of one or more predefined exon regions selected from the group consisting of transcription factor binding sites, regions of open chromatin, and specific motifs.

23. The method of claim 1, wherein the classifier genes comprise cancer genes.

24. The method of claim 1, wherein the one or more classifier genes comprise at least 50 genes from Gene Set 1.

25. The method of claim 1, wherein the one or more classifier genes comprise at least 1 gene from Gene Set 2.

26. The method of claim 1, wherein the classifying identifies the subject as being negative or positive for at least one type of cancer.

27. The method of claim 26, wherein the at least one type of cancer comprises one or more tumor sites of origin.

28. The method of claim 27, wherein the one or more tumor sites of origin comprise one or more of breast, bladder, lung, kidney, and prostate.

29. The method of claim 1, wherein the method is capable of identifying the subject as being positive for cancer at an accuracy of at least 90% in a biological sample from the subject having a ct-fraction from 0.0001 to 0.001.

30. The method of claim 1, wherein the method is capable of identifying the subject as being positive for a cancer selected from the group consisting of breast cancer, bladder cancer, lung cancer, prostate cancer, and metastatic neuroendocrine prostate cancer at an accuracy of at least 70% in a biological sample from the subject having a ct-fraction from 0.001 to 0.01

31. The method of claim 1, further comprising identifying the subject as having a cancer of a particular tissue of origin and subjecting the subject to imaging or biopsy of the particular tissue of origin.

32. The method of claim 31, wherein the particular tissue of origin is a solid tissue and wherein the imaging or biopsy is of the solid tissue.

33. The method of claim 1, further comprising identifying the subject as having cancer and treating the cancer.

34. The method of claim 1, further comprising identifying the subject as having a cancer of a particular tissue of origin and subjecting the subject to surgery on the particular tissue of origin.

35. The method of claim 34, wherein the particular tissue of origin is a solid tissue and wherein the surgery is on the solid tissue.
